SEQUENCE LISTING (233 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 10 aa, Artificial Sequence
  Synthetic sequence CDR-H1 for clone 1H5
GFTFSDAAMY

SEQ ID NO: 3 - protein, 10 aa, Artificial Sequence
  Synthetic sequence CDR-H1 for clone 20H4
GFTFSNAAMY

SEQ ID NO: 4 - protein, 10 aa, Artificial Sequence
  Synthetic sequence CDR-H1 for clone 22D11
GFTFTNAAIY

SEQ ID NO: 5 - protein, 10 aa, Artificial Sequence
  Synthetic sequence CDR-H1 for clone 16F3
GFTFSNGAMN

SEQ ID NO: 6 - protein, 10 aa, Artificial Sequence
  Synthetic sequence CDR-H1 for clone 18B4
GFTFSSFAMA

SEQ ID NO: 7 - protein, 10 aa, Artificial Sequence
  Synthetic sequence CDR-H1 for clone 22E11
GFTFSTAAMY

SEQ ID NO: 8 - protein, 19 aa, Artificial Sequence
  Synthetic sequence CDR-H2 for clones 1H5, 16F3, and 22E11
RIRTKPNNYATYYADSVKG

SEQ ID NO: 9 - protein, 19 aa, Artificial Sequence
  Synthetic sequence CDR-H2 for clone 20H4
RIRTKPNNYATYHADSVKG

SEQ ID NO: 10 - protein, 17 aa, Artificial Sequence
  Synthetic sequence CDR-H2 for clone 18B4
TITSGGSKTYYPDSVKG

SEQ ID NO: 11 - protein, 9 aa, Artificial Sequence
  Synthetic sequence CDR-H3 for clone 1H5
SADFHYFDY

SEQ ID NO: 12 - protein, 9 aa, Artificial Sequence
  Synthetic sequence CDR-H3 for clone 20H4
TADMATGDY

SEQ ID NO: 13 - protein, 9 aa, Artificial Sequence
  Synthetic sequence CDR-H3 for clone 22D11
AADASRGDY

SEQ ID NO: 14 - protein, 10 aa, Artificial Sequence
  Synthetic sequence CDR-H3 for clone 16F3
TADGATPFAY

SEQ ID NO: 15 - protein, 7 aa, Artificial Sequence
  Synthetic sequence CDR-H3 for clone 18B4
AKGYFDY

SEQ ID NO: 16 - protein, 12 aa, Artificial Sequence
  Synthetic sequence CDR-H3 for clone 22E11
TADYYDGTPFAY

SEQ ID NO: 17 - protein, 118 aa, Artificial Sequence
  Synthetic sequence VH for clone 1H5
AVQLVESGGGLVQPKESLKISCAASGFTFSDAAMYWVRQAPGKGLELVARIRTKPNNYATYYADSVKGRFTISRDDSKSMVYLQMGNLKTEDTAMYYCSADFHYFDYWGQGVMVTVSS

SEQ ID NO: 18 - protein, 118 aa, Artificial Sequence
  Synthetic sequence VH for clone 20H4
AVQLVESGGGLVQPKESLKISCAASGFTFSNAAMYWVRQAPGKGLELLARIRTKPNNYATYHADSVKGRFTISRDDSQSMVYLHMGNLKTEDTAMYYCTADMATGDYWGPGVMVTVSS

SEQ ID NO: 19 - protein, 118 aa, Artificial Sequence
  Synthetic sequence VH for clone 22D11
AVQFVESGGGLVRPKESLKISCTASGFTFTNAAIYWVRQAPGKGLECIARIRTKPNNYATYYADSVRGRFTISRDDSKPMVYLQMNELKTEDTAMYYCAADASRGDYWGQGVMVTVSS

SEQ ID NO: 20 - protein, 119 aa, Artificial Sequence
  Synthetic sequence VH for clone 16F3
AVQLVESGGGLVQPKESLKISCAASGFTFSNGAMNWVRQAPGKGLEWVARIRTKPNNYATYYADSVKGRFTISRDDSKSMVYLQMDDLKTEDTAMYYCTADGATPFAYWGQGTLVTVSS

SEQ ID NO: 21 - protein, 114 aa, Artificial Sequence
  Synthetic sequence VH for clone 18B4
EVQLVESGGGLVQPGRSLKLSCAASGFTFSSFAMAWVRQAPKKGLEWVATITSGGSKTYYPDSVKGRFTISRDNAKSTLYLQMDSLRSEDTATYYCAKGYFDYWGQGVMVTVSS

SEQ ID NO: 22 - protein, 121 aa, Artificial Sequence
  Synthetic sequence VH for clone 22E11
AVQLVESGGGLVQPKESLKISCAASGFTFSTAAMYWVRQAPGKGLEWVTRIRTKPNNYATYYADSVKGRFTISRDDSKSMVYLQMDNLKTEDTAMYYCTADYYDGTPFAYWGQGTLVTVSS

SEQ ID NO: 23 - protein, 11 aa, Artificial Sequence
  Synthetic sequence CDR-L1 for clone 1H5
KAGQNINKNLD

SEQ ID NO: 24 - protein, 11 aa, Artificial Sequence
  Synthetic sequence - CDR-L1 for clones 20H4 , 22D11, 20H4_LC_k3_2_v1, and 20H4_LC_k3_2_v2
KASQNINKNLD

SEQ ID NO: 25 - protein, 16 aa, Artificial Sequence
  Synthetic sequence CDR-L1 for clones 16F3 and 18B4
RSSQSLLHSNGNTYLH

SEQ ID NO: 26 - protein, 16 aa, Artificial Sequence
  Synthetic sequence CDR-L1 for clone 22E11
RSSQSLVHNNGITYLS

SEQ ID NO: 27 - protein, 7 aa, Artificial Sequence
  Synthetic sequence CDR-L2 for clones 1H5 and 22D11
FTNNLQT

SEQ ID NO: 28 - protein, 7 aa, Artificial Sequence
  Synthetic sequence CDR-L2 for clone 20H4 and 20H4_LC_k3_2_v2
STNNLQT

SEQ ID NO: 29 - protein, 7 aa, Artificial Sequence
  Synthetic sequence CDR-L2 for clones 16F3 and 18B4
LVSRLES

SEQ ID NO: 30 - protein, 7 aa, Artificial Sequence
  Synthetic sequence CDR-L2 for clone 22E11
KVSNRFS

SEQ ID NO: 31 - protein, 8 aa, Artificial Sequence
  Synthetic sequence CDR-L3 for clone 1H5
YQYNSGWT

SEQ ID NO: 32 - protein, 8 aa, Artificial Sequence
  Synthetic sequence CDR-L3 for clone 20H4
YQYKSAWT

SEQ ID NO: 33 - protein, 8 aa, Artificial Sequence
  Synthetic sequence CDR-L3 for clone 22D11
YQYTSGFT

SEQ ID NO: 34 - protein, 9 aa, Artificial Sequence
  Synthetic sequence CDR-L3 for clone 16F3
VQGTHAPFT

SEQ ID NO: 35 - protein, 9 aa, Artificial Sequence
  Synthetic sequence CDR-L3 for clone 18B4
VQGTHAPLT

SEQ ID NO: 36 - protein, 9 aa, Artificial Sequence
  Synthetic sequence CDR-L3 for clone 22E11
GQGTQYPYT

SEQ ID NO: 37 - protein, 106 aa, Artificial Sequence
  Synthetic sequence VL for clone 1H5
DIQMTQSPSVLSASVGDRVTLSCKAGQNINKNLDWYQQKLGEAPKLLISFTNNLQTGVPSRFSGSGSGSDYTLTISSLQPEDVATYYCYQYNSGWTFGGGTKLELK

SEQ ID NO: 38 - protein, 106 aa, Artificial Sequence
  Synthetic sequence VL for clone 20H4
DIQMTQSPPVLSVSVGDRVTLSCKASQNINKNLDWYQQKHGEAPKPLIYSTNNLQTGVPSRFSGSGSGTDYTLTISSLQPEDVATYYCYQYKSAWTFGGGTKLELR

SEQ ID NO: 39 - protein, 106 aa, Artificial Sequence
  Synthetic sequence VL for clone 22D11
DIQMTQSPSVLSASVGDRVTFSCKASQNINKNLDWYQQKLGEVPKVLIYFTNNLQTGIPSRFSGSGSGTDYTLTISSLQPEDVATYYCYQYTSGFTFGAGTKLELK

SEQ ID NO: 40 - protein, 112 aa, Artificial Sequence
  Synthetic sequence VL for clone 16F3
DVVLTQTPPTLSATIGQSVSISCRSSQSLLHSNGNTYLHWLLQRPGQSPQLLIYLVSRLESGVPNRFSGSGSGTDFTLKISGVEAEDLGVYYCVQGTHAPFTFGSGTKLEIK

SEQ ID NO: 41 - protein, 112 aa, Artificial Sequence
  Synthetic sequence VLfor clone 18B4
DVVLTQTPPTLSATIGQSVSISCRSSQSLLHSNGNTYLHWLLQRPGQSPQLLIYLVSRLESGVPNRFSGSGSGTDFTLKISGVEAEDLGVYYCVQGTHAPLTFGSGTKLEIK

SEQ ID NO: 42 - protein, 112 aa, Artificial Sequence
  Synthetic sequence VL for clone 22E11
AVVMTQTPVSLSVSLGGQVSISCRSSQSLVHNNGITYLSWYLQKPGQSPQLPIYKVSNRFSGFSDRFSGGGSGTDFTLKISRVEPDDLGLYYCGQGTQYPYTFGAGTKLELK

SEQ ID NO: 43 - protein, 10 aa, Artificial Sequence
  Synthetic sequence CDR-H1 consensus sequence
  VARIANT 3,5,6,7,9,10: Xaa = any amino acid
GFXFXXXAXX

SEQ ID NO: 44 - protein, 19 aa, Artificial Sequence
  Synthetic sequence CDR-H2 consensus sequence
  VARIANT 13,18: Xaa = any amino acid
RIRTKPNNYATYXADSVXG

SEQ ID NO: 45 - protein, 9 aa, Artificial Sequence
  Synthetic sequence CDR-H2 consensus sequence
  VARIANT 3,4,8: Xaa = any amino acid
TYXXDSVXG

SEQ ID NO: 46 - protein, 9 aa, Artificial Sequence
  Synthetic sequence CDR-H3 consensus sequence
  VARIANT 1,4,5,6,7: Xaa = any amino acid
XADXXXXDY

SEQ ID NO: 47 - protein, 11 aa, Artificial Sequence
  Synthetic sequence CDR-L1 consensus sequence
  VARIANT 3,5,8,9: Xaa = any amino acid
KAXQXINXXLD

SEQ ID NO: 48 - protein, 16 aa, Artificial Sequence
  Synthetic sequence CDR-L1 consensus sequence
  VARIANT 7,9,12,16: Xaa = any amino acid
RSSQSLXHXNGXTYLX

SEQ ID NO: 49 - protein, 7 aa, Artificial Sequence
  Synthetic sequence CDR-L2 consensus sequence
  VARIANT 1,3,4,6: Xaa = any amino acid
XTXXLXT

SEQ ID NO: 50 - protein, 7 aa, Artificial Sequence
  Synthetic sequence CDR-L2 consensus sequence
  VARIANT 1,4,5,6: Xaa = any amino acid
XVSXXXS

SEQ ID NO: 51 - protein, 8 aa, Artificial Sequence
  Synthetic sequence CDR-L3 consensus sequence
  VARIANT (3)...(7): Xaa = any amino acid
YQXXXXXT

SEQ ID NO: 52 - protein, 9 aa, Artificial Sequence
  Synthetic sequence CDR-L3 consensus sequence
  VARIANT 1,5,6,8: Xaa = any amino acid
XQGTXXPXT

SEQ ID NO: 53 - DNA, 21 bp, Artificial Sequence
  ratIgG1 VH 3′ PCR primer
aggcttgcaatcacctccaca

SEQ ID NO: 54 - DNA, 21 bp, Artificial Sequence
  ratIgG2a VH 3′ PCR primer
acaaggattgcattcccttgg

SEQ ID NO: 55 - DNA, 21 bp, Artificial Sequence
  ratIgG2b VH 3′ PCR primer
gcatttgtgtccaatgccgcc

SEQ ID NO: 56 - DNA, 21 bp, Artificial Sequence
  ratIgG2c VH 3′ PCR primer
tctgggcttgggtcttctggg

SEQ ID NO: 57 - DNA, 30 bp, Artificial Sequence
  ratCkappa VL 3′ primer
ctcattcctgttgaagctcttgacgacggg

SEQ ID NO: 58 - protein, 46 aa, Homo sapiens
VKKDQLGKNEEGAPQEGILEDMPVDPDNEAYEMPSEEGYQDYEPEA

SEQ ID NO: 59 - protein, 29 aa, Homo sapiens
ILEDMPVDPDNEAYEMPSEEGYQDYEPEA

SEQ ID NO: 60 - protein, 20 aa, Homo sapiens
GILEDMPVDPDNEAYEMPSE

SEQ ID NO: 61 - protein, 15 aa, Homo sapiens
GILEDMPVDPDNEAY

SEQ ID NO: 62 - protein, 15 aa, Homo sapiens
MPVDPDNEAYEMPSE

SEQ ID NO: 63 - protein, 15 aa, Homo sapiens
EPKSCDKTHTCPPCP

SEQ ID NO: 64 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 213 aa, Artificial Sequence
  Synthetic sequence Chimeric 20H4 (Chi 20H4) light chain
DIQMTQSPPVLSVSVGDRVTLSCKASQNINKNLDWYQQKHGEAPKPLIYSTNNLQTGVPSRFSGSGSGTDYTLTISSLQPEDVATYYCYQYKSAWTFGGGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 448 aa, Artificial Sequence
  Synthetic sequence Chimeric 20H4 (Chi 20H4) heavy chain sequence (wild-type human Fc)
AVQLVESGGGLVQPKESLKISCAASGFTFSNAAMYWVRQAPGKGLELLARIRTKPNNYATYHADSVKGRFTISRDDSQSMVYLHMGNLKTEDTAMYYCTADMATGDYWGPGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 15 aa, Homo sapiens
LEDMPVDPDNEAYEM

SEQ ID NO: 68 - protein, 15 aa, Artificial Sequence
  Synthetic sequence Peptide containing alanine substitution at position 113 of SEQ ID NO:1
AEDMPVDPDNEAYEM

SEQ ID NO: 69 - protein, 15 aa, Artificial Sequence
  Synthetic sequence Peptide containing alanine substitution at position 114 of SEQ ID NO:1
LADMPVDPDNEAYEM

SEQ ID NO: 70 - protein, 15 aa, Artificial Sequence
  Synthetic sequence Peptide containing alanine substitution at position 115 of SEQ ID NO:1
LEAMPVDPDNEAYEM

SEQ ID NO: 71 - protein, 15 aa, Artificial Sequence
  Synthetic sequence - Peptide containing alanine substitution at position 116 of SEQ ID NO:1
LEDAPVDPDNEAYEM

SEQ ID NO: 72 - protein, 15 aa, Artificial Sequence
  Synthetic sequence - Peptide containing alanine substitution at position 117 of SEQ ID NO:1
LEDMAVDPDNEAYEM

SEQ ID NO: 73 - protein, 15 aa, Artificial Sequence
  Synthetic sequence - Peptide containing alanine substitution at position 118 of SEQ ID NO:1
LEDMPADPDNEAYEM

SEQ ID NO: 74 - protein, 15 aa, Artificial Sequence
  Synthetic sequence - Peptide containing alanine substitution at position 119 of SEQ ID NO:1
LEDMPVAPDNEAYEM

SEQ ID NO: 75 - protein, 15 aa, Artificial Sequence
  Synthetic sequence - Peptide containing alanine substitution at position 120 of SEQ ID NO:1
LEDMPVDADNEAYEM

SEQ ID NO: 76 - protein, 15 aa, Artificial Sequence
  Synthetic sequence - Peptide containing alanine substitution at position 121 of SEQ ID NO:1
LEDMPVDPANEAYEM

SEQ ID NO: 77 - protein, 15 aa, Artificial Sequence
  Synthetic sequence - Peptide containing alanine substitution at position 122 of SEQ ID NO:1
LEDMPVDPDAEAYEM

SEQ ID NO: 78 - protein, 15 aa, Artificial Sequence
  Synthetic sequence - Peptide containing alanine substitution at position 123 of SEQ ID NO:1
LEDMPVDPDNAAYEM

SEQ ID NO: 79 - protein, 15 aa, Artificial Sequence
  Synthetic sequence - Peptide containing glycine substitution at position 124 of SEQ ID NO:1
LEDMPVDPDNEGYEM

SEQ ID NO: 80 - protein, 15 aa, Artificial Sequence
  Synthetic sequence - Peptide containing proline substitution at position 124 of SEQ ID NO:1
LEDMPVDPDNEPYEM

SEQ ID NO: 81 - protein, 15 aa, Artificial Sequence
  Synthetic sequence - Peptide containing alanine substitution at position 125 of SEQ ID NO:1
LEDMPVDPDNEAAEM

SEQ ID NO: 82 - protein, 15 aa, Artificial Sequence
  Synthetic sequence - Peptide containing alanine substitution at position 126 of SEQ ID NO:1
LEDMPVDPDNEAYAM

SEQ ID NO: 83 - protein, 15 aa, Artificial Sequence
  Synthetic sequence - Peptide containing alanine substitution at position 127 of SEQ ID NO:1
LEDMPVDPDNEAYEA

SEQ ID NO: 84-85 - skipped (2 entries)

SEQ ID NO: 86 - protein, 7 aa, Artificial Sequence
  Synthetic sequence - Epitope sequence (amino acids 119-125 within SEQ ID NO:1)
DPDNEAY

SEQ ID NO: 87 - protein, 10 aa, Artificial Sequence
  Synthetic sequence - CDR-H1 consensus sequence
  VARIANT 3: Xaa = T, R, K, or N
  VARIANT 5: Xaa = N, S, or T
  VARIANT 6: Xaa = N, K, R, D, S, or T
  VARIANT 7: Xaa = A, G, or F
  VARIANT 9: Xaa = I or M
  VARIANT 10: Xaa = Y, N, or A
GFXFXXXAXX

SEQ ID NO: 88 - protein, 19 aa, Artificial Sequence
  Synthetic sequence - CDR-H2 consensus sequence
  VARIANT 13: Xaa = Y or H
  VARIANT 18: Xaa = K or R
RIRTKPNNYATYXADSVXG

SEQ ID NO: 89 - protein, 9 aa, Artificial Sequence
  Synthetic sequence - CDR-H2 consensus sequence
  VARIANT 3: Xaa = Y or H
  VARIANT 4: Xaa = A or P
  VARIANT 8: Xaa = K or R
TYXXDSVXG

SEQ ID NO: 90 - protein, 9 aa, Artificial Sequence
  Synthetic sequence - CDR-H3 consensus sequence
  VARIANT 1: Xaa = T or S
  VARIANT 4: Xaa = F, M, or A
  VARIANT 5: Xaa = H, A, or S
  VARIANT 6: Xaa = Y, T, or R
  VARIANT 7: Xaa = F or G
XADXXXXDY

SEQ ID NO: 91 - protein, 11 aa, Artificial Sequence
  Synthetic sequence - CDR-L1 consensus sequence
  VARIANT 3: Xaa = G or S
  VARIANT 5: Xaa = N or I
  VARIANT 8: Xaa = K, N, Q, D, or A
  VARIANT 9: Xaa = N or I
KAXQXINXXLD

SEQ ID NO: 92 - protein, 16 aa, Artificial Sequence
  Synthetic sequence - CDR-L1 consensus sequence
  VARIANT 7: Xaa = L or V
  VARIANT 9: Xaa = S or N
  VARIANT 12: Xaa = N or I
  VARIANT 16: Xaa = H or S
RSSQSLXHXNGXTYLX

SEQ ID NO: 93 - protein, 7 aa, Artificial Sequence
  Synthetic sequence - CDR-L2 consensus sequence
  VARIANT 1: Xaa = F, R, or S
  VARIANT 3: Xaa = N or K
  VARIANT 4: Xaa = N or K
  VARIANT 6: Xaa = Q or K
XTXXLXT

SEQ ID NO: 94 - protein, 7 aa, Artificial Sequence
  Synthetic sequence - CDR-L2 consensus sequence
  VARIANT 1: Xaa =  L or K
  VARIANT 4: Xaa = R or N
  VARIANT 5: Xaa = L or R
  VARIANT 6: Xaa = E or F
XVSXXXS

SEQ ID NO: 95 - protein, 8 aa, Artificial Sequence
  Synthetic sequence - CDR-L3 consensus sequence
  VARIANT 3: Xaa = Y, D, or F
  VARIANT 4: Xaa = T, N, or K
  VARIANT 5: Xaa = S, N, R, K, Y, L, or F
  VARIANT 6: Xaa = A or G
  VARIANT 7: Xaa = W or F
YQXXXXXT

SEQ ID NO: 96 - protein, 9 aa, Artificial Sequence
  Synthetic sequence - CDR-L3 consensus sequence
  VARIANT 1: Xaa = V or G
  VARIANT 5: Xaa = H or Q
  VARIANT 6: Xaa = A or Y
  VARIANT 8: Xaa = F, L, or Y
XQGTXXPXT

SEQ ID NO: 97 - protein, 10 aa, Artificial Sequence
  Synthetic sequence - 20H4_H1_1 CDR-H1 sequence
GFTFSKAAMY

SEQ ID NO: 98 - protein, 10 aa, Artificial Sequence
  Synthetic sequence - 20H4_H1_2 CDR-H1 sequence
GFRFSKAAMY

SEQ ID NO: 99 - protein, 10 aa, Artificial Sequence
  Synthetic sequence - 20H4_H1_3 CDR-H1 sequence
GFTFSRAAMY

SEQ ID NO: 100 - protein, 10 aa, Artificial Sequence
  Synthetic sequence - 20H4_H1_4 CDR-H1 sequence
GFTFNKAAMY

SEQ ID NO: 101 - protein, 10 aa, Artificial Sequence
  Synthetic sequence - 20H4_H1_5 CDR-H1 sequence
GFKFSKAAMY

SEQ ID NO: 102 - protein, 10 aa, Artificial Sequence
  Synthetic sequence - 20H4_H1_6 CDR-H1 sequence
GFNFSKAAMY

SEQ ID NO: 103 - protein, 10 aa, Artificial Sequence
  Synthetic sequence - 20H4 CDR-H1 consensus sequence
  VARIANT 3,5,6: Xaa = any amino acid
GFXFXXAAMY

SEQ ID NO: 104 - protein, 10 aa, Artificial Sequence
  Synthetic sequence - 20H4 CDR-H1 consensus sequence
  VARIANT 3: Xaa = T, R, K, or N
  VARIANT 5: Xaa = S or N
  VARIANT 6: Xaa = N, K, or R
GFXFXXAAMY

SEQ ID NO: 105 - protein, 19 aa, Artificial Sequence
  Synthetic sequence - 20H4_H2_1 CDR-H2 sequence
RIRTKPNNFATYHADSVKG

SEQ ID NO: 106 - protein, 19 aa, Artificial Sequence
  Synthetic sequence - 20H4_H2_2 CDR-H2 sequence
RIRTQRNNYATYHADSVKG

SEQ ID NO: 107 - protein, 19 aa, Artificial Sequence
  Synthetic sequence - 20H4_H2_3 CDR-H2 sequence
RIRTERNNYATYHADSVKG

SEQ ID NO: 108 - protein, 19 aa, Artificial Sequence
  Synthetic sequence - 20H4_H2_4 CDR-H2 sequence
RIRTKRNNYATYHADSVKG

SEQ ID NO: 109 - protein, 19 aa, Artificial Sequence
  Synthetic sequence - 20H4_H2_5 CDR-H2 sequence
RIRTEPNNYATYHADSVKG

SEQ ID NO: 110 - protein, 19 aa, Artificial Sequence
  Synthetic sequence - 20H4_H2_6 CDR-H2 sequence
RIRTKPKNYATYHADSVKG

SEQ ID NO: 111 - protein, 19 aa, Artificial Sequence
  Synthetic sequence - 20H4 CDR-H2 consensus sequence
  VARIANT 5,6,7,9: Xaa = any amino acid
RIRTXXXNXATYHADSVKG

SEQ ID NO: 112 - protein, 19 aa, Artificial Sequence
  Synthetic sequence - 20H4 CDR-H2 consensus sequence
  VARIANT 5: Xaa =K, Q, or E
  VARIANT 6: Xaa = P or R
  VARIANT 7: Xaa = N or K
  VARIANT 9: Xaa = Y or F
RIRTXXXNXATYHADSVKG

SEQ ID NO: 113 - protein, 9 aa, Artificial Sequence
  Synthetic sequence - 20H4_H3_1 CDR-H3 sequence
TANMATGDY

SEQ ID NO: 114 - protein, 9 aa, Artificial Sequence
  Synthetic sequence - 20H4_H3_2 CDR-H3 sequence
TANMAKGDY

SEQ ID NO: 115 - protein, 9 aa, Artificial Sequence
  Synthetic sequence - 20H4_H3_3 CDR-H3 sequence
AAAMARGDY

SEQ ID NO: 116 - protein, 9 aa, Artificial Sequence
  Synthetic sequence - 20H4_H3_4 CDR-H3 sequence
SADFAKGDY

SEQ ID NO: 117 - protein, 9 aa, Artificial Sequence
  Synthetic sequence - 20H4_H3_5 CDR-H3 sequence
TADFARGDY

SEQ ID NO: 118 - protein, 9 aa, Artificial Sequence
  Synthetic sequence - 20H4_H3_6 CDR-H3 sequence
TANMRKGDY

SEQ ID NO: 119 - protein, 9 aa, Artificial Sequence
  Synthetic sequence - 20H4_H3_7 CDR-H3 sequence
TADMAKGDY

SEQ ID NO: 120 - protein, 9 aa, Artificial Sequence
  Synthetic sequence - 20H4 CDR-H3 consensus sequence
  VARIANT 1,3,4,5,6: Xaa = any amino acid
XAXXXXGDY

SEQ ID NO: 121 - protein, 9 aa, Artificial Sequence
  Synthetic sequence - 20H4 CDR-H3 consensus sequence
  VARIANT 1: Xaa = T, A, or S
  VARIANT 3: Xaa = D, N, or A
  VARIANT 4: Xaa = M or F
  VARIANT 5: Xaa = A or R
  VARIANT 6: Xaa = T, K, or R
XAXXXXGDY

SEQ ID NO: 122 - protein, 11 aa, Artificial Sequence
  Synthetic sequence - 20H4_L1_1 CDR-L1 and 20H4_LC_k3_1 CDR-L1 sequence
KASQNINNNLD

SEQ ID NO: 123 - protein, 11 aa, Artificial Sequence
  Synthetic sequence - 20H4_L1_2 CDR-L1 sequence
KASQIINQILD

SEQ ID NO: 124 - protein, 11 aa, Artificial Sequence
  Synthetic sequence - 20H4 CDR-L1 consensus sequence
  VARIANT 5, 8, 9: Xaa = any amino acid
KASQXINXXLD

SEQ ID NO: 125 - protein, 11 aa, Artificial Sequence
  Synthetic sequence - 20H4 CDR-L1 consensus sequence
  VARIANT 5: Xaa = N or I
  VARIANT 8: Xaa = K, N, Q, D, or A
  VARIANT 9: Xaa = N or I
KASQXINXXLD

SEQ ID NO: 126 - protein, 7 aa, Artificial Sequence
  Synthetic sequence - 20H4_L2_1 CDR-L2 sequence
STNKLKT

SEQ ID NO: 127 - protein, 7 aa, Artificial Sequence
  Synthetic sequence - 20H4_L2_2 CDR-L2 sequence
RTKNLQT

SEQ ID NO: 128 - protein, 7 aa, Artificial Sequence
  Synthetic sequence - CDR-L2 sequence 20H4_L1_1, 20H4_LC_k3_1, and 20H4_LC_k3_2_v1
STNNLKT

SEQ ID NO: 129 - protein, 7 aa, Artificial Sequence
  Synthetic sequence - 20H4 CDR-L2 consensus sequence
  VARIANT 1,3, 4, 6: Xaa = any amino acid
XTXXLXT

SEQ ID NO: 130 - protein, 7 aa, Artificial Sequence
  Synthetic sequence - 20H4 CDR-L2 consensus sequence
  VARIANT 1: Xaa = S or R
  VARIANT 3: Xaa = N or K
  VARIANT 4: Xaa = N or K
  VARIANT 6: Xaa =  Q or K
XTXXLXT

SEQ ID NO: 131 - protein, 8 aa, Artificial Sequence
  Synthetic sequence - 20H4_L3_1 CDR-L3 sequence
YQDKNGWT

SEQ ID NO: 132 - protein, 8 aa, Artificial Sequence
  Synthetic sequence - 20H4_L3_2 CDR-L3 sequence
YQDKRGWT

SEQ ID NO: 133 - protein, 8 aa, Artificial Sequence
  Synthetic sequence - 20H4_L3_3 CDR-L3 sequence
YQDKKGWT

SEQ ID NO: 134 - protein, 8 aa, Artificial Sequence
  Synthetic sequence - 20H4_L3_4 CDR-L3 sequence
YQDKSGWT

SEQ ID NO: 135 - protein, 8 aa, Artificial Sequence
  Synthetic sequence - 20H4_L3_5 CDR-L3 sequence
YQYKNAWT

SEQ ID NO: 136 - protein, 8 aa, Artificial Sequence
  Synthetic sequence - 20H4_L3_6 CDR-L3 sequence
YQYKKAWT

SEQ ID NO: 137 - protein, 8 aa, Artificial Sequence
  Synthetic sequence - 20H4_L3_7 CDR-L3 sequence
YQYKNGWT

SEQ ID NO: 138 - protein, 8 aa, Artificial Sequence
  Synthetic sequence - 20H4_L3_8 CDR-L3 sequence
YQFKKAWT

SEQ ID NO: 139 - protein, 8 aa, Artificial Sequence
  Synthetic sequence - 20H4_L3_9 CDR-L3 sequence
YQYKYAWT

SEQ ID NO: 140 - protein, 8 aa, Artificial Sequence
  Synthetic sequence - 20H4_L3_10 CDR-L3 sequence
YQYKLAWT

SEQ ID NO: 141 - protein, 8 aa, Artificial Sequence
  Synthetic sequence - 20H4_L3_11 CDR-L3 sequence
YQYKFAWT

SEQ ID NO: 142 - protein, 8 aa, Artificial Sequence
  Synthetic sequence - 20H4 CDR-L3 consensus sequence
  VARIANT 3,5,6: Xaa = any amino acid
YQXKXXWT

SEQ ID NO: 143 - protein, 8 aa, Artificial Sequence
  Synthetic sequence - 20H4 CDR-L3 consensus sequence
  VARIANT 3: Xaa = Y, D, or F
  VARIANT 5: Xaa = S, N, R, K, Y, L, or F
  VARIANT 6: Xaa = A or G
YQXKXXWT

SEQ ID NO: 144 - protein, 118 aa, Artificial Sequence
  Synthetic sequence - 20H4_HC2
EVQLVESGGGLVQPGGSLRLSCAASGFTFSNAAMYWVRQAPGKGLELVSRIRTKPNNYATYHADSVKGRFTISRDNSKNTLYLQMNSLRAEDTAVYYCTADMATGDYWGQGTLVTVSS

SEQ ID NO: 145 - protein, 118 aa, Artificial Sequence
  Synthetic sequence - 20H4_HC3
EVQLVESGGGLVQPGGSLRLSCAASGFTFSNAAMYWVRQAPGKGLELVSRIRTKPNNYATYHADSVKGRFTISRDNSKNTVYLQMNSLRAEDTAVYYCTADMATGDYWGQGTLVTVSS

SEQ ID NO: 146 - protein, 118 aa, Artificial Sequence
  Synthetic sequence - 20H4_HC4
EVQLVESGGGLVQPGGSLRLSCAASGFTFSNAAMYWVRQAPGKGLELLARIRTKPNNYATYHADSVKGRFTISRDNSKNTLYLQMNSLRAEDTAVYYCTADMATGDYWGQGTLVTVSS

SEQ ID NO: 147 - protein, 118 aa, Artificial Sequence
  Synthetic sequence - 20H4_HC5
EVQLVESGGGLVQPGGSLRLSCAASGFTFSNAAMYWVRQAPGKGLELLARIRTKPNNYATYHADSVKGRFTISRDDSKNTVYLQMNSLRAEDTAVYYCTADMATGDYWGQGTLVTVSS

SEQ ID NO: 148 - protein, 118 aa, Artificial Sequence
  Synthetic sequence - 20H4_H1_1
EVQLVESGGGLVQPGGSLRLSCAASGFTFSKAAMYWVRQAPGKGLELVSRIRTKPNNYATYHADSVKGRFTISRDNSKNTLYLQMNSLRAEDTAVYYCTADMATGDYWGQGTLVTVSS

SEQ ID NO: 149 - protein, 118 aa, Artificial Sequence
  Synthetic sequence - 20H4_H1_2
EVQLVESGGGLVQPGGSLRLSCAASGFRFSKAAMYWVRQAPGKGLELVSRIRTKPNNYATYHADSVKGRFTISRDNSKNTLYLQMNSLRAEDTAVYYCTADMATGDYWGQGTLVTVSS

SEQ ID NO: 150 - protein, 118 aa, Artificial Sequence
  Synthetic sequence - 20H4_H1_3
EVQLVESGGGLVQPGGSLRLSCAASGFTFSRAAMYWVRQAPGKGLELVSRIRTKPNNYATYHADSVKGRFTISRDNSKNTLYLQMNSLRAEDTAVYYCTADMATGDYWGQGTLVTVSS

SEQ ID NO: 151 - protein, 118 aa, Artificial Sequence
  Synthetic sequence - 20H4_H1_4
EVQLVESGGGLVQPGGSLRLSCAASGFTFNKAAMYWVRQAPGKGLELVSRIRTKPNNYATYHADSVKGRFTISRDNSKNTLYLQMNSLRAEDTAVYYCTADMATGDYWGQGTLVTVSS

SEQ ID NO: 152 - protein, 118 aa, Artificial Sequence
  Synthetic sequence - 20H4_H1_5
EVQLVESGGGLVQPGGSLRLSCAASGFKFSKAAMYWVRQAPGKGLELVSRIRTKPNNYATYHADSVKGRFTISRDNSKNTLYLQMNSLRAEDTAVYYCTADMATGDYWGQGTLVTVSS

SEQ ID NO: 153 - protein, 118 aa, Artificial Sequence
  Synthetic sequence - 20H4_H1_6
EVQLVESGGGLVQPGGSLRLSCAASGFNFSKAAMYWVRQAPGKGLELVSRIRTKPNNYATYHADSVKGRFTISRDNSKNTLYLQMNSLRAEDTAVYYCTADMATGDYWGQGTLVTVSS

SEQ ID NO: 154 - protein, 118 aa, Artificial Sequence
  Synthetic sequence - 20H4_H2_1
EVQLVESGGGLVQPGGSLRLSCAASGFTFSNAAMYWVRQAPGKGLELVSRIRTKPNNFATYHADSVKGRFTISRDNSKNTLYLQMNSLRAEDTAVYYCTADMATGDYWGQGTLVTVSS

SEQ ID NO: 155 - protein, 118 aa, Artificial Sequence
  Synthetic sequence - 20H4_H2_2
EVQLVESGGGLVQPGGSLRLSCAASGFTFSNAAMYWVRQAPGKGLELVSRIRTQRNNYATYHADSVKGRFTISRDNSKNTLYLQMNSLRAEDTAVYYCTADMATGDYWGQGTLVTVSS

SEQ ID NO: 156 - protein, 118 aa, Artificial Sequence
  Synthetic sequence - 20H4_H2_3
EVQLVESGGGLVQPGGSLRLSCAASGFTFSNAAMYWVRQAPGKGLELVSRIRTERNNYATYHADSVKGRFTISRDNSKNTLYLQMNSLRAEDTAVYYCTADMATGDYWGQGTLVTVSS

SEQ ID NO: 157 - protein, 118 aa, Artificial Sequence
  Synthetic sequence - 20H4_H2_4
EVQLVESGGGLVQPGGSLRLSCAASGFTFSNAAMYWVRQAPGKGLELVSRIRTKRNNYATYHADSVKGRFTISRDNSKNTLYLQMNSLRAEDTAVYYCTADMATGDYWGQGTLVTVSS

SEQ ID NO: 158 - protein, 118 aa, Artificial Sequence
  Synthetic sequence - 20H4_H2_5
EVQLVESGGGLVQPGGSLRLSCAASGFTFSNAAMYWVRQAPGKGLELVSRIRTEPNNYATYHADSVKGRFTISRDNSKNTLYLQMNSLRAEDTAVYYCTADMATGDYWGQGTLVTVSS

SEQ ID NO: 159 - protein, 118 aa, Artificial Sequence
  Synthetic sequence - 20H4_H2_6
EVQLVESGGGLVQPGGSLRLSCAASGFTFSNAAMYWVRQAPGKGLELVSRIRTKPKNYATYHADSVKGRFTISRDNSKNTLYLQMNSLRAEDTAVYYCTADMATGDYWGQGTLVTVSS

SEQ ID NO: 160 - protein, 118 aa, Artificial Sequence
  Synthetic sequence - 20H4_H3_1
EVQLVESGGGLVQPGGSLRLSCAASGFTFSNAAMYWVRQAPGKGLELVSRIRTKPNNYATYHADSVKGRFTISRDNSKNTLYLQMNSLRAEDTAVYYCTANMATGDYWGQGTLVTVSS

SEQ ID NO: 161 - protein, 118 aa, Artificial Sequence
  Synthetic sequence - 20H4_H3_2
EVQLVESGGGLVQPGGSLRLSCAASGFTFSNAAMYWVRQAPGKGLELVSRIRTKPNNYATYHADSVKGRFTISRDNSKNTLYLQMNSLRAEDTAVYYCTANMAKGDYWGQGTLVTVSS

SEQ ID NO: 162 - protein, 118 aa, Artificial Sequence
  Synthetic sequence - 20H4_H3_3
EVQLVESGGGLVQPGGSLRLSCAASGFTFSNAAMYWVRQAPGKGLELVSRIRTKPNNYATYHADSVKGRFTISRDNSKNTLYLQMNSLRAEDTAVYYCAAAMARGDYWGQGTLVTVSS

SEQ ID NO: 163 - protein, 118 aa, Artificial Sequence
  Synthetic sequence - 20H4_H3_4
EVQLVESGGGLVQPGGSLRLSCAASGFTFSNAAMYWVRQAPGKGLELVSRIRTKPNNYATYHADSVKGRFTISRDNSKNTLYLQMNSLRAEDTAVYYCSADFAKGDYWGQGTLVTVSS

SEQ ID NO: 164 - protein, 118 aa, Artificial Sequence
  Synthetic sequence - 20H4_H3_5
EVQLVESGGGLVQPGGSLRLSCAASGFTFSNAAMYWVRQAPGKGLELVSRIRTKPNNYATYHADSVKGRFTISRDNSKNTLYLQMNSLRAEDTAVYYCTADFARGDYWGQGTLVTVSS

SEQ ID NO: 165 - protein, 118 aa, Artificial Sequence
  Synthetic sequence - 20H4_H3_6
EVQLVESGGGLVQPGGSLRLSCAASGFTFSNAAMYWVRQAPGKGLELVSRIRTKPNNYATYHADSVKGRFTISRDNSKNTLYLQMNSLRAEDTAVYYCTANMRKGDYWGQGTLVTVSS

SEQ ID NO: 166 - protein, 118 aa, Artificial Sequence
  Synthetic sequence - 20H4_H3_7
EVQLVESGGGLVQPGGSLRLSCAASGFTFSNAAMYWVRQAPGKGLELVSRIRTKPNNYATYHADSVKGRFTISRDNSKNTLYLQMNSLRAEDTAVYYCTADMAKGDYWGQGTLVTVSS

SEQ ID NO: 167 - protein, 106 aa, Artificial Sequence
  Synthetic sequence - 20H4_LC1
DIQMTQSPSSLSASVGDRVTITCKASQNINKNLDWYQQKPGKAPKLLIYSTNNLQTGVPSRFSGSGSGTDFTLTISSLQPEDFATYYCYQYKSAWTFGQGTKVEIK

SEQ ID NO: 168 - protein, 106 aa, Artificial Sequence
  Synthetic sequence - 20H4_LC2
DIQMTQSPSSLSASVGDRVTITCKASQNINKNLDWYQQKPGKAPKPLIYSTNNLQTGVPSRFSGSGSGTDFTLTISSLQPEDFATYYCYQYKSAWTFGQGTKVEIK

SEQ ID NO: 169 - protein, 106 aa, Artificial Sequence
  Synthetic sequence - 20H4_LC3
DIQMTQSPSSLSASVGDRVTITCKASQNINKNLDWYQQKPGKAPKPLIYSTNNLQTGVPSRFSGSGSGTDYTLTISSLQPEDFATYYCYQYKSAWTFGQGTKVEIK

SEQ ID NO: 170 - protein, 106 aa, Artificial Sequence
  Synthetic sequence - 20H4_LC4
EIVMTQSPATLSLSPGERATLSCKASQNINKNLDWYQQKPGQAPRLLIYSTNNLQTGIPARFSGSGSGTDFTLTISSLQPEDFAVYYCYQYKSAWTFGQGTKVEIK

SEQ ID NO: 171 - protein, 106 aa, Artificial Sequence
  Synthetic sequence - 20H4_LC5
EIVMTQSPATLSLSPGERATLSCKASQNINKNLDWYQQKPGQAPRPLIYSTNNLQTGIPARFSGSGSGTDFTLTISSLQPEDFAVYYCYQYKSAWTFGQGTKVEIK

SEQ ID NO: 172 - protein, 106 aa, Artificial Sequence
  Synthetic sequence - 20H4_LC6
EIVMTQSPATLSLSPGERATLSCKASQNINKNLDWYQQKPGQAPRPLIYSTNNLQTGVPARFSGSGSGTDYTLTISSLQPEDFAVYYCYQYKSAWTFGQGTKVEIK

SEQ ID NO: 173 - protein, 106 aa, Artificial Sequence
  Synthetic sequence - 20H4_L1_1
DIQMTQSPSSLSASVGDRVTITCKASQNINNNLDWYQQKPGKAPKPLIYSTNNLKTGVPSRFSGSGSGTDFTLTISSLQPEDFATYYCYQYKSAWTFGQGTKVEIK

SEQ ID NO: 174 - protein, 106 aa, Artificial Sequence
  Synthetic sequence - 20H4_L1_2
DIQMTQSPSSLSASVGDRVTITCKASQIINQILDWYQQKPGKAPKPLIYSTNNLQTGVPSRFSGSGSGTDFTLTISSLQPEDFATYYCYQYKSAWTFGQGTKVEIK

SEQ ID NO: 175 - protein, 106 aa, Artificial Sequence
  Synthetic sequence - 20H4_L2_1
DIQMTQSPSSLSASVGDRVTITCKASQNINKNLDWYQQKPGKAPKPLIYSTNKLKTGVPSRFSGSGSGTDFTLTISSLQPEDFATYYCYQYKSAWTFGQGTKVEIK

SEQ ID NO: 176 - protein, 106 aa, Artificial Sequence
  Synthetic sequence - 20H4_L2_2
DIQMTQSPSSLKSSVGDRVTITCKASQNINKNLDWYQQKPGKAPKPLIYRTKNLQTGVPSRFSGSGSGTDFTLTISSLQPEDFATYYCYQYKSAWTFGQGTKVEIK

SEQ ID NO: 177 - protein, 106 aa, Artificial Sequence
  Synthetic sequence - 20H4_L3_1
DIQMTQSPSSLSASVGDRVTITCKASQNINKNLDWYQQKPGKAPKPLIYSTNNLQTGVPSRFSGSGSGTDFTLTISSLQPEDFATYYCYQDKNGWTFGQGTKVEIK

SEQ ID NO: 178 - protein, 106 aa, Artificial Sequence
  Synthetic sequence - 20H4_L3_2
DIQMTQSPSSLSASVGDRVTITCKASQNINKNLDWYQQKPGKAPKPLIYSTNNLQTGVPSRFSGSGSGTDFTLTISSLQPEDFATYYCYQDKRGWTFGQGTKVEIK

SEQ ID NO: 179 - protein, 106 aa, Artificial Sequence
  Synthetic sequence - 20H4_L3_3
DIQMTQSPSSLSASVGDRVTITCKASQNINKNLDWYQQKPGKAPKPLIYSTNNLQTGVPSRFSGSGSGTDFTLTISSLQPEDFATYYCYQDKKGWTFGQGTKVEIK

SEQ ID NO: 180 - protein, 106 aa, Artificial Sequence
  Synthetic sequence - 20H4_L3_4
DIQMTQSPSSLSASVGDRVTITCKASQNINKNLDWYQQKPGKAPKPLIYSTNNLQTGVPSRFSGSGSGTDFTLTISSLQPEDFATYYCYQDKSGWTFGQGTKVEIK

SEQ ID NO: 181 - protein, 106 aa, Artificial Sequence
  Synthetic sequence - 20H4_L3_5
DIQMTQSPSSLSASVGDRVTITCKASQNINKNLDWYQQKPGKAPKPLIYSTNNLQTGVPSRFSGSGSGTDFTLTISSLQPEDFATYYCYQYKNAWTFGQGTKVEIK

SEQ ID NO: 182 - protein, 106 aa, Artificial Sequence
  Synthetic sequence - 20H4_L3_6
DIQMTQSPSSLSASVGDRVTITCKASQNINKNLDWYQQKPGKAPKPLIYSTNNLQTGVPSRFSGSGSGTDFTLTISSLQPEDFATYYCYQYKKAWTFGQGTKVEIK

SEQ ID NO: 183 - protein, 106 aa, Artificial Sequence
  Synthetic sequence - 20H4_L3_7
DIQMTQSPSSLSASVGDRVTITCKASQNINKNLDWYQQKPGKAPKPLIYSTNNLQTGVPSRFSGSGSGTDFTLTISSLQPEDFATYYCYQYKNGWTFGQGTKVEIK

SEQ ID NO: 184 - protein, 106 aa, Artificial Sequence
  Synthetic sequence - 20H4_L3_8
DIQMTQSPSSLSASVGDRVTITCKASQNINKNLDWYQQKPGKAPKPLIYSTNNLQTGVPSRFSGSGSGTDFTLTISSLQPEDFATYYCYQFKKAWTFGQGTKVEIK

SEQ ID NO: 185 - protein, 106 aa, Artificial Sequence
  Synthetic sequence - 20H4_L3_9
DIQMTQSPSSLSASVGDRVTITCKASQNINKNLDWYQQKPGKAPKPLIYSTNNLQTGVPSRFSGSGSGTDFTLTISSLQPEDFATYYCYQYKYAWTFGQGTKVEIK

SEQ ID NO: 186 - protein, 106 aa, Artificial Sequence
  Synthetic sequence - 20H4_L3_10
DIQMTQSPSSLSASVGDRVTITCKASQNINKNLDWYQQKPGKAPKPLIYSTNNLQTGVPSRFSGSGSGTDFTLTISSLQPEDFATYYCYQYKLAWTFGQGTKVEIK

SEQ ID NO: 187 - protein, 106 aa, Artificial Sequence
  Synthetic sequence - 20H4_L3_11
DIQMTQSPSSLSASVGDRVTITCKASQNINKNLDWYQQKPGKAPKPLIYSTNNLQTGVPSRFSGSGSGTDFTLTISSLQPEDFATYYCYQYKFAWTFGQGTKVEIK

SEQ ID NO: 188 - protein, 19 aa, Artificial Sequence
  Synthetic sequence - CDR-H2 for clone 22D11
RIRTKPNNYATYYADSVRG

SEQ ID NO: 189 - skipped

SEQ ID NO: 190 - protein, 217 aa, Homo sapiens; identical to SEQ ID NO: 64

SEQ ID NO: 191 - protein, 120 aa, Artificial Sequence
  Synthetic seqeunce - Anti-BACE VH
QVQLVQSGAEVKKPGSSVKVSCKASGGTLSGYGVSWVRQAPGQGLEWMGNIIPGFGTANYAQKFQGRVTITADESTSTAYMELSSLRSEDTAVYYCARSGGTKYGMLDVWGQGTMVTVSS

SEQ ID NO: 192 - protein, 108 aa, Artificial Sequence
  Synthetic seqeunce - Anti-BACE VL
EIVMTQSPATLSVSPGERATLSCRASQSVSSNLAWYQQKPGQAPRLLIYGASTRATGIPARFSGSGSGTEFTLTISSLQSEDFAVYYCQQLILWPPRTFGGGTKVEIK

SEQ ID NO: 193 - skipped

SEQ ID NO: 194 - protein, 113 aa, Artificial Sequence
  Synthetic sequence - 20H4_HC5 Heavy chain constant domain 1 (CH1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 195 - protein, 107 aa, Artificial Sequence [sequence omitted]

SEQ ID NO: 196 - protein, 213 aa, Artificial Sequence
  Synthetic seqeunce - 20H4_LC_k3_1 light chain
EIVMTQSPATLSLSPGERATLSCKASQNINNNLDWYQQKPGQAPRPLIYSTNNLKTGIPARFSGSGSGTDFTLTISSLQPEDFAVYYCYQYKSAWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 213 aa, Artificial Sequence
  Synthetic seqeunce - 20H4_LC_k3_2_v2 light chain
EIVMTQSPATLSLSPGERATLSCKASQNINKNLDWYQQKPGQAPRPLIYSTNNLQTGIPARFSGSGSGTDFTLTISSLQPEDFAVYYCYQYKSAWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 213 aa, Artificial Sequence
  Synthetic sequence - 20H4_ L1_1 light chain
DIQMTQSPSSLSASVGDRVTITCKASQNINNNLDWYQQKPGKAPKPLIYSTNNLKTGVPSRFSGSGSGTDFTLTISSLQPEDFATYYCYQYKSAWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 213 aa, Artificial Sequence
  Synthetic seqeunce - 20H4_LC_k3_2_v1 light chain
EIVMTQSPATLSLSPGERATLSCKASQNINKNLDWYQQKPGQAPRPLIYSTNNLKTGIPARFSGSGSGTDFTLTISSLQPEDFAVYYCYQYKSAWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 106 aa, Artificial Sequence; identical to SEQ ID NO: 171
  Synthetic seqeunce - 20H4_LC_k3_2_v2

SEQ ID NO: 201 - protein, 106 aa, Artificial Sequence
  Synthetic seqeunce - 20H4_LC_k3_1
EIVMTQSPATLSLSPGERATLSCKASQNINNNLDWYQQKPGQAPRPLIYSTNNLKTGIPARFSGSGSGTDFTLTISSLQPEDFAVYYCYQYKSAWTFGQGTKVEIK

SEQ ID NO: 202 - protein, 106 aa, Artificial Sequence
  Synthetic seqeunce - 20H4_LC_k3_2_v1
EIVMTQSPATLSLSPGERATLSCKASQNINKNLDWYQQKPGQAPRPLIYSTNNLKTGIPARFSGSGSGTDFTLTISSLQPEDFAVYYCYQYKSAWTFGQGTKVEIK

SEQ ID NO: 203 - protein, 106 aa, Artificial Sequence
  Synthetic seqeunce - 20H4_LC_k3_3
EIVMTQSPATLSLSPGERATLSCKASQNINQNLDWYQQKPGQAPRPLIYSTNNLKTGIPARFSGSGSGTDFTLTISSLQPEDFAVYYCYQYKSAWTFGQGTKVEIK

SEQ ID NO: 204 - protein, 106 aa, Artificial Sequence
  Synthetic sequence - 20H4_LC_k3_4
EIVMTQSPATLSLSPGERATLSCKASQNINENLDWYQQKPGQAPRPLIYSTNNLKTGIPARFSGSGSGTDFTLTISSLQPEDFAVYYCYQYKSAWTFGQGTKVEIK

SEQ ID NO: 205 - protein, 106 aa, Artificial Sequence
  Synthetic sequence - 20H4_LC_k3_5
EIVMTQSPATLSLSPGERATLSCKASQNINKQLDWYQQKPGQAPRPLIYSTNNLKTGIPARFSGSGSGTDFTLTISSLQPEDFAVYYCYQYKSAWTFGQGTKVEIK

SEQ ID NO: 206 - protein, 106 aa, Artificial Sequence
  Synthetic sequence - 20H4_LC_k3_6
EIVMTQSPATLSLSPGERATLSCKASANINKQLDWYQQKPGQAPRPLIYSTNNLKTGIPARFSGSGSGTDFTLTISSLQPEDFAVYYCYQYKSAWTFGQGTKVEIK

SEQ ID NO: 207 - protein, 106 aa, Artificial Sequence
  Synthetic sequence - 20H4_LC_k3_7
EIVMTQSPATLSLSPGERATLSCKASQNISNYLDWYQQKPGQAPRPLIYSTNNLKTGIPARFSGSGSGTDFTLTISSLQPEDFAVYYCYQYKSAWTFGQGTKVEIK

SEQ ID NO: 208 - protein, 106 aa, Artificial Sequence
  Synthetic sequence - 20H4_LC_k3_8
EIVMTQSPATLSLSPGERATLSCKASQGISNYLDWYQQKPGQAPRPLIYSTNNLKTGIPARFSGSGSGTDFTLTISSLQPEDFAVYYCYQYKSAWTFGQGTKVEIK

SEQ ID NO: 209 - protein, 106 aa, Artificial Sequence
  Synthetic sequence - 20H4_LC_k3_9
EIVMTQSPATLSLSPGERATLSCKASQGISNYLAWYQQKPGQAPRPLIYSTNNLKTGIPARFSGSGSGTDFTLTISSLQPEDFAVYYCYQYKSAWTFGQGTKVEIK

SEQ ID NO: 210 - protein, 106 aa, Artificial Sequence
  Synthetic sequence - 20H4_LC_k3_10
EIVMTQSPATLSLSPGERATLSCKASQNISNDLDWYQQKPGQAPRPLIYSTNNLKTGIPARFSGSGSGTDFTLTISSLQPEDFAVYYCYQYKSAWTFGQGTKVEIK

SEQ ID NO: 211 - protein, 106 aa, Artificial Sequence
  Synthetic sequence - 20H4_LC_k3_11
EIVMTQSPATLSLSPGERATLSCKASQNISNQLDWYQQKPGQAPRPLIYSTNNLKTGIPARFSGSGSGTDFTLTISSLQPEDFAVYYCYQYKSAWTFGQGTKVEIK

SEQ ID NO: 212 - protein, 106 aa, Artificial Sequence
  Synthetic sequence - 20H4_LC_k3_12
EIVMTQSPATLSLSPGERATLSCKASQNINDNLDWYQQKPGQAPRPLIYSTNNLKTGIPARFSGSGSGTDFTLTISSLQPEDFAVYYCYQYKSAWTFGQGTKVEIK

SEQ ID NO: 213 - protein, 106 aa, Artificial Sequence
  Synthetic sequence - 20H4_LC_k3_13
EIVMTQSPATLSLSPGERATLSCKASQNINANLDWYQQKPGQAPRPLIYSTNNLKTGIPARFSGSGSGTDFTLTISSLQPEDFAVYYCYQYKSAWTFGQGTKVEIK

SEQ ID NO: 214 - protein, 11 aa, Artificial Sequence
  Synthetic sequence - 20H4 CDR-L1 consensus sequence
  VARIANT 2-9, 11: Xaa = any amino acid
KAXXXXXXXLX

SEQ ID NO: 215 - protein, 11 aa, Artificial Sequence
  Synthetic sequence - 20H4 CDR-L1 consensus sequence
  VARIANT 3: Xaa = S or G
  VARIANT 4: Xaa = Q or A
  VARIANT 5: Xaa = N, I, or G
  VARIANT 6: Xaa = I or N
  VARIANT 7: Xaa = N or S
  VARIANT 8: Xaa = A, D, N, K, or Q
  VARIANT 9: Xaa = N, I, Q, or Y
  VARIANT 11: Xaa = D or A
KAXXXXXXXLX

SEQ ID NO: 216 - protein, 11 aa, Artificial Sequence
  Synthetic sequence - 20H4 CDR-L1 consensus sequence
  VARIANT 4: Xaa = Q or A
  VARIANT 8: Xaa = W, E, or K
  VARIANT 9: Xaa = N or Q
KASXNNNXXLD

SEQ ID NO: 217 - protein, 11 aa, Artificial Sequence
  Synthetic sequence - 20H4 CDR-L1 consensus sequence
  VARIANT 4: Xaa = Q or A
  VARIANT 8: Xaa = W, E, or K
  VARIANT 9: Xaa = N or Q
KASXNINXXLD

SEQ ID NO: 218 - protein, 11 aa, Artificial Sequence
  Synthetic sequence - 20H4 CDR-L1 consensus sequence
  VARIANT 5: Xaa = N or G
  VARIANT 9: Xaa = Y, R, or Q
  VARIANT 11: Xaa = A or D
KASQXISNXLX

SEQ ID NO: 219 - protein, 448 aa, Artificial Sequence
  Synthetic sequence - 20H4_HC6.1 heavy chain
EVQLVESGGGLVQPGGSLRLSCAASGFTFSGAAMYWVRQAPGKGLELVSRIRTKPNAYATYHAASVKGRFTISRDDSKNTLYLQMNSLRAEDTAVYYCTADMATGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 220 - protein, 448 aa, Artificial Sequence
  Synthetic sequence - 20H4_HC6.2 heavy chain
EVQLVESGGGLVQPGGSLRLSCAASGFTFSGAAMYWVRQAPGKGLELVSRIRSKANAYATAHAASVKGRFTISRDDSKNTLYLQMNSLRAEDTAVYYCTADMATGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 221 - protein, 213 aa, Artificial Sequence
  Synthetic sequence - h20H4_LC_k3_2.1 light chain
EIVMTQSPATLSLSPGERATLSCRASQNINKNLDWYQQKPGQAPRPLIYDASNLKTGIPARFSGSGSGTDFTLTISSLQPEDFAVYYCYQYKSAWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 118 aa, Artificial Sequence
  Synthetic sequence - 20H4_HC6.1_VH
EVQLVESGGGLVQPGGSLRLSCAASGFTFSGAAMYWVRQAPGKGLELVSRIRTKPNAYATYHAASVKGRFTISRDDSKNTLYLQMNSLRAEDTAVYYCTADMATGDYWGQGTLVTVSS

SEQ ID NO: 223 - protein, 118 aa, Artificial Sequence
  Synthetic sequence - 20H4_HC6.2_VH
EVQLVESGGGLVQPGGSLRLSCAASGFTFSGAAMYWVRQAPGKGLELVSRIRSKANAYATAHAASVKGRFTISRDDSKNTLYLQMNSLRAEDTAVYYCTADMATGDYWGQGTLVTVSS

SEQ ID NO: 224 - protein, 106 aa, Artificial Sequence
  Synthetic sequence - h20H4_LC_k3_2.1_VL
EIVMTQSPATLSLSPGERATLSCRASQNINKNLDWYQQKPGQAPRPLIYDASNLKTGIPARFSGSGSGTDFTLTISSLQPEDFAVYYCYQYKSAWTFGQGTKVEIK

SEQ ID NO: 225 - skipped

SEQ ID NO: 226 - protein, 448 aa, Artificial Sequence
  Synthetic sequence - 20H4_HC5_LS heavy chain
EVQLVESGGGLVQPGGSLRLSCAASGFTFSNAAMYWVRQAPGKGLELLARIRTKPNNYATYHADSVKGRFTISRDDSKNTVYLQMNSLRAEDTAVYYCTADMATGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 227 - protein, 448 aa, Artificial Sequence
  Synthetic sequence - 20H4_HC5 heavy chain
EVQLVESGGGLVQPGGSLRLSCAASGFTFSNAAMYWVRQAPGKGLELLARIRTKPNNYATYHADSVKGRFTISRDDSKNTVYLQMNSLRAEDTAVYYCTADMATGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 448 aa, Artificial Sequence
  Synthetic sequence - h20H4_HC6 heavy chain
EVQLVESGGGLVQPGGSLRLSCAASGFTFSNAAMYWVRQAPGKGLELVSRIRTKPNNYATYHADSVKGRFTISRDDSKNTLYLQMNSLRAEDTAVYYCTADMATGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 118 aa, Artificial Sequence
  Synthetic seqeunce - h20H4_HC6_VH
EVQLVESGGGLVQPGGSLRLSCAASGFTFSNAAMYWVRQAPGKGLELVSRIRTKPNNYATYHADSVKGRFTISRDDSKNTLYLQMNSLRAEDTAVYYCTADMATGDYWGQGTLVTVSS

SEQ ID NO: 230 - protein, 116 aa, Artificial Sequence
  Synthetic seqeunce - 9E4 VH
EVKLVESGGGLVKPGASLKLSCAASGFTFSNYGMSWVRQTSDKRLEWVASISSGGGSTYYPDNVKGRFTISREDAKNTLYLQMSSLRSEDTALYYCSRGGAGIDYWGQGTTLTVSS

SEQ ID NO: 231 - protein, 113 aa, Artificial Sequence
  Synthetic seqeunce - 9E4 VL
DIVMSQSPSSLAVSVGEKVTMSCKSIQTLLYSSNQKNYLAWFQQKPGQSPKLLIYWASIRKSGVPDRFTGSGSGTDFTLTISSVKAEDLAVYYCQQYYSYPLTFGAGTKLELK

SEQ ID NO: 232 - protein, 122 aa, Artificial Sequence
  Synthetic seqeunce - aslo452 ngl3 VH
EVQLLESGGGLVQPGGSLRLSCAASGFTFSSYAMSWVRQAPGKGLEWVSSISHLGGSTYYADSVKGRFTISRDNSKNTLYLQMNSLRAEDTAVYYCAGGANHGKYYYGMDKWGQGTTVTVSS

SEQ ID NO: 233 - protein, 115 aa, Artificial Sequence
  Synthetic seqeunce - aslo452 ngl3 VL
QAVLTQPASLSASPGASASLTCTLRSGAPLPKYRIYWYQQKPGSPPQYLLRYKSDADKHQGSGVPSRFSGSKDASANAGILLISGLQSEDEADYYCMVWDHGVWYFGGGTKLTVL

(19) United States
(12) Patent Application Publication
(10) Pub. No.: US 2023/0074436 A1
(43) Pub. Date: Mar. 9, 2023

(54) ANTI-ALPHA-SYNUCLEIN ANTIBODIES AND METHODS OF USE THEREOF

(71) Applicant: DENALI THERAPEUTICS INC., South San Francisco, CA (US)

(72) Inventors: Jing GUO, South San Francisco, CA (US); Rui HAO, South San Francisco, CA (US); Do Jin KIM, South San Francisco, CA (US); Suresh PODA, South San Francisco, CA (US); Rinkan SHUKLA, South San Francisco, CA (US); Adam P. SILVERMAN, South San Francisco, CA (US)

(21) Appl. No.: 17/761,088

(22) PCT Filed: Sep. 18, 2020
(86) PCT No.: PCT/US2020/051672

§ 371 Date: Mar. 16, 2022

Related U.S. Application Data
(60) Provisional application No. 62/903,588, filed on Sep. 20, 2019.

(51) Int. Cl. C07K 16/18

(52) U.S. Cl. CPC C07K 16/18; A61K 2039/505

(57) ABSTRACT

In one aspect, antibodies that specifically bind to a human alpha-synuclein protein are provided. In some embodiments, an anti-alpha-synuclein antibody binds to monomeric human alpha-synuclein protein, oligomeric human alpha-synuclein protein, soluble human alpha-synuclein protein, human alpha-synuclein protein fibrils, and human alpha-synuclein protein that is phosphorylated at Ser129 (pSer129) with high affinity. In some embodiments, an anti-alpha-synuclein antibody can specifically bind to or immunodeplete alpha-synuclein protein. In some embodiments, an anti-alpha-synuclein antibody can prevent or inhibit alpha-synuclein seeding.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 260114_422WO_SEQUENCE_LISTING.txt. The text file is 149 KB, was created on Sep. 18, 2020, and is being submitted electronically via EFS-Web.

### BACKGROUND

Alpha-synuclein protein has been identified as a significant component of Lewy bodies and Lewy neurites, which are defining neuropathological characteristics of Parkinson's disease and other neurodegenerative diseases that are classified as synucleinopathies. In addition, the filamentous inclusions that are found in multiple system atrophy are made of alpha-synuclein. Furthermore, alpha-synuclein has been implicated in the pathogenesis of a large number of neurodegenerative diseases that are classified as tauopathies, such as Alzheimer's disease, following the discovery that there are interactions between alpha-synuclein and tau proteins.

It is believed that the aggregation of alpha-synuclein oligomers and the formation of alpha-synuclein fibrils, followed by the spreading of alpha-synuclein species, results in the formation of Lewy bodies, which in turn contribute to neural cell toxicity and death. Accordingly, there is a need for therapeutic agents that target novel epitopes and neutralize all forms of alpha-synuclein protein, thus preventing the aggregation and oligomerization of alpha-synuclein protein as well as preventing the seeding and spreading of alpha-synuclein oligomers and fibrils.

### BRIEF SUMMARY

In one aspect, the present disclosure provides engineered antibodies and antigen-binding portions thereof that can bind to human alpha-synuclein protein with high affinity. In any of the herein-disclosed embodiments, an isolated anti-alpha-synuclein antibody or antigen-binding portion thereof can comprise one or more CDRs, a VH, and/or a VL according to any of the exemplary sequences provided herein.

In some embodiments, the anti-alpha-synuclein antibody is a pan-alpha-synuclein antibody.

In certain embodiments, the antibody or antigen-binding portion binds to alpha-synuclein fibrils with an IC50/K_{D} of less than 2 nM, as measured by ELISA assay.

In certain embodiments, the antibody or antigen-binding portion binds to alpha-synuclein oligomers with an IC50/K_{D} of less than 6 nM, or less than 5.5 nM, or less than 5.4 nM, as measured by ELISA assay.

In certain embodiments, the antibody or antigen-binding portion binds to alpha-synuclein with a K_{D} of about 300 pM or less (e.g., about 300 pM, 290 pM, 280 pM, 270 pM, 260 pM, 250 pM, 240 pM, 230 pM, 220 pM, 210 pM, 200 pM, 190 pM, 180 pM, 170 pM, 160 pM, 150 pM, 140 pM, 130 pM, 120 pM, 110 pM, or less), as measured by Biacore™ SPR (e.g., Biacore™ 8 K). In some embodiments, the antibody or antigen-binding portion binds to a human alpha-synuclein monomer with a K_{D} of about 120 pM or less (including, e.g., 120 pM), as measured by Biacore™ SPR. In some embodiments, the antibody or antigen-binding portion binds to a human alpha-synuclein oligomer with a K_{D} of about 190 pM or less (including, e.g., 190 pM), as measured by Biacore™ SPR. In some embodiments, the antibody or antigen-binding portion binds to a human alpha-synuclein fibril with a K_{D} of about 270 pM or less (including, e.g., 270 pM), as measured by Biacore™ SPR. In some embodiments, affinity for a human alpha-synuclein fibril refers to a measured affinity for a sonicated human alpha-synuclein fibril.

In some embodiments, the antibody or antigen-binding portion thereof specifically binds to a human alpha-synuclein monomer with a K_{D} of less than about 20 pM, as measured by KinExA® kinetic exclusion assay. In further embodiments, the antibody or antigen-binding portion thereof specifically binds to a human alpha-synuclein monomer with a K_{D} of less than about 16 pM. In further embodiments, the antibody or antigen-binding portion thereof specifically binds to a human alpha-synuclein monomer with a K_{D} of about 15 pM. In particular embodiments, the antibody or antigen-binding portion thereof specifically binds to a human alpha-synuclein monomer with a K_{D} of about 15.7 pM.

In some embodiments, the antibody or antigen-binding portion thereof specifically binds to a human alpha-synuclein oligomer with a K_{D} of less than about 20 pM, as measured by KinExA® kinetic exclusion assay. In further embodiments, the antibody or antigen-binding portion thereof specifically binds to a human alpha-synuclein oligomer with a K_{D} of less than about 15 pM. In further embodiments, the antibody or antigen-binding portion thereof specifically binds to a human alpha-synuclein oligomer with a K_{D} of less than about 10 pM. In particular embodiments, the antibody or antigen-binding portion thereof specifically binds to a human alpha-synuclein oligomer with a K_{D} of about 9 pM, or less. In particular embodiments, the antibody or antigen-binding portion thereof specifically binds to a human alpha-synuclein oligomer with a K_{D} of about 8 pM. In particular embodiments, the antibody or antigen-binding portion thereof specifically binds to a human alpha-synuclein monomer with a K_{D} of about 8.8 pM.

In some embodiments, the antibody or antigen-binding portion thereof specifically binds to a human alpha-synuclein fibril with a K_{D} of less than about 10 pM, as measured by KinExA® kinetic exclusion assay. In certain embodiments, the antibody or antigen-binding portion thereof specifically binds to a human alpha-synuclein fibril with a K_{D} of less than about 5 pM. In certain embodiments, the antibody or antigen-binding portion thereof specifically binds to a human alpha-synuclein fibril with a K_{D} of less than about 1 pM. In further embodiments, the antibody or antigen-binding portion thereof specifically binds to a human alpha-synuclein fibril with a K_{D} of about 0.6 pM. In further embodiments, the antibody or antigen-binding portion thereof specifically binds to a human alpha-synuclein fibril with a K_{D} of 0.63 pM. In some embodiments, affinity for a human alpha-synuclein fibril refers to a measured affinity for a sonicated human alpha-synuclein fibril.

In some embodiments, the antibody or antigen-binding portion thereof binds to human alpha-synuclein protein with an EC50 value of less than about 1.5 nM, 1 nM, less than about 0.5 nM, less than about 0.25 nM, or less. In some embodiments, the antibody or antigen-binding portion thereof binds to human alpha-synuclein protein with a Bmax (O.D. at 450 nm) of less than about 1, less than about 0.9, or about 0.8, e.g., as determined using Biacore™ SPR (e.g., Biacore™ 8 K).

In certain embodiments, the antibody or antigen-binding portion comprises a first Fc polypeptide and a second Fc polypeptide. In some embodiments, the first Fc polypeptide, the second Fc polypeptide, or both, comprises (i) a mutation that reduces or ablates effector function, (ii) a mutation that increases or improves binding to FcRn, or both (i) and (ii). For example, in certain embodiments, first Fc polypeptide and/or the second Fc polypeptide comprises an Ala at position 234 and an Ala at position 235 (EU numbering). In some embodiments, the first Fc polypeptide and/or the second Fc polypeptide comprises a Leu at position 428 and a Ser at position 434. In some embodiments, the first and/or the second Fc polypeptide are or are derived from (e.g., have least 75%, or at least 80%, 90%, 92%, or 95%, amino acid sequence identity to) a human (e.g., wild-type human) IgG1, IgG2, IgG3, or IgG4 isotype.

In some embodiments, an anti-alpha-synuclein antibody described herein is a monoclonal antibody. In some embodiments, an anti-alpha-synuclein antibody described herein is a chimeric antibody. In some embodiments, an anti-alpha-synuclein antibody described herein is a humanized antibody. In some embodiments, an anti-alpha-synuclein antibody described herein is a fully human antibody. In some embodiments, the antigen-binding portion is a Fab, a F(ab′)₂, a scFv, or a bivalent scFv.

In yet another aspect, hybridoma cell lines that secrete antibodies of the present disclosure are provided. In some embodiments, the hybridoma cell line is selected from the group consisting of 1H5, 20H4, 22D11, 16F3, 18B4, and 22E11. In another aspect, antibodies that are secreted by hybridoma cell lines of the present disclosure are provided.

In another aspect, compositions are provided. In some embodiments, a composition comprises an antibody or antigen-binding portion that specifically binds to a human alpha-synuclein protein as described herein and further comprises one or more pharmaceutically acceptable carriers.

In still another aspect, isolated polynucleotides are provided. In some embodiments, the isolated polynucleotide comprises a nucleotide sequence encoding an antibody or antigen-binding portion that specifically binds to a human alpha-synuclein protein as described herein. In another aspect, vectors and host cells comprising such an isolated polynucleotide are provided.

In another aspect, kits are provided. In some embodiments, a kit comprises (i) the antibody or antigen-binding portion; (ii) the composition; (iii) the polynucleotide; (iv) the vector; and/or (v) the host cell; and (vi) instructions for using the antibody, antigen-binding portion, composition, polynucleotide, vector, and/or host cell to treat a neurodegenerative disease (e.g., which instructions can comprise instructions for formulating the antibody, antigen-binding portion, composition, polynucleotide, vector, and/or host cell for administration to a subject, and/or for administering the antibody, antigen-binding portion, composition, polynucleotide, vector, and/or host cell to a subject).

In another aspect, antibodies are provided that compete for specific binding to a human alpha-synuclein protein with an antibody as described herein.

In still another aspect, methods for neutralizing human alpha-synuclein protein in a brain of a subject are provided. In some embodiments, the method comprises administering to the subject an antibody (or antigen-binding portion thereof) that specifically binds to a human alpha-synuclein protein as described herein, or a composition comprising an anti-alpha-synuclein antibody or antigen-binding portion thereof, as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein. In some embodiments, a method of neutralizing human alpha-synuclein protein comprises preventing or reducing human alpha-synuclein protein oligomerization and/or aggregation in the brain of the subject. In some embodiments, a method of neutralizing human alpha-synuclein protein comprises preventing or reducing the spreading of human alpha-synuclein protein oligomers and/or fibrils in the brain of the subject. In some embodiments, a method of neutralizing human alpha-synuclein protein comprises preventing or reducing the seeding of human alpha-synuclein protein (e.g., alpha-synuclein fibrils and/or oligomers) in the brain of the subject.

In some embodiments, the isolated antibody, the antigen-binding portion of the antibody, or the composition neutralizes monomeric human alpha-synuclein protein, oligomeric human alpha-synuclein protein, soluble human alpha-synuclein protein, human alpha-synuclein protein fibrils, human alpha-synuclein protein that is phosphorylated at Ser129 (pSer129), or any combination thereof. In some embodiments, the pan-alpha-synuclein antibody or antigen-binding portion thereof specifically binds to each of monomeric, oligomeric, fibrillary (i.e., fibrils), and pSer129 human alpha-synuclein with high affinity, for example with affinity of less than about 20, 19, 18, 17, or 16 pM, as measured by KinExA® kinetic exclusion assay. In some embodiments, the pan-alpha synuclein antibody or antigen-binding portion thereof specifically binds to each of oligomeric, fibrillar, and pSer129 human alpha-synuclein with affinity of less than about 10 pM. In some embodiments, the subject has a neurodegenerative disease (e.g., a synucleinopathy).

In some embodiments, the isolated antibody, the antigen-binding portion, or the composition is administered during an early stage of the neurodegenerative disease.

In yet another aspect, methods for treating a neurodegenerative disease in a subject are provided. In some embodiments, the neurodegenerative disease is a synucleinopathy. In some embodiments, the method comprises administering to the subject an antibody (or antigen-binding portion thereof) that specifically binds to a human alpha-synuclein protein as described herein, or a composition comprising an anti-alpha-synuclein antibody or antigen-binding portion thereof, as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein.

In some embodiments, the neurodegenerative disease (e.g., synucleinopathy) is selected from the group consisting of Parkinson's disease, dementia with Lewy bodies, multiple system atrophy, Huntington's disease, Alzheimer's disease, primary age-related tauopathy, hereditary late-onset Parkinson's disease, dysautonomia, traumatic brain injury (TBI), cerebrovascular dementia, progressive supranuclear palsy (PSP), frontotemporal dementia, frontotemporal dementia with parkinsonism linked to chromosome 17, argyrophilic grain dementia, amyotrophic lateral sclerosis/parkinsonism-dementia complex of Guam, corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, inclusion-body myositis, myotonic dystrophy, neurofibrillary tangle-predominant dementia, Niemann-Pick disease type C, pallido-ponto-nigral degeneration, Pick's disease, postencephalitic parkinsonism, prion protein cerebral amyloid angiopathy, progressive subcortical gliosis, subacute sclerosing panencephalitis, and tangle only dementia.

In some embodiments, the subject is treated during an early stage of the neurodegenerative disease.

In another aspect, anti-alpha-synuclein antibodies (and antigen-binding portions thereof) and related compositions for use in a method of neutralizing human alpha-synuclein protein in a brain of a subject are provided. In some embodiments, an antibody or antigen-binding portion thereof that specifically binds to a human alpha-synuclein protein as described herein or a composition comprising an anti-alpha-synuclein antibody as described herein, is for use in a method of preventing or reducing human alpha-synuclein protein oligomerization and/or aggregation in the brain of the subject. In some embodiments, an antibody or antigen-binding portion thereof that specifically binds to a human alpha-synuclein protein as described herein, or a composition comprising an anti-alpha-synuclein antibody as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein, is for use in a method of preventing or reducing the spreading of human alpha-synuclein protein oligomers and/or fibrils in the brain of the subject.

In another aspect, anti-alpha-synuclein antibodies (and antigen-binding portions thereof) and related compositions for use in a method of blocking human alpha-synuclein protein seeding (e.g., seeding of alpha-synuclein fibrils and/or oligomers) in a brain of a subject are provided. In some embodiments, an antibody or antigen-binding portion thereof that specifically binds to a human alpha-synuclein protein as described herein, or a composition comprising an anti-alpha-synuclein antibody as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein, is for use in a method of reducing or inhibiting human alpha-synuclein protein seeding in a brain of a subject.

In still another aspect, anti-alpha-synuclein antibodies (and antigen-binding portions thereof) and related compositions for use in a method of treating a neurodegenerative disease are provided. In some embodiments, the neurodegenerative disease is a synucleinopathy. In some embodiments, an antibody or antigen-binding portion thereof that specifically binds to a human alpha-synuclein protein as described herein, or a composition comprising an anti-alpha-synuclein antibody as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein, is for use in a method of treating a neurodegenerative disease (e.g., a synucleinopathy). In some embodiments, an antibody or antigen-binding portion thereof that specifically binds to a human alpha-synuclein protein as described herein or a composition comprising an anti-alpha-synuclein antibody as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein, is for use in a method of treating a neurodegenerative disease (e.g., a synucleinopathy) selected from the group consisting of Parkinson's disease, dementia with Lewy bodies, multiple system atrophy, Huntington's disease, Alzheimer's disease, primary age-related tauopathy, hereditary late-onset Parkinson's disease, dysautonomia, traumatic brain injury (TBI), cerebrovascular dementia, progressive supranuclear palsy (PSP), frontotemporal dementia, frontotemporal dementia with parkinsonism linked to chromosome 17, argyrophilic grain dementia, amyotrophic lateral sclerosis/parkinsonism-dementia complex of Guam, corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, inclusion-body myositis, myotonic dystrophy, neurofibrillary tangle-predominant dementia, Niemann-Pick disease type C, pallido-ponto-nigral degeneration, Pick's disease, postencephalitic parkinsonism, prion protein cerebral amyloid angiopathy, progressive subcortical gliosis, subacute sclerosing panencephalitis, and tangle only dementia.

In yet another aspect, the use of an anti-alpha-synuclein antibody as described herein (or an antigen-binding portion thereof as described herein or a composition as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein) in the manufacture of a medicament for the neutralization of human alpha-synuclein protein in a brain of a subject is provided. In some embodiments, neutralizing comprises preventing or reducing human alpha-synuclein protein oligomerization and/or aggregation in the brain of the subject. In some embodiments, neutralizing comprises preventing or reducing the spreading of human alpha-synuclein protein oligomers and/or fibrils in the brain of the subject. In some embodiments, neutralizing comprises preventing or reducing the seeding of human alpha-synuclein protein (e.g., alpha-synuclein fibrils and/or oligomers) in the brain of the subject.

In still another aspect, the use of an anti-alpha-synuclein antibody as described herein (or an antigen-binding portion thereof as described herein or a composition as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein) in the manufacture of a medicament for the treatment of a neurodegenerative disease (e.g., a synucleinopathy) is provided. In some embodiments, the use of an anti-alpha-synuclein antibody (or the antigen-binding portion, or composition, or polynucleotide encoding an antibody, or vector or host cell comprising a polynucleotide encoding the antibody) as described herein is for the manufacture of a medicament for the treatment of a neurodegenerative disease (e.g., a synucleinopathy) selected from the group consisting of Parkinson's disease, dementia with Lewy bodies, multiple system atrophy, Huntington's disease, Alzheimer's disease, primary age-related tauopathy, hereditary late-onset Parkinson's disease, dysautonomia, traumatic brain injury (TBI), cerebrovascular dementia, progressive supranuclear palsy (PSP), frontotemporal dementia, frontotemporal dementia with parkinsonism linked to chromosome 17, argyrophilic grain dementia, amyotrophic lateral sclerosis/parkinsonism-dementia complex of Guam, corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, inclusion-body myositis, myotonic dystrophy, neurofibrillary tangle-predominant dementia, Niemann-Pick disease type C, pallido-ponto-nigral degeneration, Pick's disease, postencephalitic parkinsonism, prion protein cerebral amyloid angiopathy, progressive subcortical gliosis, subacute sclerosing panencephalitis, and tangle only dementia.

### BRIEF DESCRIPTION OF THE DRAWINGS

FIGS. 1A and 1B show normalized fibril and oligomer binding for clones 1H5, 16F3, 18B4, 20H4, 22D11, and 22E11, as measured using competition ELISA. IC50 values are shown in Table 2. FIG. 1A shows fibril binding data. FIG. 1B shows oligomer binding data.

FIGS. 2A-2F show epitope mapping by peptide ELISA. Results are summarized in Table 3. FIG. 2A shows data for clone 1H5. FIG. 2B shows data for clone 20H4. FIG. 2C shows data for clone 16F3. FIG. 2D shows data for clone 22D11. FIG. 2E shows data for clone 18B4. FIG. 2F shows data for clone 22E11.

FIGS. 3A-3C show titration curves for binding of clones 1H5, 16F3, 18B4, 20H4, 22D11, and 22E11 to human and rodent alpha-synuclein protein. Results are summarized in Table 6. FIG. 3A shows binding to human alpha-synuclein protein. FIG. 3B shows binding to mouse alpha-synuclein protein. FIG. 3C shows binding to rat alpha-synuclein protein.

FIG. 4 shows mouse in vivo plasma concentrations of an exemplary anti-alpha synuclein antibody of the present disclosure and control antibody in mouse, measured over a seven-day period following a single administration (10 mg/kg) of antibody.

FIG. 5 shows results of a 28-day single-dose PK study (plasma concentration over time) of an exemplary anti-alpha synuclein antibody of the present disclosure or control antibodies in cynomolgus monkeys. FIG. 5 shows composite concentration curves of the tested antibodies. N=3 animals for each Ab.

FIGS. 6A and 6B show the ability of an exemplary anti-alpha-synclein antibody of the present disclosure to immunodeplete alpha-synuclein from cerebrospinal fluid (CSF) obtained from patients with Parkinson's Disease (PD). (A) Picomolar concentration of alpha-synuclein in PD CSF after incubation with antibody. (B) Alpha-synuclein levels in CSF as a percentage of negative control (anti-RSV) following incubation with antibody.

FIG. 7 shows results from Meso Scale Discovery (MSD) ELISA experiments examining the ability of the indicated antibodies to immunodeplete alpha-synuclein from PD brain tissue; results show the unbound fraction of high salt fraction immunoprecipitate. Percentages are with reference to anti-RSV control.

FIGS. 8A and 8B show results from ELISA experiments examining ability of the indicated antibodies to deplete alpha-synuclein from PD brain tissue; results show the quantitated unbound fraction (sarkosyl-insoluble pellet from brain lysate) as determined by MSD ELISA (A) and by aSyn oligomer ELISA (B). Percentages are with reference to anti-RSV control.

FIGS. 9A-11 relate to an in vivo study (see Example 7 herein) in which the effects of anti-alpha-synuclein antibodies on transgenic mice expressing mutant human alpha-synuclein were investigated.

FIGS. 9A and 9B show qualitative assessment IHC (per coronal level) on the ipsilateral (injected side; 9A) and contralateral side (9B) of pSer129 load on the indicated brain regions.

FIG. 10 shows levels of free alpha-synuclein in cerebral interstitial fluid (ISF) in Tg hemizygous mice expressing human alpha-synuclein and injected with PFFs (as described in Example 7) that received anti-alpha-synuclein antibody 20H4_HC5_L1_1 at a range of concentrations, as indicated in the figure legend.

FIG. 11 shows the percentage (normalized to baseline) of unbound ISF alpha-synuclein in PFF-injected Tg mice following administration of 20H4_HC5_L1_1 at 3, 10, 30, or 50 mg/kg. Fits were achieved using 4-parameter IC50 model in Prism. Due to normalization to 100% baseline, the “Top” estimate was constrained to 100. Some data concentration data points above 5 nM are not shown on the graphs as the x-axis is limited to 5 nM to better display the origin. The “0” concentration is assumed to be 100%.

FIG. 12 shows in vivo clearance of an exemplary anti-alpha synclein antibody of the present disclosure (20H4_H5_L1_1_LS) in cynomolgus monkeys over a 28-day study following a single intravenous dose of the antibody at 10 mg/kg or 30 mg/kg. A comparator anti-alpha synuclein antibody described in the Examples (Antibody 2) was administered intravenously to reference animals at 30 mg/kg.

FIGS. 13A and 13B show the effects of 20H4_H5_L1_1_LS and Antibody 2 on CSF free alpha-synuclein levels in cynomolgus monkeys. A single dose of antibody was administered intravenously at 30 mg/kg, and free alpha-synuclein was measured at the indicated timepoints. (A) Absolute concentration (pM) of free alpha-synuclein. (B) Change in free alpha-synuclein normalized to baseline.

FIGS. 14A and 14B show the effects of 20H4_H5_L1_1_LS (10 mg/kg and 30 mg/kg) and Antibody 2 (30 mg/kg) on CSF free alpha-synuclein levels in cynomolgus monkeys. A single dose of antibody was administered intravenously at the indicated concentration, and free alpha-synuclein was measured at the indicated timepoints. (A) Absolute concentration (pM) of free alpha-synuclein. (B) Change in free alpha-synuclein normalized to baseline.

FIGS. 15A and 15B show enlarged views of the data from Day −2 to Day 3 from FIGS. 14A and 14B, respectively.

FIGS. 16A and 16B show mouse in vivo plasma concentrations of exemplary anti-alpha synuclein antibodies of the present disclosure, measured over a seven-day period following a single administration (10 mg/kg or 50 mg/kg) of antibody.

### DETAILED DESCRIPTION

### I. Introduction

The present disclosure relates to humanized, affinity-matured antibodies that recognize novel epitopes in, and have the ability to specifically bind to multiple forms of, human alpha-synuclein protein. In certain embodiments, presently disclosed antibodies comprise mutations in an Fc polypeptide that reduce or eliminate effector function and/or mutations that increase in vivo half-life, e.g., by increasing binding of antibody Fc to Fc neonatal receptor (FcRn).

In some embodiments, the anti-alpha-synuclein antibodies described herein are pan-alpha-synuclein antibodies that specifically bind to, e.g., monomeric human alpha-synuclein, oligomeric human alpha-synuclein, soluble human alpha-synuclein protein, human alpha-synuclein fibrils, and/or human alpha-synuclein that is phosphorylated at Ser129 (pSer129). In some embodiments, the anti-alpha-synuclein antibodies described herein specifically bind to both phosphorylated (e.g., pSer129) human alpha-synuclein protein and unphosphorylated human alpha-synuclein protein (e.g. with about the same affinity). In some embodiments, the anti-alpha-synuclein antibodies described herein have a higher binding affinity for human alpha-synuclein protein than for rat alpha-synuclein protein.

The anti-alpha-synuclein antibodies of the present disclosure are useful for, as non-limiting examples, neutralizing alpha-synuclein protein, inhibiting or preventing alpha-synuclein spreading, inhibiting or preventing alpha-synuclein oligomerization and/or aggregation, and preventing, reducing, or inhibiting the seeding of alpha-synuclein protein (e.g., alpha-synuclein fibrils and/or oligomers). For example, the anti-alpha-synuclein antibodies described herein can specifically bind both soluble and insoluble forms of alpha-synuclein protein (e.g., in brain lysate and cerebrospinal fluid samples from patients with neurodegenerative diseases such as Parkinson's disease) with improved efficiency. Accordingly, antibodies of the present disclosure find utility for, among other things, preventing and treating various diseases such as degenerative neurological diseases including Parkinson's disease.

### II. Definitions

As used herein, the singular forms “a,” “an,” and “the” include plural referents unless the content clearly dictates otherwise. Thus, for example, reference to “an antibody” may include one or more antibodies, two or more antibodies, or the like. The use of the alternative (e.g., “or”) should be understood to mean either one, both, or any combination thereof of the alternatives.

In the present description, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, the term “synuclein” or “synuclein protein” refers to a native (i.e., wild-type) alpha-, beta-, or gamma-synuclein protein of any vertebrate, including naturally occurring mutations, fragments, and variants thereof, such as but not limited to human, non-human primates (e.g., cynomolgus monkey), rodents (e.g., mice), and other mammals. Unless stated otherwise, synuclein protein described herein includes protein prepared or obtained (e.g., isolated) from a tissue or from a fluid sample of any vertebrate, as well as from recombinant protein encoded by recombinant DNA and expressed from any expression cell or system. The term also includes monomeric and oligomeric forms, soluble and insoluble synuclein, and synuclein fibrils. One specific example is the full-length human alpha-synuclein protein, expressed from the SNCA gene, having a length of 140 amino acids (SEQ ID NO:1, UniProt reference identifier P37840), and fragments thereof. Alpha-synuclein protein may also be referred to “aSyn,” “asyn,” “a-syn,” “a-synuclein,” “a-syn,” or the like.

In some embodiments, an anti-alpha-synuclein antibody or antigen-binding portion of the present disclosure specifically binds to one or more of: monomeric alpha-synuclein; oligomeric alpha-synuclein; soluble alpha-synuclein; alpha-synuclein fibrils; or phosphorylated alpha-synuclein (e.g., pSer129)). In other words, the anti-alpha-synuclein antibody or antigen-binding portion can specifically bind any one or any two, any three, any four, or all five of the above-referenced forms of alpha-synuclein. In some embodiments, an anti-alpha-synuclein antibody or antigen-binding portion is an antibody or antigen-binding portion that specifically binds to multiple forms of alpha-synuclein protein (e.g., monomeric alpha-synuclein, oligomeric alpha-synuclein, soluble alpha-synuclein, alpha-synuclein fibrils, and/or phosphorylated alpha-synuclein (e.g., pSer129)).

In some embodiments, an anti-alpha-synuclein antibody is a pan-alpha-synuclein antibody. As used herein, the term “pan-alpha-synuclein antibody” refers to an antibody that specifically binds to multiple forms of alpha-synuclein protein (e.g., binds to any two or more of monomeric alpha-synuclein, oligomeric alpha-synuclein, soluble alpha-synuclein, alpha-synuclein fibrils, and phosphorylated alpha-synuclein (e.g., pSer129)). In certain embodiments, a pan-alpha-synuclein antibody specifically binds to at least two of monomeric alpha-synuclein, oligomeric alpha-synuclein, soluble alpha-synuclein, alpha-synuclein fibrils, and phosphorylated alpha-synuclein (e.g., pSer129). In certain embodiments, a pan-alpha-synuclein antibody specifically binds to each of monomeric alpha-synuclein, oligomeric alpha-synuclein, soluble alpha-synuclein, alpha-synuclein fibrils, and phosphorylated alpha-synuclein (e.g., pSer129). In certain embodiments, the pan-alpha-synuclein antibody specifically binds to at least two of monomeric alpha-synuclein, oligomeric alpha-synuclein, soluble alpha-synuclein, alpha-synuclein fibrils, and phosphorylated alpha-synuclein (e.g., pSer129), but may not bind to each form with the same affinity. In one example, an alpha-synuclein antibody specifically binds to each of monomeric, oligomeric, fibril, and pSer129 human alpha-synuclein with high affinity, for example with affinity of at least about 10 nM, in another example, at least about 6 nM.

As used herein, the term “antibody” refers to a protein with an immunoglobulin fold that specifically binds to an antigen via its variable regions. The term encompasses intact polyclonal antibodies, intact monoclonal antibodies, single-chain antibodies, multispecific antibodies, monospecific antibodies, monovalent antibodies, intrabodies, peptibodies, heteroconjugate antibodies, chimeric antibodies, humanized antibodies, and human antibodies. The term “antibody,” as used herein, also includes antibody fragments that retain antigen-binding specificity via its variable regions, including but not limited to Fab, F(ab′)₂, Fv, scFv, and bivalent scFv. Antibodies can contain light chains that are typically classified as either kappa or lambda. Antibodies can contain heavy chains that are typically classified as gamma, mu, alpha, delta, or epsilon, which in turn define the immunoglobulin classes, IgG, IgM, IgA, IgD and IgE, respectively. In certain embodiments, an antibody is an intact or full-length antibody. In certain embodiments, an antibody is a functional fragment of an intact or full-length antibody.

An exemplary immunoglobulin (antibody) structural unit comprises a tetramer. In certain immunoglobulins (e.g., some naturally occurring antibodies) a tetramer is composed of two identical pairs of polypeptide chains, each pair having one “light” (about 25 kD) and one “heavy” chain (about 50-70 kD). The N-terminal portion of each heavy and light chain defines a variable region of about 100 to 110 or more amino acids; sequences within one or both of the variable regions are primarily responsible for antigen recognition. The terms “light chain variable domain” (VL) and “heavy chain variable domain” (VH) refer to these domains of antibody light and heavy chains, respectively.

The term “variable region” refers to a domain in an antibody heavy chain or light chain that is derived from a germline Variable (V) gene, Diversity (D) gene, and/or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” is synonymous with “hypervariable region” or “HVR,” and refers to sequences of amino acids within antibody variable regions which confer and/or primarily contribute to antigen specificity and/or binding affinity. In some cases, three hypervariable regions in each chain of an antibody interrupt or separate the four framework regions established by each of the light and heavy chain variable regions. The CDRs are primarily responsible for antibody binding to an epitope of an antigen. The CDRs of each heavy and light chain are typically referred to as CDR1, CDR2, and CDR3, numbered sequentially starting from the N-terminus, and are also typically identified by the chain in which the particular CDR is located. For example, a VH CDR3 or CDR-H3 is located in the variable region of the heavy chain of the antibody in which it is found (e.g., is the third CDR starting from the N-terminus of the heavy chain), whereas a VL CDR1 or CDR-L1 is the CDR1 from the variable region of the light chain of the antibody in which it is found (e.g., is the first CDR starting from the N-terminus of the light chain).

In general, the “framework regions” or “FRs” of different light or heavy chains are relatively conserved within a species. The framework region or portion of an antibody, that is the combined framework regions of the constituent light and heavy chain variable regions, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of CDRs and framework regions can be determined using various well-known definitions and resources in the art, e.g., Kabat, Chothia, international ImMunoGeneTics database (IMGT), AbM, AHo, Martin, and observed antigen contacts (“Contact”). In some embodiments, CDRs are determined according to the Contact definition. See, MacCallum et al., J. Mol. Biol., 262:732-745 (1996). In some embodiments, CDRs are determined by a combination of Kabat, Chothia, and Contact CDR definitions.

The terms “antigen-binding portion” and “antigen-binding fragment” are used interchangeably herein and refer to one or more fragments of an antibody that retain the ability to specifically bind to an antigen (e.g., an alpha-synuclein protein or epitope-containing portion thereof) via its variable region(s). Examples of antigen-binding fragments include, but are not limited to, a Fab fragment (a monovalent fragment consisting of the VL, VH, CL, and CH1 domains), a F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), a VL (light chain variable region), and a VH (heavy chain variable region).

The term “epitope” refers to the area or region of a target antigen to which the antibody or antigen-binding portion specifically binds and can include a few amino acids or portions of a few amino acids, e.g., 5 or 6, or more, e.g., 20 or more amino acids, or portions of those amino acids. Thus, for example, where the target is a protein, the epitope can be comprised of consecutive amino acids (e.g., a linear epitope), or amino acids from different parts of the protein that are brought into proximity by protein folding (e.g., a discontinuous or conformational epitope), or non-contiguous amino acids that are in close proximity irrespective of protein folding. In some embodiments, an antibody specifically binds to two distinct regions of an antigen (e.g., an alpha-synuclein protein) that are not brought into proximity by protein folding, referred to herein as a “dual epitope.” In some embodiments, the epitope is phosphorylated at one amino acid (e.g., at a serine or threonine residue).

As used herein, the phrase “recognizes an epitope,” as used with reference to an anti-alpha-synuclein antibody, means that the antibody interacts with or specifically binds to the antigen (i.e., the alpha-synuclein protein) at that epitope or a portion of the antigen containing that epitope.

As used herein, the term “multispecific antibody” refers to an antibody that comprises two or more different antigen-binding portions, in which each antigen-binding portion comprises a different variable region that recognizes a different antigen, or a fragment or portion of the antibody that binds to the two or more different antigens via its variable regions. Non-limiting examples of multispecific antibody formats are disclosed in, for example, Spiess et al., Mol. Immunol. 67(2):95 (2015), and in Brinkmann and Kontermann, mAbs 9(2):182-212 (2017).

A “monoclonal antibody” refers to an antibody produced by a single clone of cells or a single cell line and consisting of or consisting essentially of antibody molecules that are identical in their primary amino acid sequence.

A “polyclonal antibody” refers to an antibody obtained from a heterogeneous population of antibodies in which different antibodies in the population bind to different epitopes of an antigen.

A “chimeric antibody” refers to an antibody molecule in which the constant region, or a portion thereof, is altered, replaced or exchanged so that the antigen-binding site (i.e., variable region, CDR, or portion thereof) is linked to a constant region of a different or altered class, effector function and/or species, or in which the variable region, or a portion thereof, is altered, replaced or exchanged with a variable region having a different or altered antigen specificity (e.g., CDR and framework regions from different species). In some embodiments, a chimeric antibody is a monoclonal antibody comprising a variable region from one source or species (e.g., rat) and a constant region derived from a second source or species (e.g., human). Methods for producing chimeric antibodies are described in the art.

“Humanized” antibodies are chimeric immunoglobulins derived from a non-human source (e.g., murine) that contain minimal sequences derived from the non-human immunoglobulin outside of the CDRs. In general, a humanized antibody will comprise at least one (e.g., two) antigen-binding variable domain(s), in which the CDR regions substantially correspond to those of the non-human immunoglobulin (e.g., one or more CDR amino acids may be changed from those of the non-human immunoglobulin to an (e.g., corresponding) amino acid according to a human immunoglobulin, or to an immunoglobulin of a different species, or may be otherwise changed or substituted away from the non-human immunoglobulin) and the framework regions substantially correspond to those of a human immunoglobulin sequence. In some instances, certain framework region residues of a human immunoglobulin can be replaced with a corresponding residue (or residues) from a non-human species to, e.g., improve specificity, affinity, and/or serum half-life. The humanized antibody can also comprise at least a portion of an immunoglobulin constant region (Fc), typically that of a human immunoglobulin sequence. Methods of antibody humanization are known in the art.

A “human antibody” or a “fully human antibody” is an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “specifically binds” refers to a molecule (e.g., an antibody (or an antigen-binding portion thereof)) that binds to an epitope with greater affinity, greater avidity, and/or greater duration to that epitope in a sample than it binds to another epitope (e.g., a structurally different antigen). In some embodiments, an antibody (or an antigen-binding portion thereof) that specifically binds to an epitope is an antibody (or an antigen-binding portion thereof) that binds to the epitope with at least 5-fold greater affinity than to other epitopes, e.g., at least about a 5-fold, 10-fold, 50-fold, 100-fold, 1,000-fold, 10,000 or greater affinity. In some embodiments, an antibody that specifically binds to an alpha-synuclein protein binds to the alpha-synuclein protein with at least about a 5-fold, 10-fold, 50-fold, 100-fold, 1,000-fold, 10,000-fold or greater affinity as compared to the affinity with which the antibody binds to a different protein or other target. In some embodiments, an antibody that specifically binds to an alpha-synuclein protein binds to the alpha-synuclein protein with no measureable binding to beta- or gamma-synuclein. It will be recognized by one of skill that an antibody that specifically binds to an alpha-synuclein protein from one species may also specifically bind to orthologs of the alpha-synuclein protein.

The term “binding affinity” is used herein to refer to the strength of a non-covalent interaction between two molecules, e.g., between an antibody (or an antigen-binding portion thereof) and an antigen. Thus, for example, the term may refer to 1:1 interactions between an antibody (or an antigen-binding portion thereof) and an antigen, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform). As used herein, “binding affinity” includes not only formal binding affinities, such as those reflecting 1:1 interactions between an antibody (or an antigen-binding portion thereof) and an antigen, but also apparent affinities for which K_{DS} are calculated that may reflect avid binding.

In certain embodiments, monomeric alpha-synuclein interacts with and forms asyn-dimers, which may further propagate through interactions with either monomeric or dimeric alpha-synuclein. These interactions may occur in cell cytoplasm or in/at the cellular membrane. Propagating a-syn dimers can form oligomers which can interact with cell membranes and form trans-membrane pores, inducing abnormal intracellular processes, such as calcium influx. Cytoplasmic a-syn oligomers grow by the addition of soluble monomers, dimers or other oligomers to form fibrils and then longer fibrils. Accumulation of fibrils may form Lewy bodies. The oligomers and amyloid fibrils are toxic to certain cell processes, including mitochondrial and endoplasmic reticulum function, such as Golgi trafficking, protein degradation and/or synaptic transmission, which may lead to neurodegeneration and diseases such as Parkinson's disease. In certain embodiments, anti-alpha synuclein antibodies bind to and neutralize alpha-synuclein protein.

As used herein, the term “neutralizes alpha-synuclein protein,” or “neutralizing alpha-synuclein protein” means to inhibit or reduce the ability of an alpha-synuclein protein to bind to, or interact with, another moiety, such as another alpha-synuclein protein. As a non-limiting example, neutralization of an alpha-synuclein protein inhibits or reduces its ability to bind to or interact with other alpha-synuclein monomers, alpha-synuclein oligomers, alpha-synuclein fibrils, soluble forms of alpha-synuclein, or phosphorylated forms of alpha-synuclein (e.g., pSer129). As another non-limiting example, neutralization of an alpha-synuclein protein inhibits or reduces its ability to bind to or interact with a non-alpha-synuclein protein, such as a tau protein or an enzyme (e.g., a kinase). In some embodiments, neutralizing alpha-synuclein protein comprises preventing the aggregation and/or oligomerization of alpha-synuclein protein (e.g., in a brain of a subject), or inhibiting, reducing, or preventing the seeding, growth or spreading of alpha-synuclein oligomers and/or fibrils, for example in a brain of a subject, such as from one cell compartment to another, from one cell to another, or from one brain region to another.

The terms “spreading of alpha-synuclein protein” and “alpha-synuclein protein spreading” refer to transfer, diffusion or movement of alpha-synuclein protein, such as monomer, oligomer, fibril and/or combinations thereof, between cellular compartments, between two or more cells, or between two or more regions within the brain or CNS of a subject. Spreading alpha-synuclein protein may induce disease spreading between cells or brain regions, for example by transcytosis, endocytosis, membrane penetration, transynaptic transmission, or via membrane receptors, or by transfer between interconnected neurons or other cells within the brain of a subject. In certain embodiments, anti-alpha synuclein antibodies bind to and neutralize, inhibit or decrease alpha-synuclein protein spreading.

In some embodiments, alpha-synuclein protein spreading is continuous. As a non-limiting example, alpha-synuclein protein can be present at a particular site in the brain of a subject, and the size of that site can increase as alpha-synuclein protein spreads, i.e., without an associated increase in the number of sites. In other words, a site or area defined by the presence of alpha-synuclein protein can increase in surface area and/or volume as alpha-synuclein spreads continuously. In some embodiments, alpha-synuclein protein spreading is discontinuous. As a non-limiting example, alpha-synuclein protein can be present at a particular site in the brain of a subject, and as the spreading of alpha-synuclein protein progresses, the number of sites in which alpha-synuclein is present increases, but the size of the original site does not increase (though in some other embodiments, it may increase). In some embodiments, alpha-synuclein spreading is both continuous and discontinuous. As a non-limiting example, as alpha-synuclein protein progresses, the size of a particular site at which alpha-synuclein is present can increase, and the number of sites or loci can increase.

In some embodiments, alpha-synuclein protein spreading is associated with an increase in size (e.g., volume) of one more sites or loci at which alpha-synuclein is present, is associated with the spreading of alpha-synuclein beyond a particular structure or region of an organ (e.g., a brain of a subject), or a combination thereof. In some embodiments, alpha-synuclein protein spreading is associated with the onset, progression, or worsening of the signs or symptoms of a disease (e.g., a neurodegenerative disease such as a synucleinopathy or Parkinson's disease). As non-limiting examples, symptoms of a neurodegenerative disease include memory deficits, cognitive deficits, motor deficits, sensory deficits, and speech deficits. Alpha-synuclein protein spreading can refer to the spreading of oligomeric alpha-synuclein, monomeric alpha-synuclein, soluble alpha-synuclein, and/or alpha-synuclein fibrils. In particular embodiments, alpha-synuclein spreading refers to the spreading of a toxic form of alpha-synuclein. In some instances, the toxic form of alpha-synuclein is oligomeric alpha-synuclein.

In some embodiments, the term “broad spreading” refers to the spreading of human alpha-synuclein protein (e.g., a toxic form of human alpha-synuclein protein such as oligomeric human alpha-synuclein protein or human alpha-synuclein protein fibrils) beyond a single site, region, area, or structure in an organ (e.g., a brain of a subject). In some embodiments, the term refers to the spreading of human alpha-synuclein protein to a new site that is outside of a pre-existing region, area, or structure and is separate from (e.g., not in physical communication with) the pre-existing site.

In some embodiments, broad spreading is defined as when the size (e.g., volume) of a particular site or locus in which alpha-synuclein protein (e.g., a toxic form of alpha-synuclein protein (e.g., oligomeric alpha-synuclein protein)) is present exceeds a reference value. In some embodiments, broad spreading is defined as when the total amount of alpha-synuclein protein that is present in a region, area, or structure of an organ (e.g., a brain of a subject), or the total organ itself, is greater than a reference value. The reference value can be a specific value that is expressed in a particular unit (e.g., a unit of volume such as mm³), or more commonly, a range of values. In some embodiments, broad spreading is defined as when the size of a site or locus in which alpha-synuclein is present, or the total amount of alpha-synuclein that is present in a region, area, structure, or organ, is at least about 1.5-, 2-, 2.5-, 3-, 3.5-, 4-, 4.5-, 5-, 5.5-, 6-, 6.5-, 7-, 7.5-, 8-, 8.5-, 9-, 9.5-, 10-fold, or more greater than a reference value. The reference value can be derived from an individual before the occurrence of a disease (e.g., a neurodegenerative disease) or before the appearance of signs or symptoms of the disease, or can be derived from a population of unaffected individuals.

In some embodiments, broad spreading is associated with the onset and/or progression of one or more signs or symptoms of a disease (e.g., a neurodegenerative disease), examples of which are described herein.

The terms “seeding of alpha-synuclein protein” and “alpha-synuclein protein seeding” refer to processes in which soluble alpha-synuclein protein molecules are converted to aggregated alpha-synuclein protein (e.g., alpha-synuclein oligomers and/or fibrils). In certain embodiments, the conversion is catalyzed (e.g., is initiated, aided, made more efficient, or facilitated) by, for example, by the aggregated form(s) of alpha-synuclein protein. Depending on factors such as the concentration of alpha-synuclein monomers and the number or concentration of aggregated alpha-synuclein molecules that are present, seeding may favor the production of either alpha-synuclein fibrils or alpha-synuclein oligomers.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region, but does not contain a variable region of an antibody, as described herein.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The term “FcRn” refers to the neonatal Fc receptor. Binding of an Fc polypeptide to FcRn reduces clearance of the Fc polypeptide from serum and increases serum half-life of the Fc polypeptide or of a molecule comprising the Fc polypeptide, such as an antibody. The human FcRn protein is a heterodimer that is composed of a protein of about 50 kDa in size that is similar to a major histocompatibility (MHC) class I protein and a 132-microglobulin of about 15 kDa in size.

As used herein, a “FcRn binding site” refers to the region of an Fc polypeptide that binds to FcRn. In human IgG, the FcRn binding site, as numbered using the EU index, includes L251, M252, 1253, S254, R255, T256, M428, H433, N434, H435, and Y436. These positions correspond to positions 21 to 26, 198, and 203 to 206 of SEQ ID NO:64.

As used herein, a “native FcRn binding site” refers to a region of an Fc polypeptide that binds to FcRn and that has the same amino acid sequence as the region of a naturally occurring Fc polypeptide that binds to FcRn.

The term “cross-reacts,” as used herein, refers to the ability of an antibody variable region to bind to an antigen other than the antigen against which the antibody was raised or generated. In some embodiments, cross-reactivity refers to the ability of an antibody variable region to bind to an antigen from a different species than the species from which the antigen against which the antibody was raised. As a non-limiting example, an anti-alpha-synuclein antibody as described herein that is raised against a human alpha-synuclein protein can exhibit cross-reactivity with an alpha-synuclein protein from a different species (e.g., monkey).

The term “isolated,” as used with reference to a nucleic acid, protein (e.g., antibody), host cell, or vector, denotes that the nucleic acid, protein, host cell, or vector is essentially free of other cellular components with which it is associated in the natural state. In some embodiments, a composition of the present disclosure can be “isolated” in the sense that it is physically separated from and not comprised within a subject to whom the composition can be, was, or is to be administered. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid, protein (e.g., antibody), host cell, or vector is at least 85% pure, at least 90% pure, at least 95% pure, or at least 99% pure.

The term “amino acid” refers to naturally occurring and synthetic amino acids, as well as amino acid analogs and amino acid mimetics that function in a manner similar to the naturally occurring amino acids (e.g., analogs and mimetics such as homoserine, norleucine, methonine sulfoxide, methionine methyl sulfonium).

Naturally occurring amino acids are those encoded by the genetic code, as well as those amino acids that are later modified, e.g., hydroxyproline, γ-carboxyglutamate and O-phosphoserine. Naturally occurring a-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gin), serine (Ser), threonine (Thr), valine (Val), tryptophan (Trp), tyrosine (Tyr), and combinations thereof. Stereoisomers of a naturally occurring a-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i. e, two) or multimer (i.e., three or more) of single chain polypeptides. The single chain polypeptides of a protein may be joined by a covalent bond, e.g., a disulfide bond, or non-covalent interactions.

The terms “polynucleotide” and “nucleic acid” can be used interchangeably to refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The term “conservative substitution” or “conservative mutation” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide (or polynucleotide) sequences, refer to two or more sequences or subsequences that are the same or have a specified percentage of amino acid residues (or nucleotides), e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

For sequence comparison, e.g., of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two protein's sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:64, when the residue aligns with the amino acid in SEQ ID NO:64 when optimally aligned to SEQ ID NO:64. The polypeptide that is aligned to the reference sequence need not be the same length as the reference sequence.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In certain embodiments, the patient or subject is a human.

The terms “treatment,” “treating,” and the like are used herein to generally mean obtaining a desired pharmacologic and/or physiologic effect. “Treating” or “treatment” may refer to any indicia of success in the prophylaxis, treatment or amelioration of a neurodegenerative disease (e.g., a synucleinopathy), including any objective or subjective parameter such as abatement, remission, improvement in patient survival, increase in survival time or rate, diminishing of symptoms or making the disease more tolerable to the patient, slowing in the rate of disease spread or severity or of patient degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

As used herein, the term “synucleinopathy” refers to a pathological condition (e.g., a neurodegenerative disease) that is characterized by an abnormal accumulation of alpha-synuclein protein in neural tissue (e.g., brain tissue). Examples of synucleinopathies include Parkinson's disease, dementia with Lewy bodies, and multiple system atrophy. Synucleinopathies are typically characterized by the presence of Lewy bodies or Lewy neurites, which contain significant amounts of alpha-synuclein aggregates, including alpha-synuclein oligomers and/or fibrils. In some instances, synucleinopathies are characterized by the spreading of oligomers and/or fibrils of alpha-synuclein protein throughout the diseased tissue. The accumulation of alpha-synuclein protein results in, for example, mitochondrial dysfunction, ER-Golgi traffic dysfunction, inhibition of autophagy and proteasome pathways, and disruption of synaptic tissue, all of which can lead to cell death.

The term “pharmaceutically acceptable excipient” refers to a non-active pharmaceutical ingredient that is biologically or pharmacologically compatible for use in humans or animals, such as but not limited to a buffer, carrier, or preservative.

As used herein, a “therapeutic amount” or “therapeutically effective amount” or “effective amount” of an agent (e.g., an antibody as described herein) is an amount of the agent that treats, alleviates, abates, or reduces the severity of symptoms of a disease in a subject. A “therapeutic amount” or “therapeutically effective amount” or “effective amount” of an agent (e.g., an antibody as described herein) may improve patient survival, increase survival time or rate, diminish symptoms, make an injury, disease, or condition (e.g., a neurodegenerative disease) more tolerable, slow the rate of degeneration or decline, or improve a patient's physical or mental well-being.

The term “administer” refers to a method of delivering one or more agent, compound, or composition to the desired site of biological action. These methods include, but are not limited to, topical delivery, parenteral delivery, intravenous delivery, subcutaneous delivery, intradermal delivery, intramuscular delivery, intrathecal delivery, colonic delivery, rectal delivery, or intraperitoneal delivery. In one embodiment, an antibody as described herein is administered intravenously.

### III. Functional Characteristics of Anti-Alpha-Synuclein Antibodies

In one aspect, antibodies and antigen-binding portions of antibodies that specifically bind to an alpha-synuclein protein (e.g., human alpha-synuclein) are provided.

In some embodiments, the antibody or antigen-binding portion thereof specifically binds to monomeric and/or oligomeric alpha-synuclein. In some embodiments, the antibody or antigen-binding portion thereof specifically binds to monomeric alpha-synuclein. In some embodiments, the antibody or antigen-binding portion thereof specifically binds to oligomeric alpha-synuclein. In some embodiments, the antibody or antigen-binding portion thereof specifically binds to monomeric and oligomeric alpha-synuclein. In some embodiments, the antibody or antigen-binding portion thereof has a higher binding affinity for oligomeric human alpha-synuclein protein than for monomeric human alpha-synuclein protein. In some embodiments, the antibody or antigen-binding portion thereof has a higher binding affinity for monomeric human alpha-synuclein protein than for oligomeric human alpha-synuclein protein. In some embodiments, the antibody or antigen-binding portion thereof has about the same binding affinity for monomeric and oligomeric alpha-synuclein protein.

In some embodiments, the antibody or antigen-binding portion thereof specifically binds to soluble alpha-synuclein protein (e.g., human alpha-synuclein protein) and/or alpha-synuclein protein fibrils. In some embodiments, the antibody or antigen-binding portion thereof specifically binds to soluble alpha-synuclein. In some embodiments, the antibody or antigen-binding portion thereof specifically binds to alpha-synuclein fibrils. In some embodiments, the antibody or antigen-binding portion thereof specifically binds to soluble alpha-synuclein and alpha-synuclein fibrils.

In some embodiments, the antibody is a pan-alpha-synuclein antibody or antigen-binding portion thereof. In some embodiments, the pan-alpha-synuclein antibody or antigen-binding portion thereof specifically binds to monomeric human alpha-synuclein protein, oligomeric human alpha-synuclein protein, soluble human alpha-synuclein protein, human alpha-synuclein protein fibrils, and phosphorylated alpha-synuclein (e.g., pSer129).

In some embodiments, the antibody or antigen-binding portion thereof specifically binds to or immunodepletes at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9%, or more, of human alpha-synuclein protein (including soluble and/or insoluble alpha-synuclein) that is present in a sample.

In some embodiments, an anti-alpha-synuclein antibody or an antigen-binding portion thereof described herein specifically binds to or immunodepletes at least about 80% (e.g., about 80%, 85%, 90%, 91%, 92%, 93%, 94%, 95%, 96%, 97%, 98%, 99%, or more) of soluble human alpha-synuclein protein that is present in a sample. In some embodiments, the antibody or antigen-binding portion thereof specifically binds to or immunodepletes at least about 95% of soluble human alpha-synuclein protein that is present in a sample.

In some embodiments, the antibody or antigen-binding portion thereof specifically binds to or immunodepletes at least about 50% (e.g., about 50%, 55%, 60%, 65%, 70%, 75%, 80%, 85%, 90%, 91%, 92%, 93%, 94%, 95%, 96%, 97%, 98%, 99%, or more) of insoluble human alpha-synuclein protein that is present in a sample. In some embodiments, the antibody or antigen-binding portion thereof specifically binds to or immunodepletes at least about 70% of insoluble human alpha-synuclein protein that is present in a sample.

In some embodiments, an anti-alpha-synuclein antibody or an antigen-binding portion thereof described herein specifically binds to or immunodepletes at least about 70% (e.g., about 75%, 80%, 85%, 90%, 95%, 96%, 97%, 98%, 99%, 99.1%, 99.2%, 99.3%, 99.4%, 99.5%, 99.6%, 99.7%, 99.8%, 99.9%, or more) of soluble human alpha-synuclein protein and at least about 50% (e.g., about 50%, 55%, 60%, 65%, 70%, 75%, 80%, 85%, 90%, 95%, or more) of insoluble human alpha-synuclein protein that is present in a sample. In some embodiments, an anti-alpha-synuclein antibody or an antigen-binding portion thereof described herein specifically binds to and immunodepletes at least about 95% of soluble human alpha-synuclein protein and at least about 80% of insoluble human alpha-synuclein protein that is present in a sample.

In certain embodiments, the immunodepletion is determined by a decrease in an overall amount of alpha-synuclein protein (e.g., present in a sample) by comparison to a reference (e.g., a like sample that has not been contacted with an anti-alpha-synuclein antibody), or by a change in concentration of alpha-synuclein protein in a sample, or both.

Any suitable sample type can be used. In some embodiments, the sample is selected from the group consisting of an interstitial brain fluid (ISF) sample, a cerebrospinal fluid (CSF) sample, a brain tissue sample, a blood sample, and a combination thereof. In some embodiments, the sample is from a subject who has or is suspected of having Parkinson's Disease (PD). Methods for obtaining and preparing samples are known in the art and include those described herein. In certain embodiments, immunodepletion is determined using an ELISA assay, such as a Meso Scale Discovery (MSD) ELISA assay and/or an alpha-synuclein oligomer ELISA.

In certain embodiments, an anti-alpha-synuclein antibody or antigen-binding portion thereof is capable of reducing free alpha-synuclein in cerebrospinal fluid (CSF; e.g., in or from a human or non-human primate subject) by at least about 40% or at least about 50% relative to a pre-administration baseline level of CSF free alpha-synuclein for about 2, 4, 6, 8, 10, 12, 14, 16, 18, 20, or 22 days following a single intravenous dose of the antibody or antigen-binding portion at from about 10 mg/kg to about 30 mg/kg (e.g., including 10 mg/kg and 30 mg/kg, respectively). In certain embodiments, the antibody or antigen-binding portion thereof is capable of reducing free alpha-synuclein in cerebrospinal fluid (CSF; e.g., in a human or non-human primate subject) by at least about 50%, at least about 60%, at least about 70%, or at least about 80% relative to a pre-administration baseline level of CSF free alpha-synuclein for about 1, 2, or 3 days following a single intravenous dose of the antibody or antigen-binding portion at from about 10 mg/kg to about 30 mg/kg.

As a non-limiting example, in some embodiments, co-incubation of (i) the antibody or antigen-binding portion at a concentration of about 30 nM with (ii) a CSF sample obtained from a human subject diagnosed with or suspected of having PD (PD-CSF sample) reduces the amount of unbound soluble alpha-synuclein present in the PD-CSF sample by about 50% or more (e.g., about 55%, 60%, 65%, 70%, 75%, 80%, 85%, 90%, 95%, 96%, 97%, 98%, 99%, or more). In particular embodiments, the antibody or antigen-binding portion reduces the amount of unbound soluble alpha-synuclein present in the PD-CSF sample by at least about 60%.

In some embodiments, co-incubation of the antibody or antigen-binding portion with a CSF sample results in at least about a one-third-fold change (i.e., a reduction to about one-third or less of the previous concentration), a one-fourth change, a one-fifth-fold change, a one-sixth-fold change, one-seventh-fold change, one-eighth-fold change, a one-ninth-fold change, a one-tenth-fold change, or more, in the concentration of unbound soluble alpha-synuclein present in the sample.

In another aspect, antibodies and antigen-binding portions of antibodies that neutralize alpha-synuclein protein (e.g., human alpha-synuclein) are provided. In some embodiments, the antibody or antigen-binding portion thereof neutralizes human alpha-synuclein protein in a subject, e.g., in a brain of a subject. In some embodiments, the antibody or antigen-binding portion thereof neutralizes alpha-synuclein protein by preventing, reducing, or inhibiting alpha-synuclein oligomerization and/or aggregation. In some embodiments, the antibody of antigen-binding portion thereof prevents, reduces, or inhibits alpha-synuclein oligomerization and/or aggregation in a subject, e.g., in a brain of a subject. In some embodiments, the antibody or antigen-binding portion thereof prevents, reduces, or inhibits alpha-synuclein oligomerization. In some embodiments, the antibody or antigen-binding portion thereof prevents, reduces, or inhibits alpha-synuclein aggregation. In some embodiments, the antibody or antigen-binding portion thereof prevents, reduces, or inhibits alpha-synuclein oligomerization and aggregation.

In some embodiments, an anti-alpha-synuclein antibody (or antigen-binding portion thereof) prevents, reduces, or inhibits alpha-synuclein spreading by at least about 10%, 15%, 20%, 25%, 30%, 35%, 40%, 45%, 50%, 55%, 60%, 65%, 70%, 75%, 80%, 85%, 90%, or 95% (e.g., compared to the amount of alpha-synuclein spreading in the absence of the anti-alpha-synuclein antibody, such as in the absence of any exogenous anti-alpha-synuclein antibody). In some embodiments, an anti-alpha-synuclein antibody prevents, reduces, or inhibits alpha-synuclein oligomerization by at least about 10%, 15%, 20%, 25%, 30%, 35%, 40%, 45%, 50%, 55%, 60%, 65%, 70%, 75%, 80%, 85%, 90%, or 95% (e.g., compared to the amount of alpha-synuclein oligomerization in the absence of the anti-alpha-synuclein antibody). In some embodiments, an anti-alpha-synuclein antibody prevents, reduces, or inhibits alpha-synuclein aggregation by at least about 10%, 15%, 20%, 25%, 30%, 35%, 40%, 45%, 50%, 55%, 60%, 65%, 70%, 75%, 80%, 85%, 90%, or 95% (e.g., compared to the amount of alpha-synuclein aggregation in the absence of the anti-alpha-synuclein antibody). In some embodiments, an anti-alpha-synuclein antibody prevents, reduces, or inhibits alpha-synuclein seeding by at least about 10%, 15%, 20%, 25%, 30%, 35%, 40%, 45%, 50%, 55%, 60%, 65%, 70%, 75%, 80%, 85%, 90%, or 95% (e.g., compared to the amount of alpha-synuclein seeding in the absence of the anti-alpha-synuclein antibody). Methods for measuring alpha-synuclein seeding, spreading, oligomerization, and/or aggregation are known in the art.

In some embodiments, the antibody or antigen-binding portion thereof neutralizes alpha-synuclein protein by preventing, reducing, or inhibiting the seeding and/or spreading of alpha-synuclein protein oligomers and/or fibrils. In some embodiments, the antibody or antigen-binding portion thereof prevents, reduces, or inhibits the spreading of alpha-synuclein protein oligomers and/or fibrils in a subject, e.g., in the brain of a subject. In some embodiments, the antibody or antigen-binding portion thereof prevents, reduces, or inhibits the spreading of alpha-synuclein protein oligomers. In some embodiments, the antibody or antigen-binding portion thereof prevents, reduces, or inhibits the spreading of alpha-synuclein protein fibrils. In some embodiments, the antibody or antigen-binding portion thereof prevents, reduces, or inhibits the spreading of alpha-synuclein protein oligomers and fibrils. In some embodiments, the antibody or antigen-binding portion thereof prevents, reduces, or inhibits the seeding of alpha-synuclein protein (e.g., alpha-synuclein fibrils and/or oligomers); e.g., by at least about 10%, 15%, 20%, 25%, 30%, 35%, 40%, 45%, 50%, 55%, 60%, 65%, 70%, 75%, 80%, 85%, 90%, or 95% (e.g., compared to the amount of alpha-synuclein seeding in the absence of the anti-alpha-synuclein antibody).

In some embodiments, the antibody or antigen-binding portion thereof neutralizes monomeric human alpha-synuclein, oligomeric human alpha-synuclein, soluble human alpha-synuclein, human alpha-synuclein fibrils, and/or phosphorylated human alpha-synuclein (e.g., pSer129).

In some embodiments, neutralizing comprises binding to and/or immunodepleting at least about 80% (e.g., about 80%, 85%, 90%, 91%, 92%, 93%, 94%, 95%, 96%, 97%, 98%, 99%, or more) of soluble human alpha-synuclein protein that is present in a sample. In some embodiments, neutralizing comprises binding to and/or immunodepleting at least about 95% of soluble human alpha-synuclein protein that is present in a sample.

In some embodiments, neutralizing comprises binding to and/or immunodepleting at least about 50% (e.g., about 50%, 55%, 60%, 65%, 70%, 75%, 80%, 85%, 90%, 95%, or more) of insoluble human alpha-synuclein protein that is present in a sample. In some embodiments, neutralizing comprises binding to and/or immunodepleting at least about 70% of insoluble human alpha-synuclein protein that is present in a sample.

In some embodiments, administration of the antibody or antigen-binding portion thereof to a subject having an alpha-synucleinopathy (e.g., a human subject, or a non-human animal such as a primate or a mouse) reduces the amount of pSer129 alpha-synuclein present in the brain of the subject, optionally wherein the pSer129 alpha-synuclein is measured in a sample from one or more of: the hippocampus; the piriform cortex; the entorhinal cortex; and the brainstem; optionally wherein the amount of pSer129 alpha-synuclein is measured by immunohistochemistry (IHC).

In some embodiments, administration of the antibody or antigen-binding portion thereof to a subject having an alpha-synucleinopathy (e.g., a human subject, or a non-human animal such as a primate or a mouse) reduces the amount of free alpha-synuclein present in brain interstitial fluid (ISF) by about 10%, 20%, 30%, 40%, 50%, 60%, 70%, 80%, 90%, or more (e.g., as compared to a reference subject). In some embodiments, the antibody or antigen-binding portion thereof (e.g., administered via a single intravenous administration at a dose of about 10 mg/kg, about 30 mg/kg, or about 50 mg/kg) to a subject having an alpha-synucleinopathy reduces the amount of free alpha-synuclein present in brain interstitial fluid (ISF) by about 10%, 20%, 30%, 40%, 50%, 60%, 70%, 80%, 90%, or more for about 3, 6, 9, 21, or 24 hours.

In another aspect, antibodies and antigen-binding portions of antibodies that bind to alpha-synuclein protein (e.g., human alpha-synuclein) and thereby prevent or treat a neurodegenerative disease (e.g., a synucleinopathy or tauopathy) in a subject (e.g., a human subject) are provided. In some embodiments, the antibody or antigen-binding portion thereof prevents or treats a neurodegenerative disease that is characterized by the presence of Lewy bodies and/or Lewy neurites. In some embodiments, the antibody or antigen-binding portion thereof prevents or treats a neurodegenerative disease that is characterized by the presence of alpha-synuclein accumulation or aggregation (e.g., alpha-synuclein oligomers and/or fibrils).

In some embodiments, the antibody or antigen-binding portion prevents or treats a neurodegenerative disease that is selected from the group consisting of Parkinson's disease, dementia with Lewy bodies, multiple system atrophy, Huntington's disease, Alzheimer's disease, primary age-related tauopathy, hereditary late-onset Parkinson's disease, dysautonomia, traumatic brain injury (TBI), cerebrovascular dementia, progressive supranuclear palsy (PSP), frontotemporal dementia, frontotemporal dementia with parkinsonism linked to chromosome 17, argyrophilic grain dementia, amyotrophic lateral sclerosis/parkinsonism-dementia complex of Guam, corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, inclusion-body myositis, myotonic dystrophy, neurofibrillary tangle-predominant dementia, Niemann-Pick disease type C, pallido-ponto-nigral degeneration, Pick's disease, postencephalitic parkinsonism, prion protein cerebral amyloid angiopathy, progressive subcortical gliosis, subacute sclerosing panencephalitis, and tangle only dementia. In some embodiments, the antibody or antigen-binding portion thereof prevents or treats a synucleinopathy. In some embodiments, the synucleinopathy is selected from the group consisting of Parkinson's disease, dementia with Lewy bodies, and multiple system atrophy.

In some embodiments, the antibody or antigen-binding portion thereof slows the progression of symptoms of a neurodegenerative disease (e.g., in a subject). In some embodiments, the antibody or antigen-binding portion thereof causes the reversal of symptoms of a neurodegenerative disease. As non-limiting examples, symptoms of a neurodegenerative disease include memory deficits, cognitive deficits, motor deficits, sensory deficits, and speech deficits.

In some embodiments, an anti-alpha-synuclein antibody or antigen-binding portion thereof has a higher binding affinity for a human alpha-synuclein protein than for a rodent (e.g., rat or mouse) alpha-synuclein protein. In some embodiments, an anti-alpha-synuclein antibody or antigen-binding portion thereof has a higher binding affinity for a human alpha-synuclein protein than for a rat alpha-synuclein protein. In some embodiments, an anti-alpha-synuclein antibody or antigen-binding portion thereof does not bind to rat alpha-synuclein protein. In some embodiments, an anti-alpha-synuclein antibody or antigen-binding portion thereof has a binding affinity for a human alpha-synuclein protein that is at least about 10-fold, 25-fold, 50-fold, 100-fold, or more higher than a binding affinity for a rat alpha-synuclein protein (e.g., the K_{D} of the anti-alpha-synuclein antibody for a rat alpha-synuclein is at least about 10-fold, 25-fold, 50-fold, 100-fold, or more higher than the K_{D} of the anti-alpha-synuclein antibody for a human alpha-synuclein protein).

In some embodiments, an anti-alpha-synuclein antibody or antigen-binding portion thereof has a binding affinity for a human alpha-synuclein protein that is at least about 3-fold higher (e.g., at least about 3-fold, 4-fold, 5-fold, 6-fold, 7-fold, or 8-fold higher) than a binding affinity for a rat alpha-synuclein protein. In some embodiments, an anti-alpha-synuclein antibody or antigen-binding portion thereof has a binding affinity for a human alpha-synuclein protein that is at least about 8-fold higher than a binding affinity for a rat alpha-synuclein protein. In some embodiments, an anti-alpha-synuclein antibody or antigen-binding portion thereof has a binding affinity for a human alpha-synuclein protein that is between about 10-fold to about 100-fold higher than a binding affinity for a rat alpha-synuclein protein.

In some embodiments, an anti-alpha-synuclein antibody or antigen-binding portion thereof exhibits cross-reactivity with cynomolgus monkey (“cyno”) alpha-synuclein.

In some embodiments, an anti-alpha-synuclein antibody or antigen-binding portion thereof specifically binds to phosphorylated alpha-synuclein protein (e.g., phosphorylated human alpha-synuclein protein) and/or unphosphorylated alpha-synuclein protein (e.g., unphosphorylated human alpha-synuclein protein). In some embodiments, an anti-alpha-synuclein antibody or antigen-binding portion thereof specifically binds to phosphorylated alpha-synuclein protein and unphosphorylated alpha-synuclein protein. In some embodiments, the phosphorylated alpha-synuclein protein is phosphorylated at Ser129.

In some embodiments, an anti-alpha-synuclein antibody or antigen-binding portion thereof has about the same binding affinity for phosphorylated alpha-synuclein protein as for unphosphorylated alpha-synuclein protein. In some embodiments, an anti-alpha-synuclein antibody or antigen-binding portion thereof has a binding affinity for a phosphorylated alpha-synuclein protein that is less than about 10-fold (e.g., less than about 9-fold, 8-fold, 7-fold, 6-fold, 5-fold, 4-fold, 3-fold, 2.5-fold, 2.4-fold, 2.3-fold, 2.2-fold, 2.1-fold, 2-fold, 1.9-fold, 1.8-fold, 1.7-fold, 1.6-fold, 1.5-fold, 1.4-fold, 1.3-fold, 1.2-fold, or 1.1-fold) higher or lower than the binding affinity for an unphosphorylated alpha-synuclein protein (e.g., the anti-alpha-synuclein antibody has a K_{D} for an phosphorylated alpha-synuclein protein that is less than about 10-fold (e.g., less than about 9-fold, 8-fold, 7-fold, 6-fold, 5-fold, 4-fold, 3-fold, 2.5-fold, 2.4-fold, 2.3-fold, 2.2-fold, 2.1-fold, 2-fold, 1.9-fold, 1.8-fold, 1.7-fold, 1.6-fold, 1.5-fold, 1.4-fold, 1.3-fold, 1.2-fold, or 1.1-fold) lower or higher than the K_{D} for an unphosphorylated alpha-synuclein protein).

Methods for analyzing binding affinity, binding kinetics, and cross-reactivity are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA), flow cytometry, fluorescence-activated cell sorting (FACS), BioLayer interferometry (e.g., Octet™ (FortéBio, Inc., Menlo Park, Calif.)), and Western blot analysis. In some embodiments, ELISA is used to determine binding affinity and/or cross-reactivity. Methods for performing ELISA assays are known in the art, and are also described in the Examples section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

In some embodiments, an anti-alpha-synuclein antibody or antigen-binding portion thereof has a higher binding affinity for alpha-synuclein protein than for beta-synuclein protein and/or gamma-synuclein protein. In some embodiments, an anti-alpha-synuclein antibody or antigen-binding portion thereof has a higher binding affinity for alpha-synuclein protein than for beta-synuclein protein and gamma-synuclein protein. In some embodiments, an anti-alpha-synuclein antibody or antigen-binding portion thereof has a binding affinity for alpha-synuclein protein that is at least about 50-, 100-, 1,000-, 10,000-, or more, fold) than the binding affinity for beta-synuclein protein and/or gamma-synuclein protein. In some embodiments, the anti-alpha-synuclein antibody or antigen-binding portion does not bind to beta-synuclein protein and/or gamma-synuclein protein.

In some embodiments, an anti-alpha-synuclein antibody or antigen-binding portion thereof recognizes an epitope of human alpha-synuclein having, comprised within, or consisting of the sequence VKKDQLGKNEEGAPQEGILEDMPVDPDNEAYEMPSEEGYQDYEPEA (SEQ ID NO:58), which corresponds to residues 95-140 of SEQ ID NO:1. In some embodiments, an anti-alpha-synuclein antibody or antigen-binding portion thereof recognizes an epitope within residues 95-140 of SEQ ID NO:1. In some embodiments, the peptide sequence set forth in SEQ ID NO:58 is sufficient for binding to the anti-alpha-synuclein antibody.

In some embodiments, an anti-alpha-synuclein antibody or antigen-binding portion thereof recognizes an epitope of human alpha-synuclein having, comprised within, or consisting of the sequence ILEDMPVDPDNEAYEMPSEEGYQDYEPEA (SEQ ID NO:59), which corresponds to residues 112-140 of SEQ ID NO:1. In some embodiments, an anti-alpha-synuclein antibody or antigen-binding portion thereof recognizes an epitope within residues 112-140 of SEQ ID NO:1. In some embodiments, the peptide sequence set forth in SEQ ID NO:59 is sufficient for binding to the anti-alpha-synuclein antibody.

In some embodiments, an anti-alpha-synuclein antibody or antigen-binding portion thereof recognizes an epitope of human alpha-synuclein having, comprised within, or consisting of the sequence GILEDMPVDPDNEAY (SEQ ID NO:61), which corresponds to residues 111-125 of SEQ ID NO:1. In some embodiments, an anti-alpha-synuclein antibody or antigen-binding portion thereof recognizes an epitope comprised within residues 111-125 of SEQ ID NO:1. In some embodiments, the peptide sequence set forth in SEQ ID NO:61 is sufficient for binding to the anti-alpha-synuclein antibody.

In some embodiments, an anti-alpha-synuclein antibody or antigen-binding portion thereof recognizes an epitope of human alpha-synuclein having, comprised within, or consisting of the sequence MPVDPDNEAYEMPSE (SEQ ID NO:62), which corresponds to residues 116-130 of SEQ ID NO:1. In some embodiments, an anti-alpha-synuclein antibody or antigen-binding portion thereof recognizes an epitope within residues 116-130 of SEQ ID NO:1. In some embodiments, the peptide sequence set forth in SEQ ID NO:62 is sufficient for binding to the anti-alpha-synuclein antibody.

In some embodiments, an anti-alpha-synuclein antibody or antigen-binding portion thereof recognizes an epitope within residues 111-130 of SEQ ID NO:1. In some embodiments, an anti-alpha-synuclein antibody or antigen-binding portion thereof recognizes an epitope of human alpha-synuclein within residues 111-125 of SEQ ID NO:1 or recognizes an epitope within residues 116-130 of SEQ ID NO:1. In some embodiments, an anti-alpha-synuclein antibody or antigen-binding portion thereof recognizes an epitope of human alpha-synuclein comprised of, or comprised within, residues 111-130 of SEQ ID NO:1. In some embodiments, an anti-alpha-synuclein antibody or antigen-binding portion thereof recognizes an epitope of human alpha-synuclein comprising residues 111-125 of SEQ ID NO:1 or recognizes an epitope comprised of, or comprised within, residues 116-130 of SEQ ID NO:1.

In some embodiments, an anti-alpha-synuclein antibody or antigen-binding portion thereof recognizes an epitope of human alpha-synuclein having, comprised within, or consisting of the sequence DPDNEAY (SEQ ID NO:86), which corresponds to residues 119-125 of SEQ ID NO:1. In some embodiments, an anti-alpha-synuclein antibody or antigen-binding portion thereof recognizes an epitope within residues 119-125 of SEQ ID NO:1. In some embodiments, the peptide sequence set forth in SEQ ID NO:86 is sufficient for binding to the anti-alpha-synuclein antibody.

In some embodiments, binding to the human alpha-synuclein protein at residues 119, 122, and 125 of SEQ ID NO:1 is required for the antibody or antigen-binding portion thereof to specifically bind the alpha-synuclein protein. In some embodiments, binding to the human alpha-synuclein protein at residues 119, 122, 124, and 125 of SEQ ID NO:1 is required for the antibody or antigen-binding portion thereof to specifically bind the alpha-synuclein protein. In some embodiments, (a) alanine mutagenesis at any one or more of residues 119, 122, 124, and 125 of SEQ ID NO:1, and/or proline mutagenesis at residue 124 of SEQ ID NO:1, reduces or abrogates binding by the antibody or antigen-binding portion thereof to the human alpha-synuclein protein, and/or (b) glycine mutagenesis at residue 124 of SEQ ID NO:1 does not reduce or abrogate binding by the antibody or antigen-binding portion thereof to the human alpha-synuclein protein, and/or (c) alanine mutagenesis at any of residues 120, 121, and 123 of SEQ ID NO:1 does not reduce or abrogate binding by the antibody or antigen-binding portion thereof to the human alpha-synuclein protein. In some embodiments, reduction or abrogation in binding by the antibody or antigen-binding portion thereof to the human alpha-synuclein protein, or lack thereof, is determined by ELISA, with an absorbance signal quantified at 450 nM.

In some embodiments, an anti-alpha-synuclein antibody or antigen-binding portion thereof that recognizes an epitope comprised within residues 119-125 of SEQ ID NO:1, and/or requires binding to residues 119, 122, 125, and optionally 124, of SEQ ID NO.:1, in order to specifically bind an alpha-synuclein protein, has a higher affinity for human alpha-synuclein protein than for rat alpha-synuclein protein (e.g., at least an about 3-fold, 4-fold, 5-fold, or 8-fold higher affinity for human alpha-synuclein than for rat alpha-synuclein).

In some embodiments, an anti-alpha-synuclein antibody or antigen-binding portion thereof that recognizes an epitope comprised within residues 119-125 of SEQ ID NO:1, and/or requires binding to residues 119, 122, 125, and optionally 124, of SEQ ID NO.:1, in order to specifically bind an alpha-synuclein protein, specifically binds to phosphorylated anti-synuclein protein (e.g., pSer129) and/or unphosphorylated alpha-synuclein protein. In some embodiments, the anti-alpha-synuclein antibody or antigen-binding portion thereof specifically binds to both phosphorylated and unphosphorylated alpha-synuclein protein (e.g., with about the same affinity). In some embodiments, an anti-alpha-synuclein antibody or antigen-binding portion thereof that recognizes an epitope comprised within residues 119-125 of SEQ ID NO:1, and/or requires binding to residues 119, 122, 125, and optionally 124, in order to specifically bind an alpha-synuclein protein, is a pan-alpha synuclein antibody or antigen-binding portion thereof (e.g., one that specifically binds to monomeric human alpha-synuclein protein, oligomeric human alpha-synuclein protein, soluble human alpha-synuclein protein, human alpha-synuclein protein fibrils, and human alpha-synuclein protein that is phosphorylated at Ser129 (pSer129)). In some embodiments, the pan-alpha-synuclein antibody or antigen-binding portion thereof specifically binds to each of monomeric, oligomeric, fibril, and pSer129 human alpha-synuclein with high affinity, for example with affinity of about 20 pM, or less, or about 16 pM, or less, as measured by KinExA®.

In some embodiments, an anti-alpha-synuclein antibody binds to contiguous residues of SEQ ID NO:1. In some embodiments, an anti-alpha-synuclein antibody binds to non-contiguous residues of SEQ ID NO:1. In some embodiments, an anti-alpha-synuclein antibody binds to both contiguous and non-contiguous residues of SEQ ID NO:1.

In certain embodiments, the antibody or antigen-binding portion thereof has a PK clearance rate (mL/day/kg in a subject administered the antibody or antigen-binding portion (e.g., via intravenous injection at a dose from about 10 mg/kg to about 50 mg/kg)) of less than 25, less than 20, less than 15, less than 10, or less than 6, or about 5.2, or about 4.5.

Anti-Alpha-Synuclein Antibody Sequences and Humanized and Affinity-Matured Anti-Alpha-Synuclein Antibodies.

In one aspect, the present disclosure provides humanized and affinity-matured anti-alpha-synuclein antibodies, including humanized and affinity matured antibodies as described herein.

In some embodiments, an antibody or antigen-binding portion thereof that specifically binds to a human alpha-synuclein protein comprises a light chain sequence, or a portion thereof, and/or a heavy chain sequence, or a portion thereof, derived from any of the following exemplary antibodies described herein: Clone 20H4, Clone 1H5, Clone 22D11, clone 16F3, Clone 18B4, and Clone 22E11. The amino acid sequences of the light chain variable region (VL) and heavy chain variable region (VH) of these and other exemplary anti-alpha-synuclein antibodies described herein are set forth in Table 12 below.

In some embodiments, an anti-alpha-synuclein antibody comprises a heavy chain variable region comprising an amino acid sequence that has at least 90% sequence identity (e.g., at least 90%, at least 91%, at least 92%, at least 93%, at least 94%, at least 95%, at least 96%, at least 97%, at least 98%, or at least 99% sequence identity) to any one of SEQ ID NOs:17-22, 144-166, 222, 223, and 229. In some embodiments, an anti-alpha-synuclein antibody comprises a heavy chain variable region comprising the amino acid sequence of any one of SEQ ID NOs:17-22, 144-166, 222, 223, and 229. In some embodiments, a heavy chain variable region sequence having at least 90% sequence identity to a reference sequence (e.g., SEQ ID NOs:17-22, 144-166, 222, 223, and 229) contains one, two, three, four, five, six, seven, eight, nine, ten or more substitutions (e.g., conservative substitutions), insertions, or deletions relative to the reference sequence but retains the ability to specifically bind to a human alpha-synuclein protein and recognize one or more epitopes as described herein. In some embodiments, a heavy chain variable region contains one, two, or three substitutions (e.g., conservative substitutions) in any one of SEQ ID NOs:17-22, 144-166, 222, 223, and 229. In some embodiments, substitutions, insertions, and/or deletions relative to a reference variable region sequence may be present only in one or more framework region.

In some embodiments, a heavy chain variable region comprises an amino acid sequence having at least 80% identity (e.g., at least 80%, at least 85%, at least 90%, at least 91%, at least 92%, at least 93%, at least 94%, at least 95%, at least 96%, at least 97%, at least 98%, or at least 99% sequence identity) to any one of SEQ ID NOs: 17-22, 144-166, 222, 223, and 229, provided that the heavy chain variable region comprises CDRH1, CDRH2, and/or CDRH3 according to SEQ ID NO: 17-22, 144-166, 222, 223, or 229, respectively. In other words, in certain embodiments, a heavy chain variable region can comprise one or more CDR according to any one of reference heavy chain variable region SEQ ID NOs:17-22, 144-166, 222, 223, and 229, but can have differences in framework sequence, such that the heavy chain variable region has at least 80% identity to the reference heavy chain variable region.

In some embodiments, an anti-alpha-synuclein antibody comprises a light chain variable region comprising an amino acid sequence that has at least 90% sequence identity (e.g., at least 90%, at least 91%, at least 92%, at least 93%, at least 94%, at least 95%, at least 96%, at least 97%, at least 98%, or at least 99% sequence identity) to any one of SEQ ID NOs:37-42, 167-187, 200-213, and 224. In some embodiments, an anti-alpha-synuclein antibody comprises a light chain variable region comprising the amino acid sequence of any one of SEQ ID NOs:37-42, 167-187, 200-213, and 224. In some embodiments, a light chain variable region sequence having at least 90% sequence identity to a reference sequence (e.g., SEQ ID NOs:37-42, 167-187, 200-213, and 224) contains one, two, three, four, five, six, seven, eight, nine, ten or more substitutions (e.g., conservative substitutions), insertions, or deletions relative to the reference sequence but retains the ability to bind to human alpha-synuclein and recognize one or more epitopes as described herein. In some embodiments, a light chain variable region contains one, two, or three substitutions (e.g., conservative substitutions) in any one of SEQ ID NOs:37-42, 167-187, 200-213, and 224.

In some embodiments, a light chain variable region sequence comprises an amino acid sequence having at least 80% identity (e.g., at least 80%, at least 85%, at least 90%, at least 91%, at least 92%, at least 93%, at least 94%, at least 95%, at least 96%, at least 97%, at least 98%, or at least 99% sequence identity) to any one of SEQ ID NOs:37-42, 167-187, 200-213, and 224, provided that the light chain variable region comprises CDRL1, CDRL2, and/or CDRL3 according to SEQ ID NOs:37-42, 167-187, 200-213, or 224, respectively. In other words, in certain embodiments, a light chain variable region can comprise one or more CDR according to any one of reference light chain variable region SEQ ID NOs:37-42, 167-187, 200-213, and 224, but can have differences in framework sequence, such that the light chain variable region has at least 80% identity to the reference light chain variable region.

In some embodiments, an anti-alpha-synuclein antibody comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:17-22, 144-166, 222, 223, and 229 and further comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:37-42, 167-187, 200-213, and 224. In certain further embodiments, the antibody comprises one, two, or three CDRs as set forth in SEQ ID NO.: 17-22, 144-166, 222, 223, 229, 37-42, 167-187, 200-213, or 224, respectively. In some embodiments, an anti-alpha-synuclein antibody comprises a heavy chain variable region comprising the amino acid sequence of any one of SEQ ID NOs:17-22, 144-166, 222, 223, and 229 and further comprises a light chain variable region comprising the amino acid sequence of any one of SEQ ID NOs:37-42, 167-187, 200-213, and 224.

In some embodiments, an anti-alpha-synuclein antibody comprises one or more CDRs selected from the group consisting of: (a) a heavy chain CDR1 having at least 90% sequence identity to the amino acid sequence of any one of SEQ ID NOs:2-7, 43, 87, and 97-104 or having up to two amino acid substitutions relative to the amino acid sequence of any one of SEQ ID NOs:2-7, 43, 87, and 97-104; (b) a heavy chain CDR2 having at least 90% sequence identity to the amino acid sequence of any one of SEQ ID NOs:8-10, 44-45, 88-89, 105-112, and 188, or having up to two amino acid substitutions relative to the amino acid sequence of any one of SEQ ID NOs:8-10, 44-45, 88-89, 105-112, and 188; (c) a heavy chain CDR3 having at least 90% sequence identity to the amino acid sequence of any one of SEQ ID NOs:11-16, 46, 90, and 113-121 or having up to two amino acid substitutions relative to the amino acid sequence of any one of SEQ ID NOs:11-16, 46, 90, and 113-121; (d) a light chain CDR1 having at least 90% sequence identity to the amino acid sequence of any one of SEQ ID NOs:23-26, 47-48, 91-92, and 122-125 or having up to two amino acid substitutions relative to the amino acid sequence of any one of SEQ ID NOs:23-26, 47-48, 91-92, 122, and 123; (e) a light chain CDR2 having at least 90% sequence identity to the amino acid sequence of any one of SEQ ID NOs:27-30, 49-50, 93-94, and 126-130 or having up to two amino acid substitutions relative to the amino acid sequence of any one of SEQ ID NOs:27-30, 49-50, 93-94, and 126-130; and (f) a light chain CDR3 having at least 90% sequence identity to the amino acid sequence of any one of SEQ ID NOs:31-36, 51-52, 95-96, and 131-143 or having up to two amino acid substitutions relative to the amino acid sequence of any one of SEQ ID NOs:31-36, 51-52, 95-96, and 131-143.

In some embodiments, an anti-alpha-synuclein antibody comprises two, three, four, five, or all six of (a)-(f) as in the immediately preceding paragraph. In some embodiments, an anti-alpha-synuclein antibody comprises the heavy chain CDR1 of (a), the heavy chain CDR2 of (b), and the heavy chain CDR3 of (c). In some embodiments, an anti-alpha-synuclein antibody comprises the light chain CDR1 of (d), the light chain CDR2 of (e), and the light chain CDR3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions. In some embodiments, the up to two amino acid substitutions replace or remove one or more sequence liability (if present), such as, e.g., residues that are susceptible to chemical modification (e.g., asparagine deamidation motifs (NG), aspartic acid isomerization motifs (DS)), and/or potential oxidation residues (tryptophan (W) and methionine (M)). Such sequence liabilities can be addressed by making conservative amino acid substitutions and/or substitutions for germline-encoded residues.

In some embodiments, an anti-alpha-synuclein antibody comprises one or more CDRs selected from the group consisting of: (a) a heavy chain CDR1 comprising the amino acid sequence of any one of SEQ ID NOs:2-7, 43, 87, and 97-104; (b) a heavy chain CDR2 comprising the amino acid sequence of any one of SEQ ID NOs:8-10, 44-45, 88-89, 105-112, and 188; (c) a heavy chain CDR3 comprising the amino acid sequence of any one of SEQ ID NOs:11-16, 46, 90, and 113-121; (d) a light chain CDR1 comprising the amino acid sequence of any one of SEQ ID NOs:23-26, 47-48, 91-92, 122-125 and 214-218; (e) a light chain CDR2 comprising the amino acid sequence of any one of SEQ ID NOs:27-30, 49-50, 93-94, and 126-130; and (f) a light chain CDR3 comprising the amino acid sequence of any one of SEQ ID NOs:31-36, 51-52, 95-96, and 131-143.

In some embodiments, an anti-alpha-synuclein antibody comprises two, three, four, five, or all six of (a)-(f) as in the immediately preceding paragraph. In some embodiments, an anti-alpha-synuclein antibody comprises the heavy chain CDR1 of (a), the heavy chain CDR2 of (b), and the heavy chain CDR3 of (c). In some embodiments, an anti-alpha-synuclein antibody comprises the light chain CDR1 of (d), the light chain CDR2 of (e), and the light chain CDR3 of (f).

In some embodiments, an anti-alpha-synuclein antibody comprises one or more sequences that are variants of one or more consensus sequences or are encompassed by one or more consensus sequences. Consensus sequences can be identified by aligning heavy chain or light chain sequences (e.g., CDRs) for antibodies that bind to the same or similar (e.g., overlapping) epitopes to determine conserved amino acids or motifs (i.e., where alteration in sequences may alter protein function) and regions where variation occurs in alignment of sequences (i.e., where variation of sequence is not likely to significantly affect protein function). Exemplary consensus sequences include SEQ ID NOs:43-52, 87-96, 103-104, 111-112, 120-121, 124-125, 129-130, 142-143, and 214-218. In the consensus sequences of SEQ ID NOs:43-52, 103, 111, 120, 124, 129, 142, and 214, the capitalized letter represents an amino acid residue that is absolutely conserved among the aligned sequences (e.g., aligned CDR sequences), while “x” represents an amino acid residue that is not absolutely conserved among the aligned sequences. While “x” can be any amino acid, it will be appreciated that when selecting an amino acid to insert at a position marked by an “x” that in some embodiments, the amino acid is selected from those amino acids found at the corresponding position in the aligned sequences.

In some embodiments, the antibody comprises a heavy chain CDR1 sequence having the consensus sequence GFxFxxxAxx (SEQ ID NO:43). In some embodiments, the heavy chain CDR1 consensus sequence comprises the sequence GF[T/R/K/N]F[S/N/T][D/N/S/T/K/R][A/G/F]A[M/I][Y/N/A] (SEQ ID NO:87).

In some embodiments, the antibody comprises a heavy chain CDR2 sequence having the consensus sequence RIRTKPNNYATYxADSVxG (SEQ ID NO:44). In some embodiments, the heavy chain CDR2 consensus sequence comprises the sequence RIRTKPNNYATY[Y/H]ADSV[K/R]G (SEQ ID NO:88).

In some embodiments, the antibody comprises a heavy chain CDR2 sequence having the consensus sequence TYxxDSVxG (SEQ ID NO:45). In some embodiments, the heavy chain CDR2 consensus sequence comprises the sequence TY[Y/H][A/P]DSV[K/R]G (SEQ ID NO:89).

In some embodiments, the antibody comprises a heavy chain CDR3 sequence having the consensus sequence xADxxxxDY (SEQ ID NO:46). In some embodiments, the heavy chain CDR3 consensus sequence comprises the sequence [T/S]AD[M/A/F][A/S/H][T/R/Y][F/G]DY (SEQ ID NO:90).

In some embodiments, the antibody comprises a light chain CDR1 sequence having the consensus sequence KAxQxINxxLD (SEQ ID NO:47). In some embodiments, the light chain CDR1 consensus sequence comprises the sequence KA[G/S]Q[N/I]IN[K/N/Q][N/I]LD (SEQ ID NO:91).

In some embodiments, the antibody comprises a light chain CDR1 sequence having the consensus sequence RSSQSLxHxNGxTYLx (SEQ ID NO:48). In some embodiments, the light chain CDR1 consensus sequence comprises the sequence RSSQSL[L/V]H[S/N]NG[N/I]TYL[H/S] (SEQ ID NO:92).

In some embodiments, the antibody comprises a light chain CDR2 sequence having the consensus sequence xTxxLxT (SEQ ID NO:49). In some embodiments, the light chain CDR2 consensus sequence comprises the sequence [F/S/R]T[N/K][N/K]L[Q/K]T (SEQ ID NO:93).

In some embodiments, the antibody comprises a light chain CDR2 sequence having the consensus sequence xVSxxxS (SEQ ID NO:50). In some embodiments, the light chain CDR2 consensus sequence comprises the sequence [L/K]VS[R/N][L/R][E/F]S (SEQ ID NO:94).

In some embodiments, the antibody comprises a light chain CDR3 sequence having the consensus sequence YQxxxxxT (SEQ ID NO:51). In some embodiments, the light chain CDR3 consensus sequence comprises the sequence YQ[Y/D/F][N/K/T][S/N/R/K/Y/L/F][G/A][W/F]T (SEQ ID NO:95).

In some embodiments, the antibody comprises a light chain CDR3 sequence having the consensus sequence xQGTxxPxT (SEQ ID NO:52). In some embodiments, the light chain CDR3 consensus sequence comprises the sequence [V/G]QGT[H/Q][A/Y]P[F/L/Y]T (SEQ ID NO:96).

In some embodiments, the antibody comprises a heavy chain CDR1 sequence having the consensus sequence GFxFxxAAMY (SEQ ID NO:103). In some embodiments, the heavy chain CDR1 consensus sequence comprises the sequence GF[T/R/K/N]F[S/N][N/K/R]AAMY (SEQ ID NO:104).

In some embodiments, the antibody comprises a heavy chain CDR2 sequence having the consensus sequence RIRTxxxNxATYHADSVKG (SEQ ID NO:111). In some embodiments, the heavy chain CDR2 consensus sequence comprises the sequence RIRT[K/Q/E][P/R][N/K]N[Y/F]ATYHADSVKG (SEQ ID NO:112).

In some embodiments, the antibody comprises a heavy chain CDR3 sequence having the consensus sequence xAxxxxGDY (SEQ ID NO:120). In some embodiments, the heavy chain CDR3 consensus sequence comprises the sequence [T/A/S]A[D/N/A][M/F][A/R][T/K/R]GDY (SEQ ID NO:121).

In some embodiments, the antibody comprises a light chain CDR1 sequence having the consensus sequence KASQxINxxLD (SEQ ID NO:124). In some embodiments, the light chain CDR1 consensus sequence comprises the sequence KASQ[N/I]IN[K/N/Q][N/I]LD (SEQ ID NO:125).

In some embodiments, the antibody comprises a light chain CDR1 sequence having the consensus sequence KAxxxxxxxLx (SEQ ID NO:214). In some embodiments, the light chain CDR1 consensus sequence comprises the sequence KA[S/G][Q/A][N/I/G][I/N][N/S][A/D/K/N/Q][N/I/Q]L[D/A] (SEQ ID NO:215). In some embodiments, the light chain CDR1 consensus sequence comprises the sequence KAS[Q/A]NNN[W/E/K][N/Q]LD (SEQ ID NO:216). In some embodiments, the light chain CDR1 consensus sequence comprises the sequence KAS[Q/A]NIN[W/E/K][N/Q]LD (SEQ ID NO:217).

In some embodiments, the antibody comprises a light chain CDR2 sequence having the consensus sequence xTxxLxT (SEQ ID NO:129). In some embodiments, the light chain CDR2 consensus sequence comprises the sequence [S/R]T[N/K][N/K]L[Q/K]T (SEQ ID NO:130).

In some embodiments, the antibody comprises a light chain CDR3 sequence having the consensus sequence YQxKxxWT (SEQ ID NO:142). In some embodiments, the light chain CDR3 consensus sequence comprises the sequence YQ[Y/D/F]K[S/N/R/K/Y/L/F][A/G]WT (SEQ ID NO:143).

In certain embodiments, an anti-alpha-synuclein antibody or antigen-binding portion of the present disclosure comprises a heavy chain CDR1 comprising the sequence set forth in SEQ ID NO: 3, a heavy chain CDR2 comprising the sequence set forth in SEQ ID NO:9, a heavy chain CDR3 comprising the sequence set forth in SEQ ID NO:12, a light chain CDR1 comprising the sequence set forth in SEQ ID NO:122, a light chain CDR2 comprising the sequence set forth in SEQ ID NO:128, and a light chain CDR3 comprising the sequence set forth in SEQ ID NO:32.

Anti-Alpha-Synuclein Antibodies that Recognize an Epitope within Residues 111-130

In some embodiments, an anti-alpha-synuclein antibody recognizes an epitope within residues 111-130 of SEQ ID NO:1. In some embodiments, an anti-alpha-synuclein antibody recognizes an epitope within residues 111-125 of SEQ ID NO:1 or an epitope within residues 116-130 of SEQ ID NO:1. In some embodiments, an anti-alpha-synuclein antibody recognizes an epitope within residues 119-125 of SEQ ID NO:1. In some embodiments, binding to the human alpha-synuclein protein at residues 119, 122, and 125 of SEQ ID NO:1 is required in order for the anti-alpha-synuclein antibody to specifically bind the alpha-synuclein protein. In some embodiments, binding to the human alpha-synuclein protein at residues 119, 122, 124, and 125 of SEQ ID NO:1 is required in order for the anti-alpha-synuclein antibody to specifically bind the alpha-synuclein protein. In some embodiments, the antibody comprises one or more complementarity determining region (CDR), heavy chain variable region, and/or light chain variable regions as described herein (e.g., as described in Table 12 below).

20H4

In some embodiments, an anti-alpha-synuclein antibody or antigen-binding portion comprises one or more CDRs selected from the group consisting of: (a) a heavy chain CDR1 having at least 90% (e.g., 90%, 91%, 92%, 93$, 94%, 95%, 96%, 97%, 98%, 99%, or 100%) sequence identity to the amino acid sequence of anyone of SEQ ID NOs:3 and 97-102 or having up to two amino acid substitutions relative to the amino acid sequence of any one of SEQ ID NOs:3 and 97-102; (b) a heavy chain CDR2 having at least 90% sequence identity to the amino acid sequence of any one of SEQ ID NOs:9 and 105-110 or having up to two amino acid substitutions relative to the amino acid sequence of anyone of SEQ ID NOs:9 and 105-110; (c) a heavy chain CDR3 having at least 90% sequence identity to the amino acid sequence of any one of SEQ ID NOs:12 and 113-119 or having up to two amino acid substitutions relative to the amino acid sequence of any one of SEQ ID NOs:12 and 113-119; (d) a light chain CDR1 having at least 90% sequence identity to the amino acid sequence of any one of SEQ ID NOs:24 and 122-123 or having up to two amino acid substitutions relative to the amino acid sequence of any one of SEQ ID NOs:24 and 122-123; (e) a light chain CDR2 having at least 90% sequence identity to the amino acid sequence of any one of SEQ ID NOs:28 and 126-128 or having up to two amino acid substitutions relative to the amino acid sequence of any one of SEQ ID NOs:28 and 126-128; and (f) a light chain CDR3 having at least 90% sequence identity to the amino acid sequence of any one of SEQ ID NOs:32 and 131-141 or having up to two amino acid substitutions relative to the amino acid sequence of any one of SEQ ID NOs:32 and 131-141.

In some embodiments, an anti-alpha-synuclein antibody or antigen-binding portion comprises two, three, four, five, or all six of (a)-(f) above. In some embodiments, an anti-alpha-synuclein antibody comprises the heavy chain CDR1 of (a), the heavy chain CDR2 of (b), and the heavy chain CDR3 of (c). In some embodiments, an anti-alpha-synuclein antibody or antigen-binding portion comprises the light chain CDR1 of (d), the light chain CDR2 of (e), and the light chain CDR3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, an anti-alpha-synuclein antibody or antigen-binding portion comprises one or more CDRs selected from the group consisting of: (a) a heavy chain CDR1 comprising the amino acid sequence of any one of SEQ ID NOs:3 and 97-102; (b) a heavy chain CDR2 comprising the amino acid sequence of any one of SEQ ID NOs:9 and 105-110; (c) a heavy chain CDR3 comprising the amino acid sequence of any one of SEQ ID NOs:12 and 113-119; (d) a light chain CDR1 comprising the amino acid sequence of SEQ ID NOs:24 and 122-123; (e) a light chain CDR2 comprising the amino acid sequence of any one of SEQ ID NOs:28 and 126-128; and (f) a light chain CDR3 comprising the amino acid sequence of any one of SEQ ID NOs:32 and 131-141.

In some embodiments, an anti-alpha-synuclein antibody or antigen-binding portion comprises two, three, four, five, or all six of (a)-(f) above. In some embodiments, an anti-alpha-synuclein antibody or antigen-binding portion comprises the heavy chain CDR1 of (a), the heavy chain CDR2 of (b), and the heavy chain CDR3 of (c). In some embodiments, an anti-alpha-synuclein antibody or antigen-binding portion comprises the light chain CDR1 of (d), the light chain CDR2 of (e), and the light chain CDR3 of (f).

In some embodiments, an anti-alpha-synuclein antibody or antigen-binding portion comprises a heavy chain CDR1 sequence comprising the amino acid sequence of any one of SEQ ID NOs:3 and 97-102, a heavy chain CDR2 sequence comprising the amino acid sequence of any one of SEQ ID NOs:9 and 105-110, and a heavy chain CDR3 sequence comprising the amino acid sequence of any one of SEQ ID NOs:12 and 113-119. In some embodiments, an anti-alpha-synuclein antibody or antigen-binding portion comprises a light chain CDR1 sequence comprising the amino acid sequence of any one of SEQ ID NOs:24 and 122-123, a light chain CDR2 sequence comprising the amino acid sequence of any one of SEQ ID NOs:28 and 126-128, and a light chain CDR3 sequence comprising the amino acid sequence of any one of SEQ ID NOs:32 and 131-141.

In some embodiments, an anti-alpha-synuclein antibody or antigen-binding portion comprises a heavy chain CDR1 sequence comprising the amino acid sequence of any one of SEQ ID NOs:3 and 97-102, a heavy chain CDR2 sequence comprising the amino acid sequence of any one of SEQ ID NOs:9 and 105-110, a heavy chain CDR3 sequence comprising the amino acid sequence of any one of SEQ ID NOs:12 and 113-119, a light chain CDR1 sequence comprising the amino acid sequence of any one of SEQ ID NOs:24 and 122-123, a light chain CDR2 sequence comprising the amino acid sequence of any one of SEQ ID NOs:28 and 126-128, and a light chain CDR3 sequence comprising the amino acid sequence of any one of SEQ ID NOs:32 and 131-141.

In some embodiments, the anti-alpha-synuclein antibody or antigen-binding portion comprises a heavy chain CDR1 comprising the amino acid sequence of SEQ ID NO:3, a heavy chain CDR2 comprising the amino acid sequence of SEQ ID NO:9, a heavy chain CDR3 comprising the amino acid sequence of SEQ ID NO:12, a light chain CDR1 comprising the amino acid sequence of SEQ ID NO:122, a light chain CDR2 comprising the amino acid sequence of SEQ ID NO:128, and a light chain CDR3 comprising the amino acid sequence of SEQ ID NO: 32. In some embodiments, the anti-alpha-synuclein antibody or antigen-binding portion comprises a heavy chain CDR1 comprising the amino acid sequence of SEQ ID NO:3, a heavy chain CDR2 comprising the amino acid sequence of SEQ ID NO:9, a heavy chain CDR3 comprising the amino acid sequence of SEQ ID NO:12, a light chain CDR1 comprising the amino acid sequence of SEQ ID NO:24, a light chain CDR2 comprising the amino acid sequence of SEQ ID NO:128, and a light chain CDR3 comprising the amino acid sequence of SEQ ID NO: 32. In some embodiments, the anti-alpha-synuclein antibody or antigen-binding portion comprises a heavy chain CDR1 comprising the amino acid sequence of SEQ ID NO:3, a heavy chain CDR2 comprising the amino acid sequence of SEQ ID NO:9, a heavy chain CDR3 comprising the amino acid sequence of SEQ ID NO:12, a light chain CDR1 comprising the amino acid sequence of SEQ ID NO:24, a light chain CDR2 comprising the amino acid sequence of SEQ ID NO:28, and a light chain CDR3 comprising the amino acid sequence of SEQ ID NO: 32.

In some embodiments, an anti-alpha-synuclein antibody or antigen-binding portion comprises a heavy chain variable region comprising an amino acid sequence that has at least 90% sequence identity (e.g., at least 90%, at least 91%, at least 92%, at least 93%, at least 94%, at least 95%, at least 96%, at least 97%, at least 98%, or at least 99% sequence identity) to any one of SEQ ID NOs:18, 144-166, 222, 223, and 229. In some embodiments, an anti-alpha-synuclein antibody or antigen-binding portion comprises a heavy chain variable region comprising the amino acid sequence of any one of SEQ ID NOs:18, 144-166, 222, 223, and 229. In certain embodiments, the heavy chain variable region comprises a W (Trp) at position 47, a L (Leu) at position 48, an A (Ala) at position 49, an F (Phe) at position 67, a D (Asp) at position 73, a V (Val) at position 78, or any position thereof.

In some embodiments, an anti-alpha-synuclein antibody or antigen-binding portion comprises a light chain variable region comprising an amino acid sequence that has at least 90% sequence identity (e.g., at least 90%, at least 91%, at least 92%, at least 93%, at least 94%, at least 95%, at least 96%, at least 97%, at least 98%, or at least 99% sequence identity) to any one of SEQ ID NOs:38, 167-187, 200-213, and 224. In some embodiments, an anti-alpha-synuclein antibody or antigen-binding portion comprises a light chain variable region comprising the amino acid sequence of any one of SEQ ID NOs:38, 167-187, 200-213, and 224. In certain embodiments, the heavy chain variable region comprises a P (Pro) at position 46, a V (Val) at position 58, a Y (Tyr) at position 71, or any combination thereof.

In some embodiments, an anti-alpha-synuclein antibody or antigen-binding portion comprises a heavy chain variable region comprising an amino acid sequence that has at least 90% sequence identity (e.g., at least 90%, at least 91%, at least 92%, at least 93%, at least 94%, at least 95%, at least 96%, at least 97%, at least 98%, or at least 99% sequence identity) to any one of SEQ ID NOs:18, 144-166, 222, 223, and 229 and further comprises a light chain variable region comprising an amino acid sequence that has at least 90% sequence identity (e.g., at least 90%, at least 91%, at least 92%, at least 93%, at least 94%, at least 95%, at least 96%, at least 97%, at least 98%, or at least 99% sequence identity) to any one of SEQ ID NOs:38, 167-187, 200-213, and 224. In certain embodiments, the heavy chain variable region comprises a W (Trp) at position 47, a L (Leu) at position 48, an A (Ala) at position 49, an F (Phe) at position 67, a D (Asp) at position 73, a V (Val) at position 78, or any position thereof. In certain embodiments, the heavy chain variable region comprises a P (Pro) at position 46, a V (Val) at position 58, a Y (Tyr) at position 71, or any combination thereof.

In some embodiments, an anti-alpha-synuclein antibody or antigen-binding portion comprises a heavy chain variable region comprising the amino acid sequence of any one of SEQ ID NOs:18, 144-166, 222, 223, and 229 and further comprises a light chain variable region comprising the amino acid sequence of any one of SEQ ID NOs:38, 167-187, 200-213, and 224.

In some embodiments, the anti-alpha-synuclein antibody or antigen-binding portion comprises (a) a heavy chain variable region having at least 90% (e.g., 90%, 91%, 92%, 93%, 94%, 95%, 96%, 97%, 98%, 99%, or 100%) identity to SEQ ID NO:147, wherein the heavy chain variable region comprises a CDR1 sequence of SEQ ID NO:3, a CDR2 sequence of SEQ ID NO:9, and a CDR3 sequence of SEQ ID NO:12, and (b) a light chain variable region having at least 90% identity to SEQ ID NO:173, wherein the light chain variable region comprises a CDR1 sequence of SEQ ID NO:122, a CDR2 sequence of SEQ ID NO:128, and a CDR3 sequence of SEQ ID NO:32.

In some embodiments, the anti-alpha-synuclein antibody or antigen-binding portion comprises (a) a heavy chain variable region having at least 90% identity to SEQ ID NO:147, wherein the heavy chain variable region comprises a CDR1 sequence of SEQ ID NO:3, a CDR2 sequence of SEQ ID NO:9, and a CDR3 sequence of SEQ ID NO:12, and (b) a light chain variable region having at least 90% identity to SEQ ID NO:201, wherein the light chain variable region comprises a CDR1 sequence of SEQ ID NO:122, a CDR2 sequence of SEQ ID NO:128, and a CDR3 sequence of SEQ ID NO:32.

In some embodiments, the anti-alpha-synuclein antibody or antigen-binding portion comprises (a) a heavy chain variable region having at least 90% identity to SEQ ID NO:147, wherein the heavy chain variable region comprises a CDR1 sequence of SEQ ID NO:3, a CDR2 sequence of SEQ ID NO:9, and a CDR3 sequence of SEQ ID NO:12, and (b) a light chain variable region having at least 90% identity to SEQ ID NO:202, wherein the light chain variable region comprises a CDR1 sequence of SEQ ID NO:24, a CDR2 sequence of SEQ ID NO:128, and a CDR3 sequence of SEQ ID NO:32.

In some embodiments, the anti-alpha-synuclein antibody or antigen-binding portion comprises (a) a heavy chain variable region having at least 90% identity to SEQ ID NO:147, wherein the heavy chain variable region comprises a CDR1 sequence of SEQ ID NO:3, a CDR2 sequence of SEQ ID NO:9, and a CDR3 sequence of SEQ ID NO:12, and (b) a light chain variable region having at least 90% identity to SEQ ID NO:200, wherein the light chain variable region comprises a CDR1 sequence of SEQ ID NO:24, a CDR2 sequence of SEQ ID NO:28, and a CDR3 sequence of SEQ ID NO:32.

In some embodiments, the anti-alpha-synuclein antibody or antigen-binding portion comprises (a) a heavy chain variable region having at least 90% identity to SEQ ID NO:18, wherein the heavy chain variable region comprises a CDR1 sequence of SEQ ID NO:3, a CDR2 sequence of SEQ ID NO:9, and a CDR3 sequence of SEQ ID NO:12, and (b) a light chain variable region having at least 90% identity to SEQ ID NO:38, wherein the light chain variable region comprises a CDR1 sequence of SEQ ID NO:24, a CDR2 sequence of SEQ ID NO:28, and a CDR3 sequence of SEQ ID NO:32.

In some embodiments, the anti-alpha-synuclein antibody or antigen-binding portion comprises (a) a heavy chain variable region having at least 90% identity to SEQ ID NO:229, wherein the heavy chain variable region comprises a CDR1 sequence of SEQ ID NO:3, a CDR2 sequence of SEQ ID NO:9, and a CDR3 sequence of SEQ ID NO:12, and (b) a light chain variable region having at least 90% identity to SEQ ID NO:173, wherein the light chain variable region comprises a CDR1 sequence of SEQ ID NO:122, a CDR2 sequence of SEQ ID NO:128, and a CDR3 sequence of SEQ ID NO:32.

In some embodiments, the anti-alpha-synuclein antibody or antigen-binding portion comprises (a) a heavy chain variable region having at least 90% identity to SEQ ID NO:229, wherein the heavy chain variable region comprises a CDR1 sequence of SEQ ID NO:3, a CDR2 sequence of SEQ ID NO:9, and a CDR3 sequence of SEQ ID NO:12, and (b) a light chain variable region having at least 90% identity to SEQ ID NO:201, wherein the light chain variable region comprises a CDR1 sequence of SEQ ID NO:122, a CDR2 sequence of SEQ ID NO:128, and a CDR3 sequence of SEQ ID NO:32.

In some embodiments, the anti-alpha-synuclein antibody or antigen-binding portion comprises (a) a heavy chain variable region having at least 90% identity to SEQ ID NO:229, wherein the heavy chain variable region comprises a CDR1 sequence of SEQ ID NO:3, a CDR2 sequence of SEQ ID NO:9, and a CDR3 sequence of SEQ ID NO:12, and (b) a light chain variable region having at least 90% identity to SEQ ID NO:202, wherein the light chain variable region comprises a CDR1 sequence of SEQ ID NO:24, a CDR2 sequence of SEQ ID NO:128, and a CDR3 sequence of SEQ ID NO:32.

In some embodiments, the anti-alpha-synuclein antibody or antigen-binding portion comprises (a) a heavy chain variable region having at least 90% identity to SEQ ID NO:229, wherein the heavy chain variable region comprises a CDR1 sequence of SEQ ID NO:3, a CDR2 sequence of SEQ ID NO:9, and a CDR3 sequence of SEQ ID NO:12, and (b) a light chain variable region having at least 90% identity to SEQ ID NO:200, wherein the light chain variable region comprises a CDR1 sequence of SEQ ID NO:24, a CDR2 sequence of SEQ ID NO:28, and a CDR3 sequence of SEQ ID NO:32.

In some embodiments, an anti-alpha-synuclein antibody or antigen-binding portion is an antibody or antigen-binding portion that competes for binding with an antibody or antigen-binding portion as described herein (e.g., an antibody comprising a heavy chain CDR1 sequence comprising the amino acid sequence of any one of SEQ ID NOs:3 and 97-102, a heavy chain CDR2 sequence comprising the amino acid sequence of any one of SEQ ID NOs:9 and 105-110, a heavy chain CDR3 sequence comprising the amino acid sequence of any one of SEQ ID NOs:12 and 113-119, a light chain CDR1 sequence comprising the amino acid sequence of any one of SEQ ID NOs:24 and 122-123, a light chain CDR2 sequence comprising the amino acid sequence of any one of SEQ ID NOs:28 and 126-128, and a light chain CDR3 sequence comprising the amino acid sequence of any one of SEQ ID NOs:32 and 131-141, or an antibody comprising a heavy chain variable region comprising the amino acid sequence of any one of SEQ ID NOs:18, 144-166, 222, 223, and 229 and further comprising a light chain variable region comprising the amino acid sequence of any one of SEQ ID NOs:38, 167-187, 200-213, and 224).

In some embodiments, an anti-alpha-synuclein antibody or antigen-binding portion comprises a heavy chain variable region comprising or consisting of the amino acid sequence set forth in any one of SEQ ID NOs:147, 222, 223, and 229; and a light chain variable region comprising or consisting of the amino acid sequence set forth in any one of SEQ ID NOs:173, 200, 201, 202, and 224. In some embodiments, an anti-alpha-synuclein antibody or antigen-binding portion comprises a heavy chain variable region comprising or consisting of an amino acid sequence according to SEQ ID NO:147 and a light chain variable region comprising or consisting of an amino acid sequence according to SEQ ID NO:173. In some embodiments, an anti-alpha-synuclein antibody or antigen-binding portion comprises a heavy chain variable region comprising or consisting of an amino acid sequence according to SEQ ID NO:147 and a light chain variable region comprising or consisting of an amino acid sequence according to SEQ ID NO:200. In some embodiments, an anti-alpha-synuclein antibody or antigen-binding portion comprises a heavy chain variable region comprising or consisting of an amino acid sequence according to SEQ ID NO:147 and a light chain variable region comprising or consisting of an amino acid sequence according to SEQ ID NO:201. In some embodiments, an anti-alpha-synuclein antibody or antigen-binding portion comprises a heavy chain variable region comprising or consisting of an amino acid sequence according to SEQ ID NO:147 and a light chain variable region comprising or consisting of an amino acid sequence according to SEQ ID NO:202. In some embodiments, an anti-alpha-synuclein antibody or antigen-binding portion comprises a heavy chain variable region comprising or consisting of an amino acid sequence according to SEQ ID NO:147 and a light chain variable region comprising or consisting of an amino acid sequence according to SEQ ID NO:224. In some embodiments, an anti-alpha-synuclein antibody or antigen-binding portion comprises a heavy chain variable region comprising or consisting of an amino acid sequence according to SEQ ID NO:222 and a light chain variable region comprising or consisting of an amino acid sequence according to SEQ ID NO:224 or 173. In some embodiments, an anti-alpha-synuclein antibody or antigen-binding portion comprises a heavy chain variable region comprising or consisting of an amino acid sequence according to SEQ ID NO:223 and a light chain variable region comprising or consisting of an amino acid sequence according to SEQ ID NO:224 or 173. In some embodiments, an anti-alpha-synuclein antibody or antigen-binding portion comprises a heavy chain variable region comprising or consisting of an amino acid sequence according to SEQ ID NO:229 and a light chain variable region comprising or consisting of an amino acid sequence according to SEQ ID NO:224 or 173. In some embodiments, an anti-alpha-synuclein antibody or antigen-binding portion comprises a heavy chain variable region comprising or consisting of an amino acid sequence according to SEQ ID NO:229 and a light chain variable region comprising or consisting of an amino acid sequence according to SEQ ID NO:200. In some embodiments, an anti-alpha-synuclein antibody or antigen-binding portion comprises a heavy chain variable region comprising or consisting of an amino acid sequence according to SEQ ID NO:229 and a light chain variable region comprising or consisting of an amino acid sequence according to SEQ ID NO:201. In some embodiments, an anti-alpha-synuclein antibody or antigen-binding portion comprises a heavy chain variable region comprising or consisting of an amino acid sequence according to SEQ ID NO:229 and a light chain variable region comprising or consisting of an amino acid sequence according to SEQ ID NO:202.

In some embodiments, the antibody or antigen-binding portion thereof further comprises a CH1 domain, which can comprise or consist of the amino acid sequence set forth in SEQ ID NO:194. In some embodiments, an antibody or antigen-binding portion comprises a heavy chain that can, in certain embodiments, comprise or consist of the amino acid sequence set forth in SEQ ID NO:219, 220, 226, 227, or 228.

In some embodiments, the antibody or antigen-binding portion thereof comprises a CL domain, which can, in certain embodiments, comprise or consist of the amino acid sequence set forth in SEQ ID NO:195. In some embodiments, an antibody or antigen-binding portion comprises a light chain that can, in certain embodiments, comprise or consist of the amino acid sequence set forth in any one of SEQ ID NOs:196, 197, 198, 199, and 221.

In some embodiments, an isolated antibody or antigen-binding portion thereof is provided that specifically binds to a human alpha-synuclein protein, wherein the antibody or antigen-binding portion thereof comprises: (i) a first and a second heavy chain, wherein either or both of the first and the second heavy chain comprises or consists of the amino acid sequence set forth in any one of SEQ ID NOs:219, 220, 226, 227 and 228; and (ii) a first light chain and a second light chain wherein either or both of the first light chain and the second heavy light chain comprises or consists of the amino acid sequence set forth in any one of SEQ ID NOs:196, 197, 198, 199, and 221.

Anti-Alpha-Synuclein Antibodies that Recognize an Epitope within Residues 112-140

In some embodiments, an anti-alpha-synuclein antibody or antigen-binding portion recognizes an epitope within residues 112-140 of SEQ ID NO:1. In some embodiments, the antibody or antigen-binding portion comprises one or more complementarity determining region (CDR), heavy chain variable region, and/or light chain variable regions as described herein (e.g., as described in Table 12 below).

In some embodiments, an anti-alpha-synuclein antibody or antigen-binding portion comprises one or more CDRs selected from the group consisting of: (a) a heavy chain CDR1 having at least 90% sequence identity to the amino acid sequence of any one of SEQ ID NOs:2 and 4 or having up to two amino acid substitutions relative to the amino acid sequence of any one of SEQ ID NOs:2 and 4; (b) a heavy chain CDR2 having at least 90% sequence identity to the amino acid sequence of SEQ ID NO:188 or having up to two amino acid substitutions relative to the amino acid sequence of SEQ ID NO:188; (c) a heavy chain CDR3 having at least 90% sequence identity to the amino acid sequence of any one of SEQ ID NOs:11 and 13 or having up to two amino acid substitutions relative to the amino acid sequence of any one of SEQ ID NOs:11 and 13; (d) a light chain CDR1 having at least 90% sequence identity to the amino acid sequence of any one of SEQ ID NOs:23 or 24 or having up to two amino acid substitutions relative to the amino acid sequence of any one of SEQ ID NOs:23 or 24; (e) a light chain CDR2 having at least 90% sequence identity to the amino acid sequence of SEQ ID NO:27 or having up to two amino acid substitutions relative to the amino acid sequence of SEQ ID NO:27; and (f) a light chain CDR3 having at least 90% sequence identity to the amino acid sequence of any one of SEQ ID NOs:31 and 33 or having up to two amino acid substitutions relative to the amino acid sequence of any one of SEQ ID NOs:31 and 33.

In some embodiments, an anti-alpha-synuclein antibody or antigen-binding portion comprises two, three, four, five, or all six of (a)-(f). In some embodiments, an anti-alpha-synuclein antibody comprises the heavy chain CDR1 of (a), the heavy chain CDR2 of (b), and the heavy chain CDR3 of (c). In some embodiments, an anti-alpha-synuclein antibody or antigen-binding portion comprises the light chain CDR1 of (d), the light chain CDR2 of (e), and the light chain CDR3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, an anti-alpha-synuclein antibody or antigen-binding portion comprises one or more CDRs selected from the group consisting of: (a) a heavy chain CDR1 comprising the amino acid sequence of any one of SEQ ID NOs:2 and 4; (b) a heavy chain CDR2 comprising the amino acid sequence of SEQ ID NO:188; (c) a heavy chain CDR3 comprising the amino acid sequence of any one of SEQ ID NOs:11 and 13; (d) a light chain CDR1 comprising the amino acid sequence of any one of SEQ ID NOs:23 and 24; (e) a light chain CDR2 comprising the amino acid sequence of SEQ ID NO:27; and (f) a light chain CDR3 comprising the amino acid sequence of any one of SEQ ID NOs:31 and 33.

In some embodiments, an anti-alpha-synuclein antibody or antigen-binding portion comprises two, three, four, five, or all six of (a)-(f). In some embodiments, an anti-alpha-synuclein antibody comprises the heavy chain CDR1 of (a), the heavy chain CDR2 of (b), and the heavy chain CDR3 of (c). In some embodiments, an anti-alpha-synuclein antibody or antigen-binding portion comprises the light chain CDR1 of (d), the light chain CDR2 of (e), and the light chain CDR3 of (f).

In some embodiments, the antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:17 and 19. In some embodiments, the antibody or antigen-binding portion comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:37 and 39. In some embodiments, the antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:17 and 19, and a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:37 and 39.

In some embodiments, the antibody or antigen-binding portion comprises a heavy chain variable region comprising the amino acid sequence of any one of SEQ ID NOs:17 and 19. In some embodiments, the antibody or antigen-binding portion comprises a light chain variable region comprising the amino acid sequence of any one of SEQ ID NOs:37 and 39. In some embodiments, the antibody or antigen-binding portion comprises a heavy chain variable region comprising the amino acid sequence of any one of SEQ ID NOs:17 and 19, and a light chain variable region comprising the amino acid of any one of SEQ ID NOs:37 and 39.

1H5

In some embodiments, an anti-alpha-synuclein antibody or antigen-binding portion comprises a heavy chain CDR1 sequence comprising the amino acid sequence of SEQ ID NO:2, a heavy chain CDR2 sequence comprising the amino acid sequence of SEQ ID NO:8, and a heavy chain CDR3 sequence comprising the amino acid sequence of SEQ ID NO:11. In some embodiments, an anti-alpha-synuclein antibody or antigen-binding portion comprises a light chain CDR1 sequence comprising the amino acid sequence of SEQ ID NO:23, a light chain CDR2 sequence comprising the amino acid sequence of SEQ ID NO:27, and a light chain CDR3 sequence comprising the amino acid sequence of SEQ ID NO:31. In some embodiments, an anti-alpha-synuclein antibody comprises a heavy chain CDR1-3 and a light chain CDR1-3 comprising the amino acid sequences of SEQ ID NOs:2, 8, 11, 23, 27, and 31, respectively.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:17. In some embodiments, an anti-alpha-synuclein antibody or antigen-binding portion comprises a heavy chain variable region comprising the amino acid sequence of SEQ ID NO:17.

In some embodiments, an anti-alpha-synuclein antibody or antigen-binding portion comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:37. In some embodiments, an anti-alpha-synuclein antibody or antigen-binding portion comprises a light chain variable region comprising the amino acid sequence of SEQ ID NO:37.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:17 and further comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:37. In some embodiments, an anti-alpha-synuclein antibody or antigen-binding portion comprises a heavy chain variable region comprising the amino acid sequence of SEQ ID NO:17 and further comprises a light chain variable region comprising the amino acid sequence of SEQ ID NO:37.

In some embodiments, an anti-alpha-synuclein antibody or antigen-binding portion is an antibody or antigen-binding portion that competes for binding with an antibody or antigen-binding portion as described herein (e.g., an antibody comprising a heavy chain CDR1-3 and a light chain CDR1-3 comprising the amino acid sequences of SEQ ID NOs:2, 8, 11, 23, 27, and 31, respectively, or an antibody comprising a heavy chain variable region comprising the amino acid sequence of SEQ ID NO:17 and further comprising a light chain variable region comprising the amino acid sequence of SEQ ID NO:37).

22D11

In some embodiments, an anti-alpha-synuclein antibody or antigen-binding portion comprises a heavy chain CDR1 sequence comprising the amino acid sequence of SEQ ID NO:4, a heavy chain CDR2 sequence comprising the amino acid sequence of SEQ ID NO:188, and a heavy chain CDR3 sequence comprising the amino acid sequence of SEQ ID NO:13. In some embodiments, an anti-alpha-synuclein antibody or antigen-binding portion comprises a light chain CDR1 sequence comprising the amino acid sequence of SEQ ID NO:24, a light chain CDR2 sequence comprising the amino acid sequence of SEQ ID NO:27, and a light chain CDR3 sequence comprising the amino acid sequence of SEQ ID NO:33. In some embodiments, an anti-alpha-synuclein antibody or antigen-binding portion comprises a heavy chain CDR1-3 and a light chain CDR1-3 comprising the amino acid sequences of SEQ ID NOs:4, 188, 13, 24, 27, and 33, respectively.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:19. In some embodiments, an anti-alpha-synuclein antibody or antigen-binding portion comprises a heavy chain variable region comprising the amino acid sequence of SEQ ID NO:19.

In some embodiments, an anti-alpha-synuclein antibody or antigen-binding portion comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:39. In some embodiments, an anti-alpha-synuclein antibody or antigen-binding portion comprises a light chain variable region comprising the amino acid sequence of SEQ ID NO:39.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:19 and further comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:39. In some embodiments, an anti-alpha-synuclein antibody or antigen-binding portion comprises a heavy chain variable region comprising the amino acid sequence of SEQ ID NO:19 and further comprises a light chain variable region comprising the amino acid sequence of SEQ ID NO:39.

In some embodiments, an anti-alpha-synuclein antibody or antigen-binding portion is an antibody or antigen-binding portion that competes for binding with an antibody or antigen-binding portion as described herein (e.g., an antibody comprising a heavy chain CDR1-3 and a light chain CDR1-3 comprising the amino acid sequences of SEQ ID NOs:4, 8, 13, 24, 27, and 33, respectively, or an antibody comprising a heavy chain variable region comprising the amino acid sequence of SEQ ID NO:19 and further comprising a light chain variable region comprising the amino acid sequence of SEQ ID NO:39).

Anti-Alpha-Synuclein Antibodies that Recognize an Epitope within Residues 95-140

In some embodiments, an anti-alpha-synuclein antibody or antigen-binding portion recognizes an epitope within residues 95-140 of SEQ ID NO:1. In some embodiments, the antibody or antigen-binding portion comprises one or more complementarity determining region (CDR), heavy chain variable region, and/or light chain variable regions as described herein (e.g., as described in Table 12 below).

In some embodiments, an anti-alpha-synuclein antibody or antigen-binding portion comprises one or more CDRs selected from the group consisting of: (a) a heavy chain CDR1 having at least 90% sequence identity to the amino acid sequence of any one of SEQ ID NOs:5-7 or having up to two amino acid substitutions relative to the amino acid sequence of any one of SEQ ID NOs:5-7; (b) a heavy chain CDR2 having at least 90% sequence identity to the amino acid sequence of any one of SEQ ID NOs:8 and 10 or having up to two amino acid substitutions relative to the amino acid sequence of any one of SEQ ID NOs:8 and 10; (c) a heavy chain CDR3 having at least 90% sequence identity to the amino acid sequence of any one of SEQ ID NOs:14-16 or having up to two amino acid substitutions relative to the amino acid sequence of any one of SEQ ID NOs:14-16; (d) a light chain CDR1 having at least 90% sequence identity to the amino acid sequence of any one of SEQ ID NOs:25 or 26 or having up to two amino acid substitutions relative to the amino acid sequence of any one of SEQ ID NOs:25 or 26; (e) a light chain CDR2 having at least 90% sequence identity to the amino acid sequence of any one of SEQ ID NOs:29 and 30 or having up to two amino acid substitutions relative to the amino acid sequence of any one of SEQ ID NOs:29 and 30; and (f) a light chain CDR3 having at least 90% sequence identity to the amino acid sequence of any one of SEQ ID NOs:34-36 or having up to two amino acid substitutions relative to the amino acid sequence of any one of SEQ ID NOs:34-36.

In some embodiments, an anti-alpha-synuclein antibody or antigen-binding portion comprises two, three, four, five, or all six of (a)-(f). In some embodiments, an anti-alpha-synuclein antibody comprises the heavy chain CDR1 of (a), the heavy chain CDR2 of (b), and the heavy chain CDR3 of (c). In some embodiments, an anti-alpha-synuclein antibody or antigen-binding portion comprises the light chain CDR1 of (d), the light chain CDR2 of (e), and the light chain CDR3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, an anti-alpha-synuclein antibody or antigen-binding portion comprises one or more CDRs selected from the group consisting of: (a) a heavy chain CDR1 comprising the amino acid sequence of any one of SEQ ID NOs:5-7; (b) a heavy chain CDR2 comprising the amino acid sequence of any one of SEQ ID NOs:8 and 10; (c) a heavy chain CDR3 comprising the amino acid sequence of any one of SEQ ID NOs:14-16; (d) a light chain CDR1 comprising the amino acid sequence of any one of SEQ ID NOs:25 and 26; (e) a light chain CDR2 comprising the amino acid sequence of any one of SEQ ID NOs:29 and 30; and (f) a light chain CDR3 comprising the amino acid sequence of any one of SEQ ID NOs:34-36.

In some embodiments, an anti-alpha-synuclein antibody or antigen-binding portion comprises two, three, four, five, or all six of (a)-(f). In some embodiments, an anti-alpha-synuclein antibody or antigen-binding portion comprises the heavy chain CDR1 of (a), the heavy chain CDR2 of (b), and the heavy chain CDR3 of (c). In some embodiments, an anti-alpha-synuclein antibody or antigen-binding portion comprises the light chain CDR1 of (d), the light chain CDR2 of (e), and the light chain CDR3 of (f).

In some embodiments, the antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:20-22. In some embodiments, the antibody or antigen-binding portion comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:40-42. In some embodiments, the antibody comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:20-22, and a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:40-42.

In some embodiments, the antibody or antigen-binding portion comprises a heavy chain variable region comprising the amino acid sequence of any one of SEQ ID NOs:20-22. In some embodiments, the antibody or antigen-binding portion comprises a light chain variable region comprising the amino acid sequence of any one of SEQ ID NOs:40-42. In some embodiments, the antibody or antigen-binding portion comprises a heavy chain variable region comprising the amino acid sequence of any one of SEQ ID NOs:20-22, and a light chain variable region comprising the amino acid of any one of SEQ ID NOs:40-42.

16F3

In some embodiments, an anti-alpha-synuclein antibody or antigen-binding portion comprises a heavy chain CDR1 sequence comprising the amino acid sequence of SEQ ID NO:5, a heavy chain CDR2 sequence comprising the amino acid sequence of SEQ ID NO:8, and a heavy chain CDR3 sequence comprising the amino acid sequence of SEQ ID NO:14. In some embodiments, an anti-alpha-synuclein antibody or antigen-binding portion comprises a light chain CDR1 sequence comprising the amino acid sequence of SEQ ID NO:25, a light chain CDR2 sequence comprising the amino acid sequence of SEQ ID NO:29, and a light chain CDR3 sequence comprising the amino acid sequence of SEQ ID NO:34. In some embodiments, an anti-alpha-synuclein antibody or antigen-binding portion comprises a heavy chain CDR1-3 and a light chain CDR1-3 comprising the amino acid sequences of SEQ ID NOs:5, 8, 14, 25, 29, and 34, respectively.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:20. In some embodiments, an anti-alpha-synuclein antibody or antigen-binding portion comprises a heavy chain variable region comprising the amino acid sequence of SEQ ID NO:20.

In some embodiments, an anti-alpha-synuclein antibody or antigen-binding portion comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:40. In some embodiments, an anti-alpha-synuclein antibody or antigen-binding portion comprises a light chain variable region comprising the amino acid sequence of SEQ ID NO:40.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:20 and further comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:40. In some embodiments, an anti-alpha-synuclein antibody or antigen-binding portion comprises a heavy chain variable region comprising the amino acid sequence of SEQ ID NO:20 and further comprises a light chain variable region comprising the amino acid sequence of SEQ ID NO:40.

In some embodiments, an anti-alpha-synuclein antibody or antigen-binding portion is an antibody or antigen-binding portion that competes for binding with an antibody or antigen-binding portion as described herein (e.g., an antibody comprising a heavy chain CDR1-3 and a light chain CDR1-3 comprising the amino acid sequences of SEQ ID NOs:5, 8, 14, 25, 29, and 34, respectively, or an antibody comprising a heavy chain variable region comprising the amino acid sequence of SEQ ID NO:20 and further comprising a light chain variable region comprising the amino acid sequence of SEQ ID NO:40).

18B4

In some embodiments, an anti-alpha-synuclein antibody or antigen-binding portion comprises a heavy chain CDR1 sequence comprising the amino acid sequence of SEQ ID NO:6, a heavy chain CDR2 sequence comprising the amino acid sequence of SEQ ID NO:10, and a heavy chain CDR3 sequence comprising the amino acid sequence of SEQ ID NO:15. In some embodiments, an anti-alpha-synuclein antibody or antigen-binding portion comprises a light chain CDR1 sequence comprising the amino acid sequence of SEQ ID NO:25, a light chain CDR2 sequence comprising the amino acid sequence of SEQ ID NO:29, and a light chain CDR3 sequence comprising the amino acid sequence of SEQ ID NO:35. In some embodiments, an anti-alpha-synuclein antibody or antigen-binding portion comprises a heavy chain CDR1-3 and a light chain CDR1-3 comprising the amino acid sequences of SEQ ID NOs:6, 10, 15, 25, 29, and 35, respectively.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:21. In some embodiments, an anti-alpha-synuclein antibody or antigen-binding portion comprises a heavy chain variable region comprising the amino acid sequence of SEQ ID NO:21.

In some embodiments, an anti-alpha-synuclein antibody or antigen-binding portion comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:41. In some embodiments, an anti-alpha-synuclein antibody or antigen-binding portion comprises a light chain variable region comprising the amino acid sequence of SEQ ID NO:41.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:21 and further comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:41. In some embodiments, an anti-alpha-synuclein antibody or antigen-binding portion comprises a heavy chain variable region comprising the amino acid sequence of SEQ ID NO:21 and further comprises a light chain variable region comprising the amino acid sequence of SEQ ID NO:41.

In some embodiments, an anti-alpha-synuclein antibody or antigen-binding portion is an antibody or antigen-binding portion that competes for binding with an antibody or antigen-binding portion as described herein (e.g., an antibody comprising a heavy chain CDR1-3 and a light chain CDR1-3 comprising the amino acid sequences of SEQ ID NOs:6, 10, 15, 25, 29, and 35, respectively, or an antibody comprising a heavy chain variable region comprising the amino acid sequence of SEQ ID NO:21 and further comprising a light chain variable region comprising the amino acid sequence of SEQ ID NO:41).

22E11

In some embodiments, an anti-alpha-synuclein antibody or antigen-binding portion comprises a heavy chain CDR1 sequence comprising the amino acid sequence of SEQ ID NO:7, a heavy chain CDR2 sequence comprising the amino acid sequence of SEQ ID NO:8, and a heavy chain CDR3 sequence comprising the amino acid sequence of SEQ ID NO:16. In some embodiments, an anti-alpha-synuclein antibody or antigen-binding portion comprises a light chain CDR1 sequence comprising the amino acid sequence of SEQ ID NO:26, a light chain CDR2 sequence comprising the amino acid sequence of SEQ ID NO:30, and a light chain CDR3 sequence comprising the amino acid sequence of SEQ ID NO:36. In some embodiments, an anti-alpha-synuclein antibody or antigen-binding portion comprises a heavy chain CDR1-3 and a light chain CDR1-3 comprising the amino acid sequences of SEQ ID NOs:7, 8, 16, 26, 30, and 36, respectively.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:22. In some embodiments, an anti-alpha-synuclein antibody or antigen-binding portion comprises a heavy chain variable region comprising the amino acid sequence of SEQ ID NO:22.

In some embodiments, an anti-alpha-synuclein antibody or antigen-binding portion comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:42. In some embodiments, an anti-alpha-synuclein antibody or antigen-binding portion comprises a light chain variable region comprising the amino acid sequence of SEQ ID NO:42.

In some embodiments, an anti-alpha-synuclein antibody or antigen-binding portion comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:22 and further comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:42. In some embodiments, an anti-alpha-synuclein antibody or antigen-binding portion comprises a heavy chain variable region comprising the amino acid sequence of SEQ ID NO:22 and further comprises a light chain variable region comprising the amino acid sequence of SEQ ID NO:42.

In some embodiments, an anti-alpha-synuclein antibody or antigen-binding portion is an antibody or antigen-binding portion that competes for binding with an antibody or antigen-binding portion as described herein (e.g., an antibody comprising a heavy chain CDR1-3 and a light chain CDR1-3 comprising the amino acid sequences of SEQ ID NOs:7, 8, 16, 26, 30, and 36, respectively, or an antibody comprising a heavy chain variable region comprising the amino acid sequence of SEQ ID NO:22 and further comprising a light chain variable region comprising the amino acid sequence of SEQ ID NO:42).

Preparation of Antibodies

In some embodiments, antibodies are prepared by immunizing a rat with an antigen or a mixture of antigens, or a combination thereof, for the induction of an antibody response. In some embodiments, the antigen or mixture of antigens, or a combination thereof, is administered in conjugation with an adjuvant (e.g., Freund's or Ribi adjuvant).

One embodiment therefore includes an antigen composition for immunizing an animal, comprising monomeric alpha-synuclein, oligomeric alpha-synuclein, and optionally an adjuvant, for example, Freund's adjuvant. In one example, the antigen composition comprises a mixture of oligomeric alpha-synuclein protein in equilibrium with monomeric alpha-synuclein protein. In one example, the antigen composition is enriched for oligomeric alpha-synuclein protein compared to monomeric alpha-synuclein protein. In one example, the antigen composition comprises about 80% oligomeric and about 20% monomeric alpha-synuclein protein. Methods of separating or purifying oligomeric alpha-synuclein from monomeric alpha synuclein protein are known in the art, and include size exclusion chromatography. Another embodiment includes a method of producing an anti-alpha-synuclein antibody, comprising administering to an animal an antigen composition described herein, such as a mixture of monomeric alpha-synuclein, oligomeric alpha-synuclein, and optionally an adjuvant.

After an initial immunization, one or more subsequent booster injections of the antigen or antigens may be administered to improve antibody production. Following immunization, antigen-specific B cells are harvested, e.g., from the spleen and/or lymphoid tissue. For generating monoclonal antibodies, multiple methods may be used. In one embodiment, the B cells are fused with myeloma cells, which are subsequently screened for antigen specificity. Methods of preparing antibodies are also described in the Examples section below.

Genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridomas or splenocytes or lymphocytes (e.g. B cells from an immunized animal). Alternatively, phage or yeast display technology can be used to identify antibodies and fragments that specifically bind to selected antigens via generation of immune Fab or scFv libraries and screening on recombinant antigen. Antibodies can also be heteroconjugates, e.g., two covalently joined antibodies, or immunotoxins.

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a VH and VL region, the VH and VL regions may be expressed using a single vector, e.g., in a di-cistronic expression unit, or under the control of different promoters. In other embodiments, the VH and VL region may be expressed using separate vectors. A VH or VL region as described herein may optionally comprise a methionine at the N-terminus.

In some embodiments, the antibody is a chimeric antibody. Methods for making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, the antibody is a humanized antibody. Generally, a non-human antibody is humanized in order to reduce its immunogenicity. Humanized antibodies typically comprise one or more variable regions (e.g., CDRs) or portions thereof that are non-human (e.g., derived from a mouse variable region sequence), and possibly some framework regions or portions thereof that are non-human, and further comprise one or more constant regions that are derived from human antibody sequences. Methods for humanizing non-human antibodies are known in the art. Transgenic mice, or other organisms such as other mammals, can be used to express humanized or human antibodies. Other methods of humanizing antibodies include, for example, variable region resurfacing, CDR grafting, grafting specificity-determining residues (SDR), guided selection, and framework shuffling.

As an alternative to humanization, fully human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. As another example, human antibodies can be produced by hybridoma-based methods, such as by using primary human B cells for generating cell lines producing human monoclonal antibodies.

Human antibodies can also be produced using phage display or yeast display technology. In phage display, repertoires of variable heavy chain and variable light chain genes are amplified and expressed in phage display vectors. In some embodiments, the antibody library is a natural repertoire amplified from a human source. In some embodiments, the antibody library is a synthetic library made by cloning heavy chain and light chain sequences and recombining to generate a large pool of antibodies with different antigenic specificity. Phage typically display antibody fragments (e.g., Fab fragments or scFv fragments), which are then screened for binding to an antigen of interest.

In some embodiments, antibody fragments (such as a Fab, a Fab′, a F(ab′)₂, a scFv, a V_{H}, a V_{HH}, or a V_{NAR}) are generated. Various techniques have been developed for the production of antibody fragments. Traditionally, these fragments were derived via proteolytic digestion of intact antibodies. However, these fragments can now be produced directly using recombinant host cells. For example, antibody fragments can be isolated from antibody phage libraries. Alternatively, Fab′-SH fragments can be directly recovered from E. coli cells and chemically coupled to form F(ab′)₂ fragments. According to another approach, F(ab′)₂ fragments can be isolated directly from recombinant host cell culture. Other techniques for the production of antibody fragments will be apparent to those skilled in the art.

In some embodiments, the antibody or an antibody fragment is conjugated or linked to another molecule, e.g., polyethylene glycol (PEGylation) or serum albumin, to provide an extended half-life in vivo.

i. Multispecific Antibodies

In some embodiments, multispecific antibodies comprising an anti-alpha-synuclein antibody (or antigen-binding portion thereof) as described herein are provided. In some embodiments, a multispecific antibody has a variable region-binding specificity for alpha-synuclein and has a variable region-binding specificity for at least one other antigen (or, in certain embodiments, a different epitope of a same antigen).

Methods for making multispecific antibodies include, but are not limited to, recombinant co-expression of two pairs of heavy chain and light chain in a host cell, “knobs-into-holes”engineering, “intramolecular trimerization, and fusion of an antibody fragment to the N-terminus or C-terminus of another antibody, e.g., tandem variable domains.

ii. Nucleic Acids, Vectors, and Host Cells

In some embodiments, the anti-alpha-synuclein antibodies as described herein are prepared using recombinant methods. Accordingly, in some aspects, the disclosure provides isolated nucleic acids comprising a nucleic acid sequence encoding any of the anti-alpha-synuclein antibodies as described herein (e.g., any one or more of the CDRs, heavy chain variable regions, and light chain variable regions described herein); vectors comprising such nucleic acids; and host cells into which the nucleic acids are introduced that are used to replicate the antibody-encoding nucleic acids and/or to express the antibodies.

In some embodiments, a polynucleotide (e.g., an isolated polynucleotide) comprises a nucleotide sequence encoding an antibody or antigen-binding portion thereof as described herein (e.g., as described in the Section above entitled “Anti-Alpha-Synuclein Antibody Sequences”). In some embodiments, the polynucleotide comprises a nucleotide sequence encoding one or more amino acid sequences (e.g., CDR, heavy chain, light chain, and/or framework regions) disclosed in Table 12 below. In some embodiments, the polynucleotide comprises a nucleotide sequence encoding an amino acid sequence having at least 85% sequence identity (e.g., at least 85%, at least 90%, at least 91%, at least 92%, at least 93%, at least 94%, at least 95%, at least 96%, at least 97%, at least 98%, or at least 99% sequence identity) to an amino acid sequence (e.g., a CDR, variable region, heavy chain, light chain, or framework region sequence) disclosed in Table 12 below. In some embodiments, a polynucleotide as described herein is operably linked to a heterologous nucleic acid, e.g., a heterologous promoter.

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

A vector can comprise any one or more of the vectors disclosed herein. In particular embodiments, a vector is provided that comprises a DNA plasmid construct encoding the antibody or antigen-binding portion (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding portion, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding portion are encoded by a polynucleotide comprised in a single plasmid. In other embodiments, the substituent components of the antibody or antigen-binding portion are encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). In certain embodiments, a single plasmid comprises a polynucleotide encoding a heavy chain and/or a light chain from two or more antibodies or antigen-binding portion of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase).

Suitable host cells for cloning or expressing a polynucleotide or vector as described herein include eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In some embodiments, the host cell is prokaryotic. In some embodiments, the host cell is eukaryotic, e.g., Chinese Hamster Ovary (CHO) cells or lymphoid cells. In some embodiments, the host cell is a human cell, e.g., a Human Embryonic Kidney (HEK) cell.

In a further aspect, methods of making an anti-alpha-synuclein antibody as described herein are provided. In some embodiments, the method includes culturing a host cell as described herein (e.g., a host cell expressing a polynucleotide or vector as described herein) under conditions suitable for expression of the antibody. In some embodiments, the antibody is subsequently recovered from the host cell (or host cell culture medium).

iii. Hybridoma Cell Lines

In another aspect, hybridoma cell lines are provided. Methods of generating and screening hybridoma cell lines, including the selection and immunization of suitable animals, the isolation and fusion of appropriate cells to create the hybridomas, the screening of hybridomas for the secretion of desired antibodies, and characterization of the antibodies will be known to one of ordinary skill in the art. Non-limiting examples of hybridoma cell lines are also described in Example 1 herein. In some embodiments, the hybridoma cell line secretes an anti-alpha-synuclein antibody (e.g., monoclonal anti-alpha-synuclein antibody) of the present disclosure. In some embodiments, the hybridoma cell line is selected from the group consisting of 1H5, 20H4, 22D11, 16F3, 18B4, and 22E11. In some embodiments, the hybridoma cell is the hybridoma cell line designated 1H5, which secretes the anti-alpha-synuclein antibody 1H5. In some embodiments, the hybridoma cell line is the hybridoma cell line designated 20H4, which secretes the anti-alpha-synuclein antibody 20H4. In some embodiments, the hybridoma cell line is the hybridoma cell line designated 22D11, which secretes the anti-alpha-synuclein antibody 22D11. In some embodiments, the hybridoma cell line is the hybridoma cell line designated 16F3, which secretes the anti-alpha-synuclein antibody 16F3. In some embodiments, the hybridoma cell line is the hybridoma cell line designated 18B4, which secretes the anti-alpha-synuclein antibody 18B4. In some embodiments, the hybridoma cell line is the hybridoma cell line designated 22E11, which secretes the anti-alpha-synuclein antibody 22E11.

In still another aspect, antibodies (e.g., monoclonal antibodies) secreted by hybridoma cell lines of the present disclosure are provided. In some embodiments, the antibody is the anti-alpha-synuclein antibody secreted by the hybridoma cell line 1H5, 20H4, 22D11, 16F3, 18B4, or 22E11. In some embodiments, the antibody is the anti-alpha-synuclein antibody secreted by the hybridoma cell line 1H5. In some embodiments, the antibody is the anti-alpha-synuclein antibody secreted by the hybridoma cell line 20H4. In some embodiments, the antibody is the anti-alpha-synuclein antibody secreted by the hybridoma cell line 22D11. In some embodiments, the antibody is the anti-alpha-synuclein antibody secreted by the hybridoma cell line 16F3. In some embodiments, the antibody is the anti-alpha-synuclein antibody secreted by the hybridoma cell line 18B4. In some embodiments, the antibody is the anti-alpha-synuclein antibody secreted by the hybridoma cell line 22E11.

### IV. Fc Polypeptide Mutations of Anti-Alpha-Synuclein Antibodies

In some aspects, an anti-alpha-synuclein antibody of the disclosure comprises two Fc polypeptides, one or both of which may each comprise independently selected modifications (e.g., mutations) or may be a wild-type Fc polypeptide, e.g., a human IgG1 Fc polypeptide. Non-limiting examples of mutations that can be introduced into one or both Fc polypeptides include, e.g., mutations to increase serum stability, to modulate effector function, to influence glycosylation, to reduce immunogenicity in humans, and/or to provide for knob and hole heterodimerization of the Fc polypeptides.

Mutations to Promote Heterodimerization of Fc Polypeptides

In some embodiments, the Fc polypeptides present in the anti-alpha-synuclein antibody include knob and hole mutations to promote heterodimer formation and hinder homodimer formation (e.g., for a bispecific or other multispecific antibody, or for an antibody that comprises a modification in one Fc polypeptide of an Fc dimer, but does not comprise the same modification in the other Fc polypeptide of the dimer). Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

In one illustrative embodiment of a knob and hole approach for dimerization, position 366 (numbered according to the EU numbering scheme) of one of the Fc polypeptides present in the anti-alpha-synuclein antibody comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of an anti-alpha-synuclein antibody of the disclosure has the T366W knob mutation and the other Fc polypeptide has the Y407V mutation, which is typically accompanied by the T366S and L368A hole mutations.

In some embodiments, one or both Fc polypeptides may be engineered to contain other modifications for heterodimerization, e.g., electrostatic engineering of contact residues within a CH3-CH3 interface that are naturally charged or hydrophobic patch modifications.

In some embodiments, modifications to enhance serum half-life may be introduced. For example, in some embodiments, one or both Fc polypeptides present in an anti-alpha-synuclein antibody of the disclosure may comprise a tyrosine at position 252, a threonine at position 254, and a glutamic acid at position 256, as numbered according to the EU numbering scheme. Thus, one or both Fc polypeptides may have M252Y, S254T, and T256E substitutions. Alternatively, one or both Fc polypeptides may have M428L and/or N434S substitutions, according to EU numbering. Alternatively, one or both Fc polypeptides may have an N434S or N434A substitution.

Fc Effector Functions

In some embodiments, one or both Fc polypeptides may comprise modifications that reduce effector function, i.e., having a reduced ability to induce certain biological functions upon binding to an Fc receptor expressed on an effector cell that mediates the effector function. Examples of antibody effector functions include, but are not limited to, Clq binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides may include modifications that modulate effector function.

In some embodiments, one or both Fc polypeptides may comprise modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (LALA) substitutions.

Additional Fc polypeptide mutations that modulate an effector function include, but are not limited to, the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

FcRn Binding Sites and Mutations to Increase Serum Half-Life

In certain aspects, Fc polypeptides (e.g., modified Fc polypeptides) present in an anti-alpha-synuclein antibody of the disclosure, can comprise an FcRn binding site. In some embodiments, the FcRn binding site is within the Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart Fc polypeptide having the wild-type residues at the mutated positions when assayed under the same conditions). In some embodiments, an FcRn binding site has one or more amino acid residues that are substituted at positions 251-256, 428, and 433-436, according to the EU numbering scheme.

In some embodiments, one or more residues at or near an FcRn binding site are mutated, relative to a native human IgG sequence, to extend serum half-life of the polypeptide. In some embodiments, mutations are introduced into one, two, or three of positions 252, 254, and 256. In some embodiments, the mutations are M252Y, S254T, and T256E. In some embodiments, an Fc polypeptide further comprises the mutations M252Y, S254T, and T256E. In particular embodiments, one or both Fc polypeptides present in an anti-alpha-synuclein antibody of the disclosure may comprise a tyrosine at position 252, a threonine at position 254, and a glutamic acid at position 256, as numbered according to the EU numbering scheme. Thus, one or both Fc polypeptides may have M252Y, S254T, and T256E substitutions.

In some embodiments, the mutations are M428L and/or N434S. In some embodiments, an Fc polypeptide further comprises the mutation N434S with or without M428L. In some embodiments, an Fc polypeptide comprises a mutation at one, two, or all three of positions T307, E380, and N434, according to the EU numbering scheme. In some embodiments, the mutations are T307Q and N434A. In some embodiments, an Fc polypeptide comprises mutations T307A, E380A, and N434A. In some embodiments, an Fc polypeptide comprises mutations at positions T250 and M428, according to the EU numbering scheme. In some embodiments, the Fc polypeptide comprises mutations T250Q and/or M428L. In some embodiments, an Fc polypeptide comprises mutations at positions M428 and N434, according to the EU numbering scheme. In some embodiments, the Fc polypeptide comprises mutations M428L and N434S (“LS” substitutions). In some embodiments, an antibody of the present disclosure can comprise two Fc polypeptides, wherein each of the two Fc polypeptides comprises M428L and/or N434S substitutions. In some embodiments, the Fc polypeptide comprises an N434S or N434A mutation. In some embodiments, an antibody of the present disclosure can comprise two Fc polypeptides, wherein each of the two Fc polypeptides comprises an N434S or N434A substitution.

### V. Therapeutic Methods Using Anti-Alpha-Synuclein Antibodies

In another aspect, methods for the use of anti-alpha-synuclein antibodies (or antigen-binding portions, or compositions that comprise the antibody or antigen-binding portion, or polynucleotides that encode an antibody or antigen-binding portion, or vectors that comprise such a polynucleotide, or host cells that comprise such a polynucleotide) as described herein are provided. In some embodiments, an anti-alpha-synuclein antibody or antigen-binding portion as described in Section III above is used in the practice of the methods described herein.

In some embodiments, methods of neutralizing human alpha-synuclein protein are provided. In some embodiments, the method comprises neutralizing human alpha-synuclein protein in a subject, e.g., in a brain of a subject. In some embodiments, the method comprises administering to the subject an anti-alpha-synuclein antibody or antigen-binding portion thereof as described herein, a composition comprising an anti-alpha-synuclein antibody or antigen-binding portion thereof, as described herein, an antibody that competes for binding with an isolated antibody as described herein, an antibody secreted by a hybridoma cell line as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein. In some embodiments, the subject is an individual having a neurodegenerative disease (e.g., a synucleinopathy).

In some embodiments, neutralizing the human alpha-synuclein protein comprises preventing, reducing, or inhibiting alpha-synuclein oligomerization and/or aggregation in a subject, e.g., in a brain of a subject. In some embodiments, neutralizing the human alpha-synuclein protein comprises preventing, reducing, or inhibiting alpha-synuclein oligomerization in a subject, e.g., in a brain of a subject. In some embodiments, neutralizing the human alpha-synuclein protein comprises preventing, reducing, or inhibiting alpha-synuclein aggregation in a subject, e.g., in a brain of a subject. In some embodiments, neutralizing the human alpha-synuclein protein comprises preventing, reducing, or inhibiting alpha-synuclein oligomerization and aggregation in a subject, e.g., in a brain of a subject.

In some embodiments, neutralizing the human alpha-synuclein protein comprises preventing, reducing, or inhibiting the spreading of alpha-synuclein protein oligomers and/or fibrils in a subject, e.g., in a brain of a subject. In some embodiments, neutralizing the human alpha-synuclein protein comprises preventing, reducing, or inhibiting the spreading of alpha-synuclein protein oligomers in a subject, e.g., in a brain of a subject. In some embodiments, neutralizing the human alpha-synuclein protein comprises preventing, reducing, or inhibiting the spreading of alpha-synuclein protein fibrils in a subject, e.g., in a brain of a subject. In some embodiments, neutralizing the human alpha-synuclein protein comprises preventing, reducing, or inhibiting the spreading of alpha-synuclein protein oligomers and fibrils in a subject, e.g., in a brain of a subject. In some embodiments, neutralizing the human alpha-synuclein protein comprises preventing, reducing, or inhibiting the seeding of alpha-synuclein protein in a subject, e.g., in a brain of a subject.

In some embodiments, the anti-alpha-synuclein antibody, antigen-binding portion, or composition neutralizes monomeric human alpha-synuclein, oligomeric human alpha-synuclein, soluble human alpha-synuclein, human alpha-synuclein fibrils, and/or phosphorylated human alpha-synuclein (e.g., pSer129). In some embodiments, the anti-alpha-synuclein antibody is a pan-alpha-synuclein antibody.

In some embodiments, methods of treating a neurodegenerative disease (e.g., a synucleinopathy or tauopathy) are provided. In some embodiments, the method comprises administering to a subject having a neurodegenerative disease an anti-alpha-synuclein antibody or antigen-binding portion thereof as described herein, or a composition comprising an anti-alpha-synuclein antibody as described herein, an antibody that competes for binding with an isolated antibody as described herein, an antibody secreted by a hybridoma cell line as described herein, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein.

In some embodiments, the neurodegenerative disease is characterized by the presence of Lewy bodies and/or Lewy neurites. In some embodiments, the presence of alpha-synuclein accumulation or aggregation (e.g., alpha-synuclein oligomers and/or fibrils), Lewy bodies and/or Lewy neurites are identified in a subject (e.g., before a subject received treatment according to the methods described herein).

In some embodiments, the neurodegenerative disease is selected from the group consisting of Parkinson's disease, dementia with Lewy bodies, multiple system atrophy, Huntington's disease, Alzheimer's disease, primary age-related tauopathy, hereditary late-onset Parkinson's disease, dysautonomia, traumatic brain injury (TBI), cerebrovascular dementia, progressive supranuclear palsy (PSP), frontotemporal dementia, frontotemporal dementia with parkinsonism linked to chromosome 17, argyrophilic grain dementia, amyotrophic lateral sclerosis/parkinsonism-dementia complex of Guam, corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, inclusion-body myositis, myotonic dystrophy, neurofibrillary tangle-predominant dementia, Niemann-Pick disease type C, pallido-ponto-nigral degeneration, Pick's disease, postencephalitic parkinsonism, prion protein cerebral amyloid angiopathy, progressive subcortical gliosis, subacute sclerosing panencephalitis, and tangle only dementia.

In some embodiments, a synucleinopathy is treated. In some embodiments, the synucleinopathy is selected from the group consisting of Parkinson's disease, dementia with Lewy bodies, and multiple system atrophy. In some embodiments, Parkinson's disease is treated.

In some embodiments, treating the subject delays the onset of symptoms of the disease, causes the reversal of one or more disease symptoms, delays or slows the progression of one or more disease symptoms, lessons the severity of one or more disease symptoms, increases disease survival, or a combination thereof. As non-limiting examples, symptoms of a neurodegenerative disease include memory deficits, cognitive deficits, motor deficits, sensory deficits, and speech deficits.

In some embodiments, the subject to be treated is a human, e.g., a human adult or a human child.

In some embodiments, the anti-alpha-synuclein antibody, antigen-binding portion, or composition, or an isolated polynucleotide encoding an anti-alpha-synuclein antibody or antigen-binding portion thereof as described herein, and/or a vector or host cell comprising a polynucleotide encoding an anti-alpha-synuclein antibody or antigen-binding portion thereof as described herein, is administered to a subject (e.g., a human subject) prior to the broad spreading of alpha-synuclein protein (e.g., human alpha-synuclein oligomers and/or fibrils) in the brain of the subject. In some embodiments, administering prior to the broad spreading increases the ability of the isolated antibody, antigen-binding portion, or composition (or antibody-encoding isolated polynucleotide, vector, or host cell, as above) to treat the disease, prevents or delays the onset of symptoms of the disease, causes the reversal of disease symptoms, delays or slows the progression of disease symptoms, lessons the severity of disease symptoms, increases disease survival, or a combination thereof. As non-limiting examples, symptoms of a neurodegenerative disease include memory deficits, cognitive deficits, motor deficits, sensory deficits, and speech deficits.

In some embodiments, the subject receives therapy (i.e., the anti-alpha-synuclein antibody, antigen-binding portion, or composition (or antibody-encoding isolated polynucleotide, vector, or host cell, as above) is administered to the subject) during an early stage of the disease (e.g., neurodegenerative disease). One of ordinary skill in the art will appreciate that various diseases progress at different rates and exhibit different combinations of signs and symptoms at each stage. As such, the stages of the various diseases are classified according to different systems. In some diseases, such as Parkinson's disease, the early stages can be presymptomatic. Accordingly, in some embodiments, the term “early stage” as used herein refers to when the subject has not yet exhibited any signs or symptoms of the disease to be treated. In other diseases, such as amyotrophic lateral sclerosis (ALS), early stages of the disease are characterized by symptoms that are limited to a single body region or mild symptoms that affect more than one region. Accordingly, in some embodiments, the term “early stage” as used herein refers to when the subject has one or more symptoms of the disease to be treated that affect a localized region of the body, or when the subject has one or more mild symptoms of the disease to be treated that are not localized to one region of the body. Depending on the particular disease to be treated, the skilled practitioner will be able to determine whether the subject is in an early stage of the disease.

In some embodiments, the subject has not yet been treated for the disease.

In some embodiments, the amount of the isolated antibody, antigen-binding portion, or composition (or antibody-encoding isolated polynucleotide, vector, or host cell, as above) that is necessary to neutralize the alpha-synuclein protein (e.g., human alpha-synuclein protein) in the brain of the subject (e.g., the amount that is necessary to prevent or reduce the spreading of alpha-synuclein protein in the brain of the subject) is determined based on an amount of total alpha-synuclein protein in a sample, an amount of alpha-synuclein protein in a sample that is bound to the isolated antibody or antigen-binding portion and/or the amount of free alpha-synuclein protein in a sample.

In some embodiments, the amount of the isolated antibody, antigen-binding portion, or composition (or antibody-encoding isolated polynucleotide, vector, or host cell, as above) that is administered to the subject (e.g., in order to prevent or treat a disease such as a neurodegenerative disease) is determined based on an amount of total alpha-synuclein protein in a sample, an amount of alpha-synuclein protein in a sample that is bound to the isolated antibody and/or the amount of free alpha-synuclein protein in a sample.

In some embodiments, the sample is obtained from the subject. Suitable samples include, but are not limited to, interstitial brain fluid samples, cerebrospinal fluid (CSF) samples, brain lysate blood samples, or any combination thereof. When the sample(s) are obtained from the subject, the sample(s) can be obtained before treatment and/or during treatment. In some embodiments, the amount of total, bound, and/or free alpha-synuclein antibody in the sample(s) can be used to adjust treatment (e.g., increase or decrease the amount of the isolated antibody, antigen-binding portion, or composition that is administered to the subject, or to select a different isolated antibody or composition for administration to the subject).

In some embodiments, an anti-alpha-synuclein antibody or antigen-binding portion thereof that is specific for, or has a higher binding affinity for, a toxic species of alpha-synuclein (e.g., oligomeric alpha-synuclein) is used to determine the amount of bound alpha-synuclein protein.

In some embodiments, an amount of the isolated antibody, antigen-binding portion, or composition (or antibody-encoding isolated polynucleotide, vector, or host cell, as above) is administered to a subject (e.g., an animal or a human subject) such that at least about 80%, 90% or 95% or more of alpha-synuclein protein is bound by the isolated antibody or antigen-binding portion.

Higher percentages of bound alpha-synuclein protein (i.e., bound by an isolated antibody) are associated with a reduction in alpha-synuclein spreading, seeding, oligomerization, or aggregation. In some embodiments, the percentage of bound alpha-synuclein protein that is necessary to inhibit alpha-synuclein spreading, seeding, oligomerization, or aggregation is used to determine how much isolated antibody or composition (or antibody-encoding isolated polynucleotide, vector, or host cell, as above) is administered to a subject (e.g., in order to treat the subject).

Animal models can be used to determine an appropriate amount of anti-alpha-synuclein antibody (or antibody composition or antibody-encoding isolated polynucleotide, vector, or host cell, as above) to be administered to a human subject (e.g., in order to successfully inhibit alpha-synuclein spreading, seeding, oligomerization, or aggregation). As a non-limiting example, since the levels of alpha-synuclein protein in mouse interstitial fluid (ISF), CSF, brain lysate, and blood (e.g., plasma) are similar to the corresponding levels of alpha-synuclein protein in humans, a mouse model can be used to determine how much anti-alpha-synuclein protein needs to be administered to a mouse in order to achieve a desired percentage of bound alpha-synuclein, a desired reduction in alpha-synuclein spreading, seeding, oligomerization, or aggregation, or a desired therapeutic effect (e.g., a reduction in signs or symptoms of a disease). The dose can then be extrapolated from the mouse model to arrive at an appropriate dose in humans.

As a non-limiting example, a transgenic animal in which the animal overexpresses alpha-synuclein can be used as model of alpha-synuclein spreading. Such models are useful for assessing the amount of an anti-alpha-synuclein antibody that is necessary to inhibit alpha-synuclein spreading and/or establishing a relationship between a particular antibody dose and the resulting percentage of bound alpha-synuclein.

In some embodiments, the amount of the isolated antibody, antigen-binding portion, or composition that is administered to the subject (e.g., in order to treat a disease such as a neurodegenerative disease) is determined based on a level of disease progression (e.g., neurodegenerative disease progression) in the subject. In some embodiments, the amount of the isolated antibody, antigen-binding portion, or composition that is administered to the subject is determined based on a level of alpha-synuclein protein seeding or spreading (e.g., in the brain of the subject).

Various methods can be used to assess alpha-synuclein spreading. In some embodiments, immunohistochemistry (IHC) is used. For IHC methods, an antibody that is specific for phospho-129 alpha-synuclein can be used to detect the presence or extent of spreading of alpha-synuclein (e.g., in a brain of a subject such as an animal subject).

In some embodiments, the amount of alpha-synuclein protein is determined in a sample (e.g., a CSF sample, ISF, sample, brain lysate, or blood sample) in order to determine disease progression in a subject. Typically, higher amounts of alpha-synuclein protein are associated with more advanced disease progression. As a non-limiting example, ELISA can be used to measure the amount of alpha-synuclein protein that is present in a sample, e.g., using antibodies that are specific for phospho-129 alpha-synuclein.

In some embodiments, an imaging method is used to determine the level of disease progression (e.g., neurodegenerative disease progression) in the subject and/or to determine the amount of alpha-synuclein protein (e.g., human alpha-synuclein protein) spreading (e.g., in the brain of the subject). In some embodiments, two or more different imaging methods are used. Non-limiting examples of suitable imaging methods include positron emission tomography (PET) imaging, dopamine transporter (DaT) imaging, magnetic resonance imaging (MRI), and computed tomography (CT) imaging.

In some embodiments, dopamine transporter (DaT) imaging can be used to determine disease progression. DaT imaging comprises administering (e.g., intravenously administering) ¹²³I-ioflupane (N-ω-fluoropropyl-2β-carbomethoxy-3β-(4-iodophenyl) nortropane), which is a single photon emission computer tomography (SPECT) molecular imaging agent, to a subject. ²³I-ioflupane is a cocaine analog that has high affinity and good selectivity for DaT. In particular neurodegenerative diseases such as Parkinson's disease (PD), multiple system atrophy (MSA), and progressive supranuclear palsy (PSP), disease progression is associated with presynaptic dopaminergic degeneration and decreased DaT binding. In addition, ²³I-ioflupane-SPECT imaging can be used to differentiate dementia with Lewy bodies from other forms of dementia.

PET imaging can also be used to determine disease progression in a subject and/or the amount of alpha-synuclein spreading. In some embodiments, a PET probe that is specific for phospho-129 is used. In other embodiments, an antigen-binding portion or fragment of an anti-alpha-synuclein antibody described herein (e.g., an antigen-binding portion or fragment of the antibody having a short half-life) is labeled with an appropriate tracer and used as a PET probe.

Various methods can be used to assess alpha-synuclein seeding. In some embodiments, pre-formed alpha-synuclein fibrils are pre-incubated with an anti-alpha synuclein antibody and/or composition of the present disclosure, and then the pre-incubated mixture is added to a neuron preparation. Subsequently, the neurons can be fixed and stained for the presence of insoluble alpha-synuclein protein. A non-limiting example of such an assay is described in Example 2 below.

In some embodiments, the method further comprises administering to the subject one or more other therapeutic agents. In some embodiments, the method further comprises administering to the subject an agent, e.g., an antibody, that binds to amyloid beta peptides or prevents the aggregation of amyloid beta peptides. In some embodiments, the method further comprises administering to the subject an antibody against amyloid beta (Aβ), including but not limited to aducanumab, bapineuzumab, solanezumab, and gantenerumab. In some embodiments, the method further comprises administering to the subject a Beta-site Amyloid precursor protein Cleaving Enzyme 1 (BACE1) inhibitor, including but not limited to verubecestat. In some embodiments, the method further comprises administering to the subject a TDP43 antibody. In some embodiments, the method further comprises administering to the subject an A2A receptor antagonist. In some embodiments, the method further comprises administering to the subject a neuroprotective agent. In some embodiments, the neuroprotective agent is an anticholinergic agent, a dopaminergic agent, a glutamatergic agent, a histone deacetylase (HDAC) inhibitor, a cannabinoid, a caspase inhibitor, melatonin, an anti-inflammatory agent, a hormone (e.g., estrogen or progesterone), or a vitamin. In some embodiments, the method further comprises administering to the subject an agent for use in treating a cognitive or behavioral symptom of a neurodegenerative disease (e.g., an antidepressant, a dopamine agonist, or an anti-psychotic).

In some embodiments, an anti-alpha-synuclein antibody (or antigen-binding portion, or composition, or antibody-encoding isolated polynucleotide, vector, or host cell, as above) is administered to a subject at a therapeutically effective amount or dose. For an antibody or antigen-binding portion, a dose range of about 1 mg/kg to about 100 mg/kg, or about 10 mg/kg to about 50 mg/kg, can be used. The dosages, however, may be varied according to several factors, including the dose frequency, the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient.

The route of administration of an anti-alpha-synuclein antibody (or antigen-binding fragment, or pharmaceutical composition or antibody-encoding isolated polynucleotide, vector, or host cell, as above) as described herein can be oral, intraperitoneal, transdermal, subcutaneous, intravenous, intramuscular, intrathecal, inhalational, topical, intralesional, rectal, intrabronchial, nasal, transmucosal, intestinal, intraocular, ocular or otic delivery, or any other methods known in the art. In some embodiments, the antibody is administered orally, intravenously, or intraperitoneally.

Co-administered agents (e.g., the anti-alpha-synuclein antibody and another therapeutic agent) can be administered together or separately, simultaneously or at different times. When administered, the therapeutic agents independently can be administered once, twice, three, four times daily or more or less often, as needed. In some embodiments, the administered therapeutic agents are administered once daily. In some embodiments, the administered therapeutic agents are administered at the same time or times, for instance as an admixture.

In some embodiments, the anti-alpha-synuclein antibody (or antibody composition, or antibody-encoding isolated polynucleotide, vector, or host cell, as above) and another therapeutic agent are administered concurrently. In some embodiments, the anti-alpha-synuclein antibody and another therapeutic agent are administered sequentially. For example, in some embodiments an anti-alpha-synuclein antibody is administered first, prior to administering another therapeutic agent. In some embodiments, the other therapeutic agent is administered first, prior to administering an anti-alpha-synuclein antibody.

In some embodiments, the anti-alpha-synuclein antibody (and optionally another therapeutic agent) is administered to the subject over an extended period of time.

### VI. Compositions and Kits

In another aspect, compositions (also referred to as “pharmaceutical compositions”) and kits comprising an antibody (or an antigen-binding portion thereof) that specifically binds to a human alpha-synuclein protein, or an antibody composition, or an antibody-encoding isolated polynucleotide, vector, or host cell, as above, are provided. In some embodiments, the compositions and kits are for use in neutralizing human alpha-synuclein protein, e.g., in a brain of a subject. In some embodiments, neutralizing the human alpha-synuclein protein comprises preventing, reducing, or inhibiting alpha-synuclein oligomerization and/or aggregation in a subject, e.g., in a brain of a subject. In some embodiments, neutralizing the human alpha-synuclein protein comprises preventing, reducing, or inhibiting the spreading of alpha-synuclein protein oligomers and/or fibrils in a subject, e.g., in a brain of a subject. In some embodiments, neutralizing the human alpha-synuclein protein comprises preventing, reducing, or inhibiting the seeding of alpha-synuclein protein (e.g., alpha-synuclein fibrils and/or oligomers) in a subject, e.g., in a brain of a subject. In some embodiments, the pharmaceutical compositions and kits are for use in treating a neurodegenerative disease (e.g., a synucleinopathy or tauopathy).

Compositions

In some embodiments, compositions comprising an anti-alpha-synuclein antibody are provided. In some embodiments, the anti-alpha-synuclein antibody is an antibody (or an antigen-binding fragment thereof) as described herein (e.g., as described in Section III above).

In some embodiments, a composition comprises an anti-alpha-synuclein antibody as described herein and further comprises one or more pharmaceutically acceptable carriers and/or excipients. A pharmaceutically acceptable carrier includes any solvents, dispersion media, or coatings that are physiologically compatible and that do not interfere with or otherwise inhibit the activity of the active agent.

In some embodiments, the carrier is suitable for intravenous, intramuscular, oral, intraperitoneal, intrathecal, intraocular, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known in the art.

The compositions described herein can be manufactured, e.g., by means of conventional mixing, dissolving, granulating, dragee-making, emulsifying, encapsulating, entrapping or lyophilizing processes. The following methods and excipients are exemplary.

For oral administration, an anti-alpha-synuclein antibody can be formulated by combining it with pharmaceutically acceptable carriers that are well known in the art. Such carriers enable the antibody to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

An anti-alpha-synuclein antibody can be formulated for parenteral administration by injection, e.g., by bolus injection or continuous infusion. For injection, the antibody can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, the antibody can be formulated in aqueous solutions, such as physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

In some embodiments, an anti-alpha-synuclein antibody is prepared for delivery in a sustained-release, controlled release, extended-release, timed-release or delayed-release formulation, for example, in semi-permeable matrices of solid hydrophobic polymers containing the active agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients. Usually, sustained release formulations can be prepared using naturally occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

Typically, a composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the disclosure may vary depending on the particular use envisioned. Suitable dosages are also described in Section VI above.

Kits

In some embodiments, kits are provided, wherein the kits comprise: (i) the antibody or antigen-binding portion; (ii) the composition; (iii) the polynucleotide; (iv) the vector; and/or (v) the host cell; and (vi) instructions for using the antibody, antigen-binding portion, composition, polynucleotide, vector, and/or host cell to treat a neurodegenerative disease.

In other embodiments, a kit comprises an anti-alpha-synuclein antibody or antigen-binding fragment and one or more additional therapeutic agents. For example, in some embodiments, the kit comprises an anti-alpha-synuclein antibody as described herein and further comprises one or more additional therapeutic agents for use in the treatment of a neurodegenerative disease. In some embodiments, the therapeutic agent is an agent for use in treating a cognitive or behavioral symptom of a neurodegenerative disease (e.g., an antidepressant, a dopamine agonist, or an anti-psychotic). In some embodiments, the therapeutic agent is a neuroprotective agent (e.g., an anticholinergic agent, a dopaminergic agent, a glutamatergic agent, a histone deacetylase (HDAC) inhibitor, a cannabinoid, a caspase inhibitor, melatonin, an anti-inflammatory agent, a hormone (e.g., estrogen or progesterone), or a vitamin). In certain embodiments, the kit further comprises instructional materials containing directions (i.e., protocols) for the practice of the methods described herein (e.g., instructions for using the kit for treating a neurodegenerative disease (e.g., a synucleinopathy or tauopathy)).

While the instructional materials typically comprise written or printed materials, they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD-ROM), and the like. Such media may include addresses to internet sites that provide such instructional materials.

### VII. Examples

Embodiments of the present disclosure will be described in greater detail by way of specific examples. The following examples are offered for illustrative purposes only, and are not intended to limit the disclosure in any manner.

### Example 1

### Anti-Alpha-Synuclein Antibody Discovery and Screening

This example describes the generation and characterization of antibodies that specifically bind to human alpha-synuclein protein. The antibodies have about the same binding affinity for unphosphorylated alpha-synuclein protein as for alpha-synuclein protein that is phosphorylated at serine 129. In some instances, the antibodies have single-digit nanomolar binding affinity for both unphosphorylated alpha-synuclein protein and alpha-synuclein protein that is phosphorylated at serine 129.

### Preparation of Human Alpha-Synuclein Fibrils

Pre-formed human alpha-synuclein fibrils were generated according to published protocols using alpha-synuclein monomer protein (Anaspec Inc.). See, e.g., Volpicelli-Daley, L. A. et al., Nat. Protoc. 9(9):2135-46 (2014); and Polinski, N. K., et al., J. Parkinsons Dis. 2018 Jan. 30 (doi: 10.3233/JPD-171248). Lyophilized human alpha-synuclein monomer protein was reconstituted in PBS to a concentration of 5 mg/mL and incubated at 37° C. for 7 days with constant agitation at 1,000 rpm on a ThermoMixer shaker with a heated ThermoTop. Successful fibril formation was confirmed by Thioflavin T binding (Xue, C., et al., R. Soc. Open. Sci. 2017 January; 4(1): 160696). Confirmation was also obtained by sedimentation test whereby the mixture was ultracentrifuged at high speed (e.g., 100,000 g) to pellet down aggregated species. The supernatant fraction, which contained monomers and possibly oligomers, was set aside, and the pellet fraction, which contained large fibrils, was separated and analyzed on a SDS-PAGE gel. Successful fibrillization was confirmed by a finding that the majority of the protein recovered was in the pellet fraction. Prior to the competition ELISA experiments described herein, the pre-formed fibrils were sonicated for 20 seconds at 20% power using a hand-held sonicator probe with a ⅛″ tip at room temperature.

### Rat Immunization

SAS SD (Sprague Dawley) male rats, 42-50 days of age (Charles River) were immunized with recombinant human alpha-synuclein monomers, oligomers, and fibrils. Immunizations were performed via footpad by injecting 5-10 μg of antigen per animal in Ribi adjuvant (1:1 w/w) two times per week. The antigen used in the injections was alternated between monomeric and oligomeric alpha-synuclein. After 6-8 injections, the serum was screened for titer by protein indirect ELISA with alpha-synuclein monomer-coated plates (1 μg/mL) detected with anti-rat HRP (Invitrogen, No. 62-9520). Titers were measured for four rats, and once the titers measured >10⁵ dilution for one rat, the rat was selected for a final boost. The final boost was done by injecting 5 μg per animal of antigen (combination of monomers, oligomers and fibrils) without adjuvant, in PBS (1:1 w/w) via footpad. The animal was sacrificed three days later. Popliteal and inguinal lymph nodes were harvested, 18×10⁶ lymphocytes were processed into single-cell suspensions by passing through cell strainers and used for fusion.

### Generation of Hybridoma Library by ClonePix

B cells harvested from the rat lymph nodes were processed and counted. The 18×10⁶ lymphocytes were split in half and 9×10⁶ lymphocytes were mixed with P3X63Ag8 cells (ATCC) 1:1 and fused using a BTX Hybrimune Electrofusion apparatus in electrofusion buffer (BTX) at room temperature. The fused hybridomas were recovered for 24 hours at 37° C. in a recovery medium (Stem Cell Technologies) and plated in a semi-solid medium with hypoxanthine-aminopterin-thymidine (HAT) selection media and IgG FITC reagent for 8-10 days. Single-cell colonies were screened for IgG expression and about 400 clones were picked using Clonepix and transferred to 96-well plates in Hypoxanthine Thymidine (HT) medium. After a week, 50 μL/well of supernatant was collected and screened for antigen specific binding.

### Hybridoma Generation

The second half (i.e., the remaining 9×10⁶ lymphocytes) from rat lymph nodes were mixed with mouse Sp2/mIL-6 myeloma fusion cells (ATCC) at a ratio of 1:2 and fused using a BTX Hybrimune Electrofusion apparatus in electrofusion buffer (BTX) at room temperature. The fused cells were recovered in hybridoma fusion recovery medium (Stemcell Technologies) for 12 hours at room temperature, and suspended in hypoxanthine-aminopterin-thymidine (HAT) selection media and plated in 28 of 96 well plates with 5,000 B cells per well. The medium was replaced with hypoxanthine thymidine (HT) medium after a week at 37° C. Two weeks after fusion, 50 μL/well of supernatant was collected and all 28 plates (about 2240 hybridomas) were screened for antigen-specific binding.

### Primary Direct ELISA Assay for Alpha-Synuclein Binding

A direct ELISA assay was performed for the primary screening. 96-well Nunc plates were coated with 50 ng/mL of monomeric alpha-synuclein in PBS 50 μL/well overnight at 4° C. The next day, the plates were washed three times with PBST and 50 μL/well of hybridoma supernatants were transferred from fusion plates to screening plates. The plates were incubated for 1 hour at room temperature, then washed three times with PBST. Secondary detection antibody goat anti-rat HRP (obtained from Southern Biotech) was added 50 μL/well 1:5,000 diluted in PBS. Plates were incubated for one hour at room temperature. After one hour the plates were washed three times with PBST. Plates were developed with 50 μL/well of 3,3′,5,5′-tetramethylbenzidine (TMB) substrate (obtained from Thermo Fisher) and quenched with 50 μL/well of 1 N sulfuric acid. The signal was quantified on a BioTek® plate reader at A450. Wells with an OD three times the background were considered positive. About 1,424 positive clones were identified from the original 28 96-well plates and were selected for further characterization.

### Secondary Competition ELISA Assay

A competition-based ELISA assay was used to evaluate the top 158 positive clones (with OD₄₅₀>2.5, 10 times higher than the background signal) from the direct ELISA assay described above. For each hybridoma, three 50μL aliquots of cell culture supernatants were collected. The supernatants were either mixed with either PBS control, 100 nM alpha-synuclein monomer, or 50 nM alpha-synuclein fibril. After incubation at room temperature for 1 hour, the mixtures were loaded onto alpha-synuclein protein-coated Nunc plates, and an ELISA assay was performed using similar conditions as the direct ELISA described above. The presence of alpha-synuclein monomers or fibrils in the mixture reduced the level of free antibodies in the supernatant. Compared with the PBS-mixing control, 60 clones demonstrated a greater than 20% reduction with either monomeric or fibril alpha-synuclein competition and were selected for subcloning and further characterization.

Measurement of Binding Kinetics of Antibodies to Monomeric and phosphoS129 Alpha-Synuclein Using Surface Plasmon Resonance (SPR, Biacore™)

Anti-mouse Fc antibody (obtained from GE Healthcare) was immobilized on the surface of a CM5 chip (obtained from GE Healthcare) through amine-coupling to reach about 6,000 to 8,000 response units (RU). The surface was activated by injection of a mixture of 1-ethyl-3-(3-dimethylaminopropyl)-carbodiimide (EDC) and N-hydroxysuccinimide (NETS), both obtained from GE Healthcare, for 7 minutes at room temperature. Anti-mouse Fc antibody was diluted in sodium acetate (pH 5.0) at 25 μg/mL and injected for 10 minutes at a flow rate of 5 μL/minute, followed by injection of ethanolamine (obtained from GE Healthcare) for 7 minutes. Both injections were done at room temperature.

Purified rat anti alpha-synuclein antibody (10 μg/mL) was injected at room temperature and captured to reach 1,000 to 2,000 RU. To test antibody affinity for monomeric alpha-synuclein, a range of serially-diluted monomeric human alpha-synuclein (e.g., 3.4 nM to 300 nM) was injected at a flow rate of 30 μL/minute, using full-cycle kinetics methods at room temperature. Sensorgrams were fitted using a 1:1 Langmuir model to estimate kₒₙ and k_{off}. Table 2 shows that the selected antibodies had k_{off} values for alpha-synuclein monomer ranging from around 1.6×10⁻³ to 3.3×10⁻³ s⁻¹, and affinities (K_{D} values) for alpha-synuclein monomer were between about 3.0 nM and about 18 nM. Kinetics for binding to alpha-synuclein phosphorylated at serine 129 were determined using the full-cycle kinetics methods as described above for the monomers. Table 2 shows that the selected antibodies had affinities (K_{D} values) for alpha-synuclein pSer129 ranging from about 6.1 nM to about 42 nM. It was noted that the clone 20H4 has similar single-digit nM affinity for both alpha-synuclein monomers (3 nM) and pSer129 alpha-synuclein monomer peptide (6 nM).

### Competition ELISA Analysis for Fibril Binding Affinity

To illustrate the binding potentiation of selected antibodies toward alpha-synuclein fibrils, a competition ELISA analysis was performed. Purified antibody was probed with different concentrations of alpha-synuclein fibrils in solution, and the levels of unbound antibody were measured by ELISA analysis. In this experiment, 0.6 nM (0.1 μg/mL) of purified antibody was dissolved in phosphate-buffered saline (PBS) solution. Different concentrations of alpha-synuclein fibrils (ranging from 0 nM to 2,800 nM or 40 μg/mL) were added to the antibody solutions in separated wells. The mixtures were gently shaken at room temperature for 1 hour to reach equilibrium. After that, these samples were transferred to an alpha-synuclein protein-coated ELISA plate and incubated for 5 minutes. During this short incubation, antibody that was not associated to antigen in the previous solution could instead bind onto the plate, and thus be quantified using a standard ELISA technique. By this means, it was possible to determine the amount of free antibody in the initial mixture with alpha-synuclein fibrils at indicated concentrations.

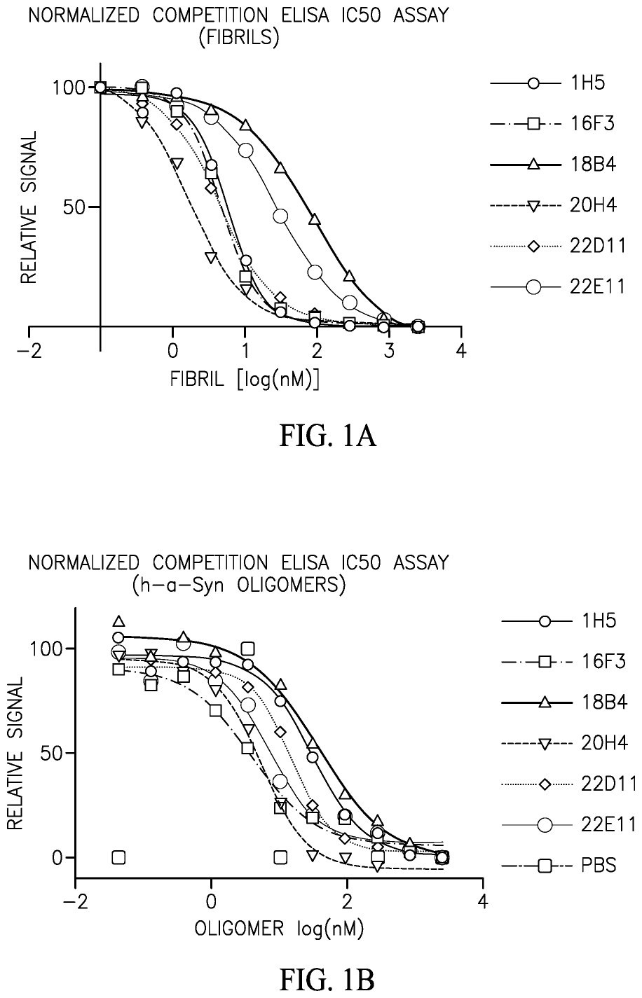

The dataset was normalized by setting up the no-fibril control for each antibody as 100%. A binding curve was generated by plotting the concentration of unbound antibody, which was represented by the ELISA signal, to the concentration of the fibrils in the initial mixture. The IC50 was represented by the fibril concentration at which 50% of the antibody was bound to the plate. According to the competition ELISA binding results (FIG. 1A), the selected rat anti-alpha-synuclein antibodies had IC50 values ranging from about 1.7 nM to about 86 nM (Table 2). Specifically, the IC50 value for clone 20H4 was less than 2 nM, and that of clones 1H5, 16F3 and 22D11 were less than 10 nM. It is important to note that the solution concentration of fibrils was estimated based on the extinction coefficient of alpha-synuclein monomers and therefore may not accurately reflect the number of oligomeric molecules in solution.

### Competition ELISA Analysis for Oligomer Binding Affinity

Human alpha-synuclein protein (Anaspec AS55555) was dissolved at a concentration of 1 mg/mL in PBS and verified to be >95% monomeric by size exclusion chromatography. Alpha-synuclein protein was concentrated to 2 mg/mL in PBS and mixed with a solution of 4-hydroxy-nonenal (Cayman, 10 mg/mL in ethanol), at a final molar ratio of 1:20 protein/4HNE and incubated overnight at 37° C. The resulting mixture was analyzed and found to consist of approximately 80% oligomers by area under the curve on analytical size exclusion chromatography, with the remainder of alpha-synuclein being monomeric species in equilibrium. The composition was used without further purification.

To measure the binding affinities of select antibodies toward oligomeric alpha-synuclein protein, purified antibodies were probed with different concentrations of human alpha-synuclein oligomers in solution, and the levels of unbound antibody were measured by ELISA analysis. In this experiment, 0.6 nM (0.1 pg/mL) of purified antibody was dissolved in phosphate-buffered saline (PBS) solution. Different concentrations of alpha-synuclein oligomer (ranging from 0 nM to 2,500 nM or 35 ug/ml) were added to the antibody solutions in separated wells. The mixtures were gently shaken at room temperature for 1 hour to reach equilibrium. After that, these samples were transferred to an alpha-synuclein protein-coated ELISA plate, and antibody not bound to antigen in the previous solution was detected by direct ELISA.

The dataset was normalized by setting up the no-oligomer control for each antibody as 100%. A binding curve was generated by plotting the concentration of unbound antibody, which is represented by the ELISA signal, to the concentration of the oligomer in the initial mixture. The IC50 was represented by the oligomer concentration at which 50% of the antibody was bound to the plate. The oligomer competition ELISA binding results are presented in FIG. 1B and Table 2.

### Epitope Mapping Using Peptide-Based ELISA

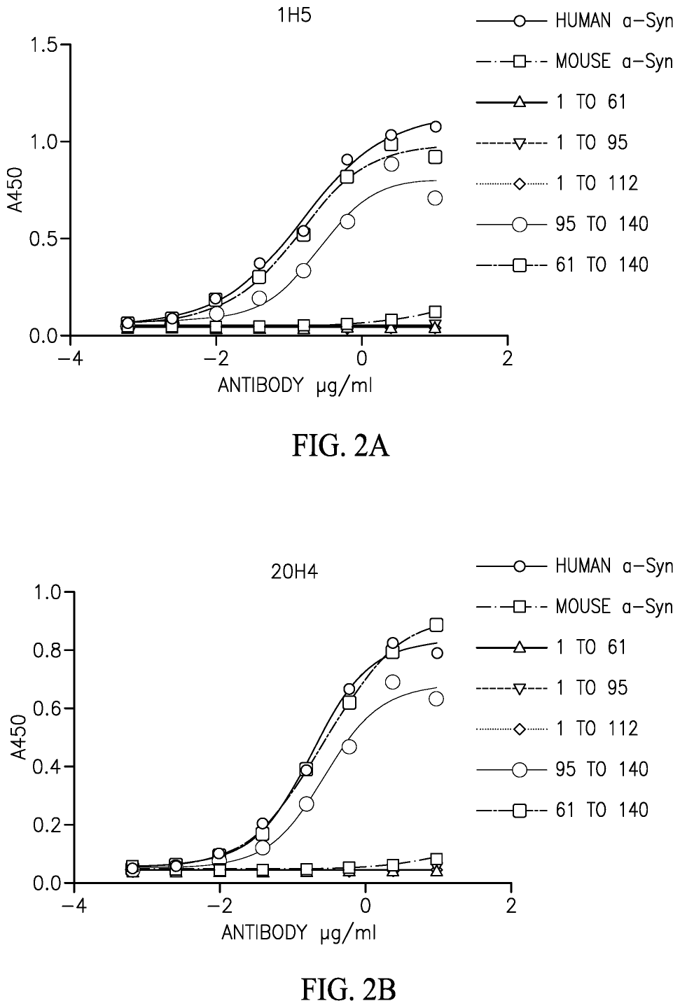

96-well Nunc plates were coated with 1 pg/mL of the indicated synthetic peptide in PBS 50 μL/well overnight. The plates were washed three times with PBST, then blocked with 150 μL of 3% BSA in PBS for 1 hour. Purified antibody was diluted by serial dilution methods, from 10 pg/mL to 0.6 ng/ml, and 50 μL of the antibody solution was added to the peptide-coated wells. The plates were incubated for 1 hour at room temperature, then washed three times with PBST. Secondary detection antibody, goat anti-rat HRP (obtained from Southern Biotech) 1:5,000 diluted in BSA-PBS, was added to a concentration of 50 μL/well. Plates were incubated for one hour and washed three times with PBST. Plates were then developed with 50 μL/well of TMB substrate (obtained from Thermo Fisher) and quenched with 50 μL/well of 1 N sulfuric acid. The signal was quantified on a BioTek® plate reader at A450. Binding curves were generated by plotting the ELISA signal to the concentration of antibody solution (FIGS. 2A-2F). The results are summarized in Table 3 (wherein, for Table 3 and Table 4, ++++ means ELISA positive with antibody concentration lower than 0.04 ug/ml, +++ means ELISA positive with antibody concentration between 0.04-0.16 ug/ml, ++ means ELISA positive with antibody concentration between 0.16-0.64 ug/ml, + means ELISA positive with antibody concentration between 0.64-2.6 ug/ml, and − means ELISA negative with antibody concentration greater than 2.6 ug/ml).

Binding to different synuclein species was tested using a method similar to the ELISA format described above. Briefly, recombinant human alpha-synuclein protein, beta-synuclein protein, and gamma-synuclein protein was coated onto 96-well Nunc plates at 1 μg/mL. The plates were washed with PBST and blocked with 150 μL of 3% BSA in PBS. The serial dilutions of purified antibodies were applied onto the plate, and the associated antibody was detected with goat anti-rat HRP secondary antibody. As shown in Table 4, the antibodies only recognized alpha-synuclein protein, and no binding was detected to either beta-synuclein protein or gamma-synuclein protein.

### Epitope Mapping Using Peptide Microarray

A peptide microarray spotted with synthetic 15-mers with an overlap of 10 amino acids (JPT, Berlin, Germany) covering the full-length human alpha-synuclein (SEQ ID NO:1; NP 000336.1) was used for epitope mapping. Spots of human IgG and mouse IgG were included as positive controls. Monoclonal antibodies (either rat monoclonal antibody or rat variable-human IgG1 chimeras) diluted to a concentration of 10m/mL in TBST with 3% BSA were incubated on glass slides for 2 hours at room temperature. After washing 5× with TBST, Alexa Fluor® 568 coupled to goat anti-human IgG (H+L) or Alexa Fluor® 647 coupled to goat anti-rat IgG (H+L) (obtained from Life technologies; 1 μg/mL in TB ST with 3% BSA) was added for 1 hour protected from light. The slides were washed 5× with TB ST and 5× with deionized water to remove excess salts, then dried under nitrogen. The slides were imaged with an Opera Phenix™ slide scanner (obtained from Perkin Elmer®, Waltham, Mass.) and analyzed using ImageJ/TrackEM software version 2.0.0.

### Fine Epitope Mapping Using Alanine Scanning Peptide ELISA

To further characterize the amino acid residues recognized by clone 20H4, each residue within residues 113-127 of human alpha synuclein was sequentially substituted with alanine, save for position 124, where alanine was substituted with glycine or proline. 17 N-terminal biotinylated peptides were designed (Table 7), and synthesized by Anaspec Inc.

96-well Nunc plates were coated with 50 μL per well of 1 μg/mL streptavidin (from Jackson Immuno Research Laboratory) in PBS at 4° C. overnight. The plates were washed three times with PBST, and blocked with PBS containing 3% BSA at room temperature for 1 hour. The N-terminal biotinylated peptides were dissolved in Milli Q water to a concentration of 2 mg/mL, then further diluted to a concentration of 2 μg/mL with PBS. 50 μl of the diluted peptide was applied to the streptavidin-coated plates and incubated at 4° C. overnight. 2 μg/mL of biotinylated human alpha-synuclein was used as the positive control. The plates were washed three times with PBST before use. Purified antibody was diluted by serial dilution methods, from 20 μg/mL to 1 ng/mL, and 50 μL of the antibody solution was added to the peptide-coated wells. The plates were incubated for 1 hour at room temperature, then washed three times with PBST. Secondary detection antibody (goat anti-rat HRP (obtained from Southern Biotech)) was added with 1:5,000 dilutions and incubated for 1 hour at room temperature, then washed three times with PBST. Plates were developed with 50 μL/well of TMB substrate (Thermo Fisher) and quenched with 50 μL/well of 1 N sulfuric acid. The signal was quantified on a BioTek® plate reader at A450. A parallel repeat was performed for each peptide in a separate plate.

Data for both parallel experiments are shown in Table 8. Particularly important residues for 20H4 binding included D119, N122, and Y125 of SEQ ID NO:1. A124 of SEQ ID NO:1 also appeared to be important for binding. Replacing A124 with proline resulted in a loss of binding, whereas replacing A124 with glycine had no effect. It is noted that the loss of binding caused by substitution of A124 with proline may have been due to structural disruption of the backbone induced by the proline residue.

### Antibody Variable Domain Sequencing

Total RNA was extracted from approximately 5×10⁶ hybridoma cells (i.e., that produced each antibody) using a Qiagen RNeasy Mini Kit. The first-strand cDNA was synthesized using a SMART RACE cDNA Amplification Kit (obtained from BD Biosciences Clontech) following the manufacturer's protocol. The variable region cDNAs for the heavy (VH) and light (VL) chains were amplified by polymerase chain reaction (PCR) using 3′ primers that annealed, respectively, to the rat gamma and kappa chain C regions (SEQ ID NOs:53-57 listed below), and a 5′ universal primer that was provided in the SMART RACE cDNA Amplification Kit.

For VH PCR, the 3′ primers were as follows:

ratIgG1
(SEQ ID NO: 53)
AGGCTTGCAATCACCTCCACA,
ratIgG2a
(SEQ ID NO: 54)
ACAAGGATTGCATTCCCTTGG,
ratIgG2b
(SEQ ID NO: 55)
GCATTTGTGTCCAATGCCGCC,
and
ratIgG2c
(SEQ ID NO: 56)
TCTGGGCTTGGGTCTTCTGGG,

For VL PCR, the 3′ primer was as follows:

ratCkappa CTCATTCCTGTTGAAGCTCTTGACGACGGG (SEQ ID NO:57).

The PCR products were separated by agarose gel electrophoresis. The DNA fragments coding for VH and VL were purified using a QIA quick Gel Extraction Kit (obtained from Qiagen), and subcloned into a pCRII-TOPO vector using a TOPO TA cloning kit (obtained from Invitrogen). Each successful clone was sequenced by Sanger sequencing and at least 16 clones were sequenced for each sample.

Cloning Rat Antibody Variable Regions into Human Chimeric Antibodies

The DNA sequences of rat antibody variable regions, including VH and VL, were codon-optimized for expression in human cell lines. The optimized VH gene was cloned into plasmid pRK5.1-hIgG1, in the same open reading frame and at the 5′-end of human IgG1 constant region. Similarly, the optimized VL gene was cloned into plasmid pRK5.1-hKappa at the 5′-end of human immunoglobulin kappa constant region. The resulting plasmids were confirmed by Sanger sequencing, and amplified by maxi-prep for transfection.

### Example 2

### Immunodepletion and Inhibition of Alpha-Synuclein Seeding

This example shows that antibodies of the present disclosure are useful for inhibiting the seeding of alpha-synuclein in neurons, and also that the antibodies can immunodeplete both soluble and aggregated alpha-synuclein protein.

### Inhibition of Alpha-Synuclein Pre-Formed Fibril-Induced Seeding in Primary Hippocampal Neurons

Primary hippocampal cultures were prepared from E16 to E18 CD1 mouse brains and maintained as described (Volpicelli-Daley et al. Neuron (2011) 72:57-71). Fibril transduction was performed at 6-11 DIV as described (Volpicelli-Daley et al. Neuron (2011) 72:57-71). For antibody treatment, 71 nM a-syn pffs were pre-incubated with 40 nM alpha-synuclein antibody (anti-RSV control; Antibody 1; Antibody 2; rat 20H4 and chimeric 20H4 (“Chi 20H4”)) at 37° C. for 30-40 minutes before being added to neurons. Antibody 1 (also referred to as clone 9E4) is described, e.g., in WO2005/047860 and includes the heavy chain and light chain variable regions represented by SEQ ID NOs:230 and 231, respectively. Antibody 2 (also referred to as as1o452 ng13) is described, e.g., in WO2017/207739 and includes the heavy chain and light chain variable regions represented by SEQ ID NOs:232 and 233, respectively. Antibody 1 and Antibody 2 were also used as comparators in other experiments described herein.

After a 7-day incubation, the neurons were fixed and stained for Triton X 100-insoluble pS129 alpha-synuclein using a rabbit monoclonal antibody specific for phosphoS129 alpha-synuclein (MJF-R13 (8-8)). The relative efficiencies of alpha-synuclein antibodies in seeding inhibition were calculated based on fluorescence intensity normalized to cell counts. Each antibody condition was performed in triplicate and the experiment was repeated at least 3 times.

### Parkinson's Disease (PD) Brain Lysate Immunodepletion

PD brain tissues were sequentially extracted using 750 mM NaCl-containing high salt RAB buffer (HS-RAB), followed by HS-RAB with 1% Triton-X100 and finally by HS-RAB with 1% sarkosyl. The sarkosyl-insoluble pellet was sonicated in PBS to create a homogeneous suspension.

For immunodepletion of soluble alpha-synuclein in PD brain lysates, antibodies (30 nM) were incubated with Protein G beads (Dynabeads; ThermoFisher) for 1 hour at room temperature. After three washes with PBS to remove unconjugated antibodies, the high salt fraction of PD brain extracts was diluted about 200-fold in PBS containing 0.02% Tween-20 and protease inhibitor cocktail (Cell Signaling Technology) and added to antibody-bead complexes in a V-bottom 96-well plate, then incubated overnight at 4° C. with constant shaking at 750-800 rpm. The unbound fraction was collected and measured using a U-PLEX Human a-Synuclein ELISA Kit (MSD #K151WKK) according to the manufacturer's instructions. Values were normalized to values from mock immunoprecipitation using an anti-RSV antibody. The amount of alpha-synuclein in the unbound fraction was normalized to that of control antibody (anti-RSV) treatment and plotted as percentage of anti-RSV.

Immunodepletion of insoluble alpha-synuclein was performed following the same procedure as above on 2-4 fold diluted sarkosyl-insoluble pellet fractions of PD brain extracts. Aggregated alpha-synuclein in the unbound fraction of immunoprecipitation was measured by sandwich ELISA using a monoclonal antibody specific for aggregated alpha-synuclein (Syn-F1; BioLegend) as the capture antibody and biotinylated Syn211 as the reporting antibody. Values were normalized to values from mock IP using an anti-RSV antibody.

### Parkinson's Disease (PD) Patient CSF Immuno-Depletion

Patient CSF was obtained from PrecisionMed. Magnetic Protein G beads (SureBeads; Bio-Rad) were first blocked with 2% BSA in TBS for 1 hour at 24° C. and then washed three times in TBS-t. 50 μL of blocked bead slurry were then incubated with 1.35 μg antibody in 150 μL of PBS for 1 hour at 24° C. and then washed three times in TBS-t. Prior to incubation with antibody conjugated beads, Tween-20 (0.02%) and EDTA-free cOmplete protease inhibitors (Roche) were added to CSF. CSF (30 μL) was then added to the antibody-conjugated beads and incubated at 4° C. for 18 hours. The unbound fraction was collected and measured using a U-PLEX Human a-Synuclein Kit (MSD #K151WKK) according to the manufacturer's instructions.

### Example 3

### Species Cross-Reactivity Analysis Using Direct ELISA

A direct ELISA assay was performed to test species cross-reactivity of clones 1H5, 16F3, 18B4, 20H4, 22D11, and 22E11. 96-well Nunc plates were coated with 50 μL of recombinant human, mouse, or rat alpha-synuclein at a concentration of 1 μg/mL in PBS at 4° C. overnight. The plates were then washed three times with PBST and blocked with PBS containing 3% BSA at room temperature for 1 hour. Purified antibody was diluted by serial dilution methods, from 10 μg/mL to 0.6 ng/mL, and 50 μL of the antibody solution was added to the alpha-synuclein-coated wells. The plates were incubated for 1 hour at room temperature, then washed three times with PBST. Secondary detection antibody (goat anti-rat HRP (obtained from Southern Biotech)) was added 50 μL/well, 1:5,000 diluted in BSA-PBS solution. Plates were incubated for 1 hour at room temperature, then washed three times with PBST. Plates were subsequently developed with 50 μL/well of TMB substrate (Thermo Fisher) and quenched with 50 μL/well of 1 N sulfuric acid. The signal was quantified on a BioTek® plate reader at A450.

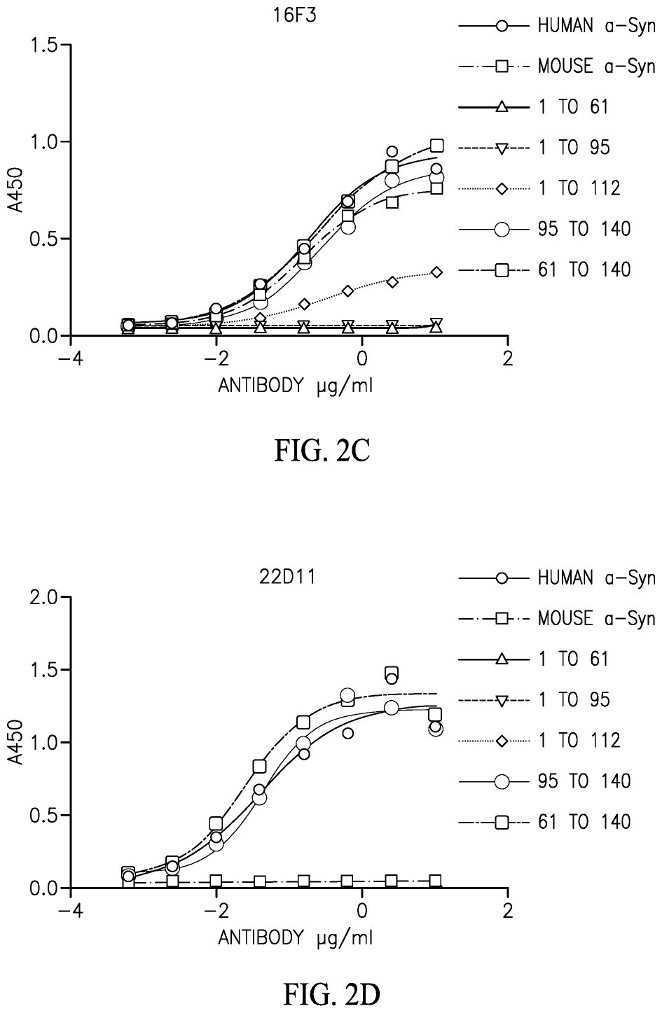

Binding curves were generated by plotting the ELISA signal to the concentration of antibody solution (FIGS. 3A, 3B, and 3C for binding to human, mouse, and rat, respectively). Results are summarized in Table 6. Of note, all six clones exhibited a higher binding affinity for human alpha-synuclein protein than for rat alpha-synuclein protein, having EC50 values that were between about 3.33-fold and about 66.67-fold higher for binding to rat alpha-synuclein, compared to human alpha-synuclein.

### Example 4

### Antibody Humanization and Affinity Maturation

This example describes humanization and subsequent affinity maturation of clone 20H4. Unless otherwise specified, residue numbers for antibody variable domains in this example are according to Kabat et al., Sequences of proteins of immunological interest, 5^{th} Ed., Public Health Service, National Institutes of Health, Bethesda, Md. (1991).

Direct Hypervariable Region Grafts onto the Acceptor Human Consensus Framework

Variants constructed during the humanization of 20H4 were assessed in the form of an IgG. The VL and VH domains from rat 20H4 were aligned with the human VL kappa I (VL_{KI}) or VL kappa III (VL_{KIII}) consensus sequences, and human VH subgroup I (VH_{I}) or VH subgroup III (VH_{III}) consensus sequences, respectively. Hypervariable regions (HVRs) from the rat 20H4 antibody were engineered into VL_{KI}, VL_{KIII}, VH_{I}, and VH_{III} acceptor frameworks to generate CDR-graft variants. From the rat 20H4 VL domain, positions 24-34 (L1), 50-56 (L2), and 89-97 (L3) were grafted into either VL_{KI} or VL_{KIII}. From the rat 20H4 VH domain, positions 26-35 (H1), 50-65 (H2), and 93-102 (H3) were grafted into either VH_{I} or VH_{III}. The HVR definitions are defined by their sequence hypervariability (Wu, T. T. & Kabat, E. A. (1970)), their structural location (Chothia, C. & Lesk, A. M. (1987)), and their involvement in antigen-antibody contacts (MacCallum et al. J. Mol. Biol. 262: 732-745 (1996)). In order to evaluate framework vernier positions that might be important, selected vernier positions were mutated back to the rat sequence. The vernier positions include 46P, 58V, and 71Y in VL, and W47, L48, A49, F67, D73, and V78 in VH. Eleven different versions of VH and six versions of VL sequences were synthesized (gblocks from Integrated DNA Technologies (IDT)) and cloned into mammalian expression vectors. Fifty-five different combinations of humanized graft variants were generated and evaluated by surface plasmon resonance (SPR) analysis.

### Affinity Maturation

The phagemid used for this work was a monovalent Fab-g3 display vector that consisted of 2 open reading frames under the control of a single lac promoter. The first open reading frame consisted of the OmpA signal sequence fused to the VL and CL domains of the acceptor light chain, and the second open reading frame consisted of the PelB signal sequence fused to the VH and CH1 domains of the acceptor heavy chain, followed by the C-terminal truncated phage coat protein P3. The CDR graft variant (20H4 HC2 LC2) was generated as the Fab displayed on phage.

To improve affinity, a total of six soft-randomized phage libraries containing changes in hypervariable regions were generated. Sequence diversity was introduced at the selected randomization regions using Kunkel mutagenesis. For library generation, positions in the hypervariable region were kept at a 50% mutation rate by using oligonucleotides with a mixture containing 70% of the base found in the wild-type sequence and 10% each of the other three bases at each position. This allowed multiple residues in the targeted region to be simultaneously randomized.

### Generation of Phage Libraries

Oligonucleotides designed to introduce diversity into each hypervariable region were phosphorylated separately in 20 μL reactions containing 660 ng of oligonucleotide, 50 mM Tris pH 7.5, 10 mM MgCl₂, 1 mM ATP, 20 mM DTT, and 5 U polynucleotide kinase for 1 hour at 37° C. From the phosphorylated oligonucleotides reactions, 2 μL was added to 500 ng Kunkel template in 50 mM Tris pH 7.5 and 10 mM MgCl₂ in a final volume of 25 μL. The mixture was annealed at 90° C. for 1 minute, 50° C. for 3 minutes and then cooled on ice. The annealed template was then filled in by adding 0.5 μL 10 mM ATP, 0.5 μL 10 mM dNTPs (10 mM each of dATP, dCTP, dGTP, and dTTP), 1 μL 100 mM DTT, 1 μL 10× TM buffer (0.5 M Tris pH 7.5, 0.1 M MgCl₂), 80 U T4 ligase, and 4 U T7 polymerase in a total volume of 30 μL for 2 hours at room temperature. These filled-in and ligated products were then each transformed into XL1-blue cells. The libraries were recovered in 10 mL SOC media for 1 hour at 37° C. Carbenicillin (50 μg/mL) and M13/K07 helper phage (MOI 10) were added. The cultures were incubated for another 30 minutes at 37° C. and transferred to 500 mL 2YT containing 50 μg/mL carbenicillin and 50 μg/mL kanamycin and grown overnight at 30° C.

### Phage Selections

Recombinant alpha-synuclein (aSyn) was biotinylated through free amines using Sulfo-NHS-LC-Biotin (Thermo Scientific). For biotinylation reactions, a 3-fold molar excess of biotin reagent was used in PBS. Reactions were followed by extensive dialysis in PBS.

Phage libraries were harvested from the cell culture supernatant and suspended in PBS containing 1% BSA. The phage libraries were incubated with biotinylated alpha-synuclein at room temperature and the phage bound to biotinylated alpha-synuclein was then captured for 5 minutes on Dynabeads M-280 Streptavidin. The beads were washed extensively with PBS containing 0.05 Tween 20 (PBST) and bound phage were eluted by incubating the wells with 100 mM glycine (pH 2.7) for 15 minutes. Eluted phage were neutralized with 1.5 M Tris, pH 8.8, and amplified using TG1 cells and M13/K07 helper phage and grown overnight at 37° C. in 2YT, 50 μg/mL carbenicillin and 50 μg/mL kanamycin. The titers of phage eluted from antigen immobilized beads were compared to titers of phage recovered from beads without antigen to assess enrichment. Selection stringency was increased by both decreasing concentration of biotin-aSyn (from 10 nM to 0.1 nM) during binding and increasing the competition time (from 0 to 2 hours at room temperature) with 1 μM of unlabeled alpha-synuclein in solution.

### SPR Assessment of Variants

The humanized and affinity matured 20H4 variants were expressed as IgG by 293 transient transfections. IgG was purified with protein A affinity chromatography. The affinity of each humanized 20H4 (hu20H4) IgG variant for alpha-synuclein monomers was determined by SPR using a Biacore™ 8 K. Biacore™ Series S CM5 sensor chips were immobilized with monoclonal mouse anti-human IgG (Fc) antibody (human antibody capture kit from GE Healthcare). Serial 3-fold dilutions of aSyn were injected at a flow rate of 30 μL/min. Each sample was analyzed using a 3-minute association and a 10-minute dissociation. After each injection, the chip was regenerated using 3 M MgCl₂. Binding response was corrected by subtracting the RU from a flow cell capturing an irrelevant IgG at similar density. A 1:1 Languir model of simultaneous fitting of kₒₙ and k_{off} was used for kinetics analysis. Data is shown in Tables 9-11.

A humanized, affinity-matured variant of 20H4 having the heavy chain variable sequence of “HC5” and the light chain variable sequence of “L1_1” was selected for further experiments and engineering. The variable domain sequences of HC5 and L1_1 are provided in SEQ ID NOs: 147 and 173, respectively. This variant is referred to herein as 20H4_HC5_L1_1. Other variants were also generated.

Table 13 also illustrates a comparison of binding kinetics (to aSyn monomers) for antibody 20H4_HC5_L1_1 (VH of SEQ ID NO.:147; VL of SEQ ID NO.:173, wt Fc) and reference anti-synuclein antibodies Antibody 1 and Antibody 2, as described in Example 2. Binding as shown in Table 13 was measured by Biacore™.

TABLE 13
Binding kinetics of Anti-Synuclein Antibodies

| Antibody | kₐ (1/Ms) | k_{d} (1/s) | K_{D} (M) |
|---|---|---|---|
| 20H4_HC5_L1_1 | 6.47E+5 | 1.36E−4 | 2.10E−10 |
| Antibody 1 | 9.79E+3 | 7.00E−5 | 7.15E−9 |
| Antibody 2 | 1.79E+5 | 1.50E−3 | 8.37E−9 |

Affinity of antibody bearing 20H4_HC5_L1_1 Fab for aSyn monomers, oligomers, and fibrils was assessed using SPR. In this experiment, the antibody bound to aSyn fibrils with a K_{D} of 270 pM, to aSyn oligomers with a K_{D} of 190 pM, and to aSyn monomers with a K_{D} of 120 pM.

Kinetic Exclusion Assay Assessment of Binding of 20H4_HC5_L1_1

Table 14 illustrates a comparison of binding kinetics (to aSyn monomers, oligomers, and fibrils) for antibody 20H4_HC5_L1_1 (VH of SEQ ID NO.:147; VL of SEQ ID NO.:173, wt Fc) and reference anti-synuclein Antibody 2, as described in Example 1. Binding was measured by KinExA™.

TABLE 14
Binding kinetics of Anti-Synuclein Antibodies

| | KinExA ™ binding KD | | | |
|---|---|---|---|---|
| Antibody | aSyn monomer | aSyn oligomer | aSyn fibril | |
| 20H4_HC5_ | 15.7 pM | 8.86 pM | 638 | fM |
| L1_1 | | | | |
| Antibody 2 | 131 pM | 1.07 pM | 6.31 | pM |

### Example 5

### In Vivo Plasma Pharmacokinetics of Engineered 20H4 Antibodies

### First Mouse Study

In a seven-day plasma pharmacokinetics study, wild-type 6-8 week old male C57B16 mice (n=3/group) were intravenously dosed with 10 mg/kg of antibody (20H4_HC5_L1_1 with LALA Fc mutations, or a control anti-RSV antibody with LALAPG (LALA plus P329G); all antibodies were expressed as human IgG1). In-life bleeds were taken at 30 min, 1 day, 4 days, and 7 days post-dose via submandibular bleeds. Blood was collected in ETDA plasma tubes and spun at 14,000 rpm for 5 min. Plasma was then isolated for subsequent pharmacokinetic analysis.

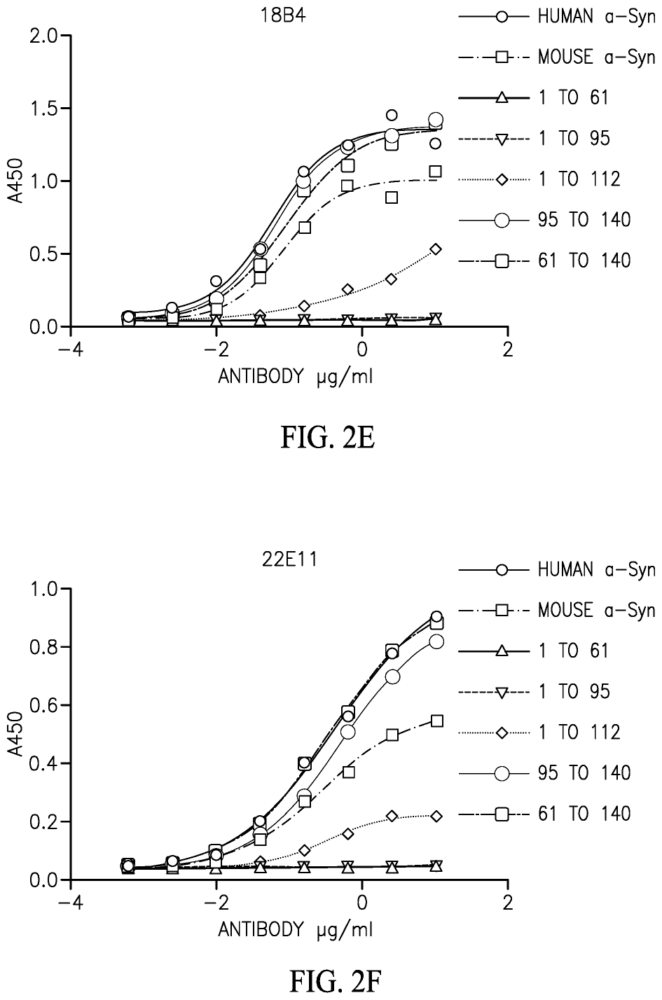

Antibody concentrations were quantified using a generic human IgG assay (MSD human IgG kit #K150JLD-4) following the manufacturer's protocol. Briefly, plates were blocked for 30 min with MSD blocker A and diluted plasma samples (1:2500) were added in duplicate to the blocked plates using a Hamilton Nimbus liquid handler. Dosing solutions were also analyzed on the same plate to confirm the correct dosage. The standard curve was fit using a four-parameter logistic regression. As shown in FIG. 4 and in Table 15 below, the anti-alpha-synuclein antibody exhibited in vivo plasma pharmacokinetic properties within the expected range and similar to control.

TABLE 15
In vivo Kinetics of Anti-Alpha-Synuclein
Antibody in Mice

| Molecule | CL (ml/day/kg) |
|---|---|
| Control anti-RSV | 10 |
| 20H4_HC5_L1_1 | 26 |

### Primate Study

In vivo plasma pharmacokinetics of anti-alpha-synuclein antibody was evaluated in cynomolgus monkeys (Macaca fascicularis) in a 28-day single-dose study.

Briefly, female animals (n=9; animals were 3-5 years old and 2.5 to 3.5 kg at initial physical exam) were acclimated for 14 days and then administered a single dose of 3 mg/kg antibody via slow intravenous bolus injection over a duration of 1-2 minutes (20H4_HC5_L1_1 with LALA mutations or control anti-RSV with LALAPG, all molecules were expressed as human IgG). N=3 animals for each test group.

Animals were housed in a temperature-(between 18 and 29° C.) and humidity-controlled environment (between 30 and 70%) on an automated 12-hour light/dark cycle. Animals were fed PMI LabDiet® Fiber-Plus® Monkey Diet 5049 biscuits twice daily. Fruit, vegetables, and dietary supplements were also provided throughout the study, and fresh water was provided ad libitum. Clinical observations were performed daily and as necessary. Animals were weighed weekly throughout acclimation and during the dosing phase, and further weighings were performed when determined necessary.

Blood samples (1 mL blood to obtain 0.5 mL plasma) were taken at 10 and 30 minutes; at 1, 6, and 12 hours; and at 1, 3, 7, 10, 14, 17, 21, 24, and 28 days following administration. Samples were maintained in 2-mL K2EDTA tubes on ice and centrifuged at 2400 rpm at 4° C. for 15 minutes, within 1 hour of collection. The resulting plasma was split into primary and duplicate aliquots of approximately equal volume, placed into labeled 0.75-mL Thermo Scientific™ Matrix Storage tubes, frozen on dry ice, and stored at −86 to −60° C. until analysis.

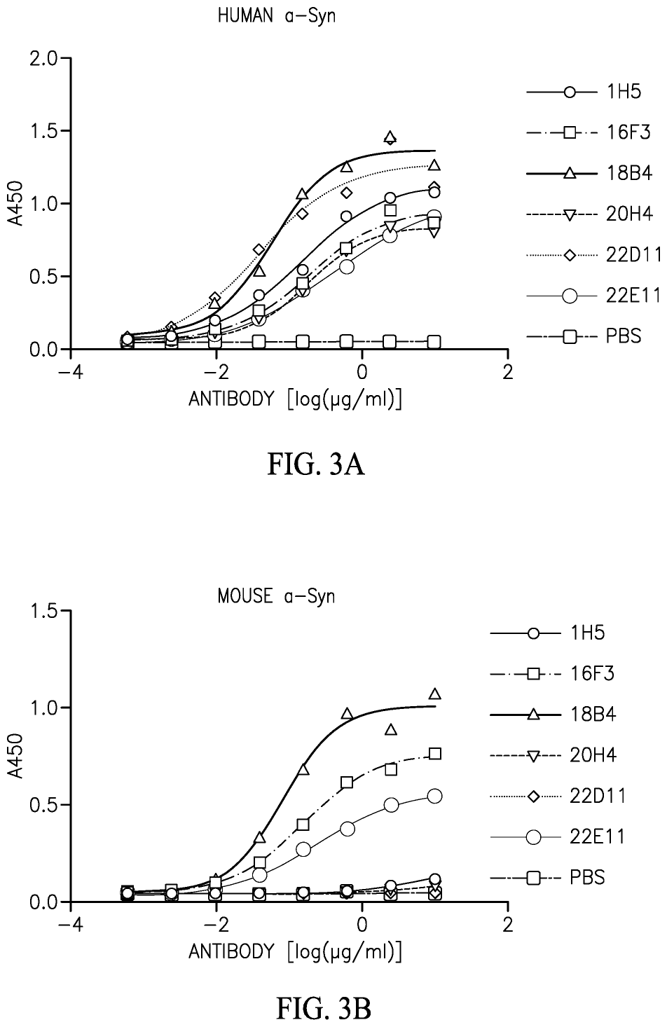

Antibody concentrations were quantified using the Gyrolab Immunoassay platform (Gyrolab Generic PK Kit, Catalog No. P0020499 following the manufacturer's protocol with optimization for the analyte and sample matrix used in this study. Analyte detection followed an anti-human Fc IgG sandwich format (CaptureSelect™ Human IgG-Fc PK Biotin Conjugate (Thermo Scientific Catalog No. 71033221500); goat polyclonal anti-human IgG-heavy and light chain antibody (Bethyl Laboratories, Catalog No. A80-319A)). Respective antibodies were used as a standard for quantification and dilutions of plasma and CSF samples were prepared in duplicate. The standard curve was fit using a five-parameter logistic curve with response weighting. Data are shown in FIG. 5 and in Table 16 below.

TABLE 16
In vivo Plasma PK of Anti-Alpha-Synuclein
Antibody in Primates

| Molecule | CL (ml/day/kg) | T_{1/2} (days) |
|---|---|---|
| Control anti-RSV | 8.8 | 7.5 |
| 20H4_HC5_L1_1 | 12.3 | 5.2 |

### Second Mouse Study

In a seven-day plasma pharmacokinetics study, wild-type 6-8 week old male C57B16 mice (n=3/group) were intravenously dosed with 10 mg/kg or 50 mg/kg of test antibodies with LALA or a control antibody with LALAPG (LALA plus P329G); all antibodies were expressed as human IgG. In-life bleeds were taken at 30 min, 1 day, 4 days, and 7 days post-dose via submandibular bleeds. Blood was collected in ETDA plasma tubes and spun at 14,000 rpm for 5 min. Plasma was then isolated for subsequent pharmacokinetic analysis. Table 17 illustrates the experimental setup and sequences (SEQ ID NOs.) of each antibody administered to each mouse cohort.

TABLE 17
Experimental Setup for Analysis of Pharmacokinetics of
Anti-Alpha-Synuclein Antibodies in Mice

| Test Article | VH | VL | Dose |
|---|---|---|---|
| Control antibody | SEQ ID NO: 191 | SEQ ID NO: 192 | 10 mg/kg |
| Control antibody | SEQ ID NO: 191 | SEQ ID NO: 192 | 50 mg/kg |
| 20H4_HC5_ | SEQ ID NO: 147 | SEQ ID NO: 201 | 10 mg/kg |
| LC_k3_1 | | | |
| 20H4_HC5_ | SEQ ID NO: 147 | SEQ ID NO: 201 | 50 mg/kg |
| LC_k3_1 | | | |
| 20H4_HC5_LC_ | SEQ ID NO: 147 | SEQ ID NO: 200 | 10 mg/kg |
| k3_2_v2 | | | |
| 20H4_HC5_LC_ | SEQ ID NO: 147 | SEQ ID NO: 200 | 50 mg/kg |
| k3_2_v2 | | | |

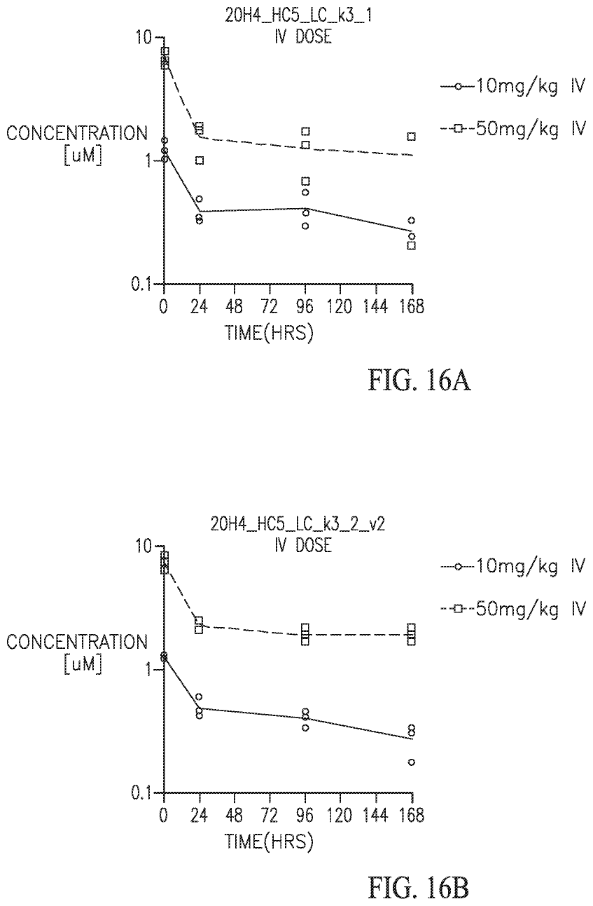

Antibody concentrations were quantified using a generic human IgG assay (MSD human IgG kit #K150JLD-4) following the manufacturer's protocol. Briefly, plates were blocked for 30 min with MSD blocker A and diluted plasma samples (1:2500) were added in duplicate to the blocked plates using a Hamilton Nimbus liquid handler. Dosing solutions were also analyzed on the same plate to confirm the correct dosage. The standard curve was fit using a four-parameter logistic regression. As shown in FIGS. 16A and 16B and in Table 18 below, the anti-alpha-synuclein antibodies exhibited in vivo plasma pharmacokinetic properties within the expected range and similar to control.

TABLE 18
Experimental Setup for Analysis of Pharmacokinetics
of Anti-Alpha-Synuclein Antibodies in Mice

| | | | AUC_{0-inf} | CL |
|---|---|---|---|---|
| | | C₀ | (μmol/ | (mL/day/ |
| Test Article | Dose | (μmol/L) | L)*hr | kg) |
| Control antibody | 10 mg/kg | 1.39 | 125.4 | 13.5 |
| Control antibody | 50 mg/kg | 5.86 | 754.1 | 11.2 |
| 20H4_HC5_LC_k3_1 | 10 mg/kg | 1.26 | 137.3 | 12.8 |
| 20H4_HC5_LC_k3_1 | 50 mg/kg | 6.80 | 757.0 | 21.6 |
| 20H4_HC5_LC_ | 10 mg/kg | 1.29 | 147.1 | 12.2 |
| k3_2_v2 | | | | |
| 20H4_HC5_LC_ | 50 mg/kg | 7.41 | 1614 | 6.9 |
| k3_2_v2 | | | | |

### Example 6

### Immunodepletion and Inhibition of Alpha-Synuclein Seeding

Experiments investigating the ability of antibodies (anti-RSV control, Antibody 1, Antibody 2, chimeric rat clone 20H4 (“20H4”), 20H4_HC5_L1_1) to immunodeplete aSyn in PD CSF and PD brain lysate (high salt and sarkosyl-insoluble pellet), and to inhibit seeding, were conducted as described in Example 2, unless indicated otherwise. Reduction values were normalized to values from mock IP using the control anti-RSV antibody. Alpha-synuclein concentrations in PD CSF following incubation were also compared.

### Parkinson's Disease (PD) Patient CSF Immunodepletion

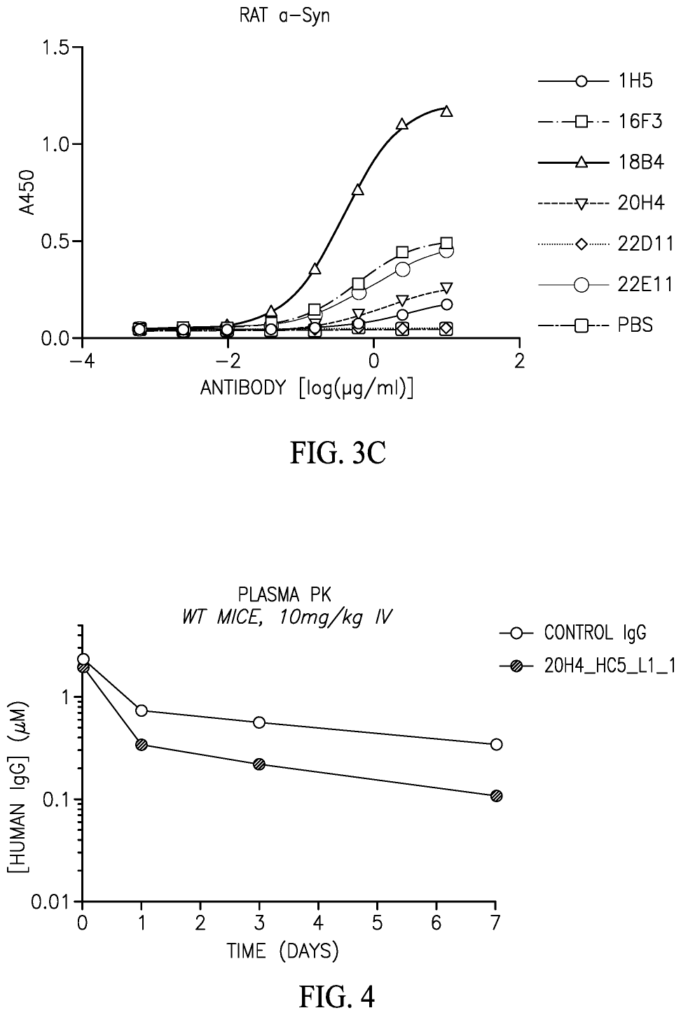

PD CSF (for each tested Ab=average of 4 PD patients±SD) was examined following incubation with 30 nM antibody. As shown in FIGS. 6A and 6B, 20H4 depleted almost all detectable aSyn from PD patient CSF, and performed significantly better than the comparator anti-alpha-synclein antibodies.

### Parkinson's Disease (PD) Brain Lysate Immunodepletion

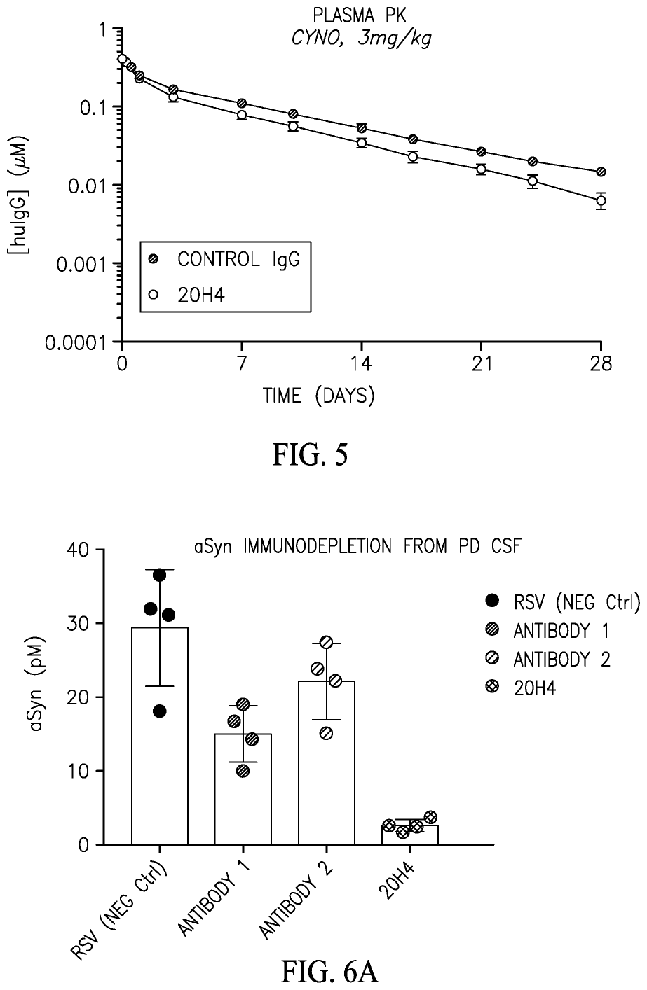

As shown in FIG. 7, 20H4 and 20H4_H5_L1_1 antibodies were highly effective in immunodepleting soluble aSyn in PD brains as measured in the high salt fraction. These two antibodies performed better than Antibody 2 and significantly better than Antibody 1 in this assay.

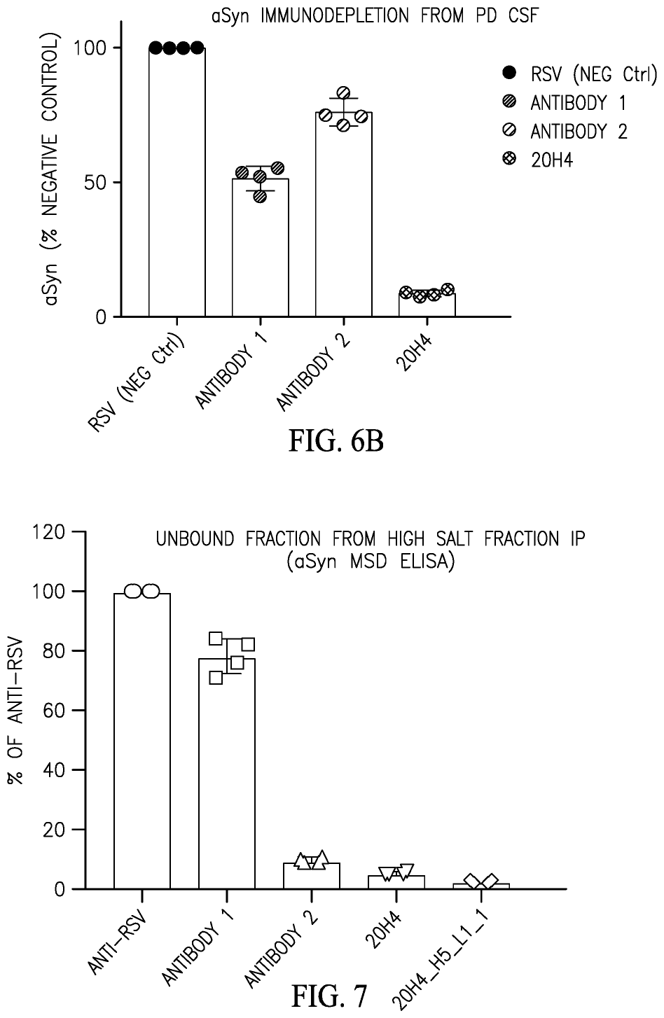

Immunodepletion was also examined in the sarkosyl-insoluble pellet fractions of PD brain extracts. As shown in FIG. 8A, MSD ELISA of the SARK-PEL fraction showed results similar to the results observed from the high salt fraction immunoprecipitate (FIG. 7). A sandwich ELISA as described in Example 2 was performed to measure immunodepletion of insoluble aggregated alpha-synuclein protein. Results are shown in FIG. 8B. These results confirm that both 20H4 and 20H4_H5_L1_1 antibodies effectively immunodeplete soluble aSyn in PD brains and inhibit seeding of alpha-synuclein, and this capability is significantly better than that of comparators Antibody 1 and Antibody 2.

### Example 7

### In Vivo Engagement of Alpha-Synuclein in an Animal Model of Human Alpha-Synucleinopathy

This study was performed in accordance with Canadian Council on Animal Care (CCAC) guidelines. Eighty-three (72+11 extra mice) male transgenic (Tg) M83 hemizygous mice expressing the mutant human A53T a-synuclein were used for this study. The A53T alpha-Synuclein transgenic line M83 mice express the mutant human A53T alpha-Synuclein under the direction of the mouse prion protein promoter (JAX strain #004479; Giasson et al. 2002. Neuron 34(4):521-533).

Aliquots of hPFFs were thawed to room temperature immediately prior to use, diluted in PBS to a final concentration of 2.5 mg/mL, and sonicated for 10-12 cycles of 5 seconds sonication followed by 30 seconds pause at high power level. This sonication generated short fibrils measuring about 100 nm or less in length upon examination by electron microscopy. Once animals reached 8 weeks-of-age, they were individually housed and randomized into dosing groups, and sixty-four 10-week old-animals received intracranial injection of human pre-formed fibrils (hPFFs) targeting the AON (Anterior Olfactory Nucleus). Thirteen (10+3 extra) mice were injected with PBS as a control injection group. The animal received a dose of 20 mg/kg of Carprofen subcutaneously prior to the craniectomy. A Hamilton syringe was introduced into the left forebrain (coordinates: +2.8 mm Anterior relative to bregma; Left 1.5 mm from midline) to target the AON, located at a depth of 2.6 mm below the dura. The hPFFs were injected at a rate of 0.1 μL/min (2.5 μL total) with the needle held in place for >10 min at the target. The animal was allowed to recover on a warming mat and had access to MediGel CPF dietary supplement (Carprofen effective dose 5 mg/kg/day) as an analgesic for 48 hours following surgery. Animals were monitored post-surgery for signs of lethargy, distress, and/or pain.

Antibody (anti-RSV control or 20H4_HC5_L1_1 on human wild-type Fc) was administered at 50 mg/kg i.p. once per week for a period of fourteen weeks, staring two weeks post-hPFFs inoculation.

Blood and plasma were collected at week −1, +3, +5, +7, +10, and +16 relative to PBS or hPFFs inoculation. From Week −1 to +10, a tail vein blood sample (about 100 μL to give about 40 μl of plasma) was collected prior to dose administration. The plasma was collected by vortexing and centrifuging blood.

At the terminal time point (16 weeks post-hPFFs inoculation and 7 days post last antibody treatment), CSF from mice was collected. A pulled capillary glass tube was used to pierce the dura matter at the site of the cisterna magna and CSF was collected by capillary action. The CSF sample was transferred to a 0.2 mL low bind microcentrifuge tube, centrifuged to check for blood contamination.

Following CSF and terminal blood collection, brains were extracted and immersion-fixed in 10% neutral-buffered formalin. The brains were grossed and dehydrated through graded ethanol and xylene, and infiltrated with paraffin wax. Formalin-fixed paraffin-embedded (FFPE) brains were sectioned at 5 μm thickness per section. Slides were stored at room temperature prior to IHC staining. Sectioning was performed in the coronal plane targeting 6 ROIs: Olfactory Bulb (OB), Anterior Olfactory Nucleus (AON), Piriform Cortex (PirCx), Entorhinal Cortex (EntCx), Hippocampus (HC), and brainstem. Two (2) sections were collected per level for anti a-Synuclein (pSer129). IHC staining was performed by manually de-paraffinized and re-hydrated slides prior to IHC staining. PhosphoSer129 a-Synuclein IHC study was performed at room temperature on a Lab Vision 360 Autostainer (Fisher Scientific, Toronto, ON, Canada) using Abcam detection reagents. The slides were heated to 100° C. in EDTA-Tris pH 9.00 1× for 10 min (Abcam ab93684) and cooled down for 25 min at room temperature. The slides were subsequently treated in 37° C. Proteinase-K (Millipore, ref. 21627), (1:60 for 10 minutes). Epitope-retrieval was followed by sequential incubations with hydrogen peroxide for 5 minutes to quench endogenous peroxidase, followed by 5 minutes in Protein Block Abcam ab156024), and then 60 minutes in the primary antibody (Rabbit Ab anti a-synuclein (pSer129; Abcam ab51253; 1:1000). Primary antibody binding was amplified using Spring Complement detection system (Spring Bioscience) and visualized using AEC Single Solution (20 min, Abcam ab64252). All IHC stained sections, were counterstained with Acid Blue 129 (Sigma-Aldrich, St. Louis, Mo.) and mounted with aqueous mounting medium.

A subset of tissue sections (5× test and control) were stained for pSer129 a-synuclein and digitized using an Axio Scan Z1 digital whole slide scanner (Carl Zeiss, Canada). Staining at Levels 4 and 6 was examined to explore the anticipated pathology spreading towards the back of the brain. The images underwent IHC quality control review and were scored by visual assessment of pSer129 a-syn. The score is an assessment of the ipsilateral or contralateral expression levels within the HC (EntCx+PirCx) and BS on a scale of 0-3, where 0=no staining, 1=minimal staining, 2=moderate staining, and 3=extensive staining.

Animals were closely monitored immediately after dosing. No adverse effects were observed post dosing. Certain animals died from unusually high levels of stress or from genital prolapse with urinary tract infection, which are well known morbidities in the M83 strain; some animals exhibited weakness and inactivity along with dehydration. Immediately upon observation of prolapse, dehydration, or weakness, animals were treated with i.p. injections of Dextrose, diet gel (and Carprofen for prolapse inflammation). If symptoms persisted, then animals were terminated.

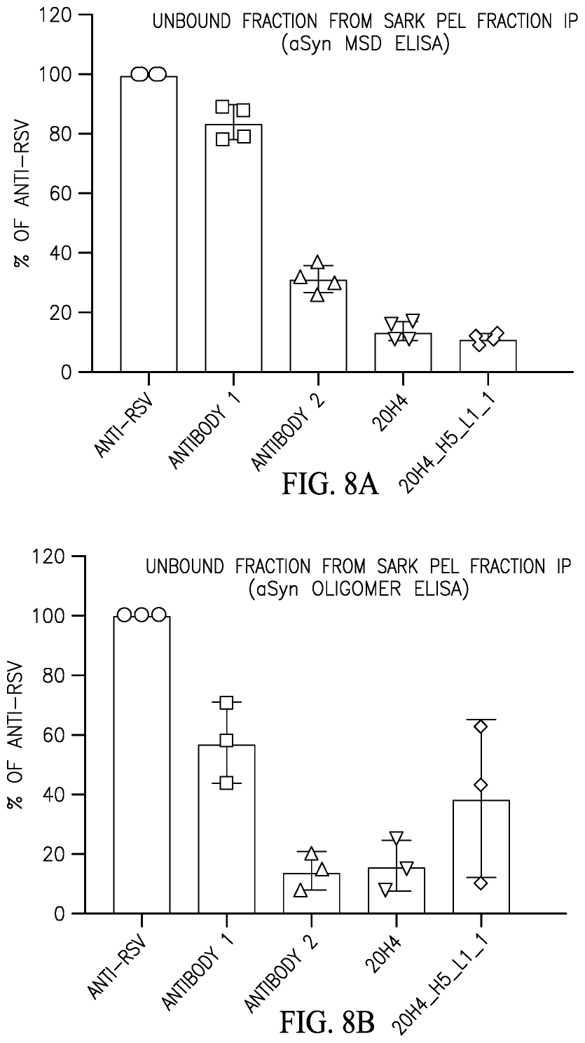

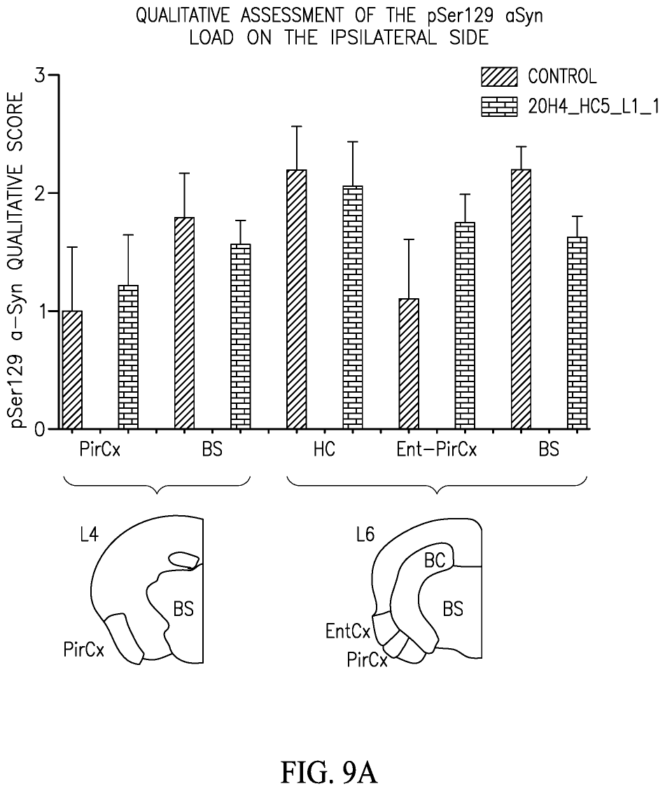

FIGS. 9A and 9B show results of the qualitative assessment IHC (per coronal level) on the Ipsilateral (injected side) and Contralateral sides of pSer129 a-syn load on the HC, Pir-EntCx, and BS regions (PBS-injected group not shown as they show no pSer129 expression) for the first eight animals tested, and demonstrate the ability of clone 20H4_HC5_L1_1 to inhibit alpha-synuclein seeding and/or spreading in-vivo compared to the control anti-RSV antibody.

In a separate animal study, A53T mice were administered antibody (anti-RSV control or 20H4_HC5_L1_1 on human wild-type Fc) as a single dose i.p. at the following doses, and target engagement in interstitial fluid (ISF) was evaluated.

TABLE 19
Dosing Groups

| | | | Dose | |
|---|---|---|---|---|
| Group | N | Substance | (mg/kg) | Route |
| A | 5 | (Control anti-RSV) | 50 | IP |
| B | 5 | hu20H4 (20H4_H5_ | 3 | IP |
| C | 6 | L1_1) | 10 | IP |
| D | 6 | | 30 | IP |
| E | 5 | | 50 | IP |

On Day 0, using standard animal surgery techniques, the mice were inserted with microdialysis probes at the striatum (Coordinates for the probe tips: (AP)=+0.8 mm to bregma, lateral (L)=−1.7 mm to midline and ventral (V)=−4.0 mm to dura, the toothbar set at 0 mm (Paxinos and Franklin, 2001, The Mouse Brain in Stereotaxic Coordinates, 2^{nd} Edition, Academic Press (San Diego)). Approximately 24 hours after surgery, ISF sampling was conducted on two consecutive days. On each microdialysis day, the probes of the animals were connected with FEP tubing to a microperfusion pump (Harvard PHD 2000 Syringe pump, Holliston, Mass. or similar). Microdialysis probes were perfused with artificial CSF containing 147 mM NaCl, 3.0 mM KCl, 1.2 mM CaCl₂) and 1.2 mM MgCl₂, and 0.15% BSA. After stabilization, microdialysis samples were collected for 60-minute periods by an automated fraction collector (820 Microsampler, Univentor, Malta) into polypropylene (300 μl) mini-vials. On Day 1, baseline ISF dialysate samples were collected for 4 hours, test articles (Table 19) were administered, and samples were collected for an additional 8 hours. At the end of Day 1, animals were disconnected overnight. On Day 2, animals were re-connected, and samples were collected for approximately 14 hours. On Day 3, the mice were sacrificed. Terminal plasma and brain tissue was collected. Left and right brain hemispheres with cerebellum were collected into separate vials. All dialysates and tissue samples were stored at −80° C. until assay.

Free Synuclein in the ISF was measured using a Mesoscale Discovery (MSD) assay. Briefly, uncoated MSD sector plates were coated overnight with anti-synuclein antibody (mouse IgG; Syn18) at a concentration 2 μg/ml in PBS. The following day, plates were washed three times in Tris Buffered Saline with tween-20 (TBS-T20) using a plate washer. Plates were blocked in assay diluent (MSD Diluent 100, #R50AA-3) for a minimum of 2 hours and subsequently washed using TBS-T20. Standard curves for synuclein were generated using recombinant human A53T-synuclein protein serially diluted in assay diluent. ISF samples collected each hour (baseline to 33 hours) were pooled in 3-hour increments, then the pooled fractions were diluted 5-fold in assay diluent, added to each well, and incubated overnight at 4° C. with shaking. The following day, plates were washed in TBS-T20, and 2 μg/ml biotinylated 20H4 antibody was added to each well for the detection of free synuclein. After incubation with labeled 20H4 detection antibody, the plates were washed with TBS-T20 and subsequently incubated with streptavidin-sulfotag. Detection signal was developed using MSD read buffer (MSD #R92TC-1). A standard curve was generated using a non-linear regression and log vs agonist 4-parameter fit and. Synuclein levels post-dose were normalized to baseline (pre-dose) values and graphed using Graphpad Prism.

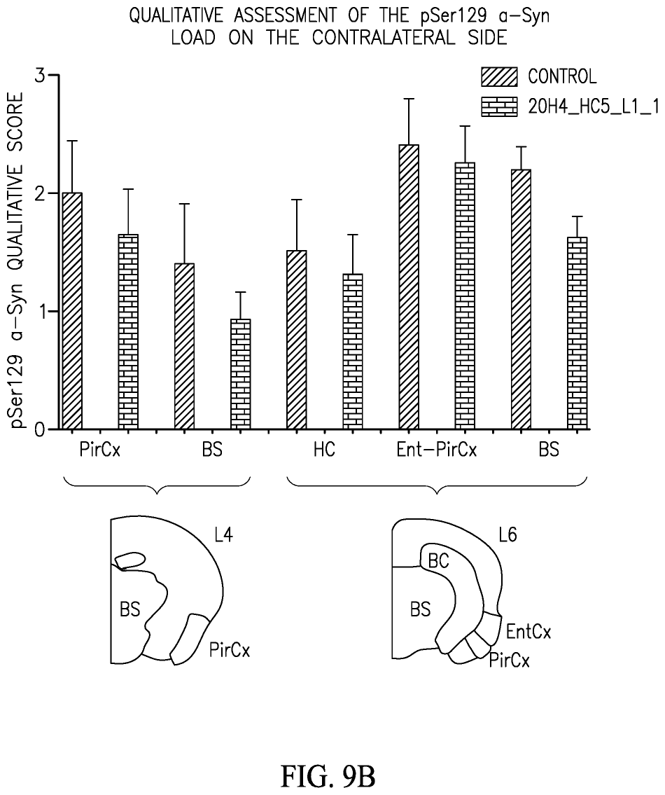

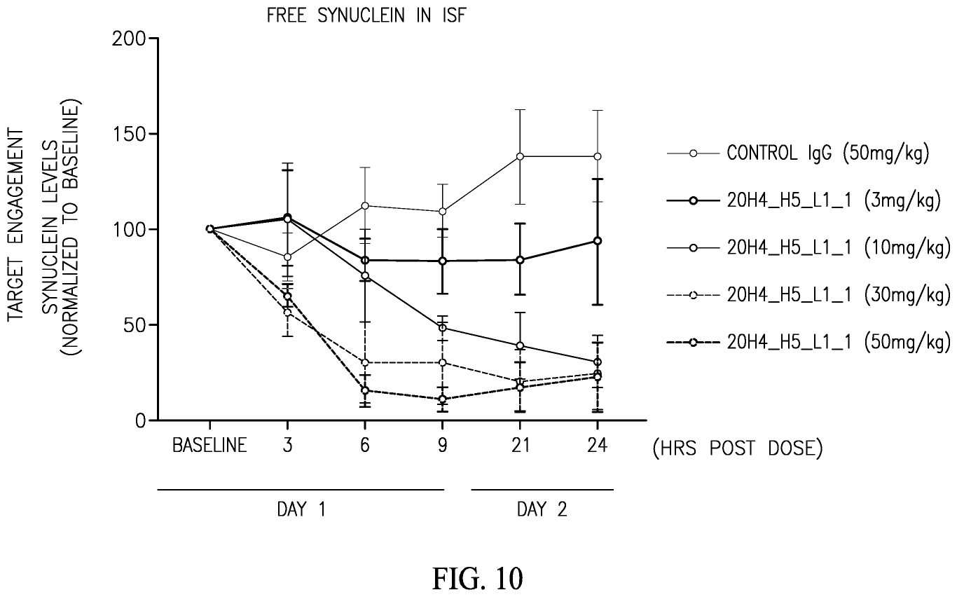

As shown in FIG. 10, a single dose of 50 mg/kg of 20H4_H5_L1_1 resulted in a greater-than 90% reduction in free alpha-synuclein. Moreover, 20H4_H5_L1_1 showed a dose-proportional reduction in free alpha-synuclein in ISF at all tested dosages. FIG. 11 shows the concentration-effect profile of 20H4_H5_L1_1 (3, 10, 30, or 50 mg/kg) in ISF dialysate, with a hill slope of 2.5 and a calculated IC50 of 166 pM.

### Example 8

### In Vivo Target Engagement of Alpha-Synuclein in Non-Human Primates

The objective of this study was to evaluate target engagement of anti-alpha-synuclein antibodies and antibody constructs in lumbar cerebrospinal fluid (CSF) of cynomolgus monkeys (Macaca fascicularis).

### Primate Study

In vivo CSF pharmacokinetics of anti-alpha-synuclein antibodies and antibody constructs were evaluated in cynomolgus monkeys (Macaca fascicularis) in a 28-day single-dose study.

Briefly, female animals (n=4; animals were 2-3 years old and 2.5 to 4.0 kg at initial physical exam) were acclimated for 4 weeks and then administered a single dose of 10 or 30 mg/kg of antibody (20H4_HC5_L1_1 with LS mutation, or “20H4_HC5_L1_1_LS,” wherein the heavy and light chain sequences correspond to SEQ ID NOs: 226 and 198, respectively) via slow intravenous bolus injection over a duration of 1-2 minutes. A comparator antibody (“Antibody 2”, described herein in Example 2) was also assessed at a single dose of 30 mg/kg. All molecules were expressed as human IgG. N=4 animals for each test group.

Animals were housed in a temperature-(between 18 and 29° C.) and humidity-controlled environment (between 30 and 70%) on an automated 12-hour light/dark cycle. Animals were fed PMI Certified Primate Diet #5048 biscuits daily during the study. Fruit, vegetables, and dietary supplements were also provided throughout the study, and fresh water was provided ad libitum. Clinical observations were performed daily and as necessary. Animals were weighed weekly throughout acclimation and during the dosing phase, and further weighings were performed when determined necessary.

Blood samples (about 1 mL blood to obtain 0.5 mL plasma) were taken at 24-hours pre-dose and at 15 minutes; at 1 and 12 hours; and at 1, 2, 3, 7, 10, 14, 17, 21, 24, and 28 days following administration. Samples were maintained in 2-mL potassium (K₂) EDTA tubes on ice and centrifuged at approximately 2400 rpm and about 4° C. for 15 minutes, within 1 hour of collection. The resulting plasma was split into primary and duplicate aliquots of approximately equal volume, placed into labeled 0.75-mL Thermo Scientific Matrix Storage tubes, frozen on dry ice, and stored at −86 to −60° C. until analysis.

CSF samples (about 1 mL) were collected from an intrathecal lumbar catheter implanted prior to study commencement at −48 hours and immediately pre-dose and at 6 hours post-dose; and also at 1, 2, 3, 7, 10, 14, 17, 21, 24, and 28 days following administration. Samples were maintained on wet ice until split into four 250 μL aliquots in labeled 0.5 mL Eppendorf® Protein LoBind tubes, frozen on dry ice, and stored at −60° C. until analysis.

### Measurement of Plasma PK

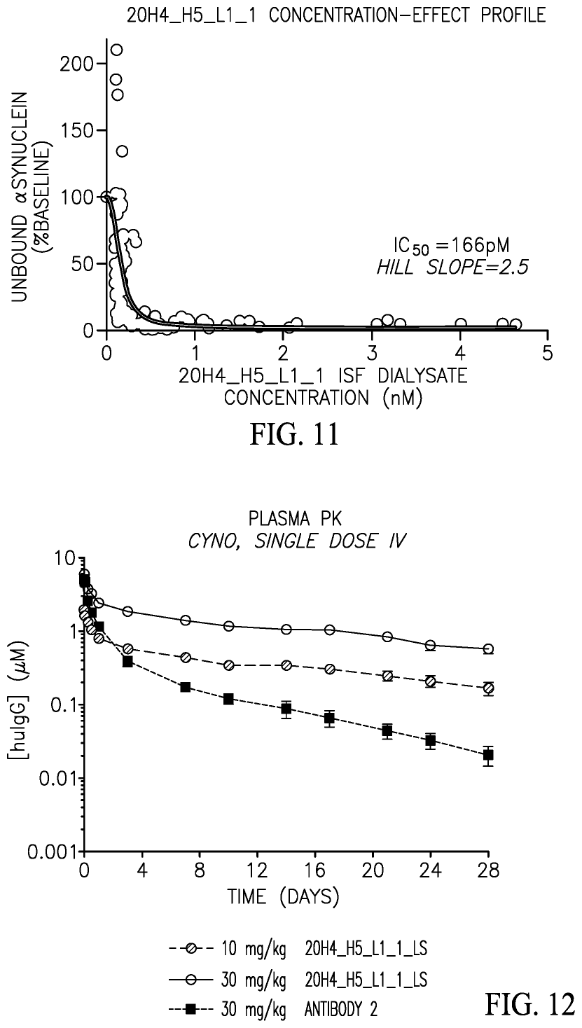

Antibody concentrations were quantified using the Gyrolab Immunoassay platform (Gyrolab Generic PK Kit, Catalog No. P0020499) following the manufacturer's protocol with optimization for the analyte and sample matrix used in this study. Analyte detection followed an anti-human Fc IgG sandwich format (CaptureSelect™ Human IgG-Fc PK Biotin Conjugate (Thermo Scientific Catalog No. 71033221500); goat polyclonal anti-human IgG-heavy and light chain antibody (Bethyl Laboratories, Catalog No. A80-319A)). Respective antibodies were used as a standard for quantification and dilutions of plasma and CSF samples were prepared in duplicate. The standard curve was fit using a five-parameter logistic curve with response weighting. Data for plasma PK are shown in FIG. 12 and illustrated in Table 20.

TABLE 20
Plasma PK of Antibodies in cynomolgus monkeys

| | Dose | Dose | CL (mL/ |
|---|---|---|---|
| Treatment | (mg/kg) | route | day/kg) |
| 20H4_H5_L1_1_LS | 10 | IV | 5.2 |
| 20H4_H5_L1_1_LS | 30 | IV | 4.5 |
| Antibody 2 | 30 | IV | 32.4 |

### Measurement of Free Synuclein Levels/Target Engagement Assessment

Free synuclein levels in the cerebrospinal fluid (CSF) of cynomolgus monkeys were measured using a combination of IgG immunodepletion followed by a total synuclein MSD assay.

### Immunodepletion Method

Dynabeads™ Protein G (Catalog No. 1009D, about 25 μL per sample) were added to 1.5-mL Eppendorf tubes and washed with Phosphate Buffered Saline+Tween20 (PBST). The beads were then blocked with 2% BSA in PBS for 2 hours at room temperature. After the blocking step, the beads were washed with PBST before being resuspended in PBST and deposited (about 25 μL per well) into individual wells of a 96 well V-bottom plate, which was then set on a DynaMag96 magnet base. CSF samples were prepared by adding 1.5 μL of protease and phosphatase inhibitor cocktail (lx CST Protase/Phosphatase Inhibitor Cocktail, Catalog No. 5872, in PBST) to 50 μL CSF. About 50 μL of CSF preparation were then added to each well and mixed with the beads in each well. The plates were then sealed and incubate at 4° C. overnight with shaking. The following day, the plate was set on the DynaMag96 magnet base, and the beads were allowed to settle. The supernatant was then removed and assayed for free synuclein.

### ‘Free’ Synuclein Assay

Synuclein after immunodepletion was measured using a commercially available human Synuclein U-PLEX Human a-Synuclein Kit (MSD, Catalog No. K151WKK). Briefly, streptavidin small spot plates were coated with biotinylated anti-synuclein antibody and incubated at room temperature for 1 hour with shaking. The plates were washed in PBST, after which immunodepleted CSF was added to an equal volume of sulfotagged anti-synuclein detection antibody solution. The mixture was added to the plates and incubated at room temperature for 2 hours. The plates were washed in PBST, followed by addition of Read buffer and measurement of signal on a Meso SECTOR S 600 plate reader (MSD Model #1201). A standard curve for synuclein was prepared according to manufacturer's instructions, and synuclein values were interpolated against the standard curve using a non-linear Log vs agonist 4-parameter fit and corrected for dilution. Graphs for absolute Synuclein values and baseline normalized values were generated using Graphpad prism.

### Results

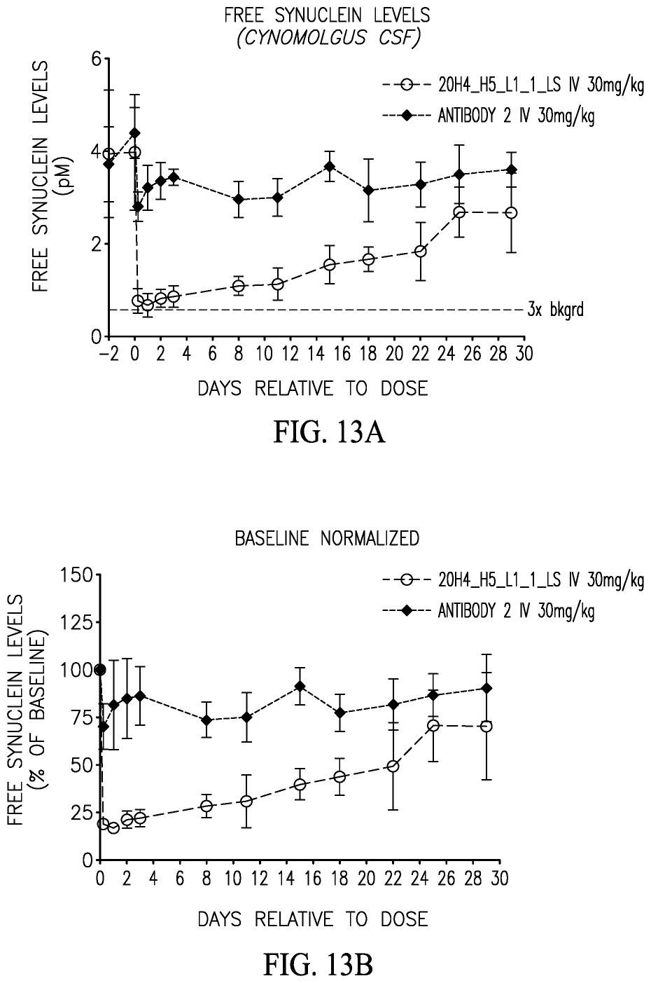

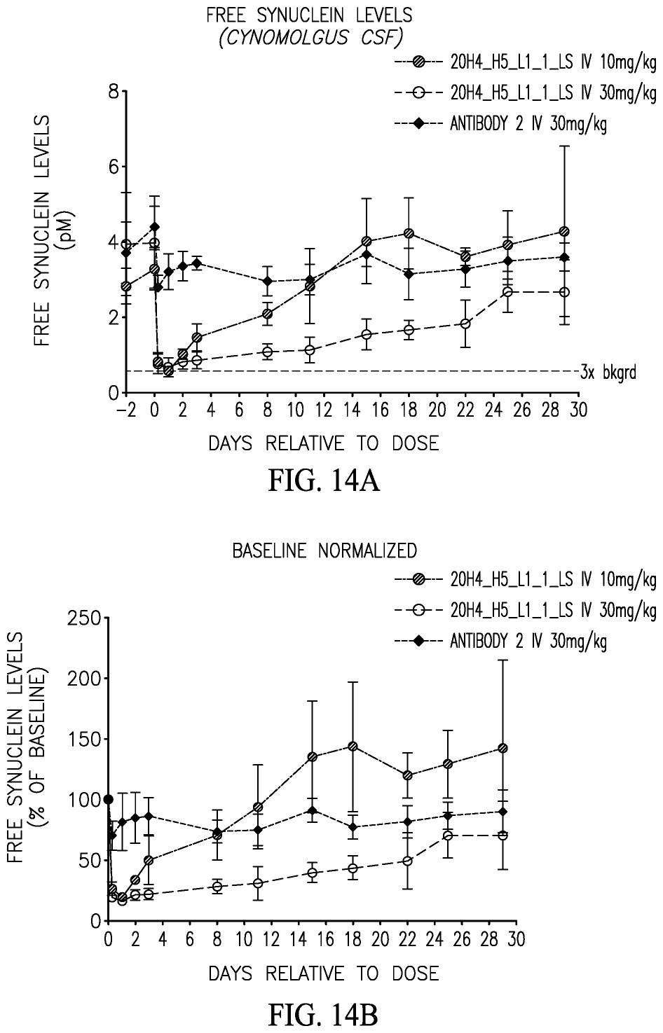

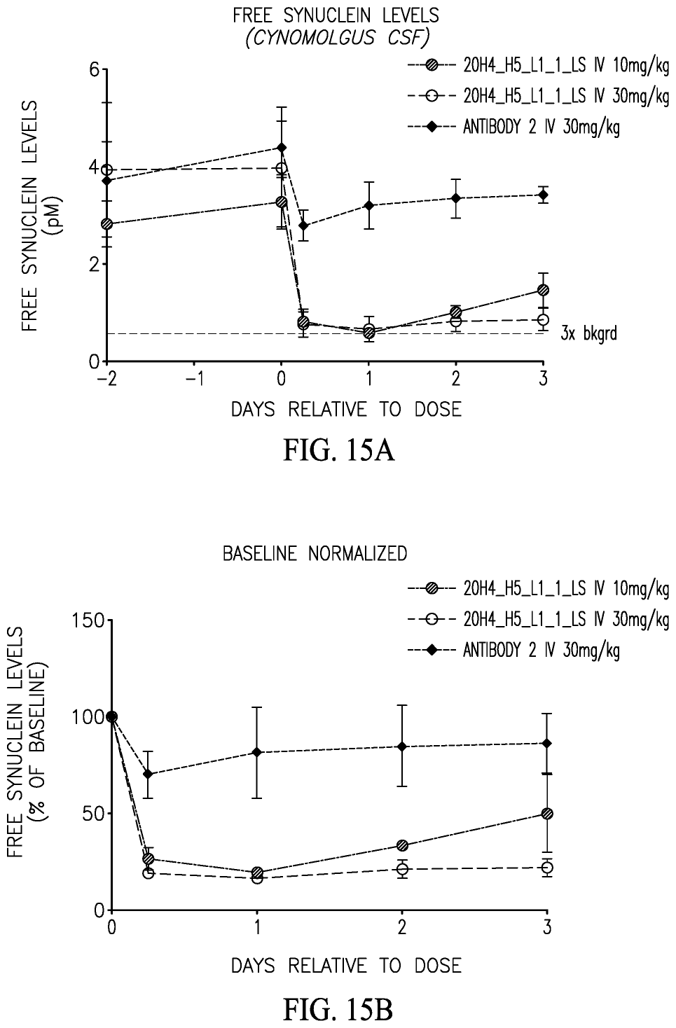

As illustrated in FIGS. 13A and 13B, 20H4_H5_L1_1_LS antibody reduced free synuclein in the CSF by about 80% within one day of dosing at 30 mg/kg. A strong reduction in free synuclein in CSF of 20H4_H5_L1_1_LS-treated animals was observed throughout the test period, with a 30% reduction in free synuclein relative to baseline observed at 29 days post-treatment. In contrast, the comparator anti-alpha-synuclein antibody (Antibody 2) only achieved a maximum 30% reduction in free synuclein in the CSF after dosing at 30 mg/kg (immediately following treatment). Even at a lower dose of 10 mg/kg, administration of 20H4_H5_L1_1_LS antibody resulted in a reduction in CSF free synuclein by about 80% within one day of dosing (FIGS. 14A and 14B). FIGS. 15A and 15B provide an enlarged view of the data in FIGS. 14A and 14B (respectively) from −2 days pre-dose to 3-days post-dose. These results clearly illustrate improved target engagement for free synuclein in the CSF by 20H4_H5_L1_1_LS antibody over the comparator antibody.

The present disclosure also provides the following embodiments.

Embodiment 1. An isolated antibody or antigen-binding portion thereof that specifically binds to a human alpha-synuclein protein, wherein the antibody or antigen-binding portion thereof recognizes an epitope comprised within residues 119-125 of SEQ ID NO:1.

Embodiment 2. The isolated antibody or antigen-binding portion of Embodiment 1, wherein the antibody or antigen-binding portion thereof has a higher binding affinity for the human alpha-synuclein protein than for a rat alpha-synuclein protein.

Embodiment 3. The isolated antibody or antigen-binding portion of Embodiment 1 or 2, wherein the antibody or antigen-binding portion thereof has a binding affinity for the human alpha-synuclein protein that is at least about 3-fold higher than the binding affinity for the rat alpha-synuclein protein.

Embodiment 4. The isolated antibody or antigen-binding portion of any one of Embodiments 1-3, wherein the antibody or antigen-binding portion thereof specifically binds to phosphorylated human alpha-synuclein protein and/or unphosphorylated human alpha-synuclein protein.

Embodiment 5. The isolated antibody or antigen-binding portion of any one of Embodiments 1-4, wherein the antibody or antigen-binding portion thereof specifically binds to phosphorylated human alpha-synuclein protein and unphosphorylated human alpha-synuclein protein.

Embodiment 6. The isolated antibody or antigen-binding portion of any one of Embodiments 1-5, wherein the antibody or antigen-binding portion thereof has about the same binding affinity for phosphorylated human alpha-synuclein protein and for unphosphorylated human alpha-synuclein protein.

Embodiment 7. The isolated antibody or antigen-binding portion of any one of Embodiments 4-6, wherein the phosphorylated human alpha-synuclein protein is phosphorylated at Ser129.

Embodiment 8. An isolated antibody or antigen-binding portion thereof that specifically binds to a human alpha-synuclein protein, wherein the antibody or antigen-binding portion thereof has about the same binding affinity for phosphorylated human alpha-synuclein protein and for unphosphorylated human alpha-synuclein protein.

Embodiment 9. The isolated antibody or antigen-binding portion of Embodiment 8, wherein the phosphorylated human alpha-synuclein protein is phosphorylated at Ser129.

Embodiment 10. An isolated antibody or antigen-binding portion thereof that specifically binds to a human alpha-synuclein protein, wherein the antibody or antigen-binding portion thereof specifically binds to human alpha-synuclein protein that is phosphorylated at Ser129 and/or to unphosphorylated human alpha-synuclein protein.

Embodiment 11. The isolated antibody or antigen-binding portion of Embodiment 10, wherein the antibody or antigen-binding portion thereof specifically binds to human alpha-synuclein protein that is phosphorylated at Ser129 and to unphosphorylated human alpha-synuclein protein.

Embodiment 12. The isolated antibody or antigen-binding portion of Embodiments 10 or 11, wherein the antibody or antigen-binding portion thereof has about the same binding affinity for human alpha-synuclein protein that is phosphorylated at Ser129 and for unphosphorylated human alpha-synuclein protein.

Embodiment 13. The isolated antibody or antigen-binding portion of any one of Embodiments 8-12, wherein the antibody or antigen-binding portion thereof has a higher binding affinity for the human alpha-synuclein protein than for a rat alpha-synuclein protein.

Embodiment 14. The isolated antibody or antigen-binding portion of any one of Embodiments 1-13, wherein the antibody or antigen-binding portion thereof has a binding affinity for the human alpha-synuclein protein that is at least about 3-fold higher than the binding affinity for the rat alpha-synuclein protein.

Embodiment 15. The isolated antibody or antigen-binding portion of any one of Embodiments 1-14, wherein the antibody or antigen-binding portion thereof recognizes an epitope comprised within residues 95-140 of SEQ ID NO:1.

Embodiment 16. The isolated antibody or antigen-binding portion of embodiments 8-15, wherein the antibody or antigen-binding portion thereof recognizes an epitope comprised within residues 112-140 of SEQ ID NO:1.

Embodiment 17. The isolated antibody or antigen-binding portion of any one of Embodiments 8-15, wherein the antibody or antigen-binding portion thereof recognizes an epitope comprised within residues 111-130 of SEQ ID NO:1.

Embodiment 18. The isolated antibody or antigen-binding portion of any one of Embodiments 8-17, wherein the antibody or antigen-binding portion thereof recognizes an epitope comprised within residues 111-125 or residues 116-130 of SEQ ID NO:1.

Embodiment 19. The isolated antibody or antigen-binding portion of Embodiment 17 or 18, wherein the antibody or antigen-binding portion thereof recognizes an epitope comprised within residues 119-125 of SEQ ID NO:1.

Embodiment 20. The isolated antibody or antigen-binding portion of any one of Embodiment 1-19, wherein binding of the antibody or antigen-binding portion thereof to the human alpha-synuclein protein requires binding to residues 119, 122, and 125 of SEQ ID NO:1.

Embodiment 21. The isolated antibody or antigen-binding portion of any one of Embodiments 1-20, wherein binding of the antibody or antigen-binding portion thereof to the human alpha-synuclein protein requires binding to residues 119, 122, 124, and 125 of SEQ ID NO:1, and/or wherein alanine mutagenesis at any one or more of residues 119, 122, 124, and 125 of SEQ ID NO:1, and/or proline mutagenesis at residue 124 of SEQ ID NO:1, reduces or abrogates binding by the antibody or antigen-binding portion thereof to the human alpha-synuclein protein, and/or wherein glycine mutagenesis at residue 124 of SEQ ID NO:1 does not reduce or abrogate binding by the antibody or antigen-binding portion thereof to the human alpha-synuclein protein; wherein, optionally, reduction or abrogation in binding by the antibody or antigen-binding portion thereof to the human alpha-synuclein protein, or lack thereof, is determined by ELISA, with an absorbance signal quantified at 450 nM.

Embodiment 22. The isolated antibody or antigen-binding portion of any one of Embodiments 1-21, wherein alanine mutagenesis of the human alpha-synuclein protein at any of residues 120, 121, and 123 of SEQ ID NO:1 does not reduce or abrogate binding by the antibody or antigen-binding portion thereof to the human alpha-synuclein protein wherein, optionally, reduction or abrogation in binding by the antibody or antigen-binding portion thereof to the human alpha-synuclein protein, or lack thereof, is determined by ELISA, with an absorbance signal quantified at 450 nM.

Embodiment 23. The isolated antibody or antigen-binding portion of any one of Embodiments 1 to 22, wherein the antibody or antigen-binding portion thereof specifically binds to monomeric and/or oligomeric human alpha-synuclein protein.

Embodiment 24. The isolated antibody or antigen-binding portion of embodiment 23, wherein the antibody or antigen-binding portion thereof specifically binds to monomeric and oligomeric human alpha-synuclein protein.

Embodiment 25. The isolated antibody or antigen-binding portion of Embodiment 23 or 24, wherein the antibody or antigen-binding portion thereof has about the same binding affinity for oligomeric human alpha-synuclein protein and for monomeric human alpha-synuclein protein.

Embodiment 26. The isolated antibody or antigen-binding portion of Embodiment 23 or 24, wherein the antibody or antigen-binding portion thereof has a higher binding affinity for oligomeric human alpha-synuclein protein than for monomeric human alpha-synuclein protein.

Embodiment 27. The isolated antibody or antigen-binding portion of any one of Embodiments 1 to 26, wherein the antibody or antigen-binding portion thereof specifically binds to soluble human alpha-synuclein protein and/or human alpha-synuclein protein fibrils.

Embodiment 28. The isolated antibody or antigen-binding portion of any one of Embodiments 1 to 27, wherein the antibody is a pan-alpha-synuclein antibody or antigen-binding portion thereof.

Embodiment 29. The isolated antibody or antigen-binding portion of Embodiment 28, wherein the antibody or antigen-binding portion thereof specifically binds to each of monomeric, oligomeric, fibril, and pSer129 human alpha-synuclein protein with an affinity of at least about 10 nM, and/or specifically binds to each of monomeric human alpha-synuclein protein, oligomeric human alpha-synuclein protein, and fibrillar human alpha-synuclein protein with Kd of about 500 pM or less, or about 300 pM or less, as measured by surface plasmon resonance (SPR).

Embodiment 30. The isolated antibody or antigen-binding portion of any one of Embodiments 1-29, wherein the antibody or antigen-binding portion thereof specifically binds to each of monomeric, oligomeric, fibril, and pSer129 human alpha-synuclein protein with an affinity of at least about 6 nM.

Embodiment 31. The isolated antibody or antigen-binding portion of any one of Embodiments 1 to 30, wherein the antibody or antigen-binding portion thereof specifically binds to at least about 95% of soluble human alpha-synuclein protein that is present in a sample.

Embodiment 32. The isolated antibody or antigen-binding portion of any one of Embodiments 1 to 30, wherein the antibody or antigen-binding portion thereof specifically binds to at least about 70% of insoluble human alpha-synuclein protein that is present in a sample.

Embodiment 33. The isolated antibody or antigen-binding portion of Embodiment 31 or 32, wherein the sample is selected from the group consisting of an interstitial brain fluid sample, a cerebrospinal fluid (CSF) sample, a brain lysate sample, a blood sample, and a combination thereof.

Embodiment 34. The isolated antibody or antigen-binding portion of any one of Embodiments 1 to 33, wherein the antibody or antigen-binding portion thereof has a higher binding affinity for alpha-synuclein protein than for beta-synuclein protein and/or gamma-synuclein protein.

Embodiment 35. The isolated antibody or antigen-binding portion of any one of embodiments 1 to 34, comprising one or more complementarity determining regions (CDRs) selected from the group consisting of: (a) a heavy chain CDR1 having at least 90% sequence identity to the amino acid sequence of any one of SEQ ID NOs:2-7, 43, 87, and 97-104 or having up to two amino acid substitutions relative to the amino acid sequence of any one of SEQ ID NOs:2-7, 43, 87, and 97-104; (b) a heavy chain CDR2 having at least 90% sequence identity to the amino acid sequence of any one of SEQ ID NOs:8-10, 44-45, 88-89, 105-112, and 188, or having up to two amino acid substitutions relative to the amino acid sequence of any one of SEQ ID NOs: 8-10, 44-45, 88-89, 105-112, and 188; (c) a heavy chain CDR3 having at least 90% sequence identity to the amino acid sequence of any one of SEQ ID NOs:11-16, 46, 90, and 113-121 or having up to two amino acid substitutions relative to the amino acid sequence of any one of SEQ ID NOs:11-16, 46, 90, and 113-121; (d) a light chain CDR1 having at least 90% sequence identity to the amino acid sequence of any one of SEQ ID NOs:23-26, 47-48, 91-92, and 122-125 or having up to two amino acid substitutions relative to the amino acid sequence of any one of SEQ ID NOs:23-26, 47-48, 91-92, and 122-125; (e) a light chain CDR2 having at least 90% sequence identity to the amino acid sequence of any one of SEQ ID NOs:27-30, 49-50, 93-94, and 126-130 or having up to two amino acid substitutions relative to the amino acid sequence of any one of SEQ ID NOs:27-30, 49-50, 93-94, and 126-130; and (f) a light chain CDR3 having at least 90% sequence identity to the amino acid sequence of any one of SEQ ID NOs:31-36, 51-52, 95-96, and 131-143 or having up to two amino acid substitutions relative to the amino acid sequence of any one of SEQ ID NOs:31-36, 51-52, 95-96, and 131-143.

Embodiment 36. The isolated antibody or antigen-binding portion of any one of Embodiments 1 to 34, comprising one or more CDRs selected from the group consisting of: (a) a heavy chain CDR1 comprising the amino acid sequence of any one of SEQ ID NOs:2-7, 43, 87, and 97-104; (b) a heavy chain CDR2 comprising the amino acid sequence of any one of SEQ ID NOs: 8-10, 44-45, 88-89, 105-112, and 188; (c) a heavy chain CDR3 comprising the amino acid sequence of any one of SEQ ID NOs:11-16, 46, 90, and 113-121; (d) a light chain CDR1 comprising the amino acid sequence of any one of SEQ ID NOs:23-26, 47-48, 91-92, and 122-125; (e) a light chain CDR2 comprising the amino acid sequence of any one of SEQ ID NOs:27-30, 49-50, 93-94, and 126-130; and (f) a light chain CDR3 comprising the amino acid sequence of any one of SEQ ID NOs:31-36, 51-52, 95-96, and 131-143.

Embodiment 37. The isolated antibody or antigen-binding portion of Embodiment 36, comprising: (a) a heavy chain CDR1 comprising the amino acid sequence of SEQ ID NO:2; (b) a heavy chain CDR2 comprising the amino acid sequence of SEQ ID NO:8; and (c) a heavy chain CDR3 comprising the amino acid sequence of SEQ ID NO:11.

Embodiment 38. The isolated antibody or antigen-binding portion of Embodiment 36, comprising: (a) a light chain CDR1 comprising the amino acid sequence of SEQ ID NO:23; (b) a light chain CDR2 comprising the amino acid sequence of SEQ ID NO:27; and (c) a light chain CDR3 comprising the amino acid sequence of SEQ ID NO:31.

Embodiment 39. The isolated antibody or antigen-binding portion of Embodiment 36, comprising a heavy chain CDR1 comprising the amino acid sequence of SEQ ID NO:2, a heavy chain CDR2 comprising the amino acid sequence of SEQ ID NO:8, a heavy chain CDR3 comprising the amino acid sequence of SEQ ID NO:11, a light chain CDR1 comprising the amino acid sequence of SEQ ID NO:23, a light chain CDR2 comprising the amino acid sequence of SEQ ID NO:27, and a light chain CDR3 comprising the amino acid sequence of SEQ ID NO:31.

Embodiment 40. The isolated antibody or antigen-binding portion of Embodiment 36, comprising: (a) a heavy chain CDR1 comprising the amino acid sequence of any one of SEQ ID NOs:3 and 97-102; (b) a heavy chain CDR2 comprising the amino acid sequence of any one of SEQ ID NOs:9 and 105-110; and (c) a heavy chain CDR3 comprising the amino acid sequence of any one of SEQ ID NOs:12 and 113-119.

Embodiment 41. The isolated antibody or antigen-binding portion of Embodiment 36, comprising: (a) a light chain CDR1 comprising the amino acid sequence of any one of SEQ ID NOs:24 and 122-123; (b) a light chain CDR2 comprising the amino acid sequence of any one of SEQ ID NOs:28 and 126-128; and (c) a light chain CDR3 comprising the amino acid sequence of any one of SEQ ID NOs:32 and 131-141.

Embodiment 42. The isolated antibody or antigen-binding portion of Embodiment 36, comprising a heavy chain CDR1 comprising the amino acid sequence of any one of SEQ ID NOs:3 and 97-102, a heavy chain CDR2 comprising the amino acid sequence of any one of SEQ ID NOs:9 and 105-110, a heavy chain CDR3 comprising the amino acid sequence of any one of SEQ ID NOs:12 and 113-119, a light chain CDR1 comprising the amino acid sequence of any one of SEQ ID NOs:24 and 122-123, a light chain CDR2 comprising the amino acid sequence of any one of SEQ ID NOs:28 and 126-128, and a light chain CDR3 comprising the amino acid sequence of any one of SEQ ID NOs:32 and 131-141.

Embodiment 43. The isolated antibody or antigen-binding portion of Embodiment 36, comprising: (a) a heavy chain CDR1 comprising the amino acid sequence of SEQ ID NO:4; (b) a heavy chain CDR2 comprising the amino acid sequence of SEQ ID NO:188; and (c) a heavy chain CDR3 comprising the amino acid sequence of SEQ ID NO:13.

Embodiment 44. The isolated antibody or antigen-binding portion of Embodiment 36, comprising: (a) a light chain CDR1 comprising the amino acid sequence of SEQ ID NO:24; (b) a light chain CDR2 comprising the amino acid sequence of SEQ ID NO:27; and (c) a light chain CDR3 comprising the amino acid sequence of SEQ ID NO:33.

Embodiment 45. The isolated antibody or antigen-binding portion of Embodiment 36, comprising a heavy chain CDR1 comprising the amino acid sequence of SEQ ID NO:4, a heavy chain CDR2 comprising the amino acid sequence of SEQ ID NO:188, a heavy chain CDR3 comprising the amino acid sequence of SEQ ID NO:13, a light chain CDR1 comprising the amino acid sequence of SEQ ID NO:24, a light chain CDR2 comprising the amino acid sequence of SEQ ID NO:27, and a light chain CDR3 comprising the amino acid sequence of SEQ ID NO:33.

Embodiment 46. The isolated antibody or antigen-binding portion of Embodiment 36, comprising: (a) a heavy chain CDR1 comprising the amino acid sequence of SEQ ID NO:5; (b) a heavy chain CDR2 comprising the amino acid sequence of SEQ ID NO:8; and (c) a heavy chain CDR3 comprising the amino acid sequence of SEQ ID NO:14.

Embodiment 47. The isolated antibody or antigen-binding portion of Embodiment 36, comprising: (a) a light chain CDR1 comprising the amino acid sequence of SEQ ID NO:25; (b) a light chain CDR2 comprising the amino acid sequence of SEQ ID NO:29; and (c) a light chain CDR3 comprising the amino acid sequence of SEQ ID NO:34.

Embodiment 48. The isolated antibody or antigen-binding portion of Embodiment 36, comprising a heavy chain CDR1 comprising the amino acid sequence of SEQ ID NO:5, a heavy chain CDR2 comprising the amino acid sequence of SEQ ID NO:8, a heavy chain CDR3 comprising the amino acid sequence of SEQ ID NO:14, a light chain CDR1 comprising the amino acid sequence of SEQ ID NO:25, a light chain CDR2 comprising the amino acid sequence of SEQ ID NO:29, and a light chain CDR3 comprising the amino acid sequence of SEQ ID NO:34.

Embodiment 49. The isolated antibody or antigen-binding portion of Embodiment 36, comprising: (a) a heavy chain CDR1 comprising the amino acid sequence of SEQ ID NO:6; (b) a heavy chain CDR2 comprising the amino acid sequence of SEQ ID NO:10; and (c) a heavy chain CDR3 comprising the amino acid sequence of SEQ ID NO:15.

Embodiment 50. The isolated antibody or antigen-binding portion of Embodiment 36, comprising: (a) a light chain CDR1 comprising the amino acid sequence of SEQ ID NO:25; (b) a light chain CDR2 comprising the amino acid sequence of SEQ ID NO:29; and (c) a light chain CDR3 comprising the amino acid sequence of SEQ ID NO:35.

Embodiment 51. The isolated antibody or antigen-binding portion of Embodiment 36, comprising a heavy chain CDR1 comprising the amino acid sequence of SEQ ID NO:6, a heavy chain CDR2 comprising the amino acid sequence of SEQ ID NO:10, a heavy chain CDR3 comprising the amino acid sequence of SEQ ID NO:15, a light chain CDR1 comprising the amino acid sequence of SEQ ID NO:25, a light chain CDR2 comprising the amino acid sequence of SEQ ID NO:29, and a light chain CDR3 comprising the amino acid sequence of SEQ ID NO:35.

Embodiment 52. The isolated antibody or antigen-binding portion of Embodiment 36, comprising: (a) a heavy chain CDR1 comprising the amino acid sequence of SEQ ID NO:7; (b) a heavy chain CDR2 comprising the amino acid sequence of SEQ ID NO:8; and (c) a heavy chain CDR3 comprising the amino acid sequence of SEQ ID NO:16.

Embodiment 53. The isolated antibody or antigen-binding portion of Embodiment 36, comprising: (a) a light chain CDR1 comprising the amino acid sequence of SEQ ID NO:26; (b) a light chain CDR2 comprising the amino acid sequence of SEQ ID NO:30; and (c) a light chain CDR3 comprising the amino acid sequence of SEQ ID NO:36.

Embodiment 54. The isolated antibody or antigen-binding portion of Embodiment 36, comprising a heavy chain CDR1 comprising the amino acid sequence of SEQ ID NO:7, a heavy chain CDR2 comprising the amino acid sequence of SEQ ID NO:8, a heavy chain CDR3 comprising the amino acid sequence of SEQ ID NO:16, a light chain CDR1 comprising the amino acid sequence of SEQ ID NO:26, a light chain CDR2 comprising the amino acid sequence of SEQ ID NO:30, and a light chain CDR3 comprising the amino acid sequence of SEQ ID NO:36.

Embodiment 55. The isolated antibody or antigen-binding portion of any one of Embodiments 1 to 54, comprising a heavy chain variable region comprising an amino acid sequence that has at least 90% sequence identity to any one of SEQ ID NOs:17-22 and 144-166.

Embodiment 56. The isolated antibody or antigen-binding portion of any one of Embodiments 1 to 54, comprising a light chain variable region comprising an amino acid sequence that has at least 90% sequence identity to any one of SEQ ID NOs:37-42 and 167-187.

Embodiment 57. The isolated antibody or antigen-binding portion of any one of Embodiments 1 to 54, comprising a heavy chain variable region comprising an amino acid sequence that has at least 90% sequence identity to any one of SEQ ID NOs:17-22 and 144-166 and a light chain variable region comprising an amino acid sequence that has at least 90% sequence identity to any one of SEQ ID NOs:37-42 and 167-187.

Embodiment 58. The isolated antibody or antigen-binding portion of any one of Embodiments 1 to 54, comprising a heavy chain variable region comprising an amino acid sequence that has at least 90% sequence identity to SEQ ID NO:17 and a light chain variable region comprising an amino acid sequence that has at least 90% sequence identity to SEQ ID NO:37.

Embodiment 59. The isolated antibody or antigen-binding portion of any one of Embodiments 1 to 54, comprising a heavy chain variable region comprising an amino acid sequence that has at least 90% sequence identity to any one of SEQ ID NOs:18, 144-166, 222, 223, and 229 and a light chain variable region comprising an amino acid sequence that has at least 90% sequence identity to any one of SEQ ID NOs:38, 167-187, 200-213, and 224.

Embodiment 60. The isolated antibody or antigen-binding portion of any one of Embodiments 1 to 54, comprising a heavy chain variable region comprising an amino acid sequence that has at least 90% sequence identity to SEQ ID NO:19 and a light chain variable region comprising an amino acid sequence that has at least 90% sequence identity to SEQ ID NO:39.

Embodiment 61. The isolated antibody or antigen-binding portion of any one of Embodiments 1 to 54, comprising a heavy chain variable region comprising an amino acid sequence that has at least 90% sequence identity to SEQ ID NO:20 and a light chain variable region comprising an amino acid sequence that has at least 90% sequence identity to SEQ ID NO:40.

Embodiment 62. The isolated antibody or antigen-binding portion of any one of Embodiments 1 to 54, comprising a heavy chain variable region comprising an amino acid sequence that has at least 90% sequence identity to SEQ ID NO:21 and a light chain variable region comprising an amino acid sequence that has at least 90% sequence identity to SEQ ID NO:41.

Embodiment 63. The isolated antibody or antigen-binding portion of any one of Embodiments 1 to 54, comprising a heavy chain variable region comprising an amino acid sequence that has at least 90% sequence identity to SEQ ID NO:22 and a light chain variable region comprising an amino acid sequence that has at least 90% sequence identity to SEQ ID NO:42.

Embodiment 64. The isolated antibody or antigen-binding portion of any one of embodiments 1 to 63, wherein the antibody comprises a first Fc polypeptide and optionally a second Fc polypeptide.

Embodiment 65. The isolated antibody or antigen-binding portion of embodiment 64, wherein the antibody comprises the first Fc polypeptide and the second Fc polypeptide.

Embodiment 66. The isolated antibody or antigen-binding portion of embodiment 64 or 65, wherein the first Fc polypeptide is a modified Fc polypeptide and/or the second Fc polypeptide is a modified Fc polypeptide.

Embodiment 67. The isolated antibody or antigen-binding portion of any one of embodiments 1 to 63, wherein the antibody comprises: (a) a first antigen-binding portion comprising a first variable region that specifically binds to the human alpha-synuclein protein, wherein the first antigen-binding portion comprises (i) a first heavy chain comprising a first Fc polypeptide and (ii) a first light chain; and (b) a second antigen-binding portion comprising a second variable region that specifically binds to the human alpha-synuclein protein, wherein the second antigen-binding portion comprises (i) a second heavy chain comprising a second Fc polypeptide and (ii) a second light chain, wherein the first Fc polypeptide and the second Fc polypeptide form an Fc dimer.

Embodiment 68. The isolated antibody or antigen-binding portion of embodiment 67, wherein the first Fc polypeptide is a modified Fc polypeptide and/or the second Fc polypeptide is a modified Fc polypeptide.

Embodiment 69. The isolated antibody or antigen-binding portion of embodiment 67 or 68, wherein the first and second variable regions recognize the same epitope in the human alpha-synuclein protein.

Embodiment 70. The isolated antibody or antigen-binding portion of embodiment 67 or 68, wherein the first and second variable regions recognize different epitopes in the human alpha-synuclein protein.

Embodiment 71. The isolated antibody or antigen-binding portion of any one of embodiments 66 to 70, wherein the first Fc polypeptide and the second Fc polypeptide each contain one or more modifications that promote heterodimerization.

Embodiment 72. The isolated antibody or antigen-binding portion of embodiment 71, wherein one of the Fc polypeptides has a T366W substitution and the other Fc polypeptide has T366S, L368A, and Y407V substitutions, according to EU numbering.

Embodiment 73. The isolated antibody or antigen-binding portion of any one of embodiments 66 to 72, wherein the first Fc polypeptide and/or the second Fc polypeptide comprises a native FcRn binding site.

Embodiment 74. The isolated antibody or antigen-binding portion of any one of embodiments 66 to 72, wherein the first Fc polypeptide and/or the second Fc polypeptide comprises a modification that alters FcRn binding.

Embodiment 75. The isolated antibody or antigen-binding portion of any one of embodiments 66 to 74, wherein the first Fc polypeptide and the second Fc polypeptide do not have effector function.

Embodiment 76. The isolated antibody or antigen-binding portion of any one of embodiments 66 to 74, wherein the first Fc polypeptide and/or the second Fc polypeptide comprises a modification that reduces effector function.

Embodiment 77. The isolated antibody or antigen-binding portion of embodiment 76, wherein the modification that reduces effector function comprises the substitutions of Ala at position 234 and Ala at position 235, according to EU numbering.

Embodiment 78. The isolated antibody or antigen-binding portion of any one of embodiments 66 to 77, wherein the first Fc polypeptide and/or the second Fc polypeptide comprises amino acid changes relative to the native Fc sequence that extend serum half-life.

Embodiment 79. The isolated antibody or antigen-binding portion of embodiment 78, wherein the amino acid changes comprise substitutions of Tyr at position 252, Thr at position 254, and Glu at position 256, according to EU numbering.

Embodiment 80. The isolated antibody or antigen-binding portion of embodiment 78, wherein the amino acid changes comprise substitutions of Leu at position 428 and Ser at position 434, according to EU numbering.

Embodiment 81. The isolated antibody or antigen-binding portion of embodiment 78, wherein the amino acid changes comprise a substitution of Ser or Ala at position 434, according to EU numbering.

Embodiment 82. The isolated antibody or antigen-binding portion of any one of embodiments 64 to 81, wherein the first Fc polypeptide and/or the second Fc polypeptide has an amino acid sequence identity of at least 75%, or at least 80%, 90%, 92%, or 95%, as compared to the corresponding wild-type Fc polypeptide.

Embodiment 83. The isolated antibody or antigen-binding portion of embodiment 82, wherein the corresponding wild-type Fc polypeptide is a human IgG1, IgG2, IgG3, or IgG4 Fc polypeptide.

Embodiment 84. The isolated antibody or antigen-binding portion of any one of embodiments 1 to 83, wherein the antibody is a monoclonal antibody.

Embodiment 85. The isolated antibody or antigen-binding portion of any one of embodiments 1 to 83, wherein the antibody is a chimeric antibody.

Embodiment 86. The isolated antibody or antigen-binding portion of any one of embodiments 1 to 83, wherein the antibody is a humanized antibody.

Embodiment 87. The isolated antibody or antigen-binding portion of any one of embodiments 1 to 83, wherein the antibody is a fully human antibody.

Embodiment 88. The isolated antibody or antigen-binding portion of any one of embodiments 1 to 87, wherein the antigen-binding portion is a Fab, a F(ab′)2, a scFv, or a bivalent scFv.

Embodiment 89. A hybridoma cell line that secretes the antibody or antigen-binding portion of any one of embodiments 1 to 88, wherein the hybridoma cell line is selected from the group consisting of 1H5, 20H4, 22D11, 16F3, 18B4, and 22E11.

Embodiment 90. An antibody or antigen-binding portion secreted by the hybridoma cell line of embodiment 89.

Embodiment 91. A composition comprising the antibody or antigen-binding portion of any one of embodiments 1 to 88 or 90 and a pharmaceutically acceptable carrier.

Embodiment 92. An isolated polynucleotide comprising a nucleotide sequence encoding the antibody or antigen-binding portion of any one of embodiments 1 to 88.

Embodiment 93. A vector comprising the polynucleotide of embodiment 92.

Embodiment 94. A host cell comprising the polynucleotide of embodiment 92 or the vector of embodiment 93.

Embodiment 95. An antibody or antigen-binding portion that competes with the antibody or antigen-binding portion of any one of embodiments 1 to 88 or 90 for binding to the human alpha-synuclein protein.

Embodiment 96. A method for neutralizing human alpha-synuclein protein in a brain of a subject, the method comprising administering to the subject the antibody or antigen-binding portion of any one of embodiments 1 to 88, 90, or 95 or the composition of embodiment 91.

Embodiment 97. The method of embodiment 96, wherein neutralizing comprises preventing or reducing human alpha-synuclein protein oligomerization and/or aggregation in the brain of the subject.

Embodiment 98. The method of embodiment 96, wherein neutralizing comprises preventing or reducing the spreading of human alpha-synuclein protein oligomers and/or fibrils in the brain of the subject.

Embodiment 99. The method of embodiment 98, wherein the antibody or antigen-binding portion or the composition is administered prior to the broad spreading of human alpha-synuclein protein oligomers and/or fibrils in the brain of the subject.

Embodiment 100. The method of embodiment 99, wherein administering the antibody or antigen-binding portion or the composition prior to the broad spreading of human alpha-synuclein protein oligomers and/or fibrils in the brain of the subject increases the ability of the antibody or the composition to treat a neurodegenerative disease.

Embodiment 101. The method of any one of embodiments 96 to 100, wherein the antibody or antigen-binding portion or the composition neutralizes monomeric human alpha-synuclein protein, oligomeric human alpha-synuclein protein, soluble human alpha-synuclein protein, human alpha-synuclein protein fibrils, human alpha-synuclein protein that is phosphorylated at Ser129, or a combination thereof.

Embodiment 102. The method of embodiment 101, wherein the antibody is a pan-alpha-synuclein antibody.

Embodiment 103. The method of any one of embodiments 96 to 102, wherein the subject has a neurodegenerative disease.

Embodiment 104. The method of embodiment 103, wherein the neurodegenerative disease is a synucleinopathy.

Embodiment 105. The method of embodiment 104, wherein the synucleinopathy is selected from the group consisting of Parkinson's disease, dementia with Lewy bodies, and multiple system atrophy.

Embodiment 106. The method of any one of embodiments 103 to 105, wherein the antibody or the composition is administered during an early stage of the neurodegenerative disease.

Embodiment 107. The method of any one of embodiments 96 to 106, wherein the amount of the antibody or antigen-binding portion or the composition that is necessary to neutralize the human alpha-synuclein protein in the brain of the subject is determined based on an amount of total human alpha-synuclein protein in a sample and/or an amount of human alpha-synuclein protein in a sample that is bound to the antibody.

Embodiment 108. The method of embodiment 107, wherein the sample is obtained from the subject.

Embodiment 109. The method of embodiment 107 or 108, wherein the sample is selected from the group consisting of an interstitial brain fluid sample, a cerebrospinal fluid (CSF) sample, a brain lysate sample, a blood sample, and a combination thereof.

Embodiment 110. The method of any one of embodiments 96 to 109, wherein the amount of the antibody or antigen-binding portion or the composition that is necessary to neutralize the human alpha-synuclein protein in the brain of the subject is determined based on a level of neurodegenerative disease progression in the subject and/or an amount of human alpha-synuclein protein spreading in the brain of the subject.

Embodiment 111. The method of embodiment 110, wherein an imaging method is used to determine the level of neurodegenerative disease progression in the subject and/or the amount of human alpha-synuclein protein spreading in the brain of the subject.

Embodiment 112. The method of embodiment 111, wherein the imaging method is selected from the group consisting of positron emission tomography (PET) imaging, dopamine transporter (DaT) imaging, magnetic resonance imaging (MRI), and computed tomography (CT) imaging.

Embodiment 113. The method of embodiment 112, wherein PET imaging comprises using a PET probe that comprises an antigen-binding portion or fragment of the antibody of any one of embodiments 1 to 88, 90, or 95.

Embodiment 114. A method for treating a neurodegenerative disease in a subject, the method comprising administering to the subject the antibody or antigen-binding portion of any one of embodiments 1 to 88, 90, or 95 or the composition of embodiment 91.

Embodiment 115. The method of embodiment 103 or 114, wherein the neurodegenerative disease is selected from the group consisting of Parkinson's disease, dementia with Lewy bodies, multiple system atrophy, Huntington's disease, Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis/parkinsonism-dementia complex of Guam, corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, inclusion-body myositis, myotonic dystrophy, neurofibrillary tangle-predominant dementia, Niemann-Pick disease type C, pallido-ponto-nigral degeneration, Pick's disease, postencephalitic parkinsonism, prion protein cerebral amyloid angiopathy, progressive subcortical gliosis, subacute sclerosing panencephalitis, and tangle only dementia.

Embodiment 116. An isolated antibody or antigen-binding portion thereof that specifically binds to a human alpha-synuclein protein, the antibody or antigen-binding portion thereof comprising: (i) a first and a second heavy chain each comprising a heavy chain variable region that comprises or consists of the amino acid sequence set forth in SEQ ID NO:147; and (ii) a first and a second light chain each comprising a light chain variable region that comprises or consists of the amino acid sequence set forth in SEQ ID NO:173, wherein each of the first and the second heavy chain comprises an Fc polypeptide, wherein the Fc polypeptide, wherein the Fc polypeptide is a wild-type IgG1, a wild-typ IgG2, a wild-type IgG3, or a wild-type IgG4 isotype.

Embodiment 117. An isolated antibody or antigen-binding portion thereof that specifically binds to a human alpha-synuclein protein, the antibody or antigen-binding portion thereof comprising: (i) a first and a second heavy chain each comprising a heavy chain variable region that comprises or consists of the amino acid sequence set forth in SEQ ID NO:147; and (ii) a first and a second light chain each comprising a light chain variable region that comprises or consists of the amino acid sequence set forth in SEQ ID NO:173, wherein each of the first and the second heavy chain comprises an Fc polypeptide, wherein one or both Fc polypeptides comprise (a) an Ala at position 234 and an Ala at position 235, and/or (b) a Leu at position 428 and a Ser at position 434 (EU numbering).

The various embodiments described above can be combined to provide further embodiments. All of the U.S. patents, U.S. patent application publications, U.S. patent applications, foreign patents, foreign patent applications and non-patent publications referred to in this specification, including U.S. Patent Application No. 62/903,588, filed Sep. 20, 2019, are incorporated herein by reference, in their entirety. The sequences of the sequence accession numbers cited herein are also hereby incorporated by reference. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

TABLE 1
Unique Hit Sequences

| | SEQ ID NO: | | | | | | | |
|---|---|---|---|---|---|---|---|---|
| Clone | CDR-H1 | CDR-H2 | CDR-H3 | VH | CDR-L1 | CDR-L2 | CDR-L3 | VL |
| 1H5 | 2 | 8 | 11 | 17 | 23 | 27 | 31 | 37 |
| 20H4 | 3 | 9 | 12 | 18 | 24 | 28 | 32 | 38 |
| 22D11 | 4 | 8 | 13 | 19 | 24 | 27 | 33 | 39 |
| 16F3 | 5 | 8 | 14 | 20 | 25 | 29 | 34 | 40 |
| 18B4 | 6 | 10 | 15 | 21 | 25 | 29 | 35 | 41 |
| 22E11 | 7 | 8 | 16 | 22 | 26 | 30 | 36 | 42 |
| 20H4_HC2 | 3 | 9 | 12 | 144 | — | — | — | — |
| 20H4_HC3 | 3 | 9 | 12 | 145 | — | — | — | — |
| 20H4_HC4 | 3 | 9 | 12 | 146 | — | — | — | — |
| 20H4_HC5 | 3 | 9 | 12 | 147 | — | — | — | — |
| 20H4_H1_1 | 97 | 9 | 12 | 148 | — | — | — | — |
| 20H4_H1_2 | 98 | 9 | 12 | 149 | — | — | — | — |
| 20H4_H1_3 | 99 | 9 | 12 | 150 | — | — | — | — |
| 20H4_H1_4 | 100 | 9 | 12 | 151 | — | — | — | — |
| 20H4_H1_5 | 101 | 9 | 12 | 152 | — | — | — | — |
| 20H4_H1_6 | 102 | 9 | 12 | 153 | — | — | — | — |
| 20H4_H2_1 | 3 | 105 | 12 | 154 | — | — | — | — |
| 20H4_H2_2 | 3 | 106 | 12 | 155 | — | — | — | — |
| 20H4_H2_3 | 3 | 107 | 12 | 156 | — | — | — | — |
| 20H4_H2_4 | 3 | 108 | 12 | 157 | — | — | — | — |
| 20H4_H2_5 | 3 | 109 | 12 | 158 | — | — | — | — |
| 20H4_H2_6 | 3 | 110 | 12 | 159 | — | — | — | — |
| 20H4_H3_1 | 3 | 9 | 113 | 160 | — | — | — | — |
| 20H4_H3_2 | 3 | 9 | 114 | 161 | — | — | — | — |
| 20H4_H3_3 | 3 | 9 | 115 | 162 | — | — | — | — |
| 20H4_H3_4 | 3 | 9 | 116 | 163 | — | — | — | — |
| 20H4_H3_5 | 3 | 9 | 117 | 164 | — | — | — | — |
| 20H4_H3_6 | 3 | 9 | 118 | 165 | — | — | — | — |
| 20H4_H3_7 | 3 | 9 | 119 | 166 | — | — | — | — |
| 20H4_LC1 | — | — | — | — | 24 | 28 | 32 | 167 |
| 20H4_LC2 | — | — | — | — | 24 | 28 | 32 | 168 |
| 20H4_LC3 | — | — | — | — | 24 | 28 | 32 | 169 |
| 20H4_LC4 | — | — | — | — | 24 | 28 | 32 | 170 |
| 20H4_LC5 | — | — | — | — | 24 | 28 | 32 | 171 |
| 20H4_LC6 | — | — | — | — | 24 | 28 | 32 | 172 |
| 20H4_L1_1 | — | — | — | — | 122 | 128 | 32 | 173 |
| 20H4_L1_2 | — | — | — | — | 123 | 28 | 32 | 174 |
| 20H4_L2_1 | — | — | — | — | 24 | 126 | 32 | 175 |
| 20H4_L2_2 | — | — | — | — | 24 | 127 | 32 | 176 |
| 20H4_L3_1 | — | — | — | — | 24 | 28 | 131 | 177 |
| 20H4_L3_2 | — | — | — | — | 24 | 28 | 132 | 178 |
| 20H4_L3_3 | — | — | — | — | 24 | 28 | 133 | 179 |
| 20H4_L3_4 | — | — | — | — | 24 | 28 | 134 | 180 |
| 20H4_L3_5 | — | — | — | — | 24 | 28 | 135 | 181 |
| 20H4_L3_6 | — | — | — | — | 24 | 28 | 136 | 182 |
| 20H4_L3_7 | — | — | — | — | 24 | 28 | 137 | 183 |
| 20H4_L3_8 | — | — | — | — | 24 | 28 | 138 | 184 |
| 20H4_L3_9 | — | — | — | — | 24 | 28 | 139 | 185 |
| 20H4_L3_10 | — | — | — | — | 24 | 28 | 140 | 186 |
| 20H4_L3_11 | — | — | — | — | 24 | 28 | 141 | 187 |

TABLE 2
Summary of Binding Data

| | | | | | | Fibril | Oligomer | | | |
|---|---|---|---|---|---|---|---|---|---|---|
| | | | | | | binding by | binding by | | | |
| | | | Biacore ™ for monomer | | | competition | competition | Biacore ™ for p129-Syn | | |
| | | | binding | | | ELISA | ELISA | binding | | |
| Primary | | Isoform | Kₐ | K_{d} | | IC50/KD | IC50/KD | Kₐ | | |
| Clone | Origin | Type | (1/Ms) | (1/s) | K_{D} (M) | (nM) | (nM) | (1/Ms) | K_{d} (1/s) | K_{D} (M) |
| 1H5 | Clonepix | rigG2b k | 1.042E+5 | 1.685E−3 | 1.617E−8 | 5.855 | 30.74 | 4.750E+4 | 1.126E−3 | 2.370E−8 |
| 20H4 | Hybridoma | rIgG2b k | 6.741E+5 | 2.026E−3 | 3.006E−9 | 1.728 | 5.376 | 2.984E+5 | 1.832E−3 | 6.140E−9 |
| 22D11 | Hybridoma | rIgG1 k | 1.858E+5 | 3.373E−3 | 1.815E−8 | 4.528 | 15.23 | 8.159E+4 | 3.498E−3 | 4.288E−8 |

| | | | | | | |
|---|---|---|---|---|---|---|
| 16F3 | Hybridoma | IgG2a k | N/A (biphasic | 4.649 | 3.924 | N/A (biphasic interaction) |
| | | | interaction) | | | |
| 18B4 | Hybridoma | IgG2b k | N/A (biphasic | 85.69 | 37.64 | N/A (biphasic interaction) |
| | | | interaction) | | | |
| 22E11 | Hybridoma | IgG1 k | N/A (biphasic | 28.08 | 6.962 | N/A (biphasic interaction) |
| | | | interaction) | | | |

TABLE 3
Summary of Epitope Mapping by Peptide ELISA

| | 1- 61 | 1- 95 | 1- 112 | 95- 140 | 61- 140 | human | mouse | con- clusion |
|---|---|---|---|---|---|---|---|---|
| 1H5 | − | − | − | ++++ | ++++ | ++++ | + | 112-140 |
| 16F3 | − | − | ++ | ++++ | ++++ | ++++ | +++ | 95-140 |
| 18B4 | − | − | ++ | ++++ | ++++ | ++++ | +++ | 95-140 |
| 20H4 | − | − | − | ++++ | ++++ | ++++ | − | 112-140 |
| 22D11 | − | − | − | ++++ | ++++ | ++++ | − | 112-140 |
| 22E11 | − | − | ++ | ++++ | ++++ | ++++ | +++ | 95-140 |

TABLE 4
Species Specificity Analysis

| | human | | |
|---|---|---|---|
| | alpha- | beta- | gamma- |
| | Syn | Syn | Syn |
| PBS | − | − | − |
| 20H4 | ++++ | − | − |
| 22D11 | ++++ | − | − |
| 1H5 | ++++ | − | − |

TABLE 5
Epitope Sequences

| | | SEQ | Amino acids |
|---|---|---|---|
| | | ID | within SEQ |
| Clone | Sequence | NO: | ID NO: 1 |
| 16F3. 18B4, 22E11 | VKKDQLGKNEEGAPQEGILEDMPVDPDNEA | 58 | 95-140 |
| | YEMPSEEGYQDYEPEA | | |
| 1H5, 22D11 | ILEDMPVDPDNEAYEMPSEEGYQDYEPEA | 59 | 112-140 |
| 20H4 | GILEDMPVDPDNEAYEMPSE | 60 | 111-130 |
| 20H4 | GILEDMPVDPDNEAY | 61 | 111-125 |
| 20H4 | MPVDPDNEAYEMPSE | 62 | 116-130 |
| 20H4 | DPDNEAY | 86 | 119-125 |

TABLE 6
Human and Rodent Alpha-synuclein Binding

| | EC50 (Bmax) | | | | | |
|---|---|---|---|---|---|---|
| Species | 1H5 | 16F3 | 18B4 | 20H4 | 22D11 | 22E11 |
| Human | 0.15 nM | 0.18 nM | 0.05 nM | 0.18 nM | 0.03 nM | 0.4 nM |
| | (1.14) | (0.94) | (1.36) | (0.83) | (1.2) | (1.05) |
| Rat | 2.97 nM | 0.6 nM | 0.3 nM | 1.5 nM | 2.0 nM | 1 nM |
| | (0.2) | (0.5) | (1.21) | (0.2) | (0.05) | (0.5) |
| Mouse | 5.86 nM | 0.15 nM | 0.08 nM | 4.3 nM | NB | 0.26 nM |
| | (0.16) | (0.76) | (i.oi) | (0.1) | | (0.58) |

Bmax: O.D. at 450 nm. For Bmax, O.D. of 0.1 or less considered to be weak binding.
NB denotes no binding.

TABLE 7
Alanine Scan Peptide Sequences

| Pep No. | Property | Sequence | SEQ ID NO: | Binding |
|---|---|---|---|---|
| 1 | asyn_113-127 | LEDMPVDPDNEAYEM | 67 | + |
| 2 | asyn_113-127_113A | AEDMPVDPDNEAYEM | 68 | + |
| 3 | asyn_113-127_114A | LADMPVDPDNEAYEM | 69 | + |
| 4 | asyn_113-127_115A | LEAMPVDPDNEAYEM | 70 | + |
| 5 | asyn_113-127_116A | LEDAPVDPDNEAYEM | 71 | + |
| 6 | asyn_113-127_117A | LEDMAVDPDNEAYEM | 72 | + |
| 7 | asyn_113-127_118A | LEDMPADPDNEAYEM | 73 | + |
| 8 | asyn_113-127_119A | LEDMPVAPDNEAYEM | 74 | - |
| 9 | asyn_113-127_120A | LEDMPVDADNEAYEM | 75 | + |
| 10 | asyn_113-127_121A | LEDMPVDPANEAYEM | 76 | + |
| 11 | asyn_113-127_122A | LEDMPVDPDAEAYEM | 77 | - |
| 12 | asyn_113-127_123A | LEDMPVDPDNAAYEM | 78 | + |
| 13 | asyn_113-127_124G | LEDMPVDPDNEGYEM | 79 | + |
| 14 | asyn_113-127_124P | LEDMPVDPDNEPYEM | 80 | - |
| 15 | asyn_113-127_125A | LEDMPVDPDNEAAEM | 81 | - |
| 16 | asyn_113-127_126A | LEDMPVDPDNEAYAM | 82 | + |
| 17 | asyn_113-127_127A | LEDMPVDPDNEAYEA | 83 | + |

TABLE 8
Alanine Scan Results

| | pep1 | pep2 | pep3 | pep4 | pep5 | pep6 | pep7 | pep8 | pep9 | pep10 | pep11 | pep12 |
|---|---|---|---|---|---|---|---|---|---|---|---|---|
| | 1 | 2 | 3 | 4 | 5 | 6 | 7 | 8 | 9 | 10 | 11 | 12 |
| A | 0.545 | 0.529 | 0.505 | 0.502 | 0.524 | 0.55 | 0.504 | 0.063 | 0.414 | 0.496 | 0.06 | 0.533 |
| B | 0.501 | 0.462 | 0.471 | 0.486 | 0.501 | 0.527 | 0.509 | 0.058 | 0.393 | 0.49 | 0.058 | 0.546 |
| C | 0.48 | 0.498 | 0.417 | 0.417 | 0.48 | 0.494 | 0.403 | 0.059 | 0.323 | 0.443 | 0.052 | 0.476 |
| D | 0.421 | 0.422 | 0.387 | 0.332 | 0.414 | 0.431 | 0.415 | 0.051 | 0.277 | 0.358 | 0.051 | 0.43 |
| E | 0.297 | 0.302 | 0.273 | 0.183 | 0.296 | 0.304 | 0.324 | 0.05 | 0.2 | 0.249 | 0.051 | 0.282 |
| F | 0.149 | 0.151 | 0.153 | 0.089 | 0.172 | 0.174 | 0.169 | 0.05 | 0.132 | 0.153 | 0.05 | 0.171 |
| G | 0.089 | 0.095 | 0.083 | 0.058 | 0.098 | 0.102 | 0.107 | 0.048 | 0.08 | 0.091 | 0.049 | 0.093 |
| H | 0.063 | 0.068 | 0.061 | 0.051 | 0.071 | 0.071 | 0.069 | 0.048 | 0.063 | 0.068 | 0.049 | 0.068 |
| | pep13 | pep14 | pep15 | pep16 | pep17 | bio-a-Syn | pep1 | pep2 | pep3 | pep4 | pep5 | pep6 |
| | 1 | 2 | 3 | 4 | 5 | 6 | 7 | 8 | 9 | 10 | 11 | 12 |
| A | 0.371 | 0.055 | 0.053 | 0.371 | 0.338 | 0.66 | 0.374 | 0.44 | 0.401 | 0.405 | 0.418 | 0.462 |
| B | 0.366 | 0.047 | 0.049 | 0.35 | 0.333 | 0.711 | 0.394 | 0.407 | 0.387 | 0.387 | 0.433 | 0.459 |
| C | 0.34 | 0.051 | 0.049 | 0.371 | 0.379 | 0.588 | 0.307 | 0.377 | 0.367 | 0.306 | 0.393 | 0.441 |
| D | 0.336 | 0.044 | 0.047 | 0.375 | 0.357 | 0.491 | 0.331 | 0.339 | 0.348 | 0.266 | 0.366 | 0.405 |
| E | 0.214 | 0.044 | 0.045 | 0.235 | 0.248 | 0.383 | 0.242 | 0.237 | 0.243 | 0.145 | 0.247 | 0.267 |
| F | 0.145 | 0.044 | 0.044 | 0.149 | 0.144 | 0.243 | 0.148 | 0.159 | 0.139 | 0.075 | 0.165 | 0.163 |
| G | 0.099 | 0.044 | 0.046 | 0.098 | 0.094 | 0.141 | 0.089 | 0.095 | 0.093 | 0.057 | 0.101 | 0.108 |
| H | 0.065 | 0.049 | 0.047 | 0.07 | 0.066 | 0.086 | 0.064 | 0.066 | 0.066 | 0.052 | 0.073 | 0.083 |
| | pep7 | pep8 | pep9 | pep10 | pep11 | pep12 | pep13 | pep14 | pep15 | pep16 | pep17 | bio-a-Syn |
| | 1 | 2 | 3 | 4 | 5 | 6 | 7 | 8 | 9 | 10 | 11 | 12 |
| A | 0.441 | 0.06 | 0.314 | 0.391 | 0.08 | 0.441 | 0.396 | 0.069 | 0.078 | 0.469 | 0.504 | 0.907 |
| B | 0.382 | 0.056 | 0.24 | 0.317 | 0.06 | 0.378 | 0.326 | 0.058 | 0.057 | 0.392 | 0.429 | 0.931 |
| C | 0.44 | 0.046 | 0.223 | 0.281 | 0.051 | 0.377 | 0.295 | 0.055 | 0.052 | 0.369 | 0.443 | 0.976 |
| D | 0.357 | 0.046 | 0.18 | 0.335 | 0.051 | 0.321 | 0.287 | 0.05 | 0.05 | 0.342 | 0.414 | 0.767 |
| E | 0.256 | 0.045 | 0.15 | 0.223 | 0.05 | 0.259 | 0.215 | 0.047 | 0.049 | 0.272 | 0.279 | 0.531 |
| F | 0.172 | 0.045 | 0.111 | 0.143 | 0.049 | 0.151 | 0.133 | 0.047 | 0.051 | 0.169 | 0.162 | 0.284 |
| G | 0.109 | 0.05 | 0.072 | 0.097 | 0.048 | 0.103 | 0.095 | 0.048 | 0.049 | 0.101 | 0.092 | 0.152 |
| H | 0.071 | 0.046 | 0.057 | 0.069 | 0.051 | 0.068 | 0.066 | 0.046 | 0.049 | 0.073 | 0.066 | 0.087 |

TABLE 9
Binding Data for Humanized 20H4 Variants

| Clone | VH | VL | K_{D} (M) |
|---|---|---|---|
| chimeric | | | 2.96E−09 |
| 20H4 control | | | |
| HC2_LC1 | SEQ ID NO: 144 | SEQ ID NO: 167 | 3.21E−09 |
| HC2_LC2 | SEQ ID NO: 144 | SEQ ID NO: 168 | 2.33E−09 |
| HC2_LC3 | SEQ ID NO: 144 | SEQ ID NO: 169 | 6.59E−09 |
| HC2_LC4 | SEQ ID NO: 144 | SEQ ID NO: 170 | 9.78E−09 |
| HC2_LC5 | SEQ ID NO: 144 | SEQ ID NO: 171 | 4.11E−09 |
| HC2_LC6 | SEQ ID NO: 144 | SEQ ID NO: 172 | 5.27E−09 |
| HC3_LC1 | SEQ ID NO: 145 | SEQ ID NO: 167 | 2.12E−09 |
| HC3_LC2 | SEQ ID NO: 145 | SEQ ID NO: 168 | 1.63E−09 |
| HC3_LC3 | SEQ ID NO: 145 | SEQ ID NO: 169 | 5.20E−09 |
| HC3_LC4 | SEQ ID NO: 145 | SEQ ID NO: 170 | 9.08E−09 |
| HC3_LC5 | SEQ ID NO: 145 | SEQ ID NO: 171 | 2.40E−09 |
| HC3_LC6 | SEQ ID NO: 145 | SEQ ID NO: 172 | 2.74E−09 |
| HC5_LC1 | SEQ ID NO: 147 | SEQ ID NO: 167 | 1.97E−09 |
| HC5_LC2 | SEQ ID NO: 147 | SEQ ID NO: 168 | 1.56E−09 |
| HC5_LC3 | SEQ ID NO: 147 | SEQ ID NO: 169 | 5.17E−09 |
| HC5_LC4 | SEQ ID NO: 147 | SEQ ID NO: 170 | 8.48E−09 |
| HC5_LC5 | SEQ ID NO: 147 | SEQ ID NO: 171 | 2.46E−09 |
| HC5_LC6 | SEQ ID NO: 147 | SEQ ID NO: 172 | 2.78E−09 |

TABLE 10
Binding Data for Affinity-matured Humanized 20H4 Variants

| Clone | VH | VL | K_{D} (M) |
|---|---|---|---|
| HC2_LC2 | SEQ ID NO: 144 | SEQ ID NO: 168 | 3.95E−09 |
| H1_1_LC2 | SEQ ID NO: 148 | SEQ ID NO: 168 | 3.46E−09 |
| H1_2_LC2 | SEQ ID NO: 149 | SEQ ID NO: 168 | 3.53E−09 |
| H1_3_LC2 | SEQ ID NO: 150 | SEQ ID NO: 168 | 4.04E−09 |
| H1_4_LC2 | SEQ ID NO: 151 | SEQ ID NO: 168 | 3.55E−09 |
| H1_5_LC2 | SEQ ID NO: 152 | SEQ ID NO: 168 | 4.61E−09 |
| H1_6_LC2 | SEQ ID NO: 153 | SEQ ID NO: 168 | 3.91E−09 |
| H2_1_LC2 | SEQ ID NO: 154 | SEQ ID NO: 168 | 6.18E−09 |
| H2_2_LC2 | SEQ ID NO: 155 | SEQ ID NO: 168 | 5.93E−09 |
| H2_3_LC2 | SEQ ID NO: 156 | SEQ ID NO: 168 | 4.76E−09 |
| H2_4_LC2 | SEQ ID NO: 157 | SEQ ID NO: 168 | 8.35E−09 |
| H2_5_LC2 | SEQ ID NO: 158 | SEQ ID NO: 168 | 5.50E−09 |
| H2_6_LC2 | SEQ ID NO: 159 | SEQ ID NO: 168 | 7.75E−09 |
| H3_1_LC2 | SEQ ID NO: 160 | SEQ ID NO: 168 | 4.79E−09 |
| H3_2_LC2 | SEQ ID NO: 161 | SEQ ID NO: 168 | 4.92E−09 |
| H3_3_LC2 | SEQ ID NO: 162 | SEQ ID NO: 168 | 5.30E−09 |
| H3_4_LC2 | SEQ ID NO: 163 | SEQ ID NO: 168 | 4.64E−09 |
| H3_5_LC2 | SEQ ID NO: 164 | SEQ ID NO: 168 | 3.84E−09 |
| H3_6_LC2 | SEQ ID NO: 165 | SEQ ID NO: 168 | 1.03E−08 |
| HC2_L1_1 | SEQ ID NO: 144 | SEQ ID NO: 173 | 1.20E−09 |
| HC2_L1_2 | SEQ ID NO: 144 | SEQ ID NO: 174 | 2.45E−09 |
| HC2_L2_1 | SEQ ID NO: 144 | SEQ ID NO: 175 | 1.78E−09 |
| HC2_L2_2 | SEQ ID NO: 144 | SEQ ID NO: 176 | 2.01E−09 |
| HC2_L3_1 | SEQ ID NO: 144 | SEQ ID NO: 177 | 4.20E−09 |
| HC2_L3_2 | SEQ ID NO: 144 | SEQ ID NO: 178 | 4.52E−09 |
| HC2_L3_3 | SEQ ID NO: 144 | SEQ ID NO: 179 | 5.28E−09 |
| HC2_L3_4 | SEQ ID NO: 144 | SEQ ID NO: 180 | 2.84E−09 |
| HC2_L3_5 | SEQ ID NO: 144 | SEQ ID NO: 181 | 3.24E−09 |
| HC2_L3_6 | SEQ ID NO: 144 | SEQ ID NO: 182 | 3.28E−09 |
| HC2_L3_7 | SEQ ID NO: 144 | SEQ ID NO: 183 | 4.59E−09 |
| HC2_L3_8 | SEQ ID NO: 144 | SEQ ID NO: 184 | 3.01E−09 |
| HC2_L3_9 | SEQ ID NO: 144 | SEQ ID NO: 185 | 2.98E−09 |
| HC2_L3_10 | SEQ ID NO: 144 | SEQ ID NO: 186 | 2.93E−09 |
| HC2_L3_11 | SEQ ID NO: 144 | SEQ ID NO: 187 | 3.40E−09 |

TABLE 11
Binding Data for Additional Affinity-matured
Humanized 20H4 Variants

| Clone | VH | VL | K_{D} (M) |
|---|---|---|---|
| HC6_LC_k3_1 | SEQ ID NO: 229 | SEQ ID NO: 201 | 1.60E−10 |
| HC6_LC_k3_2_ | SEQ ID NO: 229 | SEQ ID NO: 202 | 2.20E−10 |
| v1 | | | |
| HC6_LC_k3_3 | SEQ ID NO: 229 | SEQ ID NO: 203 | 1.60E−10 |
| HC6_LC_k3_4 | SEQ ID NO: 229 | SEQ ID NO: 204 | 1.80E−10 |
| HC6_LC_k3_5 | SEQ ID NO: 229 | SEQ ID NO: 205 | 2.10E−10 |
| HC6_LC_k3_6 | SEQ ID NO: 229 | SEQ ID NO: 206 | 1.40E−10 |
| HC6_LC_k3_7 | SEQ ID NO: 229 | SEQ ID NO: 207 | 2.20E−10 |
| HC6_LC_k3_8 | SEQ ID NO: 229 | SEQ ID NO: 208 | 2.20E−10 |
| HC6_LC_k3_9 | SEQ ID NO: 229 | SEQ ID NO: 209 | 1.70E−10 |
| HC6_LC_k3_10 | SEQ ID NO: 229 | SEQ ID NO: 210 | 1.70E−10 |
| HC6_LC_k3_11 | SEQ ID NO: 229 | SEQ ID NO: 211 | 2.40E−10 |
| HC6_LC_k3_12 | SEQ ID NO: 229 | SEQ ID NO: 212 | 1.50E−10 |
| HC6_LC_k3_13 | SEQ ID NO: 229 | SEQ ID NO: 213 | 1.70E−10 |
| HC5_L1_1 | SEQ ID NO: 147 | SEQ ID NO: 201 | 1.00E−10 |
| HC5_LC_k3_1 | SEQ ID NO: 147 | SEQ ID NO: 201 | 1.30E−10 |
| HC5_LC_k3_2_ | SEQ ID NO: 147 | SEQ ID NO: 202 | 1.70E−10 |
| v1 | | | |
| HC5_LC_k3_2_ | SEQ ID NO: 147 | SEQ ID NO: 200 | 1.30E−9 |
| v2 | | | |
| HC5_LC_k3_3 | SEQ ID NO: 147 | SEQ ID NO: 203 | 1.70E−10 |
| HC5_LC_k3_4 | SEQ ID NO: 147 | SEQ ID NO: 204 | 1.50E−10 |
| HC5_LC_k3_5 | SEQ ID NO: 147 | SEQ ID NO: 205 | 1.30E−10 |
| HC5_LC_k3_6 | SEQ ID NO: 147 | SEQ ID NO: 206 | 1.90E−10 |

TABLE 12
Informal Sequence Listing

| SEQ ID | | |
|---|---|---|
| NO: | Sequence | Description |
| 1 | MDVFMKGLSKAKEGVVAAAEKTKQGVAEAAGKTKEGVLY | Alpha-synuclein full |
| | VGSKTKEGVVHGVATVAEKTKEQVTNVGGAVVTGVTAVA | amino acid sequence |
| | QKTVEGAGSIAAATGFVKKDQLGKNEEGAPQEGILEDMPVD | |
| | PDNEAYEMPSEEGYQDYEPEA | |
| 2 | GFTFSDAAMY | CDR-H1 for clone 1H5 |
| 3 | GFTFSNAAMY | CDR-H1 for clone 20H4 |
| 4 | GFTFTNAAIY | CDR-H1 for clone 22D11 |
| 5 | GFTFSNGAMN | CDR-H1 for clone 16F3 |
| 6 | GFTFSSFAMA | CDR-H1 for clone 18B4 |
| 7 | GFTFSTAAMY | CDR-H1 for clone 22E11 |
| 8 | RIRTKPNNYATYYADSVKG | CDR-H2 for clones 1H5, |
| | | 16F3, and 22E11 |
| 9 | RIRTKPNNYATYHADSVKG | CDR-H2 for clone 20H4 |
| 10 | TITSGGSKTYYPDSVKG | CDR-H2 for clone 18B4 |
| 11 | SADFHYFDY | CDR-H3 for clone 1H5 |
| 12 | TADMATGDY | CDR-H3 for clone 20H4 |
| 13 | AADASRGDY | CDR-H3 for clone 22D11 |
| 14 | TADGATPFAY | CDR-H3 for clone 16F3 |
| 15 | AKGYFDY | CDR-H3 for clone 18B4 |
| 16 | TADYYDGTPFAY | CDR-H3 for clone 22E11 |
| 17 | AVQLVESGGGLVQPKESLKISCAASGFTFSDAAMYWVRQAP | VH for clone 1H5 |
| | GKGLELVARIRTKPNNYATYYADSVKGRFTISRDDSKSMVY | |
| | LQMGNLKTEDTAMYYCSADFHYFDYWGQGVMVTVSS | |
| 18 | AVQLVESGGGLVQPKESLKISCAASGFTFSNAAMYWVRQAP | VH for clone 20H4 |
| | GKGLELLARIRTKPNNYATYHADSVKGRFTISRDDSQSMVY | |
| | LHMGNLKTEDTAMYYCTADMATGDYWGPGVMVTVSS | |
| 19 | AVQFVESGGGLVRPKESLKISCTASGFTFTNAAIYWVRQAPG | VHfor clone 22D11 |
| | KGLECIARIRTKPNNYATYYADSVRGRFTISRDDSKPMVYLQ | |
| | MNELKTEDTAMYYCAADASRGDYWGQGVMVTVSS | |
| 20 | AVQLVESGGGLVQPKESLKISCAASGFTFSNGAMNWVRQAP | VH for clone 16F3 |
| | GKGLEWVARIRTKPNNYATYYADSVKGRFTISRDDSKSMVY | |
| | LQMDDLKTEDTAMYYCTADGATPFAYWGQGTLVTVSS | |
| 21 | EVQLVESGGGLVQPGRSLKLSCAASGFTFSSFAMAWVRQAP | VH for clone 18B4 |
| | KKGLEWVATITSGGSKTYYPDSVKGRFTISRDNAKSTLYLQ | |
| | MDSLRSEDTATYYCAKGYFDYWGQGVMVTVSS | |
| 22 | AVQLVESGGGLVQPKESLKISCAASGFTFSTAAMYWVRQAP | VHfor clone 22E11 |
| | GKGLEWVTRIRTKPNNYATYYADSVKGRFTISRDDSKSMVY | |
| | LQMDNLKTEDTAMYYCTADYYDGTPFAYWGQGTLVTVSS | |
| 23 | KAGQNINKNLD | CDR-L1 fordone 1H5 |
| 24 | KASQNINKNLD | CDR-L1 for clones 20H4, |
| | | 22D11, |
| | | 20H4_LC_k3_2_v1, and |
| | | 20H4_LC_k3_2_v2 |
| 25 | RSSQSLLHSNGNTYLH | CDR-L1 for clones 16F3 |
| | | and 18B4 |
| 26 | RSSQSLVHNNGITYLS | CDR-L1 for clone 22E11 |
| 27 | FTNNLQT | CDR-L2 for clones 1H5 |
| | | and 22D11 |
| 28 | STNNLQT | CDR-L2 for clone 20H4 |
| | | and 20H4_LC_k3_2_v2 |
| 29 | LVSRLES | CDR-L2 for clones 16F3 |
| | | and 18B4 |
| 30 | KVSNRFS | CDR-L2 for clone 22E11 |
| 31 | YQYNSGWT | CDR-L3 for clone 1H5 |
| 32 | YQYKSAWT | CDR-L3 for clone 20H4 |
| 33 | YQYTSGFT | CDR-L3 for clone 22D11 |
| 34 | VQGTHAPFT | CDR-L3 for clone 16F3 |
| 35 | VQGTHAPLT | CDR-L3 for clone 18B4 |
| 36 | GQGTQYPYT | CDR-L3 for clone 22E11 |
| 37 | DIQMTQSPSVLSASVGDRVTLSCKAGQNINKNLDWYQQKLG | VL for clone 1H5 |
| | EAPKLLISFTNNLQTGVPSRFSGSGSGSDYTLTISSLQPEDVAT | |
| | YYCYQYNSGWTFGGGTKLELK | |
| 38 | DIQMTQSPPVLSVSVGDRVTLSCKASQNINKNLDWYQQKHG | VL for clone 20H4 |
| | EAPKPLIYSTNNLQTGVPSRFSGSGSGTDYTLTISSLQPEDVA | |
| | TYYCYQYKSAWTFGGGTKLELR | |
| 39 | DIQMTQSPSVLSASVGDRVTFSCKASQNINKNLDWYQQKLG | VL for clone 22D11 |
| | EVPKVLIYFTNNLQTGIPSRFSGSGSGTDYTLTISSLQPEDVAT | |
| | YYCYQYTSGFTFGAGTKLELK | |
| 40 | DVVLTQTPPTLSATIGQSVSISCRSSQSLLHSNGNTYLHWLLQ | VL for clone 16F3 |
| | RPGQSPQLLIYLVSRLESGVPNRFSGSGSGTDFTLKISGVEAE | |
| | DLGVYYCVQGTHAPFTFGSGTKLEIK | |
| 41 | DVVLTQTPPTLSATIGQSVSISCRSSQSLLHSNGNTYLHWLLQ | VLfor clone 18B4 |
| | RPGQSPQLLIYLVSRLESGVPNRFSGSGSGTDFTLKISGVEAE | |
| | DLGVYYCVQGTHAPLTFGSGTKLEIk | |
| 42 | AVVMTQTPVSLSVSLGGQVSISCRSSQSLVHNNGITYLSWYL | VL for clone 22E11 |
| | QKPGQSPQLPIYKVSNRFSGFSDRFSGGGSGTDFTLKISRVEP | |
| | DDLGLYYCGQGTQYPYTFGAGTKLELK | |
| 43 | GFxFxxxAxx | CDR-H1 consensus |
| | | sequence |
| 44 | RIRTKPNNYATYxADSVxG | CDR-H2 consensus |
| | | sequence |
| 45 | TYxxDSVxG | CDR-H2 consensus |
| | | sequence |
| 46 | xADxxxxDY | CDR-H3 consensus |
| | | sequence |
| 47 | KAxQxINxxLD | CDR-L1 consensus |
| | | sequence |
| 48 | RSSQSLxHxNGxTYLx | CDR-L1 consensus |
| | | sequence |
| 49 | xTxxLxT | CDR-L2 consensus |
| | | sequence |
| 50 | xVSxxxS | CDR-L2 consensus |
| | | sequence |
| 51 | YQxxxxxT | CDR-L3 consensus |
| | | sequence |
| 52 | xQGTxxPxT | CDR-L3 consensus |
| | | sequence |
| 53 | AGGCTTGCAATCACCTCCACA | ratIgG1 VH 3’ PCR |
| | | primer |
| 54 | ACAAGGATTGCATTCCCTTGG | ratIgG2a VH 3’ PCR |
| | | primer |
| 55 | GCATTTGTGTCCAATGCCGCC | ratIgG2b VH 3’ PCR |
| | | primer |
| 56 | TCTGGGCTTGGGTCTTCTGGG | ratIgG2c VH 3’ PCR |
| | | primer |
| 57 | CTCATTCCTGTTGAAGCTCTTGACGACGGG | ratCkappa VL 3’ primer |
| 58 | VKKDQLGKNEEGAPQEGILEDMPVDPDNEAYEMPSEEGYQ | Epitope sequence (amino |
| | DYEPEA | acids 95-140 within SEQ |
| | | ID NO: 1) |
| 59 | ILEDMPVDPDNEAYEMPSEEGYQDYEPEA | Epitope sequence (amino |
| | | acids (112-140 within |
| | | SEQ ID NO: 1) |
| 60 | GILEDMPVDPDNEAYEMPSE | Epitope sequence (amino |
| | | acids 111-130 within SEQ |
| | | ID NO: 1) |
| 61 | GILEDMPVDPDNEAY | Epitope sequence (amino |
| | | acids 111-125 within SEQ |
| | | ID NO: 1) |
| 62 | MPVDPDNEAYEMPSE | Epitope sequence (amino |
| | | acids 116-130 within SEQ |
| | | ID NO: 1) |
| 63 | EPKSCDKTHTCPPCP | Human IgG1 hinge |
| | | sequence |
| 64 | APELLGGPSVFLFPPKPKDTLMISRTPEVTCVVVDVSHEDPEV | Wild-type human Fc |
| | KFNWYVDGVEVHNAKTKPREEQYNSTYRVVSVLTVLHQD | sequence |
| | WLNGKEYKCKVSNKALPAPIEKTISKAKGQPREPQVYTLPPS | |
| | RDELTKNQVSLTCLVKGFYPSDIAVEWESNGQPENNYKTTPP | positions 231-447 EU |
| | VLDSDGSFFLYSKLTVDKSRWQQGNVFSCSVMHEALHNHY | index numbering |
| | TQKSLSLSPGK | |
| 65 | DIQMTQSPPVLSVSVGDRVTLSCKASQNINKNLDWYQQKHG | Chimeric 20H4 (Chi |
| | EAPKPLIYSTNNLQTGVPSRFSGSGSGTDYTLTISSLQPEDVA | 20H4) light chain |
| | TYYCYQYKSAWTFGGGTKLELRRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| 66 | AVQLVESGGGLVQPKESLKISCAASGFTFSNAAMYWVRQAP | Chimeric 20H4 (Chi |
| | GKGLELLARIRTKPNNYATYHADSVKGRFTISRDDSQSMVY | 20H4) heavy chain |
| | LHMGNLKTEDTAMYYCTADMATGDYWGPGVMVTVSSAST | sequence (wild-type |
| | KGPSVFPLAPSSKSTSGGTAALGCLVKDYFPEPVTVSWNSGA | human Fc) |
| | LTSGVHTFPAVLQSSGLYSLSSVVTVPSSSLGTQTYICNVNHK | |
| | PSNTKVDKKVEPKSCDKTHTCPPCPAPELLGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSRDELTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPGK | |
| 67 | LEDMPVDPDNEAYEM | Amino acids 113-127 of |
| | | SEQ ID NO: 1 |
| 68 | AEDMPVDPDNEAYEM | Peptide containing alanine |
| | | substitution at position |
| | | 113 of SEQ ID NO: 1 |
| 69 | LADMPVDPDNEAYEM | Peptide containing alanine |
| | | substitution at position |
| | | 114 of SEQ ID NO: 1 |
| 70 | LEAMPVDPDNEAYEM | Peptide containing alanine |
| | | substitution at position |
| | | 115 of SEQ ID NO: 1 |
| 71 | LEDAPVDPDNEAYEM | Peptide containing alanine |
| | | substitution at position |
| | | 116 of SEQ ID NO: 1 |
| 72 | LEDMAVDPDNEAYEM | Peptide containing alanine |
| | | substitution at position |
| | | 117 of SEQ ID NO: 1 |
| 73 | LEDMPADPDNEAYEM | Peptide containing alanine |
| | | substitution at position |
| | | 118 of SEQ ID NO: 1 |
| 74 | LEDMPVAPDNEAYEM | Peptide containing alanine |
| | | substitution at position |
| | | 119 of SEQ ID NO: 1 |
| 75 | LEDMPVDADNEAYEM | Peptide containing alanine |
| | | substitution at position |
| | | 120 of SEQ ID NO: 1 |
| 76 | LEDMPVDPANEAYEM | Peptide containing alanine |
| | | substitution at position |
| | | 121 of SEQ ID NO: 1 |
| 77 | LEDMPVDPDAEAYEM | Peptide containing alanine |
| | | substitution at position |
| | | 122 of SEQ ID NO: 1 |
| 78 | LEDMPVDPDNAAYEM | Peptide containing alanine |
| | | substitution at position |
| | | 123 of SEQ ID NO: 1 |
| 79 | LEDMPVDPDNEGYEM | Peptide containing glycine |
| | | substitution at position |
| | | 124 of SEQ ID NO: 1 |
| 80 | LEDMPVDPDNEPYEM | Peptide containing proline |
| | | substitution at position |
| | | 124 of SEQ ID NO: 1 |
| 81 | LEDMPVDPDNEAAEM | Peptide containing alanine |
| | | substitution at position |
| | | 125 of SEQ ID NO: 1 |
| 82 | LEDMPVDPDNEAYAM | Peptide containing alanine |
| | | substitution at position |
| | | 126 of SEQ ID NO: 1 |
| 83 | LEDMPVDPDNEAYEA | Peptide containing alanine |
| | | substitution at position |
| | | 127 of SEQ ID NO: 1 |
| 84 | [reserved] | |
| 85 | [reserved] | |
| 86 | DPDNEAY | Epitope sequence (amino |
| | | acids 119-125 within SEQ |
| | | ID NO: 1) |
| 87 | GFX₁FX₂X₃X₄AX₅X₆, wherein X₁ is T, R, K, or N; X₂ is | CDR-H1 consensus |
| | N, S, or T; X₃ is N, K, R, D, S, or T; X₄ is A, G, | sequence |
| | or F; X₅ is 1 or M; and X is Y, N, or A | |
| 88 | RIRTKPNNYATYX₁ADSVX₂G, wherein X₁ is Y or H and X₂ | CDR-H2 consensus |
| | is K or R | sequence |
| 89 | TYX₁X₂DSVX₃G, wherein X₁ is Y or H; X₂ is A or P; | CDR-H2 consensus |
| | and X₃ is K or R | sequence |
| 90 | X₁ADX₂X₃X₄X₅DY, wherein X₁ is T or S; X₂ is F, M, or | CDR-H3 consensus |
| | A; X₃ is H, A, or S; X₄ is Y, T, or R; and X₅ is F | sequence |
| | or G | |
| 91 | KAX₁QX₂INX₃X₄LD, wherein X₁ is G or S; X₂ is N or I; | CDR-L1 consensus |
| | X₃ is K, N, Q, D, or A; and X₄ is N or I | sequence |
| 92 | RSSQSLX₁HX₂NGX₃TYLX₄, wherein X₁ is L or V; X₂ is S | CDR-L1 consensus |
| | or N ;X₃ is N or I; and X₄ is H or S | sequence |
| 93 | X₁TX₂X₄LX₄T, wherein X₁ is F, R, or S; X₂ is N or K, | CDR-L2 consensus |
| | X₃ is N or K; and X₄ is Q or K | sequence |
| 94 | X₁VSX₂X₃X₄S, wherein X₁ is L or K; X₂ is R or N; X₃ | CDR-L2 consensus |
| | is L or R; and X₄ is E or F | sequence |
| 95 | YQX₁X₂X₃X₄X₅T, wherein X₁ is Y, D, or F; X₂ is T, N, | CDR-L3 consensus |
| | or K; X₃ is S, N, R, K, Y, L, or F; X₄ is A or G; | sequence |
| | and X₅ is W or F | |
| 96 | X₁QGTX₂X₃PX₄T, wherein X₁ is V or G; X₂ is H or Q; | CDR-L3 consensus |
| | X₃ is A or Y; and X₄ is F, L, or Y | sequence |
| 97 | GFTFSKAAMY | 20H4_H1_1_CDR-H1 |
| | | sequence |
| 98 | GFRFSKAAMY | 20H4_H1_2_CDR-H1 |
| | | sequence |
| 99 | GFTFSRAAMY | 20H4_H1_3 CDR-H1 |
| | | sequence |
| 100 | GFTFNKAAMY | 20H4_H1_4 CDR-H1 |
| | | sequence |
| 101 | GFKFSKAAMY | 20H4_H1_5 CDR-H1 |
| | | sequence |
| 102 | GFNFSKAAMY | 20H4_H1_6 CDR-H1 |
| | | sequence |
| 103 | GFxFxxAAMY | 20H4 CDR-H1 consensus |
| | | sequence |
| 104 | GFX₁FX2X3AAMY, wherein X₁ is T, R, K, or N; X₂ is S | 20H4 CDR-H1 consensus |
| | or N; and X₃ is N, K, orR | sequence |
| 105 | RIRTKPNNFATYHADSVKG | 20H4_H2_1 CDR-H2 |
| | | sequence |
| 106 | RIRTQRNNYATYHADSVKG | 20H4_H2_2 CDR-H2 |
| | | sequence |
| 107 | RIRTERNNYATYHADSVKG | 20H4_H2_3 CDR-H2 |
| | | sequence |
| 108 | RIRTKRNNYATYHADSVKG | 20H4_H2_4 CDR-H2 |
| | | sequence |
| 109 | RIRTEPNNYATYHADSVKG | 20H4_H2_5 CDR-H2 |
| | | sequence |
| 110 | RIRTKPKNYATYHADSVKG | 20H4_H2_6 CDR-H2 |
| | | sequence |
| 111 | RIRTxxxNxATYHADSVKG | 20H4 CDR-H2 consensus |
| | | sequence |
| 112 | RIRTX₁X₂X₃NX₄ATYHADSVKG, wherein X₁ is K, Q, or E; | 20H4 CDR-H2 consensus |
| | X₂ is P or R; X₃ is N or K; and X₄ is Y or F | sequence |
| 113 | TANMATGDY | 20H4_H3_1 CDR-H3 |
| | | sequence |
| 114 | TANMAKGDY | 20H4_H3_2 CDR-H3 |
| | | sequence |
| 115 | AAAMARGDY | 20H4_H3_3 CDR-H3 |
| | | sequence |
| 116 | SADFAKGDY | 20H4_H3_4 CDR-H3 |
| | | sequence |
| 117 | TADFARGDY | 20H4_H3_5 CDR-H3 |
| | | sequence |
| 118 | TANMRKGDY | 20H4_H3_6 CDR-H3 |
| | | sequence |
| 119 | TADMAKGDY | 20H4_H3_7 CDR-H3 |
| | | sequence |
| 120 | xAxxxxGDY | 20H4 CDR-H3 consensus |
| | | sequence |
| 121 | X₁AX₂X₃X₄X₅GDY, wherein X₁ is T, A, or S; X₂ is D, | 20H4 CDR-H3 consensus |
| | N, or A; X₃ is M or F; X₄ is A or R; and Xs is T, | sequence |
| | K, or R | |
| 122 | KASQNINNNLD | 20H4_L1_1 CDR-L1 and |
| | | 20H4_LC_k3_1 CDR-L1 |
| | | sequence |
| 123 | KASQIINQILD | 20H4_L1_2 CDR-L1 |
| | | sequence |
| 124 | KASQxINxxLD | 20H4 CDR-L1 consensus |
| | | sequence |
| 125 | KASQX₁INX₂X₃LD, wherein X₁ is N or I; X₂ is K, N, Q, | 20H4 CDR-L1 consensus |
| | D, or A; and X₃ is N or I | sequence |
| 126 | STNKLKT | 20H4_L2_1 CDR-L2 |
| | | sequence |
| 127 | RTKNLQT | 20H4_L2_2 CDR-L2 |
| | | sequence |
| 128 | STNNLKT | CDR-L2 sequence |
| | | 20H_4L1_1, |
| | | 20H4_LC_k3_1, and |
| | | 20H4_LC_k3_2_v1 |
| 129 | xTxxLxT | 20H4 CDR-L2 consensus |
| | | sequence |
| 130 | X₁TX₂X₃LX₄T, wherein X₁ is S or R; X₂ is N or K; X₃ | 20H4 CDR-L2 consensus |
| | is N or K; and X₄ is Q or K | sequence |
| 131 | YQDKNGWT | 20H4_L3_1 CDR-L3 |
| | | sequence |
| 132 | YQDKRGWT | 20H4_L3_2 CDR-L3 |
| | | sequence |
| 133 | YQDKKGWT | 20H4_L3_3 CDR-L3 |
| | | sequence |
| 134 | YQDKSGWT | 20H4_L3_4 CDR-L3 |
| | | sequence |
| 135 | YQYKNAWT | 20H4_L3_5 CDR-L3 |
| | | sequence |
| 136 | YQYKKAWT | 20H4_L3_6 CDR-L3 |
| | | sequence |
| 137 | YQYKNGWT | 20H4_L3_7 CDR-L3 |
| | | sequence |
| 138 | YQFKKAWT | 20H4_L3_8 CDR-L3 |
| | | sequence |
| 139 | YQYKYAWT | 20H4_L3_9 CDR-L3 |
| | | sequence |
| 140 | YQYKLAWT | 20H4_L3_10 CDR-L3 |
| | | sequence |
| 141 | YQYKFAWT | 20H4_L3_11 CDR-L3 |
| | | sequence |
| 142 | YQxKxxWT | 20H4 CDR-L3 consensus |
| | | sequence |
| 143 | YQX₁KX₂X₃WT, wherein X₁ is Y, D, or F; X₂ is S, N, | 20H4 CDR-L3 consensus |
| | R, K, Y, L, or F; and X₃ is A or G | sequence |
| 144 | EVQLVESGGGLVQPGGSLRLSCAASGFTFSNAAMYWVRQAP | 20H4_HC2 |
| | GKGLELVSRIRTKPNNYATYHADSVKGRFTISRDNSKNTLYL | |
| | QMNSLRAEDTAVYYCTADMATGDYWGQGTLVTVSS | |
| 145 | EVQLVESGGGLVQPGGSLRLSCAASGFTFSNAAMYWVRQAP | 20H4_HC3 |
| | GKGLELVSRIRTKPNNYATYHADSVKGRFTISRDNSKNTVYL | |
| | QMNSLRAEDTAVYYCTADMATGDYWGQGTLVTVSS | |
| 146 | EVQLVESGGGLVQPGGSLRLSCAASGFTFSNAAMYWVRQAP | 20H4_HC4 |
| | GKGLELLARIRTKPNNYATYHADSVKGRFTISRDNSKNTLYL | |
| | QMNSLRAEDTAVYYCTADMATGDYWGQGTLVTVSS | |
| 147 | EVQLVESGGGLVQPGGSLRLSCAASGFTFSNAAMYWVRQAP | 20H4_HC5 |
| | GKGLELLARIRTKPNNYATYHADSVKGRFTISRDDSKNTVYL | |
| | QMNSLRAEDTAVYYCTADMATGDYWGQGTLVTVSS | |
| 148 | EVQLVESGGGLVQPGGSLRLSCAASGFTFSKAAMYWVRQAP | 20H4_H1_1 |
| | GKGLELVSRIRTKPNNYATYHADSVKGRFTISRDNSKNTLYL | |
| | QMNSLRAEDTAVYYCTADMATGDYWGQGTLVTVSS | |
| 149 | EVQLVESGGGLVQPGGSLRLSCAASGFRFSKAAMYWVRQA | 20H4_H1_2 |
| | PGKGLELVSRIRTKPNNYATYHADSVKGRFTISRDNSKNTLY | |
| | LQMNSLRAEDTAVYYCTADMATGDYWGQGTLVTVSS | |
| 150 | EVQLVESGGGLVQPGGSLRLSCAASGFTFSRAAMYWVRQAP | 20H4_H1_3 |
| | GKGLELVSRIRTKPNNYATYHADSVKGRFTISRDNSKNTLYL | |
| | QMNSLRAEDTAVYYCTADMATGDYWGQGTLVTVSS | |
| 151 | EVQLVESGGGLVQPGGSLRLSCAASGFTFNKAAMYWVRQA | 20H4_H1_4 |
| | PGKGLELVSRIRTKPNNYATYHADSVKGRFTISRDNSKNTLY | |
| | LQMNSLRAEDTAVYYCTADMATGDYWGQGTLVTVSS | |
| 152 | EVQLVESGGGLVQPGGSLRLSCAASGFKFSKAAMYWVRQA | 20H4_H1_5 |
| | PGKGLELVSRIRTKPNNYATYHADSVKGRFTISRDNSKNTLY | |
| | LQMNSLRAEDTAVYYCTADMATGDYWGQGTLVTVSS | |
| 153 | EVQLVESGGGLVQPGGSLRLSCAASGFNFSKAAMYWVRQA | 20H4_H1_6 |
| | PGKGLELVSRIRTKPNNYATYHADSVKGRFTISRDNSKNTLY | |
| | LQMNSLRAEDTAVYYCTADMATGDYWGQGTLVTVSS | |
| 154 | EVQLVESGGGLVQPGGSLRLSCAASGFTFSNAAMYWVRQAP | 20H4_H2_1 |
| | GKGLELVSRIRTKPNNFATYHADSVKGRFTISRDNSKNTLYL | |
| | QMNSLRAEDTAVYYCTADMATGDYWGQGTLVTVSS | |
| 155 | EVQLVESGGGLVQPGGSLRLSCAASGFTFSNAAMYWVRQAP | 20H4_H2_2 |
| | GKGLELVSRIRTQRNNYATYHADSVKGRFTISRDNSKNTLYL | |
| | QMNSLRAEDTAVYYCTADMATGDYWGQGTLVTVSS | |
| 156 | EVQLVESGGGLVQPGGSLRLSCAASGFTFSNAAMYWVRQAP | 20H4_H2_3 |
| | GKGLELVSRIRTERNNYATYHADSVKGRFTISRDNSKNTLYL | |
| | QMNSLRAEDTAVYYCTADMATGDYWGQGTLVTVSS | |
| 157 | EVQLVESGGGLVQPGGSLRLSCAASGFTFSNAAMYWVRQAP | 20H4_H2_4 |
| | GKGLELVSRIRTKRNNYATYHADSVKGRFTISRDNSKNTLYL | |
| | QMNSLRAEDTAVYYCTADMATGDYWGQGTLVTVSS | |
| 158 | EVQLVESGGGLVQPGGSLRLSCAASGFTFSNAAMYWVRQAP | 20H4_H2_5 |
| | GKGLELVSRIRTEPNNYATYHADSVKGRFTISRDNSKNTLYL | |
| | QMNSLRAEDTAVYYCTADMATGDYWGQGTLVTVSS | |
| 159 | EVQLVESGGGLVQPGGSLRLSCAASGFTFSNAAMYWVRQAP | 20H4_H2_6 |
| | GKGLELVSRIRTKPKNYATYHADSVKGRFTISRDNSKNTLYL | |
| | QMNSLRAEDTAVYYCTADMATGDYWGQGTLVTVSS | |
| 160 | EVQLVESGGGLVQPGGSLRLSCAASGFTFSNAAMYWVRQAP | 20H4_H3_1 |
| | GKGLELVSRIRTKPNNYATYHADSVKGRFTISRDNSKNTLYL | |
| | QMNSLRAEDTAVYYCTANMATGDYWGQGTLVTVSS | |
| 161 | EVQLVESGGGLVQPGGSLRLSCAASGFTFSNAAMYWVRQAP | 20H4_H3_2 |
| | GKGLELVSRIRTKPNNYATYHADSVKGRFTISRDNSKNTLYL | |
| | QMNSLRAEDTAVYYCTANMAKGDYWGQGTLVTVSS | |
| 162 | EVQLVESGGGLVQPGGSLRLSCAASGFTFSNAAMYWVRQAP | 20H4_H3_3 |
| | GKGLELVSRIRTKPNNYATYHADSVKGRFTISRDNSKNTLYL | |
| | QMNSLRAEDTAVYYCAAAMARGDYWGQGTLVTVSS | |
| 163 | EVQLVESGGGLVQPGGSLRLSCAASGFTFSNAAMYWVRQAP | 20H4_H3_4 |
| | GKGLELVSRIRTKPNNYATYHADSVKGRFTISRDNSKNTLYL | |
| | QMNSLRAEDTAVYYCSADFAKGDYWGQGTLVTVSS | |
| 164 | EVQLVESGGGLVQPGGSLRLSCAASGFTFSNAAMYWVRQAP | 20H4_H3_5 |
| | GKGLELVSRIRTKPNNYATYHADSVKGRFTISRDNSKNTLYL | |
| | QMNSLRAEDTAVYYCTADFARGDYWGQGTLVTVSS | |
| 165 | EVQLVESGGGLVQPGGSLRLSCAASGFTFSNAAMYWVRQAP | 20H4_H3_6 |
| | GKGLELVSRIRTKPNNYATYHADSVKGRFTISRDNSKNTLYL | |
| | QMNSLRAEDTAVYYCTANMRKGDYWGQGTLVTVSS | |
| 166 | EVQLVESGGGLVQPGGSLRLSCAASGFTFSNAAMYWVRQAP | 20H4_H3_7 |
| | GKGLELVSRIRTKPNNYATYHADSVKGRFTISRDNSKNTLYL | |
| | QMNSLRAEDTAVYYCTADMAKGDYWGQGTLVTVSS | |
| 167 | DIQMTQSPSSLSASVGDRVTITCKASQNINKNLDWYQQKPGK | 20H4_LC1 |
| | APKLLIYSTNNLQTGVPSRFSGSGSGTDFTLTISSLQPEDFATY | |
| | YCYQYKSAWTFGQGTKVEIK | |
| 168 | DIQMTQSPSSLSASVGDRVTITCKASQNINKNLDWYQQKPGK | 20H4_LC2 |
| | APKPLIYSTNNLQTGVPSRFSGSGSGTDFTLTISSLQPEDFATY | |
| | YCYQYKSAWTFGQGTKVEIK | |
| 169 | DIQMTQSPSSLSASVGDRVTITCKASQNINKNLDWYQQKPGK | 20H4_LC3 |
| | APKPLIYSTNNLQTGVPSRFSGSGSGTDYTLTISSLQPEDFATY | |
| | YCYQYKSAWTFGQGTKVEIK | |
| 170 | EIVMTQSPATLSLSPGERATLSCKASQNINKNLDWYQQKPGQ | 20H4_LC4 |
| | APRLLIYSTNNLQTGIPARFSGSGSGTDFTLTISSLQPEDFAVY | |
| | YCYQYKSAWTFGQGTKVEIK | |
| 171 | EIVMTQSPATLSLSPGERATLSCKASQNINKNLDWYQQKPGQ | 20H4_LC5 |
| | APRPLIYSTNNLQTGIPARFSGSGSGTDFTLTISSLQPEDFAVY | |
| | YCYQYKSAWTFGQGTKVEIK | |
| 172 | EIVMTQSPATLSLSPGERATLSCKASQNINKNLDWYQQKPGQ | 20H4_LC6 |
| | APRPLIYSTNNLQTGVPARFSGSGSGTDYTLTISSLQPEDFAV | |
| | YYCYQYKSAWTFGQGTKVEIK | |
| 173 | DIQMTQSPSSLSASVGDRVTITCKASQNINNNLDWYQQKPGK | 2OH4_L1_1 |
| | APKPLIYSTNNLKTGVPSRFSGSGSGTDFTLTISSLQPEDFATY | |
| | YCYQYKSAWTFGQGTKVEIK | |
| 174 | DIQMTQSPSSLSASVGDRVTITCKASQIINQILDWYQQKPGKA | 20H4_L1_2 |
| | PKPLIYSTNNLQTGVPSRFSGSGSGTDFTLTISSLQPEDFATYY | |
| | CYQYKSAWTFGQGTKVEIK | |
| 175 | DIQMTQSPSSLSASVGDRVTITCKASQNINKNLDWYQQKPGK | 20H4_L2_1 |
| | APKPLIYSTNKLKTGVPSRFSGSGSGTDFTLTISSLQPEDFATY | |
| | YCYQYKSAWTFGQGTKVEIK | |
| 176 | DIQMTQSPSSLKSSVGDRVTITCKASQNINKNLDWYQQKPGK | 20H4_L2_2 |
| | APKPLIYRTKNLQTGVPSRFSGSGSGTDFTLTISSLQPEDFATY | |
| | YCYQYKSAWTFGQGTKVEIK | |
| 177 | DIQMTQSPSSLSASVGDRVTITCKASQNINKNLDWYQQKPGK | 20H4_L3_1 |
| | APKPLIYSTNNLQTGVPSRFSGSGSGTDFTLTISSLQPEDFATY | |
| | YCYQDKNGWTFGQGTKVEIK | |
| 178 | DIQMTQSPSSLSASVGDRVTITCKASQNINKNLDWYQQKPGK | 20H4_L3_2 |
| | APKPLIYSTNNLQTGVPSRFSGSGSGTDFTLTISSLQPEDFATY | |
| | YCYQDKRGWTFGQGTKVEIK | |
| 179 | DIQMTQSPSSLSASVGDRVTITCKASQNINKNLDWYQQKPGK | 20H4_L3_3 |
| | APKPLIYSTNNLQTGVPSRFSGSGSGTDFTLTISSLQPEDFATY | |
| | YCYQDKKGWTFGQGTKVEIK | |
| 180 | DIQMTQSPSSLSASVGDRVTITCKASQNINKNLDWYQQKPGK | 20H4_L3_4 |
| | APKPLIYSTNNLQTGVPSRFSGSGSGTDFTLTISSLQPEDFATY | |
| | YCYQDKSGWTFGQGTKVEIK | |
| 181 | DIQMTQSPSSLSASVGDRVTITCKASQNINKNLDWYQQKPGK | 20H4_L3_5 |
| | APKPLIYSTNNLQTGVPSRFSGSGSGTDFTLTISSLQPEDFATY | |
| | YCYQYKNAWTFGQGTKVEIK | |
| 182 | DIQMTQSPSSLSASVGDRVTITCKASQNINKNLDWYQQKPGK | 20H4_L3_6 |
| | APKPLIYSTNNLQTGVPSRFSGSGSGTDFTLTISSLQPEDFATY | |
| | YCYQYKKAWTFGQGTKVEIK | |
| 183 | DIQMTQSPSSLSASVGDRVTITCKASQNINKNLDWYQQKPGK | 20H4_L3_7 |
| | APKPLIYSTNNLQTGVPSRFSGSGSGTDFTLTISSLQPEDFATY | |
| | YCYQYKNGWTFGQGTKVEIK | |
| 184 | DIQMTQSPSSLSASVGDRVTITCKASQNINKNLDWYQQKPGK | 20H4_L3_8 |
| | APKPLIYSTNNLQTGVPSRFSGSGSGTDFTLTISSLQPEDFATY | |
| | YCYQFKKAWTFGQGTKVEIK | |
| 185 | DIQMTQSPSSLSASVGDRVTITCKASQNINKNLDWYQQKPGK | 20H4_L3_9 |
| | APKPLIYSTNNLQTGVPSRFSGSGSGTDFTLTISSLQPEDFATY | |
| | YCYQYKYAWTFGQGTKVEIK | |
| 186 | DIQMTQSPSSLSASVGDRVTITCKASQNINKNLDWYQQKPGK | 20H4_L3_10 |
| | APKPLIYSTNNLQTGVPSRFSGSGSGTDFTLTISSLQPEDFATY | |
| | YCYQYKLAWTFGQGTKVEIK | |
| 187 | DIQMTQSPSSLSASVGDRVTITCKASQNINKNLDWYQQKPGK | 20H4_L3_11 |
| | APKPLIYSTNNLQTGVPSRFSGSGSGTDFTLTISSLQPEDFATY | |
| | YCYQYKFAWTFGQGTKVEIK | |
| 188 | RIRTKPNNYATYYADSVRG | CDR-H2 for clone 22D11 |
| 189 | [reserved] | |
| 190 | APELLGGPSVFLFPPKPKDTLMISRTPEVTCVVVDVSHEDPEV | Wild-type human Fc |
| | KFNWYVDGVEVHNAKTKPREEQYNSTYRVVSVLTVLHQD | sequence |
| | WLNGKEYKCKVSNKALPAPIEKTISKAKGQPREPQVYTLPPS | positions 231-447 EU |
| | RDELTKNQVSLTCLVKGFYPSDIAVEWESNGQPENNYKTTPP | index numbering |
| | VLDSDGSFFLYSKLTVDKSRWQQGNVFSCSVMHEALHNHY | |
| | TQKSLSLSPGK | |
| 191 | QVQLVQSGAEVKKPGSSVKVSCKASGGTLSGYGVSWVRQA | Anti-BACE VH |
| | PGQGLEWMGNIIPGFGTANYAQKFQGRVTITADESTSTAYM | |
| | ELSSLRSEDTAVYYCARSGGTKYGMLDVWGQGTMVTVSS | |
| 192 | EIVMTQSPATLSVSPGERATLSCRASQSVSSNLAWYQQKPGQ | Anti-BACE VL |
| | APRLLIYGASTRATGIPARFSGSGSGTEFTLTISSLQSEDFAVY | |
| | YCQQLILWPPRTFGGGTKVEIK | |
| 193 | [reserved] | |
| 194 | ASTKGPSVFPLAPSSKSTSGGTAALGCLVKDYFPEPVTVSWN | 20H4_HC5 |
| | SGALTSGVHTFPAVLQSSGLYSLSSVVTVPSSSLGTQTYICNV | Heavy chain constant |
| | NHKPSNTKVDKKVEPKSCDKTHTCPPCP | domain 1 (CH1) |
| 195 | RTVAAPSVFIFPPSDEQLKSGTASVVCLLNNFYPREAKVQWK | 20H4_L11 |
| | VDNALQSGNSQESVTEQDSKDSTYSLSSTLTLSKADYEKHK | light chain constant |
| | VYACEVTHQGLSSPVTKSFNRGEC | domain (CL) |
| 196 | EIVMTQSPATLSLSPGERATLSCKASQNINNNLDWYQQKPGQ | 20H4_LC_k3_1 light |
| | APRPLIYSTNNLKTGIPARFSGSGSGTDFTLTISSLQPEDFAVY | chain |
| | YCYQYKSAWTFGQGTKVEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| 197 | EIVMTQSPATLSLSPGERATLSCKASQNINKNLDWYQQKPGQ | 20H4_LC_k3_2_v2 light |
| | APRPLIYSTNNLQTGIPARFSGSGSGTDFTLTISSLQPEDFAVY | chain |
| | YCYQYKSAWTFGQGTKVEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| 198 | DIQMTQSPSSLSASVGDRVTITCKASQNINNNLDWYQQKPGK | 20H4_L1_1 |
| | APKPLIYSTNNLKTGVPSRFSGSGSGTDFTLTISSLQPEDFATY | light chain |
| | YCYQYKSAWTFGQGTKVEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| 199 | EIVMTQSPATLSLSPGERATLSCKASQNINKNLDWYQQKPGQ | 20H4_LC_k3_2_v1 light |
| | APRPLIYSTNNLKTGIPARFSGSGSGTDFTLTISSLQPEDFAVY | chain |
| | YCYQYKSAWTFGQGTKVEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| 200 | EIVMTQSPATLSLSPGERATLSCKASQNINKNLDWYQQKPGQ | 20H4_LC_k3_2_v2 VL |
| | APRPLIYSTNNLQTGIPARFSGSGSGTDFTLTISSLQPEDFAVY | |
| | YCYQYKSAWTFGQGTKVEIK | |
| 201 | EIVMTQSPATLSLSPGERATLSCKASQNINNNLDWYQQKPGQ | 20H4_LC_k3_1 VL |
| | APRPLIYSTNNLKTGIPARFSGSGSGTDFTLTISSLQPEDFAVY | |
| | YCYQYKSAWTFGQGTKVEIK | |
| 202 | EIVMTQSPATLSLSPGERATLSCKASQNINKNLDWYQQKPGQ | 20H4_LC_k3_2_v1 VL |
| | APRPLIYSTNNLKTGIPARFSGSGSGTDFTLTISSLQPEDFAVY | |
| | YCYQYKSAWTFGQGTKVEIK | |
| 203 | EIVMTQSPATLSLSPGERATLSCKASQNINQNLDWYQQKPGQ | 20H4_LC_k3_3 VL |
| | APRPLIYSTNNLKTGIPARFSGSGSGTDFTLTISSLQPEDFAVY | |
| | YCYQYKSAWTFGQGTKVEIK | |
| 204 | EIVMTQSPATLSLSPGERATLSCKASQNINENLDWYQQKPGQ | 20H4_LC_k3_4 VL |
| | APRPLIYSTNNLKTGIPARFSGSGSGTDFTLTISSLQPEDFAVY | |
| | YCYQYKSAWTFGQGTKVEIK | |
| 205 | EIVMTQSPATLSLSPGERATLSCKASQNINKQLDWYQQKPGQ | 20H4_LC_k3_5 VL |
| | APRPLIYSTNNLKTGIPARFSGSGSGTDFTLTISSLQPEDFAVY | |
| | YCYQYKSAWTFGQGTKVEIK | |
| 206 | EIVMTQSPATLSLSPGERATLSCKASANINKQLDWYQQKPGQ | 20H4_LC_k3_6 VL |
| | APRPLIYSTNNLKTGIPARFSGSGSGTDFTLTISSLQPEDFAVY | |
| | YCYQYKSAWTFGQGTKVEIK | |
| 207 | EIVMTQSPATLSLSPGERATLSCKASQNISNYLDWYQQKPGQ | 20H4_LC_k3_7 VL |
| | APRPLIYSTNNLKTGIPARFSGSGSGTDFTLTISSLQPEDFAVY | |
| | YCYQYKSAWTFGQGTKVEIK | |
| 208 | EIVMTQSPATLSLSPGERATLSCKASQGISNYLDWYQQKPGQ | 20H4_LC_k3_8 VL |
| | APRPLIYSTNNLKTGIPARFSGSGSGTDFTLTISSLQPEDFAVY | |
| | YCYQYKSAWTFGQGTKVEIK | |
| 209 | EIVMTQSPATLSLSPGERATLSCKASQGISNYLAWYQQKPGQ | 20H4_LC_k3_9 VL |
| | APRPLIYSTNNLKTGIPARFSGSGSGTDFTLTISSLQPEDFAVY | |
| | YCYQYKSAWTFGQGTKVEIK | |
| 210 | EIVMTQSPATLSLSPGERATLSCKASQNISNDLDWYQQKPGQ | 20H4_LC_k3_10 VL |
| | APRPLIYSTNNLKTGIPARFSGSGSGTDFTLTISSLQPEDFAVY | |
| | YCYQYKSAWTFGQGTKVEIK | |
| 211 | EIVMTQSPATLSLSPGERATLSCKASQNISNQLDWYQQKPGQ | 20H4_LC_k3_11 VL |
| | APRPLIYSTNNLKTGIPARFSGSGSGTDFTLTISSLQPEDFAVY | |
| | YCYQYKSAWTFGQGTKVEIK | |
| 212 | EIVMTQSPATLSLSPGERATLSCKASQNINDNLDWYQQKPGQ | 20H4_LC_k3_12 VL |
| | APRPLIYSTNNLKTGIPARFSGSGSGTDFTLTISSLQPEDFAVY | |
| | YCYQYKSAWTFGQGTKVEIK | |
| 213 | EIVMTQSPATLSLSPGERATLSCKASQNINANLDWYQQKPGQ | 20H4_LC_k3_13 VL |
| | APRPLIYSTNNLKTGIPARFSGSGSGTDFTLTISSLQPEDFAVY | |
| | YCYQYKSAWTFGQGTKVEIK | |
| 214 | KAxxxxxxxLx | 20H4 CDR-L1 consensus |
| | | sequence |
| 215 | KAX₁X₂X₃X₄X₅X₆X₇LX₈, wherein X₁ is S or G; X₂ is Q or | 20H4 CDR-L1 consensus |
| | A; X₃ is N, I, or G; X₄ is I or N; X₅ is N or S; X₆ | sequence |
| | is A, D, N, K, or Q; X₇ is N, I, Q, or Y; and X₈ is | |
| | D or A | |
| 216 | KASX₁NNNX₂X₃LD, wherein X₁ is Q or A; X₂ is W, E, | 20H4 CDR-L1 consensus |
| | or K; and X₃ is N or Q | sequence |
| 217 | KASX₁NINX₂X₃LD, wherein X₁ is Q or A; X₂ is W, E, or | 20H4 CDR-L1 consensus |
| | K; and X₃ is N or Q | sequence |
| 218 | KASQX₁ISNX₂LX₃, wherein X₁ is N or G; X₂ is Y, R, or | 20H4 CDR-L1 consensus |
| | Q; and X₃ is A or D | sequence |
| 219 | EVQLVESGGGLVQPGGSLRLSCAASGFTFSGAAMYWVRQAP | 20H4_HC6.1 heavy chain |
| | GKGLELVSRIRTKPNAYATYHAASVKGRFTISRDDSKNTLYL | |
| | QMNSLRAEDTAVYYCTADMATGDYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSRDELTKNQVSLTCLVKG | |
| | FYPSDIAVEWESNGQPENNYKTTPPVLDSDGSFFLYSKLTVD | |
| | KSRWQQGNVFSCSVLHEALHSHYTQKSLSLSPGK | |
| 220 | EVQLVESGGGLVQPGGSLRLSCAASGFTFSGAAMYWVRQAP | 20H4_HC6.2 |
| | GKGLELVSRIRSKANAYATAHAASVKGRFTISRDDSKNTLYL | heavy chain |
| | QMNSLRAEDTAVYYCTADMATGDYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSRDELTKNQVSLTCLVKG | |
| | FYPSDIAVEWESNGQPENNYKTTPPVLDSDGSFFLYSKLTVD | |
| | KSRWQQGNVFSCSVLHEALHSHYTQKSLSLSPGK | |
| 221 | EIVMTQSPATLSLSPGERATLSCRASQNINKNLDWYQQKPGQ | h20H4_LC_k3_2.1 |
| | APRPLIYDASNLKTGIPARFSGSGSGTDFTLTISSLQPEDFAVY | light chain |
| | YCYQYKSAWTFGQGTKVEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| 222 | EVQLVESGGGLVQPGGSLRLSCAASGFTFSGAAMYWVRQAP | 20H4_HC6.1_VH |
| | GKGLELVSRIRTKPNAYATYHAASVKGRFTISRDDSKNTLYL | |
| | QMNSLRAEDTAVYYCTADMATGDYWGQGTLVTVSS | |
| 223 | EVQLVESGGGLVQPGGSLRLSCAASGFTFSGAAMYWVRQAP | 20H4_HC6.2_VH |
| | GKGLELVSRIRSKANAYATAHAASVKGRFTISRDDSKNTLYL | |
| | QMNSLRAEDTAVYYCTADMATGDYWGQGTLVTVSS | |
| 224 | EIVMTQSPATLSLSPGERATLSCRASQNINKNLDWYQQKPGQ | h20H4_LC_k3_2.1_VL |
| | APRPLIYDASNLKTGIPARFSGSGSGTDFTLTISSLQPEDFAVY | |
| | YCYQYKSAWTFGQGTKVEIK | |
| 225 | [reserved] | |
| 226 | EVQLVESGGGLVQPGGSLRLSCAASGFTFSNAAMYWVRQAP | 20H4_HC5_LS |
| | GKGLELLARIRTKPNNYATYHADSVKGRFTISRDDSKNTVYL | heavy chain |
| | QMNSLRAEDTAVYYCTADMATGDYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSRDELTKNQVSLTCLVKG | |
| | FYPSDIAVEWESNGQPENNYKTTPPVLDSDGSFFLYSKLTVD | |
| | KSRWQQGNVFSCSVLHEALHSHYTQKSLSLSPGK | |
| 227 | EVQLVESGGGLVQPGGSLRLSCAASGFTFSNAAMYWVRQAP | 20H4_HC5 |
| | GKGLELLARIRTKPNNYATYHADSVKGRFTISRDDSKNTVYL | heavy chain |
| | QMNSLRAEDTAVYYCTADMATGDYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSRDELTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPGK | |
| 228 | EVQLVESGGGLVQPGGSLRLSCAASGFTFSNAAMYWVRQAP | h20H4_HC6 |
| | GKGLELVSRIRTKPNNYATYHADSVKGRFTISRDDSKNTLYL | heavy chain |
| | QMNSLRAEDTAVYYCTADMATGDYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCDKTHTCPPCPAPEAAGGPSVFLFPPKPK | |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSRDELTKNQVSLTCLVKG | |
| | FYPSDIAVEWESNGQPENNYKTTPPVLDSDGSFFLYSKLTVD | |
| | KSRWQQGNVFSCSVMHEALHNHYTQKSLSLSPGK | |
| 229 | EVQLVESGGGLVQPGGSLRLSCAASGFTFSNAAMYWVRQAP | h20H4_HC6_VH |
| | GKGLELVSRIRTKPNNYATYHADSVKGRFTISRDDSKNTLYL | |
| | QMNSLRAEDTAVYYCTADMATGDYWGQGTLVTVSS | |
| 230 | EVKLVESGGGLVKPGASLKLSCAASGFTFSNYGMSWVRQTS | 9E4 VH |
| | DKRLEWVASISSGGGSTYYPDNVKGRFTISREDAKNTLYLQ | |
| | MSSLRSEDTALYYCSRGGAGIDYWGQGTTLTVSS | |
| 231 | DIVMSQSPSSLAVSVGEKVTMSCKSIQTLLYSSNQKNYLAWF | 9E4 VL |
| | QQKPGQSPKLLIYWASIRKSGVPDRFTGSGSGTDFTLTISSVK | |
| | AEDLAVYYCQQYYSYPLTFGAGTKLELK | |
| 232 | EVQLLESGGGLVQPGGSLRLSCAASGFTFSSYAMSWVRQAP | aslo452 ngl3 VH |
| | GKGLEWVSSISHLGGSTYYADSVKGRFTISRDNSKNTLYLQ | |
| | MNSLRAEDTAVYYCAGGANHGKYYYGMDKWGQGTTVTV | |
| | SS | |
| 233 | QAVLTQPASLSASPGASASLTCTLRSGAPLPKYRIYWYQQKP | aslo452 ngl3 VL |
| | GSPPQYLLRYKSDADKHQGSGVPSRFSGSKDASANAGILLIS | |
| | GLQSEDEADYYCMVWDHGVWYFGGGTKLTVL | |

1.-113. (canceled)

114. An isolated antibody, or antigen binding portion thereof, that specifically binds human alpha-synuclein protein, wherein the antibody or antigen-binding portion thereof recognizes an epitope comprised within residues 119-125 of SEQ ID NO.:1, and wherein:
(a) alanine mutagenesis at any one or more of residues 119, 122, 124, and 125 of SEQ ID NO.:1, and/or proline mutagenesis at residue 124 of SEQ ID NO.:1, reduces or abrogates binding by the antibody or antigen-binding portion thereof to the human alpha-synuclein protein;
(b) glycine mutagenesis at residue 124 of SEQ ID NO.:1 does not reduce or abrogate binding by the antibody or antigen-binding portion thereof to the human alpha-synuclein protein; and/or
(c) alanine mutagenesis at any of residues 120, 121, and 123 of SEQ ID NO.:1 does not reduce or abrogate binding by the antibody or antigen-binding portion thereof to the human alpha-synuclein protein.

115. The antibody or antigen-binding portion of claim 114, wherein reduction or abrogation in binding by the antibody or antigen-binding portion thereof to the human alpha-synuclein protein, or lack thereof, is determined by ELISA, with an absorbance signal quantified at 450 nM.

116. The antibody or antigen-binding portion of claim 114, wherein the antibody or antigen-binding portion thereof specifically binds to each of monomeric human alpha-synuclein protein, oligomeric human alpha-synuclein protein, and fibrillar human alpha-synuclein protein with K_{D} of about 500 pM or less, as measured by surface plasmon resonance (SPR).

117. The antibody or antigen-binding portion of claim 116, wherein the antibody or antigen-binding portion thereof specifically binds to each of monomeric human alpha-synuclein protein, oligomeric human alpha-synuclein protein, and fibrillar human alpha-synuclein protein with K_{D} of about 300 pM or less.

118. The antibody or antigen-binding portion of claim 114, comprising:
(a) a heavy chain CDR1 comprising a sequence of the consensus sequence of SEQ ID NO.:103;
(b) a heavy chain CDR2 comprising a sequence of the consensus sequence of SEQ ID NO.:111; and
(c) a heavy chain CDR3 comprising a sequence of the consensus sequence of SEQ ID NO.:120; and/or
(d) a light chain CDR1 comprising a sequence of the consensus sequence of SEQ ID NO.:47 or 214;
(e) a light chain CDR2 comprising a sequence of the consensus sequence of SEQ ID NO.:49; and
(f) a light chain CDR3 comprising a sequence of the consensus sequence of SEQ ID NO.:51.

119. The antibody or antigen-binding portion of claim 114, comprising:
(a) a heavy chain CDR1 comprising a sequence of the consensus sequence of SEQ ID NO.:104,
(b) a heavy chain CDR2 comprising a sequence of the consensus sequence of SEQ ID NO.:112, and
(c) a heavy chain CDR3 comprising a sequence of the consensus sequence of SEQ ID NO.:121; and/or
(d) a light chain CDR1 comprising a sequence of the consensus sequence of SEQ ID NO.:215,
(e) a light chain CDR2 comprising a sequence of the consensus sequence of SEQ ID NO.:93, and
(f) a light chain CDR3 comprising a sequence of the consensus sequence of SEQ ID NO.:95.

120. The antibody or antigen-binding portion of claim 114, comprising:
(a) a light chain CDR1 comprising the amino acid sequence of SEQ ID NO.:24 or 122, or a light chain CDR1 according to the light chain variable region amino acid sequence of any one of SEQ ID NOs.: 204-213;
(b) a light chain CDR2 comprising the amino acid sequence of SEQ ID NO.:128 or 28; and
(c) a light chain CDR3 comprising the amino acid sequence of SEQ ID NO.:32.

121. The antibody or antigen-binding portion of claim 114, comprising:
(i) a heavy chain variable region comprising an amino acid sequence having at least 90% identity to the amino acid sequence set forth in any one of SEQ ID NOs.:229, 222, 223, and 147; and/or
(ii) a light chain variable region variable region comprising an amino acid sequence having at least 90% identity to the amino acid sequence set forth in any one of SEQ ID NOs.:202, 173, 201, 224, and 200.

122. An isolated antibody or antigen-binding portion thereof that specifically binds to a human alpha-synuclein protein, the antibody or antigen-binding portion thereof comprising:
(a) a heavy chain CDR1 comprising the amino acid sequence of SEQ ID NO:3;
(b) a heavy chain CDR2 comprising the amino acid sequence of SEQ ID NO:9;
(c) a heavy chain CDR3 comprising the amino acid sequence of SEQ ID NO:12;
(d) a light chain CDR1 comprising the amino acid sequence of SEQ ID NO.:24 or 122, or a light chain CDR1 according to the light chain variable region amino acid sequence of any one of SEQ ID NOs.: 204-213;
(e) a light chain CDR2 comprising the amino acid sequence of SEQ ID NO.:128 or 28; and
(f) a light chain CDR3 comprising the amino acid sequence of SEQ ID NO.:32.

123. The antibody or antigen-binding portion of claim 122, comprising:
(i) a heavy chain variable region comprising an amino acid sequence having at least 90% identity to the amino acid sequence set forth in any one of SEQ ID NOs.:229, 222, 223, and 147; and/or
(ii) a light chain variable region variable region comprising an amino acid sequence having at least 90% identity to the amino acid sequence set forth in any one of SEQ ID NOs.:202, 173, 201, 224, and 200.

124. The isolated antibody or antigen-binding portion of claim 122, comprising:
(i) a heavy chain variable region comprising or consisting of the amino acid sequence set forth in any one of SEQ ID NOs.:229, 147, 222, and 223; and
(ii) a light chain variable region comprising or consisting of the amino acid sequence set forth in any one of SEQ ID NOs.:202, 173, 200, 201, 202, and 224.

125. The antibody or antigen-binding portion of claim 124, comprising:
(i) a heavy chain variable region comprising or consisting of the amino acid sequence set forth in SEQ ID NO.:229; and
(ii) a light chain variable region comprising or consisting of the amino acid sequence set forth in any one of SEQ ID NOs.:202, 173, 200, 201, 202, and 224.

126. The antibody or antigen-binding portion of claim 124, comprising:
(i) a heavy chain variable region comprising or consisting of the amino acid sequence set forth in any one of SEQ ID NOs.:229, 147, 222, and 223; and
(ii) a light chain variable region comprising or consisting of the amino acid sequence set forth in SEQ ID NO.:202.

127. The antibody or antigen-binding portion of claim 114, comprising a first Fc polypeptide and a second Fc polypeptide, wherein:
(1) the first Fc polypeptide and/or the second Fc polypeptide comprises an Ala at position 234 and an Ala at position 235; and/or
(2) the first Fc polypeptide and/or the second Fc polypeptide comprises a Leu at position 428 and a Ser at position 434.

128. The antibody or antigen-binding portion of claim 114, comprising a first Fc polypeptide and a second Fc polypeptide, wherein the first Fc polypeptide and/or the second Fc polypeptide is or is derived from a human IgG1, IgG2, IgG3, or IgG4 isotype.

129. The antibody or antigen-binding portion of claim 114, wherein the antigen-binding portion is a Fab, a F(ab′)2, a scFv, or a bivalent scFv.

130. The isolated antibody or antigen-binding portion thereof of claim 122, comprising:
(i) a heavy chain comprising or consisting of the amino acid sequence set forth in any one of SEQ ID NOs.:228, 226, and 227; and
(ii) a light chain comprising or consisting of the amino acid sequence set forth in any one of SEQ ID NOs.:199, 198, 197, and 196.

131. The antibody or antigen-binding portion of claim 130, comprising:
(i) a heavy chain comprising or consisting of the amino acid sequence set forth in SEQ ID NO.:228; and
(ii) a light chain comprising or consisting of the amino acid sequence set forth in any one of SEQ ID NOs.:199, 198, 197, and 196.

132. The antibody or antigen-binding portion of claim 130:
(i) a heavy chain comprising or consisting of the amino acid sequence set forth in any one of SEQ ID NOs.:228, 226, and 227; and
(ii) a light chain comprising or consisting of the amino acid sequence set forth in SEQ ID NO.:199.

133. The antibody or antigen-binding portion of claim 130, comprising:
(i) a first heavy chain (HC) that comprises or consists of the amino acid sequence set forth in any one of SEQ ID NOs.:228, 226, and 227;
(ii) a second HC that comprises or consists of the amino acid sequence set forth in any one of SEQ ID NOs.:228, 226, and 227; and
(iii) a first and a second light chain (LC) that each comprise or consist of the amino acid sequence set forth in any one of SEQ ID NOs.:199, 198, 197, and 196.

134. The antibody or antigen-binding portion of claim 114: (1) which is a pan-alpha-synuclein antibody or antigen-binding portion; (ii) wherein the antibody or antigen-binding portion selectively binds to human alpha-synuclein over human beta-synuclein and human gamma-synuclein; (iii) wherein the antibody or antigen-binding portion binds to alpha-synuclein with a K_{D} of about 230 pM or less, as measured by BiaCore™ SPR; (iv) wherein the antibody or antigen-binding portion binds to alpha-synuclein monomers with a K_{D} of less than about 20 pM, as measured by KinExA® kinetic exclusion assay; (v) wherein the antibody or antigen-binding portion binds to sonicated alpha-synuclein fibrils with a K_{D} of less than about 5 pM, as measured by KinExA® kinetic exclusion assay; (vi) wherein the antibody or antigen-binding portion binds to alpha-synuclein oligomers with a K_{D} of less than about 10 pM, as measured by KinExA® kinetic exclusion assay; (vii) wherein the antibody or antigen-binding portion specifically binds to or immunodepletes at least about 50% of insoluble human alpha-synuclein protein and at least 50% soluble human alpha-synuclein protein from a sample; (viii) wherein the antibody or antigen-binding portion specifically binds to or immunodepletes at least about 70% of insoluble human alpha-synuclein protein from a sample; (ix) wherein the antibody or antigen-binding portion reduces the amount of unbound soluble alpha-synuclein present in a sample by at least about 60%, wherein the sample comprises cerebrospinal fluid (CSF) obtained from a human subject having or suspected of having Parkinson's Disease (PD); (x) wherein co-incubation of the antibody or antigen-binding portion with a PD-C SF sample results in at least about a one-tenth-fold change in the concentration of unbound soluble alpha-synuclein present in the PD-CSF sample; (xi) wherein the antibody or antigen-binding portion prevents, reduces, or inhibits alpha-synuclein seeding by at least about 10% as compared to the amount of alpha-synuclein seeding in the absence of the anti-alpha-synuclein antibody; (xii) wherein the antibody or antigen-binding portion reduces the amount of pSer129 alpha-synuclein present in a brain of a subject having an alpha-synucleinopathy; (xiii) wherein the antibody or antigen-binding portion reduces the amount of free alpha-synuclein present in brain interstitial fluid (ISF) of a subject having an alpha-synucleinopathy by about 10% or more; (xiv) wherein the antibody or antigen-binding portion has a CL value of less than 25 ml/day/kg in a subject; (xv) wherein the antibody or antigen-binding portion, when administered intravenously in a single dose of 3 mg/kg of the antibody or antigen-binding portion to a primate, has a T_{1/2} of at least about 5.2 days; (xvi) wherein the antibody or antigen-binding portion, when administered intravenously in a single dose of 10 mg/kg of the antibody or antigen-binding portion to a primate, has a CL value of about 5.2 ml/day/kg; (xvii) wherein the antibody or antigen-binding portion, when administered intravenously to a primate in a single dose of 30 mg/kg of the antibody or antigen-binding portion, has a CL value of about 4.5 ml/day/kg; (xviii) wherein the antibody or antigen-binding portion is capable, when administered intravenously to a primate in a single dose of about 30 mg/kg, of reducing free alpha-synuclein levels in CSF by about 25%; (xix) wherein the antibody or antigen-binding portion is capable, when administered intravenously to a primate in a single dose of about 10 mg/kg, of reducing free alpha-synuclein levels in CSF by at least about 25% for at least 1 day following administration of the antibody or antigen-binding portion; and/or (xx) wherein the antibody or antigen-binding portion is capable, when administered intravenously to a primate in a single dose of about 10 mg/kg or about 30 mg/kg, of reducing free alpha-synuclein levels in CSF by at least about 60% for at least 1 day following administration of the antibody or antigen-binding portion.

135. A composition comprising the antibody or antigen-binding portion of claim 114 and a pharmaceutically acceptable carrier.

136. An isolated polynucleotide that encodes the antibody or antigen-binding portion of claim 114.

137. A host cell, comprising the polynucleotide of claim 136.

138. A kit, comprising the antibody or antigen-binding portion of claim 114 and instructions for using the antibody or antigen-binding portion to treat a neurodegenerative disease.

139. A method for neutralizing human alpha-synuclein protein in a brain of a subject, the method comprising administering to the subject an effective amount of the antibody or antigen-binding portion of claim 114.

140. The method of claim 139, wherein the neurodegenerative disease is a synucleinopathy.

141. The method of claim 140, wherein the synucleinopathy is selected from the group consisting of Parkinson's disease, dementia with Lewy bodies, and multiple system atrophy.

142. A method for treating a neurodegenerative disease in a subject, the method comprising administering to the subject an effective amount of the antibody or antigen-binding portion of claim 114.

143. The method of claim 142, wherein the neurodegenerative disease is selected from the group consisting of Parkinson's disease, dementia with Lewy bodies, multiple system atrophy, Huntington's disease, Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis/parkinsonism-dementia complex of Guam, corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, inclusion-body myositis, myotonic dystrophy, neurofibrillary tangle-predominant dementia, Niemann-Pick disease type C, pallido-ponto-nigral degeneration, Pick's disease, postencephalitic parkinsonism, prion protein cerebral amyloid angiopathy, progressive subcortical gliosis, subacute sclerosing panencephalitis, and tangle only dementia.
